(19) United States
(12) Patent Application Publication
(10) Pub. No.: US 2006/0116400 A1
(43) Pub. Date: Jun. 1, 2006

(54) Oxazolidinone and/or isoxazoline derivatives as antibacterial agents

(75) Inventors: Daniel Robert Carcanague, Waltham, MA (US); Michael Barry Gravestock, Waltham, MA (US); Sheila Irene Hauck, Waltham, MA (US); Thomas Peter Weber, Waltham, MA (US); Neil James Hales, Macclesfield (GB)

Correspondence Address: ASTRAZENECA R&D BOSTON, 35 GATEHOUSE DRIVE, WALTHAM, MA, 02451-1215, US

(73) Assignee: AstraZeneca AB, Sodertaije (SE)

(21) Appl. No.: 10/536,686

(22) PCT Filed: Nov. 24, 2003
(86) PCT No.: PCT/GB03/05087

§ 371 Date: May 27, 2005

(30) Foreign Application Priority Data
Nov. 28, 2002 (GB) 0227702.8
Mar. 1, 2003 (GB) 0304725.5
Aug. 8, 2003 (GB) 0318608.7

(51) Int. Cl. A61K 31/444; A61K 31/4439; A61K 31/422; A61K 31/42; C07D 413/14; C07D 413/02

(52) U.S. Cl. 514/332; 514/376; 514/378; 546/256; 546/271.4; 546/272.1; 548/229; 548/240

(57) ABSTRACT

A compound of the formula (I), or a pharmaceutically-acceptable salt, or in-vivo hydrolysable ester thereof formula (I) wherein in (I) C is for example formula (D), formula (E), formula (H) wherein A and B are independently selected from (i) formula (J) and (ii) formula (K) m is 1 or 2; R₂b and R₆b, R₂a and R₆a, R₃a and R₅a, are for example selected from H, F, OMe and Me; R₂b′ and R₆b′, R₂a′ and R₆a′, R₃a′, R₅a′ are for example selected from H, OMe and Me; R₁a is for example optionally substituted (1-10C)alkyl; R₁b is for example selected from —NR₅C(═W)R₄, formula (a), or formula (b) wherein HET-1 is for example isoxazolyl and HET-2 is for example triazolyl or tetrazolyl. Methods for making compounds of the formula (I), compositions containing them and their use as antibacterial agents are also described.

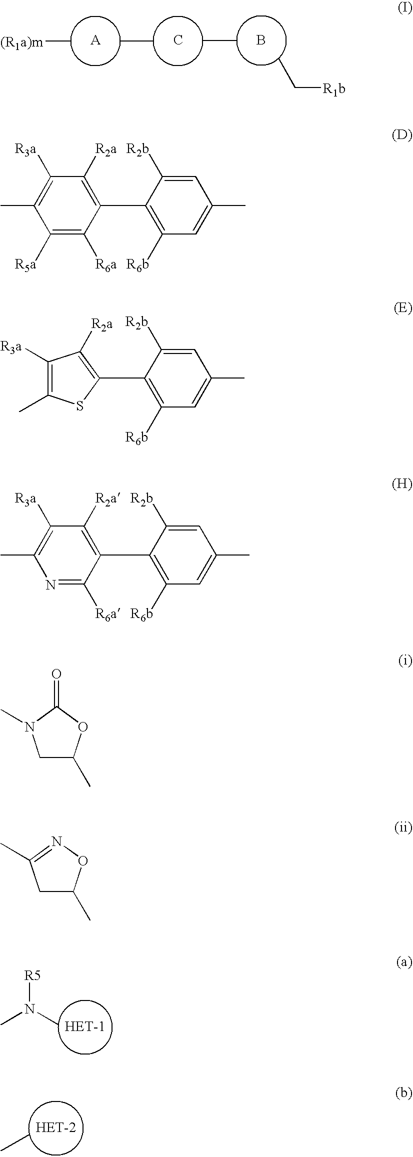

The present invention relates to antibiotic compounds and in particular to antibiotic compounds containing substituted oxazolidinone and/or isoxazoline rings. This invention further relates to processes for their preparation, to intermediates useful in their preparation, to their use as therapeutic agents and to pharmaceutical compositions containing them.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus (MRSA), methicillin resistant coagulase negative staphylococci (RCNS), penicillin resistant Streptococcus pneumoniae and multiply resistant Enterococcus faecium.

The major clinically effective antibiotic for treatment of such resistant Gram-positive pathogens is vancomycin. Vancomycin is a glycopeptide and is associated with various toxicities including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Certain antibacterial compounds containing an oxazolidinone ring have been described in the art (for example, Walter A Gregory et al in J. Med. Chem. 1990, 33, 2569-2578 and 1989, 32(8), 1673-81; Chung-Ho Park et al in J. Med. Chem. 1992, 35, 1156-1165). Bacterial resistance to known antibacterial agents may develop, for example, by (i) the evolution of active binding sites in the bacteria rendering a previously active pharmacophore less effective or redundant, and/or (ii) the evolution of means to chemically deactivate a given pharmacophore, and/or (iii) the evolution of efflux pathways. Therefore, there remains an ongoing need to find new antibacterial agents with a favourable pharmacological profile, in particular for compounds containing new, more potent, pharmacophores.

We have discovered a class of bi-aryl antibiotic compounds containing two substituted oxazolidinone and/or isoxazoline rings which has useful activity against Gram-positive pathogens including MRSA and MRCNS and, in particular, against various strains exhibiting resistance to vancomycin and/or linezolid and against E. faecium strains resistant to both aminoglycosides and clinically used β-lactams, but also to fastidious Gram negative strains such as H. influenzae, M. catarrhalis, mycoplasma spp. and chlamydial strains. The compounds of the invention contain two groups capable of acting as pharmacophores. The two groups may independently bind at pharmacophore binding sites where the sites may be similar or different, where the similar or different sites may be occupied simultaneously or not simultaneously within a single organism, or where the relative importance of different binding modes to the similar or different sites may vary between two organisms of different genus. Alternatively one of the groups may bind at a pharmacophore binding site whilst the other group fulfills a different role in the mechanism of action.

Accordingly the present invention provides a compound of the formula (I), or a pharmaceutically-acceptable salt, or an in-vivo-hydrolysable ester thereof, 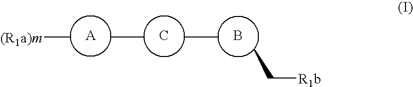
wherein in (I) C is a biaryl group C′-C″ 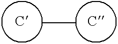
where C′ and C″ are independently aryl or heteroaryl rings such that the group C is represented by any one of the groups D to O below: 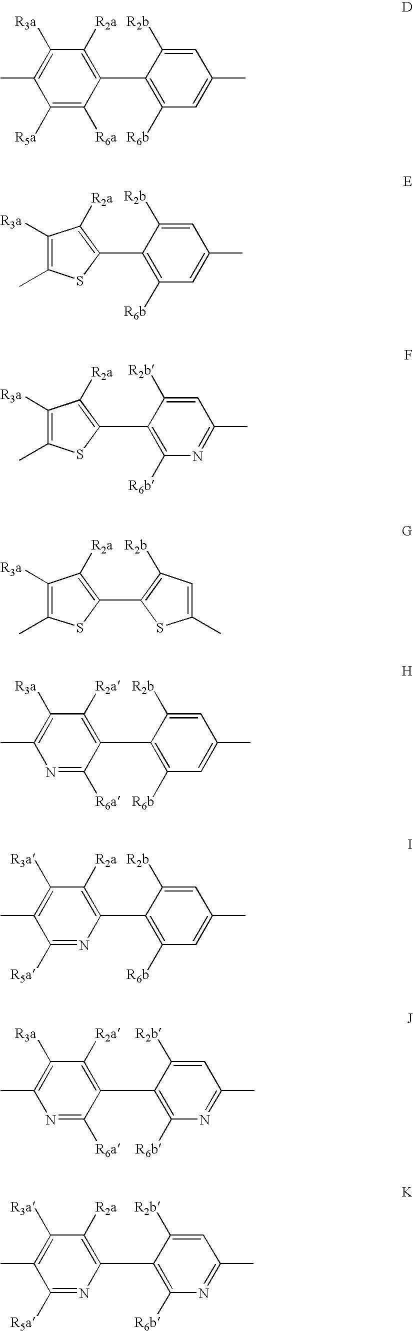 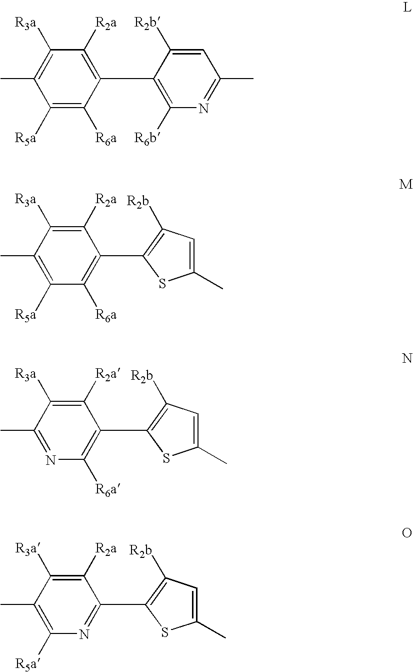
wherein the groups D to O are attached to rings A and B in the orientation [(A-C′) and (C″-B)] shown;
wherein A and B are independently selected from 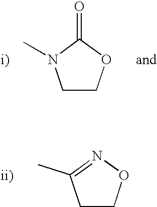
wherein A is linked as shown in (I) via the 3-position to ring C′ of group C and independently substituted in the 4 and 5 positions as shown in (I) by one or more substituents —(R₁a)m; and wherein B is linked as shown in (I) via the 3-position to ring C″ of group C and independently substituted in the 5 position as shown in (I) by substituent —CH₂—R₁b; R₂b and R₆b are independently selected from H, F, Cl, OMe, Me, Et and CF₃, and additionally SMe;
R₂b′ and R₆b′ are independently selected from H, OMe, Me, Et and CF₃;
R₂a and R₆a are independently selected from H, Br; F, Cl, OMe, SMe; Me, Et and CF₃;
R₂a′ and R₆a′ are independently selected from H, OMe, SMe; Me, Et and CF₃;
R₃a and R₅a are independently selected from H, (1-4C)alkyl, Br, P, Cl, OH, (1-4C)alkoxy, —S(O)ₙ(1-4C)alkyl (wherein n=0, 1, or 2), amino, (1-4C)alkylcarbonylamino-, nitro, cyano, —CHO, —CO(1-4C) alkyl, —CONH₂ and —CONH(1-4C)alkyl;
R₃a′, R₅a′ are independently selected from H, (1-4C)alkyl, OH, (1-4C)alkoxy, (1-4C)alkylthio, amino, (1-4C)alkylcarbonylamino-, nitro, cyano, —CHO, —CO(1-4C)alkyl, —CONH₂ and —CONH(1-4C)alkyl;
wherein one of R₃a, R₅a, R₃a′, R₅a taken together with a substituent R₁a at position 4 of ring A and rings A and C′ may form a 5-7 membered ring;
wherein any (1-4C)alkyl group may be optionally substituted with F, OH, (1-4C)alkoxy, —S(O)ₙ(1-4C)alkyl (wherein n=0, 1, or 2) or cyano;
wherein when ring C′ is a pyridine ring (ie when group C is group H, I, J, K, N or O) the ring nitrogen may optionally be oxidised to an N-oxide;
R₁a is independently selected from R₁a1 to R₁a5 below:
R₁a1: AR1, AR2, AR2a, AR2b, AR3, AR3a, AR3b, AR4, AR4a, CY1, CY2;
R₁a2: cyano, carboxy, (1-4C)alkoxycarbonyl, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl) and —C(═S)O(1-4C)alkyl; wherein any (1-4C)alkyl, (1-4C)alkanoyl and (3-6C)cycloalkyl substituent may itself be substituted by cyano, hydroxy or halo, provided that, such a substituent is not on a carbon adjacent to a nitrogen atom of the piperazine ring], ethenyl, 2-(1-4C)alkylethenyl, 2-cyanoethenyl, 2-cyano-2-((1-4C)alkyl)ethenyl, 2-nitroethenyl, 2-nitro-2-((1-4C)alkyl)ethenyl, 2-((1-4C)alkylaminocarbonyl)ethenyl, 2-((1-4C)alkoxycarbonyl)ethenyl, 2-(AR1)ethenyl, 2-(AR2)ethenyl, 2-(AR2a)ethenyl; R₁a3: (1-10C)alkyl
{optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, phosphonate [phosphono, —P(O)(OH)₂, and mono- and di-(1-4C)alkoxy derivatives thereof], phosphinate [—P(OH)₂ and mono- and di-(1-4C)alkoxy derivatives thereof], cyano, halo, trifluoromethyl (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N-(1-4C)alkyl-N-(1-6C)alkanoylamino-, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl], (═NORv) wherein Rv is as hereinbefore defined, (1-4C)alkylS(O)ₚNH—, (1-4C)alkylS(O)ₚ-((1-4C)alkyl)N—, fluoro(1-4C)alkylS(O)ₚNH—, fluoro(1-4C)alkylS(O)ₚ((1-4C)alkyl)N—, (1-4C)alkylS(O)_{q}—, CY1, CY2, AR1, AR2, AR3, AR1-O—, AR2-O—, AR3-O—, AR1-S(O)_{q}—, AR2-S(O)_{q}—, AR³—S(O)_{q}—, AR1-NH—, AR2-NH—, AR3-NH— (p is 1 or 2 and q is 0, 1 or 2), and also AR2a, AR2b, AR3a and AR3b versions of AR2 and AR3 containing groups, and additionally (1-6C)alkanoyloxy(1-4C)alkoxy, carboxy(1-4C)alkoxy, halo(1-4C)alkoxy, dihalo(1-4C)alkoxy, trihalo(1-4C)alkoxy, morpholino-ethoxy, (N′-methyl)piperazino-ethoxy, 2-, 3-, or 4-pyridyl(1-6C)alkoxy, N-methyl(imidazo-2 or 3-yl)(1-4C)alkoxy, imidazo-1-yl(1-6C)alkoxy}; wherein any (1-4C)alkyl, (1-4C)alkanoyl and (3-6C)cycloalkyl present in any substituent on R₁a3 may itself be substituted by one or two groups selected from cyano, hydroxy, halo, amino, (1-4C)alkylamino and di(1-4C)alkylamino, provided that such a substituent is not on a carbon adjacent to a heteroatom atom if present;
R₁a4: R¹⁴C(O)O(1-6C)alkyl [wherein R¹⁴ is AR1, AR2, AR2a, AR2b, (1-4C)alkylamino, or (1-10C)alkyl {optionally substituted as defined for (R₁a3)}, or alternatively R¹⁴ is benzyloxy-(1-4C)alkyl, naphthylmethyl, (1-4C)alkoxy-(1-4C)alkoxy-(1-4C)alkoxy-(1-4C)alkoxy-(1-4C)alkoxy-(1-4C)alkoxy, (1-4C)alkoxy-(1-4C)alkoxy-(1-4C)alkoxy-(1-4)alkoxy-(1-4C)alkoxy, (1-4C)alkoxy-(1-4C)alkoxy-(1-4)alkoxy-(1-4C)alkoxy, (1-4C)alkoxy-(1-4C)alkoxy-(1-4C)alkoxy, (1-4C)alkoxy-(1-4C)alkoxy, imidazo-1-yl(1-6C)alkyoxy(1-4C)alkyl, morpholino-ethoxy(1-4C)alkyl, (N′-methyl)piperazino-ethoxy(1-4C)alkyl, 2-, 3-, or 4-pyridyl(1-6C)alkyloxy(1-4C)alkyl, 2-, 3-, or 4-pyridyl(1-6C)alkylamino(1-4C)alkyl, 2-, 3-, or 4-pyridyl(1-6C)alkylsulfonyl(1-4C)alkyl, or N-methyl(imidazo-2 or 3-yl)(1-4C)alkyloxy(1-4C)alkyl];
R₁a5: F, Cl, hydroxy, mercapto, (1-4C)alkylS(O)p- (p=0, 1 or 2), —OSO₂(1-4C)alkyl, —NR₁₂R₁₃, —O(1-4C)alkanoyl, —OR₁a3;
m is 0, 1 or 2;
wherein two substituents R₁a both at the 4 or 5 position of ring A taken together may form a 5 to 7 membered spiro ring;
wherein two substituents R₁a at the 4 and 5 positions of ring A taken together may form a 5 to 7 membered fused ring;
provided that if (R₁a)m is a single substituent R₁a at the 5 position of ring A then R₁a is not —CH₂X wherein X is selected from R₁b;
R₁b is independently selected from hydroxy, —OSi(tri-(1-6C)alkyl) (wherein the 3 (1-6C)alkyl groups are independently selected from all possible (1-6C)alkyl groups), —NR₅C(═W)R₄, —OC(═O)R₄, 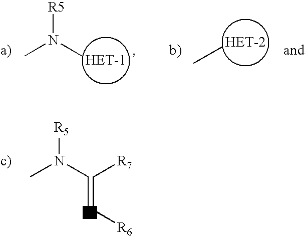
wherein W is O or S;
provided that if group C is group H or group I, and if one of substituents R₂b and R₆b is H and the other is F, and if all of substituents R₂a, R₆a, R₂a′, R₆a′, R₃a, R₅a, R₃a′, R₅a′ are H at each occurrence, then R₁b is not —NHC(═O)Me;
R₄ is selected from hydrogen, amino, (1-8C)alkyl, —NHR₁₂, —N(R₁₂)(R₁₃), —OR₁₂ or —SR₁₂, (2-4C)alkenyl, -(1-8C)alkylaryl, mono-, di-, tri- and per-halo(1-8C)alkyl, —(CH₂)p(3-6C)cycloalkyl and —(CH₂)ₚ(3-6C)cycloalkenyl wherein p is 0, 1 or 2, and additionally (2-6C)alkyl (substituted by 1, 2 or 3 substituents independently selected from methyl, chloro, bromo, fluoro, methoxy, methylthio, azido and cyano), and methyl (substituted by 1, 2 or 3 substituents independently selected from methyl, chloro, bromo, fluoro, methoxy, methylthio, hydroxy, benzyloxy, ethynyl, (1-4C)alkoxycarbonyl, azido and cyano);
R₅ is selected from hydrogen, (3-6C)cycloalkyl, phenyloxycarbonyl tert-butoxycarbonyl, fluorenyloxycarbonyl, benzyloxycarbonyl, (1-6C)alkyl (optionally substituted by cyano or (1-4C)alkoxycarbonyl), —CO₂R₈, —C(═O)R₈, —C(═O)SR₈, —C(═S)R₈, P(O)(OR₉)(OR₁₀) and —SO₂R₁₁, wherein R₈, R₉, R₁₀ and R₁₁ are as defined hereinbelow;
HET-1 is selected from HET-1A and HET-1B wherein:
HET-1A is a C-linked 5-membered heteroaryl ring containing 2 to 4 heteroatoms independently selected from N, O and S; which ring is optionally substituted on a C atom by an oxo or thioxo group; and/or which ring is optionally substituted on any available C atom by one or two substituents selected from RT as hereinafter defined and/or on an available nitrogen atom, (provided that the ring is not thereby quaternised) by (1-4C)alkyl;
HET-1B is a C-linked 6-membered heteroaryl ring containing 2 or 3 nitrogen heteroatoms, which ring is optionally substituted on a C atom by an oxo or thioxo group; and/or which ring is optionally substituted on any available C atom by one, two or three substituents selected from RT as hereinafter defined and/or on an available nitrogen atom, (provided that the ring is not thereby quaternised) by (1-4C)alkyl;
HET-2 is selected from HET-2A and HET-2B wherein
HET-2A is an N-linked 5-membered, fully or partially unsaturated heterocyclic ring, containing either (i) 1 to 3 further nitrogen heteroatoms or (ii) a further heteroatom selected from O and S together with an optional further nitrogen heteroatom; which ring is optionally substituted on a C atom, other than a C atom adjacent to the linking N atom, by an oxo or thioxo group; and/or which ring is optionally substituted on any available C atom, other than a C atom adjacent to the linking N atom, by a substituent selected from RT as hereinafter defined and/or on an available nitrogen atom, other than a N atom adjacent to the linking N atom, (provided that the ring is not thereby quaternised) by (1-4C)alkyl;
HET-2B is an N-linked 6-membered di-hydro-heteroaryl ring containing up to three nitrogen heteroatoms in total (including the linking heteroatom), which ring is substituted on a suitable C atom, other than a C atom adjacent to the linking N atom, by oxo or thioxo and/or which ring is optionally substituted on any available C atom, other than a C atom adjacent to the linking N atom, by one or two substituents independently selected from RT as hereinafter defined and/or on an available nitrogen atom, other than a N atom adjacent to the linking N atom, (provided that the ring is not thereby quaternised) by (1-4C)alkyl;
RT is selected from a substituent from the group:
(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro, and additionally (1-4C)alkoxycarbonyl; or
(RTa2) (1-4C)alkylamino, di-(1-4C)alkylamino, and (2-4C)alkenylamino; or RT is selected from the group
(RTb1) (1-4C)alkyl group which is optionally substituted by one substituent selected from hydroxy, (1-4C)alkoxy, (1-4C)alkylthio, cyano and azido; or
(RTb2) (1-4C)alkyl group which is optionally substituted by one substituent selected from (2-4C)alkenyloxy, (3-6C)cycloalkyl, and (3-6C)cycloalkenyl;
or RT is selected from the group
(RTc) a fully saturated 4-membered monocyclic ring containing 1 or 2 heteroatoms independently selected from O, N and S (optionally oxidised), and linked via a ring nitrogen or carbon atom;
and wherein at each occurrence of an RT substituent containing an alkyl, alkenyl, alkynyl, cycloalkyl or cycloalkenyl moiety in (RTa1) or (RTa2), (RTb1) or (RTb2), or (RTc) each such moiety is optionally substituted on an available carbon atom with one, two, three or more substituents independently selected from F, Cl, Br, OH and CN;
R₆ is cyano, —COR₁₂, —COOR₁₂, —CONHR₁₂, —CON(R₁₂)(R₁₃), —SO₂R₁₂, —SO₂NHR₁₂, —SO₂N(R₁₂)(R₁₃) or NO₂, wherein R₁₂ and R₁₃ are as defined hereinbelow;
R₇ is hydrogen, amino, (1-8C)alkyl, —NHR₁₂, —N(R₁₂)(R₁₃), —OR₁₂ or —SR₁₂, (2-4C)alkenyl, -(1-8C)alkylaryl, mono-, di-, tri- and per-halo(1-8C)alkyl, —(CH₂)p(3-6C)cycloalkyl or —(CH₂)p(3-6C)cycloalkenyl wherein p is 0, 1 or 2;
R₈ is hydrogen, (3-6C)cycloalkyl, phenyl, benzyl, (1-5C)alkanoyl, (1-6C)alkyl (optionally substituted by substituents independently selected from (1-5C)alkoxycarbonyl, hydroxy, cyano, up to 3 halogen atoms and —NR₁₅R₁₆ (wherein R₁₅ and R₁₆ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₅)(R₁₆) group, R₁₅ and R₁₆ may additionally be taken together with the nitrogen atom to which they are attached to form a pyrrolidinyl, piperidinyl or morpholinyl ring);
R₉ and R₁₀ are independently selected from hydrogen and (1-4C)alkyl;
R₁₁ is (1-4C)alkyl or phenyl;
R₁₂ and R₁₃ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₂)(R₁₃) group, R₁₂ and R₁₃ may additionally be taken together with the nitrogen atom to which they are attached to form a pyrrolidinyl, piperidinyl or morpholinyl ring, which ring may be optionally substituted by a group selected from (1-4C)alkyl, (3-6C)cycloalkyl, (1-4C)alkanoyll, —COO(1-4C)alkyl, S(O)n(1-4C)alkyl (wherein n=1 or 2), —COOAR1, —CS(1-4C)alkyl and C(═S)O(1-4C)alkyl;
AR1 is an optionally substituted phenyl or optionally substituted naphthyl;
AR2 is an optionally substituted 5- or 6-membered, fully unsaturated (ie with the maximum degree of unsaturation) monocyclic heteroaryl ring containing up to four heteroatoms independently selected from O, N and S (but not containing any O—O, O—S or S—S bonds), and linked via a ring carbon atom, or a ring nitrogen atom if the ring is not thereby quaternised;
AR2a is a partially hydrogenated version of AR2 (ie. AR2 systems retaining some, but not the full, degree of unsaturation), linked via a ring carbon atom or linked via a ring nitrogen atom if the ring is not thereby quaternised;
AR2b is a fully hydrogenated version of AR2 (ie. AR2 systems having no unsaturation), linked via a ring carbon atom or linked via a ring nitrogen atom;
AR3 is an optionally substituted 8-, 9- or 10-membered, fully unsaturated (i.e with the maximum degree of unsaturation) bicyclic heteroaryl ring containing up to four heteroatoms independently selected from O, N and S (but not containing any O—O, O—S or S—S bonds), and linked via a ring carbon atom in either of the rings comprising the bicyclic system;
AR3a is a partially hydrogenated version of AR3 (i.e. AR3 systems retaining some, but not the full degree of unsaturation), linked via a ring carbon atom, or linked via a ring nitrogen atom if the ring is not thereby quaternised, in either of the rings comprising the bicyclic system;
AR3b is a fully hydrogenated version of AR3 (i.e. AR3 systems having no unsaturation), linked via a ring carbon atom, or linked via a ring nitrogen atom, in either of the rings comprising the bicyclic system;
AR4 is an optionally substituted 13- or 14-membered, fully unsaturated (i.e with the maximum degree of unsaturation) tricyclic heteroaryl ring containing up to four heteroatoms independently selected from O, N and S (but not containing any O—O, O—S or S—S bonds), and linked via a ring carbon atom in any of the rings comprising the tricyclic system;
AR4a is a partially hydrogenated version of AR4 (ie. AR4 systems retaining some, but not the full, degree of unsaturation), linked via a ring carbon atom, or linked via a ring nitrogen atom if the ring is not thereby quatemised, in any of the rings comprising the tricyclic system;
CY1 is an optionally substituted cyclobutyl, cyclopentyl or cyclohexyl ring;
CY2 is an optionally substituted cyclopentenyl or cyclohexenyl ring; wherein; optional substituents on AR1, AR2, AR2a, AR2b, AR3, AR3a, AR3b, AR4, AR4a, CY1 and CY2 are (on an available carbon atom) up to three substituents independently selected from (1-4C)alkyl {optionally substituted by substituents selected independently from hydroxy, trifluoromethyl, (1-4C)alkyl S(O)q- (q is 0, 1 or 2), (1-4C)alkoxy, (1-4C)alkoxycarbonyl, cyano, nitro, (1-4C)alkanoylamino, —CONRvRw or —NRvRw}, trifluoromethyl, hydroxy, halo, nitro, cyano, thiol, (1-4C)alkoxy, (1-4C)alkanoyloxy, dimethylaminomethyleneaminocarbonyl, di(N-(1-4C)alkyl)aminomethylimino, carboxy, (1-4C)alkoxycarbonyl, (1-4C)alkanoyl, (1-4C)alkylSO₂amino, (2-4C)alkenyl {optionally substituted by carboxy or (1-4C)alkoxycarbonyl), (2-4C)alkynyl, (1-4C)alkanoylamino, oxo (═O), thioxo (═S), (1-4C)alkanoylamino {the (1-4C)alkanoyl group being optionally substituted by hydroxy}, (1-4C)alkyl S(O)q- (q is 0, 1 or 2) {the (1-4C)alkyl group being optionally substituted by one or more groups independently selected from cyano, hydroxy and (1-4C)alkoxy}, —CONRvRw or —NRvRw [wherein Rv is hydrogen or (1-4C)alkyl; Rw is hydrogen or (1-4C)alkyl];
and further optional substituents on AR1, AR2, AR2a, AR2b, AR3, AR3a, AR3b, AR4, AR4a, CY1 and CY2 (on an available carbon atom), and also on alkyl groups (unless indicated otherwise) are up to three substituents independently selected from trifluoromethoxy, benzoylamino, benzoyl, phenyl {optionally substituted by up to three substituents independently selected from halo, (1-4C)alkoxy or cyano}, furan, pyrrole, pyrazole, imidazole, triazole, pyrimidine, pyridazine, pyridine, isoxazole, oxazole, isothiazole, thiazole, thiophene, hydroxyimino(1-4C)alkyl, (1-4C)alkoxyimino(1-4C)alkyl, halo-(1-4C)alkyl, (1-4C)alkanesulfonamido, —SO₂NRvRw [wherein Rv is hydrogen or (1-4C)alkyl; Rw is hydrogen or (1-4C)alkyl]; and
optional substituents on AR2, AR2a, AR2b, AR3, AR3a, AR3b, AR4 and AR4a are (on an available nitrogen atom, where such substitution does not result in quaternization) (1-4C)alkyl, (1-4C)alkanoyl [wherein the (1-4C)alkyl and (1-4C)alkanoyl groups are optionally substituted by (preferably one) substituents independently selected from cyano, hydroxy, nitro, trifluoromethyl, (1-4C)alkyl S(O)q- (q is 0, 1 or 2), (1-4C)alkoxy, (1-4C)alkoxycarbonyl, (1-4C)alkanoylamino, —CONRvRw or —NRvRw [wherein Rv is hydrogen or (1-4C)alkyl; Rw is hydrogen or (1-4C)alkyl]}, (2-4C)alkenyl, (2-4C)alkynyl, (1-4C)alkoxycarbonyl or oxo (to form an N-oxide).

In this specification, HET-1A and HET-1B are fully unsaturated ring systems.

In this specification, HET-2A may be a fully or partially unsaturated heterocyclic ring, provided there is some degree of unsaturation in the ring.

Examples of 5-membered heteroaryl rings containing 2 to 4 heteroatoms independently selected from N, O and S (with no O—O, O—S or S—S bonds) are pyrazole, imidazole, 1,2,3-triazole, 1,2,4-triazole, oxazole, isoxazole, thiazole, 1,2,3-oxadiazole, 1,2,4-oxadiazole, 1,2,5-oxadiazole, 1,3,4-oxadiazole, isothiazole, 1,2,5-thiadiazole, 1,2,4-thiadiazole and 1,2,3-thiadiazole.

Examples of 6-membered heteroaryl ring systems containing up to three nitrogen heteroatoms are pyrimidine, pyridazine, pyrazine, 1,2,3-triazine, 1,2,4-triazine and 1,3,5-triazine.

Examples of N-linked 5-membered, fully or partially unsaturated heterocyclic rings, containing either (i) 1 to 3 further nitrogen heteroatoms or (ii) a further heteroatom selected from O and S together with an optional further nitrogen heteroatom include, for example, pyrazole, imidazole, 1,2,3-triazole (preferably 1,2,3-triazol-1-yl), 1,2,4-triazole (preferably 1,2,4-triazol-1-yl), tetrazole (preferably tetrazol-2-yl) and furazan.

Examples of N-linked 6-membered di-hydro-heteroaryl rings containing up to three nitrogen heteroatoms in total (including the linking heteroatom) include di-hydro versions of pyrimidine, pyridazine, pyrazine, 1,2,3-triazine, 1,2,4-triazine, 1,3,5-triazine and pyridine.

Particular examples of halogen-substituted alkyl substituents in HET-1 and HET-2 are monofluoromethyl, difluoromethyl, chloromethyl, dichloromethyl and trifluoromethyl.

A particular example of R₈ as a halogen-substituted alkyl group is trifluoromethyl.

In this specification the term ‘alkyl’ includes straight chain and branched structures. For example, (1-4C)alkyl includes propyl and isopropyl. However, references to individual alkyl groups such as “propyl” are specific for the straight chain version only, and references to individual branched chain alkyl groups such as “isopropyl” are specific for the branched chain version only. A similar convention applies to other radicals, for example halo(1-4C)alkyl includes 1-bromoethyl and 2-bromoethyl.

In this specification, the terms ‘alkenyl’ and ‘cycloalkenyl’ include all positional and geometrical isomers.

In this specification, the term ‘aryl’ is an unsubstituted carbocyclic aromatic group, in particular phenyl, 1- and 2-naphthyl.

In this specification, where it is stated that a ring may be linked via an sp² carbon atom it is to be understood that the ring is linked via one of the carbon atoms in a C═C double bond.

For the avoidance of doubt, reference to a carbon atom in HET1 or HET2 being substituted by an oxo or thioxo group means replacement of a CH2 by C═O or C═S respectively.

Within this specification composite terms are used to describe groups comprising more that one functionality such as (1-4C)alkoxy-(1-4C)alkoxy-(1-4C)alkyl. Such terms are to be interpreted in accordance with the meaning which is understood by a person skilled in the art for each component part. For example (1-4C)alkoxy-(1-4C)alkoxy-(1-4C)alkyl includes methoxymethoxymethyl, ethoxymethoxypropyl and propxyethoxymethyl.

It will be understood that where a group is defined such that is optionally substituted by more than one substituent, then substitution is such that chemically stable compounds are formed. For example, a trifluoromethyl group may be allowed but not a trihydroxymethyl group. This convention is applied wherever optional substituents are defined.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

Examples of (1-4C)alkyl and (1-5C)alkyl include methyl, ethyl, propyl, isopropyl and t-butyl; examples of (1-6C)alkyl include methyl, ethyl, propyl, isopropyl, t-butyl, pentyl and hexyl; examples of (1-10C)alkyl include methyl, ethyl, propyl, isopropyl, pentyl, hexyl, heptyl, octyl and nonyl; examples of (1-4C)alkanoylamino-(1-4C)alkyl include formamidomethyl, acetamidomethyl and acetamidoethyl; examples of hydroxy(1-4C)alkyl and hydroxy(1-6C)alkyl include hydroxymethyl, 1-hydroxyethyl, 2-hydroxyethyl and 3-hydroxypropyl; examples of hydroxy(2-4C)alkyl include 1-hydroxyethyl, 2-hydroxyethyl, 2-hydroxypropyl, 3-hydroxypropyl, 1-hydroxyisopropyl and 2-hydroxyisopropyl; examples of dihydroxy(1-4C)alkyl include 1,2-dihydroxyethyl, 1,2-dihydroxypropyl, 2,3-dihydroxypropyl and 1,3-dihydroxypropyl; examples of trihydroxy(1-4C)alkyl include 1,2,3-trihydroxypropyl; examples of (1-4C)alkoxycarbonyl include methoxycarbonyl, ethoxycarbonyl and propoxycarbonyl; examples of (1-5C)alkoxycarbonyl include methoxycarbonyl, ethoxycarbonyl, propoxycarbonyl and pentoxycarbonyl; examples of 2-((1-4C)alkoxycarbonyl)ethenyl include 2-(methoxycarbonyl)ethenyl and 2-(ethoxycarbonyl)ethenyl; examples of 2-cyano-2-((1-4C)alkyl)ethenyl include 2-cyano-2-methylethenyl and 2-cyano-2-ethylethenyl; examples of 2-nitro-2-((1-4C)alkyl)ethenyl include 2-nitro-2-methylethenyl and 2-nitro-2-ethylethenyl; examples of 2-((1-4C)alkylaminocarbonyl)ethenyl include 2-(methylaminocarbonyl)ethenyl and 2-(ethylaminocarbonyl)ethenyl; examples of (2-4C)alkenyl include allyl and vinyl; examples of (2-4C)alkynyl include ethynyl and 2-propynyl; examples of (1-4C)alkanoyl include formyl, acetyl and propionyl; examples of (1-4C)alkoxy include methoxy, ethoxy and propoxy; examples of (1-6C)alkoxy and (1-10C)alkoxy include methoxy, ethoxy, propoxy and pentoxy; examples of (1-4C)alkylthio include methylthio and ethylthio; examples of (1-4C)alkylamino include methylamino, ethylamino and propylamino; examples of di-((1-4C)alkyl)amino include dimethylamino, N-ethyl-N-methylamino, diethylamino, N-methyl-N-propylamino and dipropylamino; examples of halo groups include fluoro, chloro and bromo; examples of (1-4C)alkylsulfonyl include methylsulfonyl and ethylsulfonyl; examples of (1-4C)alkoxy-(1-4C)alkoxy and (1-6C)alkoxy-(1-6C)alkoxy include methoxymethoxy, 2-methoxyethoxy, 2-ethoxyethoxy and 3-methoxypropoxy; examples of (1-4C)alkoxy-(1-4C)alkoxy-(1-4C)alkoxy include 2-(methoxymethoxy)ethoxy, 2-(2-methoxyethoxy)ethoxy; 3-(2-methoxyethoxy)propoxy and 2-(2-ethoxyethoxy)ethoxy; examples of (1-4C)alkoxy-(1-4C)alkoxy-(1-4C)alkoxy-(1-4C)alkoxy-(1-4C)alkoxy-(1-4C)alkoxy include methoxyethoxyethoxyethoxyethoxyethoxy; examples of (1-4C)alkoxy-(1-4C)alkoxy-(1-4C)alkoxy-(1-4C)alkoxy-(1-4C)alkoxy include methoxyethoxyethoxyethoxyethoxy; examples of (1-4C)alkoxy-(1-4C)alkoxy-(1-4C)alkoxy-(1-4C)alkoxy include methoxyethoxyethoxyethoxy; examples of (1-4C)alkylS(O)₂amino include methylsulfonylamino and ethylsulfonylamino; examples of (1.4C)alkanoylamino and (1-6C)alkanoylamino include formamido, acetamido and propionylamino; examples of (1-4C)alkoxycarbonylamino include methoxycarbonylamino and ethoxycarbonylamino; examples of N-(1-4C)alkyl-N-(1-6C)alkanoylamino include N-methylacetamido, N-ethylacetamido and N-methylpropionamido; examples of (1-4C)alkylS(O)ₚNH— wherein p is 1 or 2 include methylsulfinylamino, methylsulfonylamino, ethylsulfinylamino and ethylsulfonylamino; examples of (1-4C)alkylS(O)ₚ((1-4C)alkyl)N— wherein p is 1 or 2 include methylsulfinylmethylamino, methylsulfonylmethylamino, 2-(ethylsulfinyl)ethylamino and 2-(ethylsulfonyl)ethylamino; examples of fluoro(1-4C)alkylS(O)ₚNH— wherein p is 1 or 2 include trifluoromethylsulfinylamino and trifluoromethylsulfonylamino; examples of fluoro(1-4C)alkylS(O)ₚ((1-4C)alkyl)NH— wherein p is 1 or 2 include trifluoromethylsulfylmethylamino and trifluoromethylsulfonylmethylamino examples of (1-4C)alkoxy(hydroxy)phosphoryl include methoxy(hydroxy)phosphoryl and ethoxy(hydroxy)phosphoryl; examples of di-(1-4C)alkoxyphosphoryl include di-methoxyphosphoryl di-ethoxyphosphoryl and ethoxy(methoxy)phosphoryl; examples of (1-4C)alkylS(O)_{q}— wherein q is 0, 1 or 2 include methylthio, ethylthio, methylsulfinyl, ethylsulfinyl, methylsulfonyl and ethylsulfonyl; examples of phenyls(O)_{q} and naphthylS(O)_{q}— wherein q is 0, 1 or 2 are phenylthio, phenylsulfinyl, phenylsulfonyl and naphthylthio, naphthylsulfinyl and naphthylsulfonyl respectively; examples of benzyloxy-(1-4C)alkyl include benzyloxymethyl and benzyloxyethyl; examples of a (3-4C)alkylene chain are trimethylene or tetramethylene; examples of hydroxy-(2-6C)alkoxy include 2-hydroxyethoxy and 3-hydroxypropoxy; e examples of (1-6C)alkoxy-(1-6C)alkyl and (1-4C)alkoxy(1-4C)alkyl include methoxymethyl ethoxymethyl and propoxyethyl; examples of di(1-4C)alkoxy(1-4C)alkyl include dimethoxymethyl diethoxymethyl 1,2-dimethoxyethyl, 1,2-diethoxyethyl, 2,3-dimethoxypropyl and 1,3-dimethoxypropyl; examples of (1-4C)alkoxy-hydroxy(1-4C)alkyl include 3-methoxy-2-hydroxypropyl, 3-hydroxy-2-methoxypropyl, 3-ethoxy-2-hydroxypropyl and 2-methoxy-2-hydroxyethyl; examples of halomethoxy(1-4C)alkyl include chloromethoxymethyl, chloromethoxyethyl, chloromethoxypropyl, chloromethoxybutyl fluoromethoxymethyl, fluoromethoxyethyl, fluoromethoxypropyl and fluoromethoxybutyl; examples of difluoromethoxy(1-4C)alkyl include difluoromethoxymethyl, difluoromethoxyethyl and difluoromethoxypropyl; examples of dihalomethoxy(1-4C)alkyl include difluoromethoxy(1-4C)alkyl; examples of trifluoromethoxy(1-4C)alkyl include trifluoromethoxymethyl, trifluoromethoxyethyl and trifluoromethoxypropyl; examples of tnhalomethoxy(1-4C)alkyl include trifluoromethoxy(1-4C)alkyl; examples of halomethoxy include chloromethoxy, chloromethoxypropyl, and fluoromethoxymethyl; examples of dihalomethoxy include difluoromethoxy; examples of trihalomethoxy include trifluoromethoxy; examples of (1-4C)alkylamino-(2-6C)alkoxy include 2-methylaminoethoxy and 2-ethylaminoethoxy; examples of di-(1-4C)alkylamino-(2-6C)alkoxy include 2-dimethylaminoethoxy and 2-diethylaminoethoxy; examples of -(1-8C)alkylaryl include benzyl and phenethyl; examples of (1-4C)alkylcarbamoyl include methylcarbamoyl and ethylcarbamoyl; examples of di((1-4C)alkyl)carbamoyl include di(methyl)carbamoyl and di(ethyl)carbamoyl; examples of hydroxyimino(1-4C)alkyl include hydroxyiminomethyl, 2-(hydroxyimino)ethyl and 1-(hydroxyimino)ethyl; examples of (1-4C)alkoxyimino-(1-4C)alkyl include methoxyiminomethyl, ethoxyiminomethyl, 1-(methoxyimino)ethyl and 2-(methoxyimino)ethyl; examples of halo groups include fluoro, chloro and bromo; examples of halo(1-4C)alkyl include, halomethyl, 1-haloethyl, 2-haloethyl, and 3-halopropyl; examples of dihalo(1-4C)alkyl include difluoromethyl and dichloromethyl; examples of txihalo(1-4C)alkyl include trifluoromethyl; examples of nitro(1-4C)alkyl include nitromethyl, 1-nitroethyl, 2-nitroethyl and 3-nitropropyl; examples of amino(1-4C)alkyl include aminomethyl, 1-aminoethyl, 2-aminoethyl and 3-aminopropyl; examples of cyano(1-4C)alkyl include cyanomethyl, 1-cyanoethyl, 2-cyanoethyl and 3-cyanopropyl; examples of (1-4C)alkanesulfonamido include methanesulfonamido and ethanesulfonamido; examples of (1-4C)alkylaminosulfonyl include methylaminosulfonyl and ethylaminosulfonyl; and examples of di-(1-4C)alkylaminosulfonyl include dimethylaminosulfonyl, diethylaminosulfonyl and N-methyl-N-ethylaminosulfonyl; examples of (1-4C)alkanesulfonyloxy include methylsulfonyloxy, ethylsulfonyloxy and propylsulfonyloxy; examples of (1-4C)alkanoyloxy include acetoxy, propanoyloxy; examples of (1-6C)alkanoyloxy include acetoxy, propanoyloxy and tert-butanoyloxy; examples of (1-6C)alkanoyloxy(1-4C)alkoxy include acetoxymethoxy, propanoyloxyethoxy and tert-butylcarbonyloxymethoxy; examples of carboxy(1-4C)alkoxy include carboxymethoxy, carboxyethoxy and carboxypropoxy; examples of (1-4C)alkylaminocarbonyl include methylaminocarbonyl and ethylaminocarbonyl; examples of di((1-4C)alkyl)aminocarbonyl include dimethylaminocarbonyl and diethylaminocarbonyl; examples of (3-8C)cycloalkyl include cyclopropyl, cyclobutyl, cyclopentyl and cyclohexyl; examples of (4-7C)cycloalkyl include cyclobutyl, cyclopentyl and cyclohexyl; examples of (3-6C)cycloalkenyl include cyclopropenyl, cyclobutenyl, cyclopentenyl and cyclohexenyl; examples of di(N-(1-4C)alkyl)aminomethylimino include dimethylaminomethylimino and diethylaminomethylimino; examples of (1-4C)alkyl-S(O)q -hydroxy(1-4C)alkyl where q is 0, 1 or 2 include 3-(methylthio)-2-hydroxypropyl, 2-(methylthio)-3-hydroxypropyl, 3-(methylsulfinyl)-2-hydroxypropyl and 3-(methylsulfonyl)-2-hydroxypropyl; examples of cyano-(hydroxy)(1-4C)alkyl include 2-cyano-3-hydroxypropyl, 3-cyano-2-hydroxypropyl, Examples of morpholino-ethoxy(1-4C)alkyl and (N′-methyl)piperazino-ethoxy(1-4C)alkyl are illustrated by: 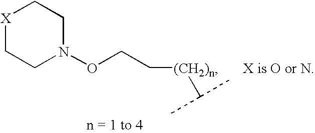
Examples of 2-, 3-, or 4-pyridyl(1-6C)alkyloxy(1-4C)alkyl are illustrated by 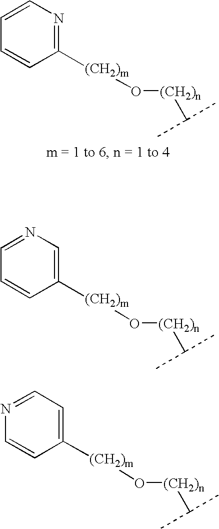
Examples of 2-, 3-, or 4-pyridyl(1-4C)alkyloxy(1-4C)alkyl are as illustrated above for 2-, 3-, or 4-pyridyl(1-6C)alkyloxy(1-4C)alkyl but wherein m=1 to 4. Examples of 2-, 3-, or 4-pyridyl(1-6C)alkylamino(1-4C)alkyl, are analogous to the alkyloxy compounds above, with NH replacing the O; similarly, examples of 2-, 3-, or 4-pyridyl(1-6C)alkylsulfonyl(1-4C)alkyl are compounds as shown above with SO₂ replacing the O.
Examples of N-methyl(imidazo -2 or 3-yl)(1-4C)alkyloxy(1-4C)alkyl are illustrated by 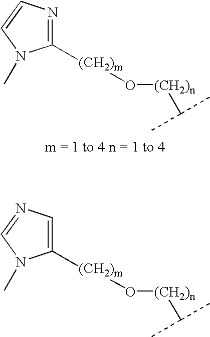
Examples of imidazo-1-yl(1-6C)alkyoxy(1-4C)alkyl are illustrated by

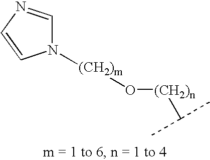

Examples of 5- and 6-membered ring acetals and methyl and phenyl derivatives thereof are 3-dioxolan-4-yl, 2-methyl-1,3-dioxolan-4-yl, 2,2-dimethyl-1,3-dioxolan-4-yl, 2,2-dimethyl-1,3-dioxan-4-yl, 2,2-dimethyl-1,3-dioxan-5-yl, 1,3-dioxan-2-yl, 2-phenyl-1,3-dioxolan-4-yl and 2-(4-methylphenyl)-1,3-dioxolan-4-yl.

Particular values for AR2 include, for example, for those AR2 containing one heteroatom, furan, pyrrole, thiophene; for those AR2 containing one to four N atoms, pyrazole, imidazole, pyridine, pyrimidine, pyrazine, pyridazine, 1,2,3- & 1,2,4-triazole and tetrazole; for those AR2 containing one N and one O atom, oxazole, isoxazole and oxazine; for those AR2 containing one N and one S atom, thiazole and isothiazole; for those AR2 containing two N atoms and one S atom, 1,2,4- and 1,3,4-thiadiazole.

Particular examples of AR2a include, for example, dihydropyrrole (especially 2,5-dihydropyrrol-4-yl) and tetrahydropyridine (especially 1,2,5,6-tetrahydropyrid-4-yl).

Particular examples of AR2b include, for example, tetrahydrofuran, pyrrolidine, morpholine (preferably morpholino), thiomorpholine (preferably thiomorpholino), piperazine (preferably piperazino), imidazoline and piperidine, 1,3-dioxolan-4-yl, 1,3-dioxan-4-yl, 1,3-dioxan-5-yl and 1,4-dioxan-2-yl. Further particular examples are 5- and 6-membered ring acetals as hereinbefore defined.

Particular values for AR3 include, for example, bicyclic benzo-fused systems containing a 5- or 6-membered heteroaryl ring containing one nitrogen atom and optionally 1-3 further heteroatoms chosen from oxygen, sulfur and nitrogen. Specific examples of such ring systems include, for example, indole, benzofuran, benzothiophene, benzimidazole, benzothiazole, benzisothiazole, benzoxazole, benzisoxazole, quinoline, quinoxaline, quinazoline, phthalazine and cinnoline.

Other particular examples of AR3 include 5/5-, 5/6 and 6/6 bicyclic ring systems containing heteroatoms in both of the rings. Specific examples of such ring systems include, for example, purine and naphthyridine.

Further particular examples of AR3 include bicyclic heteroaryl ring systems with at least one bridgehead nitrogen and optionally a further 1-3 heteroatoms chosen from oxygen, sulfur and nitrogen. Specific examples of such ring systems include, for example, 3H-pyrrolo[1,2-a]pyrrole, pyrrolo[2,1-b]thiazole, 1H-imidazo[1,2-a]pyrrole, 1H-imidazo[1,2-a]imidazole, 1H,3H-pyrrolo[1,2-c]oxazole, 1H-imidazo[1,5-a]pyrrole, pyrrolo[1,2-b]isoxazole, imidazo[5,1-b]thiazole, imidazo[2,1-b]thiazole, indolizine, imidazo[1,2-a]pyridine, imidazo[1,5-a]pyridine, pyrazolo[1,5-a]pyridine, pyrrolo[1,2-b]pyridazine, pyrrolo[1,2-c]pyrimidine, pyrrolo[1,2-a]pyrazine, pyrrolo[1,2-a]pyrimidine, pyrido[2,1-c]-s-triazole, s-triazole[1,5-a]pyridine, imidazo[1,2-c]pyrimidine, imidazo[1,2-a]pyrazine, imidazo[1,2-a]pyrimidine, imidazo[1,5-a]pyrazine, imidazo[1,5-a]pyrimidine, imidazo[1,2-b]-pyridazine, s-triazolo[4,3-a]pyrimidine, imidazo[5,1-b]oxazole and imidazo[2,1-b]oxazole. Other specific examples of such ring systems include, for example, [1H]-pyrrolo[2,1-c]oxazine, [3H]-oxazolo[3,4-a]pyridine, [6H]-pyrrolo[2,1-c]oxazine and pyrido[2,1-c][1,4]oxazine. Other specific examples of 5/5-bicyclic ring systems are imidazooxazole or imidazothiazole, in particular imidazo[5,1-b]thiazole, imidazo[2,1-b]thiazole, imidazo[5,1-b]oxazole or imidazo[2,1-b]oxazole.

Particular examples of AR3a and AR3b include, for example, indoline, 1,3,4,6,9,9a-hexahydropyrido[2,1c][1,4]oxazin-8-yl, 1,2,3,5,8,8a-hexahydroimidazo[1,5a]pyridin-7-yl, 1,5,8,8a-tetrahydrooxazolo[3,4a]pyridin-7-yl, 1,5,6,7,8,8a-hexahydrooxazolo[3,4a]pyridin-7-yl, (7aS)[3H,5H]-1,7a-dihydropyrrolo[1,2c]oxazol-6-yl, (7aS)[5H]-1,2,3,7a-tetrahydropyrrolo[1,2c]imidazol-6-yl, (7aR)[3H,5H]-1,7a-dihydropyrrolo[1,2c]oxazol-6-yl, [3H,5H]-pyrrolo[1,2-c]oxazol-6-yl, [5H]-2,3-dihydropyrrolo[1,2-c]imidazol-6-yl, [3H,5H]-pyrrolo[1,2-c]thiazol-6-yl, [3H,5H]-1,7a-dihydropyrrolo[1,2-c]thiazol-6-yl, [5H]-pyrrolo[1,2-c]imidazol-6-yl, [1H]-3,4,8,8a-tetrahydropyrrolo[2,1-c]oxazin-7-yl, [3H]-1,5,8,8a-tetrahydrooxazolo-[3,4-a]pyrid-7-yl, [311]-5,8-dihydroxazolo[3,4-a]pyrid-7-yl and 5,8-dihydroimidazo-[1,5-a]pyrid-7-yl.

Particular values for AR4 include, for example, pyrrolo[a]quinoline, 2,3-pyrroloisoquinoline, pyirolo[a]isoquinoline, 1H-pyrrolo[1,2-a]benzimidazole, 9H-imidazo[1,2-a]indole, 5H-imidazo[2,1-a]isoindole, 1H-imidazo[3,4-a]indole, imidazo[1,2-a]quinoline, imidazo[2,1-a]isoquinoline, imidazo[1,5-a]quinoline and imidazo[5,1-a]isoquinoline.

The nomenclature used is that found in, for example, “Heterocyclic Compounds (Systems with bridgehead nitrogen), W. L. Mosby (Interscience Publishers Inc., New York), 1961, Parts 1 and 2.

Where optional substituents are listed such substitution is preferably not geminal disubstitution unless stated otherwise. If not stated elsewhere, suitable optional substituents for a particular group are those as stated for similar groups herein.

Preferable optional substituents on Ar2b as 1,3-dioxolan-4-yl, 1,3-dioxan-4-yl, 1,3-dioxan-5-yl or 1,4-dioxan-2-yl are mono- or disubstitution by substituents independently selected from (1-4C)alkyl (including geminal disubstitution), (1-4C)alkoxy, (1-4C)alkylthio, acetamido, (1-4C)alkanoyl, cyano, trifluoromethyl and phenyl].

Preferable optional substituents on CY1 & CY2 are mono- or disubstitution by substituents independently selected from (1-4C)alkyl (including geminal disubstitution), hydroxy, (1-4C)alkoxy, (1-4C)alkylthio, acetamido, (1-4C)alkanoyl, cyano, and trifluoromethyl.

Suitable pharmaceutically-acceptable salts include acid addition salts such as methanesulfonate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

The compounds of the invention may be administered in the form of a pro-drug which is broken down in the human or animal body to give a compound of the invention. A prodrug may be used to alter or improve the physical and/or pharmacokinetic profile of the parent compound and can be formed when the parent compound contains a suitable group or substituent which can be derivatised to form a prodrug. Examples of pro-drugs include in-vivo hydrolysable esters of a compound of the invention or a pharmaceutically-acceptable salt thereof.

Various forms of prodrugs are known in the art, for examples see:
a) Design of Prodrugs, edited by H. Bundgaard, (Elsevier, 1985) and Methods in Enzymology, Vol, 42, p. 309-396, edited by K Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

Suitable pro-drugs for pyridine or triazole derivatives include acyloxymethyl pyridinium or triazolium salts eg halides; for example a pro-drug such as: 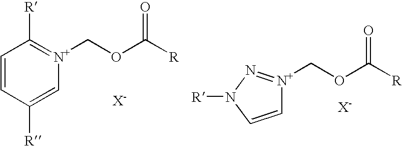
(Ref: T. Yamazaki et al. 42^{nd} Interscience Conference on Antimicrobial Agents and Chemotherapy, San Diego, 2002; Abstract F820).

Suitable pro-drugs of hydroxyl groups are acyl esters of acetal-carbonate esters of formula RCOOC(R,R′)OCO—, where R is (1-4C)alkyl and R′ is (1-4C)alkyl or H. Further suitable prodrugs are carbonate and carabamate esters RCOO— and RNHCOO—.

An in-vivo hydrolysable ester of a compound of the invention or a pharmaceutically-acceptable salt thereof containing a carboxy or hydroxy group is, for example, a pharmaceutically-acceptable ester which is hydrolysed in the human or animal body to produce the parent alcohol.

Suitable pharmaceutically-acceptable esters for carboxy include (1-6C)alkoxymethyl esters for example methoxymethyl, (1-6C)alkanoyloxymethyl esters for example pivaloyloxymethyl, phthalidyl esters, (3-8C)cycloalkoxycarbonyloxy(1-6C)alkyl esters for example 1-cyclohexylcarbonyloxyethyl; 1,3-dioxolan-2-onylmethyl esters for example 5-methyl-1,3-dioxolan-2-ylmethyl; and (1-6C)alkoxycarbonyloxyethyl esters for example 1-methoxycarbonyloxyethyl and may be formed at any carboxy group in the compounds of this invention.

An in-vivo hydrolysable ester of a compound of the invention or a pharmaceutically-acceptable salt thereof containing a hydroxy group or groups includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxymethoxy. A selection of in-vivo hydrolysable ester forming groups for hydroxy include (1-10C)alkanoyl, benzoyl, phenylacetyl and substituted benzoyl and phenylacetyl, (1-10C)alkoxycarbonyl (to give alkyl carbonate esters), di-(1-4C)alkylcarbamoyl and N-(di-(1-4C)alkylaminoethyl)-N-(1-4C)alkylcarbamoyl (to give carbamates), di-(1-4C)alkylaminoacetyl, carboxy(2-5C)alkylcarbonyl and carboxyacetyl. Examples of ring substituents on phenylacetyl and benzoyl include chloromethyl or aminomethyl (1-4C)alkylaminomethyl and di-((1-4C)alkyl)aminomethyl, and morpholino or piperazino linked from a ring nitrogen atom via a methylene linking group to the 3- or 4-position of the benzoyl ring. Other interesting in-vivo hydrolysable esters include, for example, R^{A}C(O)O(1-6C)alkyl-CO— (wherein R^{A} is for example, optionally substituted benzyloxy-(1-4C)alkyl, or optionally substituted phenyl; suitable substituents on a phenyl group in such esters include, for example, 4-(1-4C)piperazino-(1-4C)alkyl, piperazino-(1-4C)alkyl and morpholino-(1-4C)alkyl.

Suitable in-vivo hydrolysable esters of a compound of the formula (I) are described as follows. For example, a 1,2-diol may be cyclised to form a cyclic ester of formula (PD1) or a pyrophosphate of formula (PD2), and a 1,3-diol may be cyclised to form a cyclic ester of the formula (PD3):

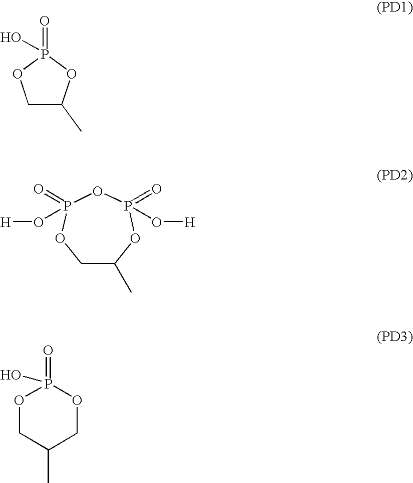

Esters of compounds of formula (I) wherein the HO— function/s in (PD1), (PD2) and (PD3) are protected by (1-4C)alkyl, phenyl or benzyl are useful intermediates for the preparation of such pro-drugs.

Further in-vivo hydrolysable esters include phosphoramidic esters, and also compounds of invention in which any free hydroxy group independently forms a phosphoryl (npd is 1) or phosphiryl (npd is 0) ester of the formula (PD4):

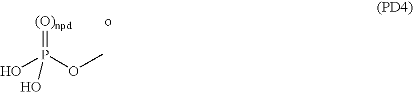

For the avoidance of doubt, phosphono is —P(O)(OH)₂; (1-4C)alkoxy(hydroxy)-phosphoryl is a mono-(1-4C)alkoxy derivative of —O—P(O)(OH)₂; and di-(1-4C)alkoxyphosphoryl is a di-(1-4C)alkoxy derivative of —O—P(O)(OH)₂.

Useful intermediates for the preparation of such esters include compounds containing a group/s of formula (PD4) in which either or both of the —OH groups in (PD1) is independently protected by (1-4C)alkyl (such compounds also being interesting compounds in their own right), phenyl or phenyl-(1-4C)alkyl (such phenyl groups being optionally substituted by 1 or 2 groups independently selected from (1-4C)alkyl, nitro, halo and (1-4C)alkoxy).

Thus, prodrugs containing groups such as (PD1), (PD2), (PD3) and (PD4) may be prepared by reaction of a compound of invention containing suitable hydroxy group/s with a suitably protected phosphorylating agent (for example, containing a chloro or dialkylamino leaving group), followed by oxidation (if necessary) and deprotection.

Other suitable prodrugs include phosphonooxymethyl ethers and their salts, for example a prodrug of R—OH such as:

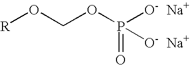

When a compound of invention contains a number of free hydroxy group, those groups not being converted into a prodrug functionality may be protected (for example, using a t-butyl-dimethylsilyl group), and later deprotected. Also, enzymatic methods may be used to selectively phosphorylate or dephosphorylate alcohol functionalities.

Where pharmaceutically-acceptable salts of an in-vivo hydrolysable ester may be formed this is achieved by conventional techniques. Thus, for example, compounds containing a group of formula (PD1), (PD2), (PD3) and/or (PD4) may ionise (partially or fully) to form salts with an appropriate number of counter-ions. Thus, by way of example, if an in-vivo hydrolysable ester prodrug of a compound of invention contains two (PD4) groups, there are four HO—P— functionalities present in the overall molecule, each of which may form an appropriate salt (i.e. the overall molecule may form, for example, a mono-, di-, tri- or tetra-sodium salt).

The compounds of the present invention have a chiral centre at the C-5 position of the oxazolidinone or isoxazoline ring B. Where m>0 there may be additional chiral centres at C-4 and/or C-5 position of Ring A The pharmaceutically active diastereomers are of the formula (Ia): 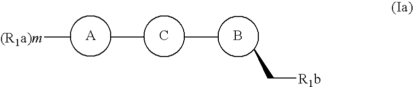
wherein the chiral centre of ring B is fixed in the orientation shown (generally the (5R) configuration, depending on the nature of R₁b, C and B) and ring B is acting as a pharmacophoric group; and wherein the substitution pattern and orientation of the chiral centre(s) at ring A may vary and may influence whether ring A also independently binds to a pharmacophore binding site.

For example when ring A is an isoxazoline ring and ring B is an oxazolidinone, the compounds of the present invention have a chiral centre at the C-5 positions of the oxazolidinone ring and, at the C-4 and/or C-5 position of the isoxazoline ring depending on the value of n (and provided that if n is 2, the isoxazoline ring is not geminally disubstituted by identical substituents). The pharmaceutically active diastereomer is then of the formula (Ib) (illustrated where group C is represented by group H): 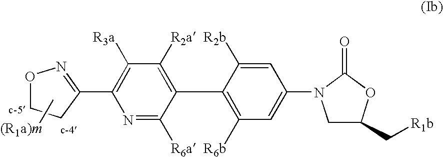
and a preferred diastereomer is of the formula (Ic):

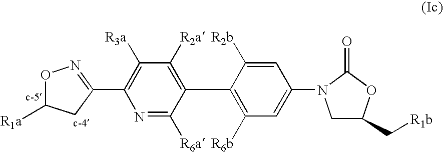

The present invention includes the pure diastereomer (Ic) depicted above, or a mixture of diastereomers wherein the substituent on the isoxazoline ring (C-5′ in structure (Ic)) is a mixture of epimers.

Where R₁b is N-linked-1,2,3-triazole, the pure diastereomer represented by (Ic) has the (5R) configuration on the oxazolidinone ring. Where R₁b is —NH(C═O)R₄, the pure diastereomer represented by (Ic) has the (5S) configuration on the oxazolidinone ring. The diasteromer (Ic) depicted above generally has the (5′S) configuration on the isoxazoline ring, although certain compounds (dependant on the nature of R₁a) have the (5′R) configuration on the isoxazoline ring.

Where R₁b is N-linked-1,2,3-triazole, a mixture of diastereomers represented by (Ic) is described herein as a mixture of the (5R,5′S) and (5R,5′R) diastereomers. Where R₁b is —NH(C═O)R₄, a mixture of diastereomers represented by (Ic) is described herein as a mixture of the (5S,5′S) and (5S,5′R) diastereomers.

If a mixture of epimers on the oxazolidinone chiral center is used, a larger amount (depending upon the ratio of the diastereoisomers) will be required to achieve the same effect as the same weight of the pharmaceutically active enantiomer.

Furthermore, some compounds of the invention may have other chiral centres, for example at C-4′. Where the substituent on an isoxazoline ring is at C-4′, a similar convention applies to that described above for substituents at C-5′. There is also, for example, the possibility of a substituent at both C-4′ and C-5′, and the possibility that such substituents may themselves contain chiral centres. It is to be understood that the invention encompasses all such optical and diastereoisomers, and racemic mixtures, that possess antibacterial activity. It is well known in the art how to prepare optically-active forms (for example by resolution of the racemic form by recrystallisation techniques, by chiral synthesis, by enzymatic resolution, by biotransformation or by chromatographic separation) and how to determine antibacterial activity as described hereinafter.

Some compounds of the invention may have more favourable MAO profiles than other compounds of the invention, which may arise from the stereochemistry and/or steric bulk of the substituent(s) on the isoxazoline ring. This is illustrated by the following examples, wherein the MAO activity is dependent on the stereochemical configration of the substituent R₄ on the isoxazoline ring. These examples illustrate that their (5′S) epimer has the higher Ki value (lower potency).

| Example | | MAO-A KI |
|---|---|---|
| No | Structure | (μM) |
| 51 | 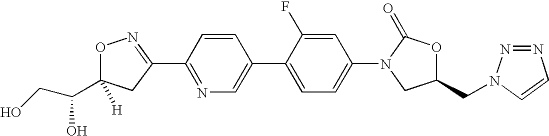 | 60* |
| 52 | 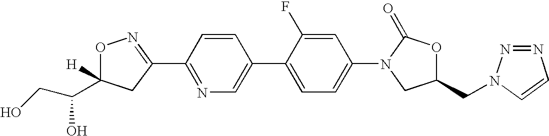 | 35* |
| 53 | 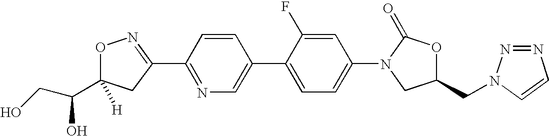 | 60 |
| 54 | 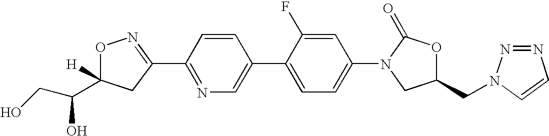 | 8 |

*= approximate values

The invention relates to all tautomeric forms of the compounds of the invention that possess antibacterial activity.

It is also to be understood that certain compounds of the invention can exist in solvated as well as unsolvated forms such as, for example, hydrated forms. It is to be understood that the invention encompasses all such solvated forms which possess antibacterial activity.

It is also to be understood that certain compounds of the invention may exhibit polymorphism, and that the invention encompasses all such forms which possess antibacterial activity.

As stated before, we have discovered a range of compounds that have good activity against a broad range of Gram-positive pathogens including organisms known to be resistant to most commonly used antibiotics, together with activity against fastidious Gram negative pathogens such as H. influenzae, M. catarrhalis, Mycoplasma and Chlamydia strains. The following compounds possess preferred pharmaceutical and/or physical and/or pharmacokinetic properties.

In one embodiment of the invention are provided compounds of formula (I), in an alternative embodiment are provided pharmaceutically-acceptable salts of compounds of formula (I), in a further alternative embodiment are provided in-vivo hydrolysable esters of compounds of formula (I), and in a further alternative embodiment are provided pharmaceutically-acceptable salts of in-vivo hydrolysable esters of compounds of formula (I).

In one aspect, an in-vivo hydrolysable ester of a compound of the formula (I) is a phosphoryl ester (as defined by formula (PD4) with npd as 1).

Compounds of the formula (I), or a pharmaceutically-acceptable salt or an in-vivo hydrolysable ester thereof, wherein C is selected from any one of groups D to 0 represent separate and independent aspects of the invention.

Particularly preferred compounds of the invention comprise a compound of the invention, or a pharmaceutically-acceptable salt or an in-vivo hydrolysable ester thereof, wherein the substituents A, B, C, RT, R₁a, R₁b, R₂a, R₂b, R₃a, R₃b R₅a, R₅a′, R₆a and R₆a′and other substituents mentioned above have values disclosed hereinbefore, or any of the following values (which may be used where appropriate with any of the definitions and embodiments disclosed hereinbefore or hereinafter):

In one embodiment are provided compounds as defined herein in formula (I) or a pharmaceutically-acceptable salt or an in-vivo hydrolysable ester thereof, in which group C is represented by group D.

In another embodiment are provided compounds as defined herein in formula (I) or a pharmaceutically-acceptable salt or an in-vivo hydrolysable ester thereof, in which group C is represented by group E.

In another embodiment are provided compounds as defined herein in formula (I) or a pharmaceutically-acceptable salt or an in-vivo hydrolysable ester thereof, in which group C is represented by group F.

In another embodiment are provided compounds as defined herein in formula (I) or a pharmaceutically-acceptable salt or an in-vivo hydrolysable ester thereof, in which group C is represented by group G.

In another embodiment are provided compounds as defined herein in formula (I) or a pharmaceutically-acceptable salt or an in-vivo hydrolysable ester thereof, in which group C is represented by group H.

In another embodiment are provided compounds as defined herein in formula (I) or a pharmaceutically-acceptable salt or an in-vivo hydrolysable ester thereof, in which group C is represented by group I.

In another embodiment are provided compounds as defined herein in formula (I) or a pharmaceutically-acceptable salt or an in-vivo hydrolysable ester thereof, in which group C is represented by group J.

In another embodiment are provided compounds as defined herein in formula (I) or a pharmaceutically-acceptable salt or an in-vivo hydrolysable ester thereof, in which group C is represented by group K.

In another embodiment are provided compounds as defined herein in formula (I) or a pharmaceutically-acceptable salt or an in-vivo hydrolysable ester thereof, in which group C is represented by group L.

In another embodiment are provided compounds as defined herein in formula (I) or a pharmaceutically-acceptable salt or an in-vivo hydrolysable ester thereof, in which group C is represented by group M.

In another embodiment are provided compounds as defined herein in formula (I) or a pharmaceutically-acceptable salt or an in-vivo hydrolysable ester thereof, in which group C is represented by group N.

In another embodiment are provided compounds as defined herein in formula (I) or a pharmaceutically-acceptable salt or an in-vivo hydrolysable ester thereof, in which group C is represented by group O.

In another embodiment are provided compounds of formula (I) or a pharmaceutically-acceptable salt or an in-vivo hydrolysable ester thereof, in which group C is represented by a group selected from groups D, E, H and I as hereinbefore defined.

In a further embodiment are provided compounds of formula (I) or a pharmaceutically-acceptable salt or an in-vivo hydrolysable ester thereof, in which group C is represented by a group selected from groups D and E as hereinbefore defined.

In a further embodiment are provided compounds of formula (I) or a pharmaceutically-acceptable salt or an in-vivo hydrolysable ester thereof, in which group C is represented by a group selected from groups D and H as hereinbefore defined.

In a most particular aspect group C is represented by group H.

In one aspect both A and B are oxazolidinone rings.

In another aspect, either A or B is an oxazolidinone ring and the other is an isoxazoline ring.

In a further aspect both A and B are isoxazoline rings.

In a most particular aspect A is an isoxazoline ring and B is an oxazolidinone ring.

In a most particular aspect, R₂b and R₆b are independently selected from H, F, Cl, CF₃, OMe, SMe, Me and Et.

In one aspect, R₂b and R₆b are independently selected from H, F, Cl, CF₃, OMe, Me and Et.

In another aspect, R₂b and R₆b are independently H or F.

In one aspect R₂b′ and R₆b′ are both H.

In a most particular aspect R₂a′ and R₆a′ are both H.

In a most particular aspect R₃a and R₅a are both H.

In a most particular aspect R₃a′, R₅a′ are both H.

In one aspect R₁a is selected from R₁a1 to R₁a4.

When m=1, in one aspect R₁a is selected from R₁a1; in another aspect R₁a is selected from R₁a2; in a further aspect R₁a is selected from R₁a3 and in a further aspect R₁a is selected from R₁a4.

When m=2, in one aspect both groups R₁a are independently selected from the same group R₁a1 to R₁a4. In a further aspect when m=2, each R₁a is independently selected from different groups R₁a1 to R₁a4.

Conveniently, m is 1 or 2. In one embodiment preferably m is 1. In another embodiment, preferably m is 2.

In one aspect, when m is 2, both substituents R₁a are attached to position 4 of ring A and joined together to form a 5-7 membered spiro-ring.

In one aspect, when m is 2, both substituents R₁a are attached to position 5 of ring A and joined together to form a 5-7 membered spiro-ring.

In another aspect, when m is 2, one substituent R₁a is attached to position 4 of ring A, and the other is attached to position 5 of ring A, such that taken together with A they form a 5-7 membered fused-ring.

In a particular aspect when m is 2, the two substituents R₁a are identical to each other, preferably selected from R₁a3 and are attached to the same position (4 or 5) of ring A such that ring A does not have a chiral centre. Suitably both R₁a are hydroxymethyl.

In a particular aspect is provided a compound of formula (Ib) as hereinbefore defined, wherein:

a) m is 1 and R₁a is a substituent on C-4′ (in one embodiment the isoxazoline ring is of the (4′S) configuration; in another the isoxazoline ring is of the (4′R) configuration); or

b) m is 1 and R₁a is a substituent on C-5′ (in one embodiment the isoxazoline ring is of the (5′S) configuration, in another the isoxazoline ring is of the (5′R) configuration); or

c) m is 2 and both substituents R₁a are substituents on C-4′; or

d) m is 2 and both substituents R₁a are substituents on C-5′; or

e) m is 2, one substituent R₁a is on C-4′ and the other is on C-5′; in one embodiment, both substituents R₄ are the same; in another the substituents R₁a are not the same;

f) when m is 2 and one R₁a is a substituent on C-4′ and the other R₁a is a substituent on C-5′, in one aspect the isoxazoline ring is of the (5′S) configuration.

Particular values for R₁a when selected from R₁a1 are AR1 and AR2, more particularly AR2.

Particular values for R₁a when selected from R₁a2 are cyano and —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl (optionally substituted on a carbon not adjacent to the nitrogen), (3-6C)cycloalkyl, (1-4C)alkanoyl, —COO(1-4C)alkyl, —S(O)ₙ(1-4C)alkyl (wherein n=1 or 2;), —COOAR1, —CS(1-4C)alkyl and —C(═S)O(1-4C)alkyl; wherein any (1-4C)alkyl, (1-4C)alkanoyl and (3-6C)cycloalkyl is optionally substituted by cyano, hydroxy or halo]. More particular values for R₁a when selected from R₁a2 are cyano, formyl, —COO(1-4C)alkyl, —C(═O)NH₂, —(C═O)piperazine and —(C═O)morpholine.

Particular values for R₁a when selected from R₁a3 are (1-10C)alkyl {optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)akylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N-(1-4C)alkyl-N-(1-6C)alkanoylamino-, —C(═W)NRvRw [wherein W is O, Rv and Rw are independently H, or (1-4C)alkyl and wherein Rv and Rw taken together with the amide nitrogen to which they are attached can form a morpholine, pyrrolidine, piperidine or piperazine ring; wherein when said ring is a piperazine ring, the ring may be optionally substituted on the additional nitrogen by a group selected from (1-4C)alkyl and (1-4C)alkanoyl], (1-4C)alkylS(O)_{q}—, (q is 0, 1 or 2), AR2, AR2-O—, AR2-NH—, and also AR2a, AR2b versions of AR2 containing groups}; wherein any (1-4C)alkyl and (1-4C)acyl present in any substituent on R₁a3 may itself be substituted by one or two groups independently selected from cyano, hydroxy, halo, amino, (1-4C)alkylamino and di(1-4C)alkylamino, provided that such a substituent is not on a carbon adjacent to a heteroatom atom if present;

More particular values for R₁a when selected from R₁a3 are (1-10C)alkyl {optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, phosphoryl [—O—P(O)(OH)₂, and mono- and di-(1-4C)alkoxy derivatives thereof), phosphiryl [—O—P(OH)₂ and mono- and di-(1-4C)alkoxy derivatives thereof), carboxy, amino, (1-4C)alkylamino, di(1-4C)alkylamino, (1-4C)alkylS(O)q (preferably where q=2), AR2 and AR2b. More particular values for R₁a when selected from R₁a3 are (1-6C)alkyl substituted as hereinbefore described. Even more particular values for R₁a when selected from R₁a3 are (1-4C)alkyl substituted as hereinbefore described.

In one aspect R₁a4: is R¹⁴C(O)O(1-6C)alkyl [wherein R¹⁴ is AR1, AR2, AR2a, AR2b, (1-4C)alkylamino, benzyloxy-(1-4C)alkyl or (1-10C)alkyl {optionally substituted as defined for (R₁a3)].

Particular values for R₁a when selected from R₁a4 are R¹⁴C(O)O(1-6C)alkyl—wherein R¹⁴ is selected from AR1, AR2, AR2a, AR2b and (1-10C)alkyl (optionally substituted by one or more substituents independently selected from OH and di (1-4C)alkylamino. More particular vales for R¹⁴ are AR2a, AR2b and (1-6C)alkyl substituted with hydroxy. More particular values for R¹⁴ are AR2a, AR2b and (1-4C)alkyl substituted with hydroxy.

Particular values for R₁a when selected from R₁a5 are fluoro, chloro and hydroxy.

In a most particular aspect, R₁a is selected from (1-4C)alkyl (optionally substituted on an available carbon atom with one, two, three or more substituents independently selected from F, Cl and Br), hydroxy(2-4C)alkyl, dihydroxy(1-4C)alkyl, trihydroxy(1-4C)alkyl, (1-4C)alkoxy(1-4C)alkyl, trifluoromethoxy(1-4C)alkyl, difluoromethoxy(1-4C)alkyl, halomethoxy(1-4C)alkyl, di[(1-4C)alkoxy](1-4C)alkyl, (1-4C)alkoxy-(hydroxy)(1-4C)alkyl, (1-4C)alkyl-S(O)_{q}-hydroxy(1-4C)alkyl (where q is 0, 1 or 2), cyano(hydroxy)(1-4C)alkyl, morpholino-ethoxy(1-4C)alkyl, (N′-methyl)piperazino-ethoxy(1-4C)alkyl, 2-, 3-, or 4-pyridyl(1-6C)alkoxy(1-4C)alkyl, N-methyl(imidazo-2 or 3-yl)(1-4C)alkoxy(1-4C)alkyl, imidazo-1-yl(1-6C)alkoxy(1-4C)alkyl, and 5- and 6-membered ring acetals (optionally substituted with one or two substituents independently selected from methyl and phenyl (wherein the phenyl group is itself optionally substituted with one or two substituents selected from methyl, methoxy, chloro and bromo)).

In an alternative most particular aspect, R₁a is selected from (1-4C)alkyl, hydroxy(2-4C)alkyl, dihydroxy(1-4C)alkyl, trihydroxy(1-4C)alkyl, (1-4C)alkoxy(1-4C)alkyl, di[(1-4C)alkoxy](1-4C)alkyl, (1-4C)alkoxy-(hydroxy)(1-4C)alkyl, (1-4C)alkyl-S(O)q-hydroxy(1-4C)alkyl (where q is 0, 1 or 2), cyano-(hydroxy)(1-4C)alkyl, morpholino-ethoxy(1-4C)alkyl, (N′-methyl)piperazino-ethoxy(1-4C)alkyl, 2-, 3-, or 4-pyridyl(1-6C)alkoxymethyl, N-methyl(imidazo-2 or 3-yl)(1-6C) alkoxymethyl, imidazo-1-yl(1-6C)alkyl, 5- and 6-membered ring acetals (optionally substituted with one or two substituents independently selected from methyl and phenyl (wherein the phenyl group is itself optionally substituted with one or two substituents selected from methyl methoxy, chloro and bromo)).

Further particular values for R₁a are (1-4C)alkylS(O)_{q}—, where q is 0, 1 or 2 and wherien the (1-4C)alkyl group is optionally substitued with hydroxy.

When R₁a is selected from 2-, 3-, or 4-pyridyl(1-4C)alkyloxy(1-4C)alkyl, N-methyl(imidazo-2 or 3-yl)(1-4C)alkyloxy(1-4C)alkyl, and imidazo-1-yl(1-6C)alkyoxy(1-4C)alkyl, it is preferably selected from 2-, 3-, or 4-pyridyl(1-4C)alkyloxymethyl, N-methyl(imidazo-2 or 3-yl)(1-4C)alkyloxymethyl, and imidazo-1-yl(1-6C)alkyoxymethyl.

References hereinafter to R₁a being selected from (1-4C)alkyl include (1-4C)alkyl optionally substituted on an available carbon atom with one, two, three or more substituents independently selected from P, Cl and Br. In one embodiment, such a (1-4C)alkyl group is optionally substituted by one, two or three substituents independently selected from F, Cl and Br. In another embodiment, such a (1-4C)alkyl group is optionally substituted by one, two or three substituents independently selected from F and Cl, so that R₁a is selected from, for example, chloromethyl fluoromethyl, difluoromethyl, trifluoromethyl, chloroethyl and fluoroethyl.

When m is 1:

in one aspect R₁a is selected from (1-4C)alkyl hydroxy(2-4C)alkyl, dihydroxy(1-4C)alkyl and trihydroxy(1-4C)alkyl;

in another aspect, R₁a is selected from (1-4C)alkoxy(1-4C)alkyl, di[(1-4C)alkoxy](1-4C)alkyl, 3-dioxolan-4-yl, 2-methyl-1,3-dioxolan-4-yl, 2,2-dimethyl-1,3-dioxolan-4-yl, 2,2-dimethyl-1,3-dioxan-4-yl, 2,2-dimethyl-1,3-dioxan-5-yl and 1,3-dioxan-2-yl;

in a further aspect, R₁a is selected from halomethoxy(1-4C)allyl and 2-, 3-, or 4-pyridyl(1-4C)alkyloxymethyl;

in a further aspect, R₁a is selected from trifluoromethoxy(1-4C)alkyl, difluoromethoxy(1-4C)alkyl and fluoromethoxy(1-4C)alkyl;

in a further aspect, R₁a is selected from morpholino-ethoxy(1-4C)alkyl, (N′-methyl)piperazino-ethoxy(1-4C)alkyl, 2-, 3-, or 4-pyridyl(1-4C)alkyloxy(1-4C)alkyl, N-ethyl(imidazo-2 or 3-yl)(1-4C)alkyloxy(1-4C)alkyl, and imidazo-1-yl(1-6C)alkyoxy(1-4C)alkyl,

When m is 1, suitably R₁a is selected from hydroxy(2-4C)alkyl and dihydroxy(1-4C)alkyl, More suitably, R₁a is selected from hydroxyethyl and 1,2-dihydroxyethyl, Preferably, when m is 1, R₁a is 1,2-dihydroxyethyl.

When m is 2:

in one aspect each R₁a is independently selected from (1-4C)alkyl, hydroxy(1-4C)alkyl, dihydroxy(1-4C)alkyl and trihydroxy(1-4C)alkyl;

in another aspect, each R₁a is independently selected from (1-4C)alkoxy(1-4C)alkyl and di[(1-4C)alkoxy](1-4C)alkyl;

in a further aspect, at least one R₁a is selected from halomethoxy(1-4C)alkyl and 2-, 3- or 4-pyridyl(1-4C)alkyloxymethyl;

in a further aspect, at least one R₁a is selected from trifluoromethoxy(1-4C)alkyl, difluoromethoxy(1-4C)alkyl and fluoromethoxy(1-4C)alkyl;

in a further aspect, one R₁a is selected from (1-4C)alkyl, hydroxy(1-4C)alkyl, dihydroxy(1-4C)alkyl and trihydroxy(1-4C)alkyl; and the other R₁a is selected from (1-4C)alkoxy(1-4C)alkyl and di[(1-4C)alkoxy](1-4C)alkyl;

in a further aspect, one R₁a is selected from (1-4C)alkyl, hydroxy(1-4C)alkyl, dihydroxy(1-4C)alkyl and trihydroxy(1-4C)alkyl; and the other R₁a is selected from halomethoxy(1-4C)alkyl and 2-, 3-, or 4-pyridyl(1-4C)alkyloxymethyl.

When m is 2, preferably both R₁a are hydroxymethyl or both hydroxyethyl. In another aspect, when m is 2, preferably one R₁a is hydroxymethyl and the other is methoxymethyl.

In all of the embodiments, aspects and preferable values for R₁b defined hereinbefore or hereinafter, any (1-4C)alkyl group may be optionally substituted as hereinbefore defined. Particular substituents for (1-4C)alkyl groups in definitions for R₁b are one or two halogen groups, particularly geminal disubstitution (provided that such substitution is not on a carbon atom attached to an oxygen) and cyano. Examples of di-halosubstituted groups are —NHCOCF₂H and —NHCSCCl₂H.

When R₁b is —N(R₅)HET-1, R₅ is preferably hydrogen.

In one embodiment R₁b is selected from hydroxy, —NHCO(1-4C)alkyl, 5-NHCO(3-6C)cycloalkyl, —NHCS(1-4C)alkyl, —NHCOO(1-4C)alkyl, —NH(C═S)O(1-4C)alkyl, —OCO(1-4C)alkyl, —N(R₅)-HET-1 and HET-2.

In another embodiment R₁b is selected from —NHCO(1-4C)alkyl, —NHCO(3-6C)cycloalkyl, —NHCS(1-4C)alkyl, —N(R₅)-HET-I and HET-2.

More preferably R₁b is selected from —NHCO(1-4C)alkyl, —NHCS(1-4C)alkyl, —N(R₅)-HET-1 and HET-2.

In one aspect, R₁b is selected from OH, —NR₅C(═W)R₄ and —OC(═O)R₄, in particular OH, —NHCOMe and —NHCOOMe.

In a further aspect, R₁b is selected from —N(R₅)-HET-1 and HET-2, in particular HET-1 as isoxazolyl, 1,2,5-thiadiazolyl or isothiazolyl and HET-2 as 1,2,3-triazol-1-yl (optionally substituted) or tetrazol-2-yl, In a most particular aspect, R₄ is selected from the values given hereinbefore.

In one aspect R₄ is selected from hydrogen, amino, (1-8C)alkyl, —NHR₁₂, —N(R₁₂)(R₁₃), —OR₁₂ or —SR₁₂, (2-4C)alkenyl, -(1-8C)alkylaryl, mono-, di-, tri- and per-halo(1-8C)alkyl, —(CH₂)ₚ(3-6C)cycloalkyl and —(CH₂)p(3-6C)cycloalkenyl wherein p is 0, 1 or 2;

In one embodiment R₁b is selected from hydroxy, —NHC(═W)R₄, —OC(═O)R₄, and

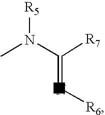

wherein W, R₅ and R₆ are as defined hereinbefore, R₄ is selected from hydrogen, amino, (1-4C)alkyl, —NH(1-4C)alkyl, —N(di-(1-4C)alkyl), —O(1-4C)alkyl, —S(1-4C)alkyl, (2-4C)alkenyl, —(CH₂)ₚ(3-6C)cycloalkyl and —(CH₂)ₚ(3-6C)cycloalkenyl wherein p is 0, 1 or 2; and R₇ is selected from hydrogen, (1-8C)alkyl, —OR₁₂, —SR₁₂, amino, NHR₁₂, N(R₁₂)(R₁₃), (1-8C)alkylaryl and mono-, di-, tri- and per-halo(1-8C)alkyl.

In another embodiment, R₁b is selected from hydroxy, —NHC(═W)R₄, —OC(═O)R₄, and 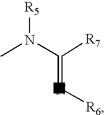
wherein W, R₄, R₅, R₆ and R₇ are as defined hereinbefore, especially wherein R₄ is (1-4C)alkyl, (1-4C)alkoxy, cycloalkyl (particularly cyclopropyl) or haloalkyl (particularly dichloromethyl).

In another embodiment, R₁b is selected from hydroxy, —NHC(═W)₄, —OC(═O)R₄, and 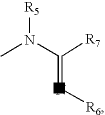
wherein W, R₄, R₅, R₆ and R₇ are as defined hereinbefore, especially wherein R is (1-4C)alkyl or (1-4C)alkoxy.

Particular values for R₅ (which may be used as appropriate with any of the definitions and embodiments disclosed hereinbefore or hereinafter) are hydrogen, tert-butoxycarbonyl and benzyloxycarbonyl. More particularly, R₅ is hydrogen.

In one aspect R₁₂ and R₁₃ are independently selected from hydrogen, alkyl and aryl, or for any N(R₁₂)(R₁₃) group, R₁₂ and R₁₃ may additionally be taken together with the nitrogen atom to which they are attached to form a pyrrolidinyl, piperidinyl or morpholinyl group, optionally substituted as hereinbefore described. In one aspect R₁₅ and R₁₆ are independently selected from hydrogen, phenyl and (1-4C)alkyl).

In a most particular aspect, R₁₂ and R₁₃ are independently selected from hydrogen and methyl

In one embodiment HET-1 and HET-2 are unsubstituted. When substituted, preferred substituents are selected from halo (particularly chloro), (1-4C)alkyl, especially methyl, mono- and di-halo methyl (wherein halo is preferably fluoro, chloro or bromo), trifluoromethyl and cyanomethyl.

Preferred are HET-1 and HET-2 as 5-membered rings, ie HET-1 as HET-1A and HET_{—}2 as HET-2A, in particular HET-1A as isoxazolyl, 1,2,5-thiadiazolyl or isothiazolyl and HET-2A as 1,2,3-triazol-1-yl or tetrazol-2-yl.

In one aspect, HET-2A as 1,2,3-triazol-1-yl is substituted, preferably by halo (particularly chloro), methyl difluoromethyl, fluoromethyl chloromethyl, cyanomethyl or trifluoromethyl.

In one embodiment HET-2A is selected from the structures (Za) to (Zf) below: 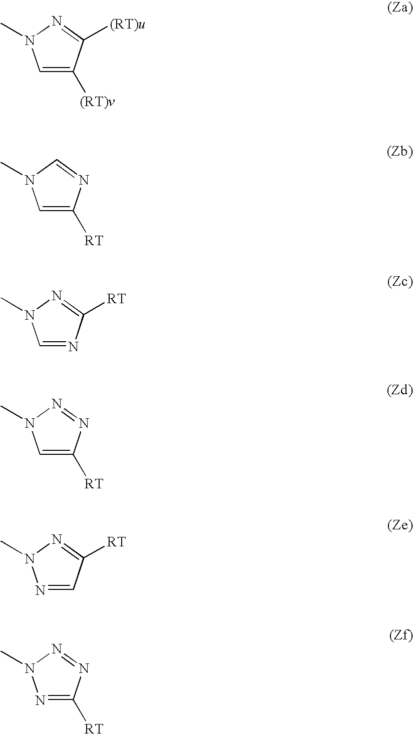
wherein u and v are independently 0 or 1 and RT is as defined in any of the embodiments or aspects defined hereinbefore or hereinafter.

In one embodiment HET-2A is selected from 1,2,3-triazole (especially 1,2,3-triazol-1-yl (Zd)), 1,2,4-triazole (especially 1,2,4-triazol-1-yl (Zc)) and tetrazole (preferably tetrazol-2-yl (Zf)) and wherein u and v are independently 0 or 1 and RT is as defined in any of the embodiments or aspects defined hereinbefore or hereinafter.

In another embodiment HET-2A is selected from 1,2,3-triazol-1-yl (Zd) and tetrazol-2-yl (Zf) and wherein u and v are independently 0 or 1 and RT is as defined in any of the embodiments or aspects defined hereinbefore or hereinafter.

In another embodiment HET-2A is 1,2,3-triazol-1-yl (Zd) and wherein u and v are independently 0 or 1 and RT is as defined in any of the embodiments or aspects defined hereinbefore or hereinafter.

In one embodiment HET-2B is a di-hydro version of pyrimidine, pyridazine, pyrazine, 1,2,3-triazine, 1,2,4-triazine, 1,3,5-triazine and pyridine and wherein RT is as defined in any of the embodiments or aspects defined hereinbefore or hereinafter.

In another embodiment HET-2B is selected from pyrimidone, pyridazinone, pyrazinone, 1,2,3-triazinone, 1,2,4-triazinone, 1,3,5-triazinone and pyridone and wherein RT is as defined in any of the embodiments or aspects defined hereinbefore or hereinafter.

In another embodiment HET-2B is selected from thiopyrimidone, thiopyridazinone, thiopyrazinone, thio-1,2,3-triazinone, thio-1,2,4-triazinone, thio-1,3,5-triazinone and thiopyridone and wherein RT is as defined in any of the embodiments or aspects defined hereinbefore or hereinafter.

In a most particular aspect, R₁b is —NH(C═W)R₄ or (Zd).

In one aspect R₁b is —NH(C═O)R₄.

In another aspect R₁b is (Zd).

When W is O, suitably R is selected from methyl ethyl, dichloromethyl and cyclopropyl.

When W is S, suitably R₄ is selected from (1-4C)alkyl (optionally substituted by 1, 2 or 3 substituents independently selected from methyl, chloro, bromo, fluoro and methoxy), —N(R₁₂)(R₁₃) and —OR₁₂. More suitably, when W is S, R₄ is selected from —NH₂, —NHMe, —OMe, —SMe and methyl.

In one aspect (RTa1) is selected from hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro.

In one aspect RT is preferably selected from a substituent from the group

(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro; or,

(RTa2) (1-4C)alkylamino, di-(1-4C)alkylamino and (2-4C)alkenylamino;

(RTb1) a (1-4C)alkyl group which is optionally substituted by one substituent selected from hydroxy, (1-4C)alkoxy, (1-4C)alkylthio, cyano and azido; or

(RTb2) a (1-4C)alkyl group which is optionally substituted by one substituent selected from (2-4C)alkenyloxy, (3-6C)cycloalkyl and (3-6C)cycloalkenyl;

and wherein at each occurrence of an RT substituent containing an alkyl, alkenyl alkynyl, cycloalkyl or cycloalkenyl moiety in (RTa1) or (RTa2), or (RTb1) or (RTb2) each such moiety is optionally substituted on an available carbon atom with one, two, three or more substituents independently selected from F, Cl, Br, OH and CN.

In another aspect RT is preferably selected from a substituent from the group:

(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano, and nitro; or

(RTb1) a (1-4C)alkyl group which is optionally substituted by one substituent selected from hydroxy, (1-4C)alkoxy, (1-4C)alkylthio, cyano and azido;

and wherein at each occurrence of an RT substituent containing an alkyl, alkenyl, alkynyl, cycloalkyl or cycloalkenyl moiety in (RTa1) or (RTb1) each such moiety is optionally substituted on an available carbon atom with one, two, three or more substituents independently selected from F, CL Br, and CN.

In a further aspect RT is most preferably
(a) hydrogen; or
(b) halogen, in particular fluorine, chlorine, or bromine; or
(c) cyano; or
(d) (1-4C)alkyl, in particular methyl; or
(e) monosubstituted (1-4C)alkyl, in particular fluoromethyl, choromethyl, bromomethyl, cyanomethyl, azidomethyl, hydroxymethyl; or
(f) disubstituted (1-4C)alkyl, for example difluoromethyl, or trisubstituted (1-4C)alkyl, for example trifluoromethyl.

In a most particular aspect, RT is selected from hydrogen, halogen, cyano, (1-4C)alkyl, cyano(1-4C)alkyl, halo(1-4C)alkyl, dihalo(1-4C)alkyl, trihalo(1-4C)alkyl, amino, (1-4C)alkylamino, di-(1-4C)alkylamino, (1-4C)alkylthio, (1-4C)alkoxy, 1-4C)alkoxy(1-4C)alkyl, (2-4C)alkenyloxy, (2-4C)alkenyl, (2-4C)alkynyl, (3-6C)cycloalkyl, (3-6C)cycloalkenyl and (1-4C)alkoxycarbonyl; and wherein at each occurrence of an RT substituent containing an alkyl, alkenyl, alkynyl, cycloalkyl or cycloalkenyl moiety each such moiety is optionally substituted on an available carbon atom with one, two, three or more substituents independently selected from F, Cl, Br, OH and CN.

In one embodiment of this most particular aspect, RT is selected from hydrogen, halogen, cyano, (1-4C)alkyl, halo(1-4C)alkyl, dihalo(1-4C)alkyl and (2-4C)alkynyl; suitably, RT is selected from hydrogen, chloro, bromo, fluoro, methyl, fluoromethyl, chloromethyl, bromomethyl, difluoromethyl and dichloromethyl, ethynyl and propynyl; more suitably, RT is selected from hydrogen, chloro, bromo, methyl and fluoromethyl.

In one embodiment is provided a compound of formula (I) or a pharmaceutically acceptable salt thereof wherein each of the groups A, B, C, RT, R₄, R₁₂, R₁₃, R₁a, R₁b, R₂a′, R₂b, R₃a, R₆b and R₆a′ is selected from the most particular aspect for that group as described hereinbefore.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both oxazolidinones; m=0; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, B and H; R₂b and R₆b are independently H or F; A and B are both oxazolidinones; m=0; and R₁b is selected from —N(R₅)-HET-1A and HET-2A, in particular HET-1A as isoxazolyl 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both isoxazolines; m=0; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or P; A and B are both isoxazolines; m=0; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; either A or B is an oxazolidinone and the other is an isoxazoline; m=0; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; either A or B is an oxazolidinone and the other is an isoxazoline; m=0; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl. In one embodiment is provided a compound of formula (I) or a pharmaceutically-acceptable salt or an in-vivo hydrolysable ester thereof, wherein group C is represented by any one of groups D, B and H; R₂b and R₆b are independently H or P; A and B are both oxazolidinones; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or P; A and B are both oxazolidinones; m=1; R₁a is selected from R₁a1; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both isoxazolines; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both isoxazolines; m=1; R₁a is selected from R₁a1; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; either A or B is an oxazolidinone and the other is an isoxazoline; m=1; R₁a is selected from R₁a1; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; either A or B is an oxazolidinone and the other is an isoxazoline; m=1; R₁a is selected from R₁a1; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both oxazolidinones; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both oxazolidinones; m=1; R₁a is selected from R₁a2; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both isoxazolines; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, 1 and H; R₂b and R₆b are independently H or F; A and B are both isoxazolines; m=1; R₁a is selected from R₁a2; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; either A or B is an oxazolidinone and the other is an isoxazoline; m=1; R₁a is selected from R₁a2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; either A or B is an oxazolidinone and the other is an isoxazoline; m=1; R₁a is selected from R₁a2; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, B and H; R₂b and R₅b are independently H or F; A and B are both oxazolidinones; m=1; R₁a is selected from R₁a3; and R₁b is selected from OH, —NHCOMe, —NHCOcyclopropyl, —NH(C—S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both oxazolidinones; m=1; R₁a is selected from R₁a3; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both isoxazolines; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both isoxazolines; m=1; R₁a is selected from R₁a3; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; either A or B is an oxazolidinone and the other is an isoxazoline; m=1; R₁a is selected from R₁a3; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; either A or B is an oxazolidinone and the other is an isoxazoline; m=1; R₁a is selected from R₁a3; and R₁b is selected from —N(R₅)—HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both oxazolidinones; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both oxazolidinones; m=2; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both isoxazolines; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; A and B are both isoxazolines; m=2; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In one embodiment is provided a compound of formula (I) or a pharmaceutically-acceptable salt or an in-vivo hydrolysable ester thereof, wherein group C is represented by any one of groups D, E and H; R₂b and R₆b are independently H or F; either A or B is an oxazolidinone and the other is an isoxazoline; m=2; and R₁b is selected from OH, —NHCOMe, —NHCOcyclopropyl, —NH(C═S)OMe and —NHCOOMe.

In one embodiment is provided a compound of formula (I) or a pharmaceutically-acceptable salt or an in-vivo hydrolysable ester thereof, wherein group C is represented by any one of groups D, B and H; R₂b and R₆b are independently H or F; either A or B is an oxazolidinone and the other is an isoxazoline; m=2; and R₁b is selected from —N(R₅)-HET-1A and HET-2A, in particular HET-1A as isoxazolyl, 1,2,5-thiadiazolyl or isothiazolyl and HET-2A as 1,2,3-triazol-1-yl (optionally substituted) or tetrazol-2-yl.

In all of the above definitions the preferred compounds are as shown in formula (Ia).

In one embodiment is provided a compound of the formula (Id), or a pharmaceutically-acceptable salt or in-vivo hydrolysable ester thereof:

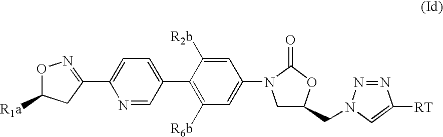

wherein

R₂b and R₆b are independently selected from hydrogen and fluorine;

RT is selected from hydrogen, halogen, (1-4C)alkyl, halo(1-4C)alkyl, dihalo(1-4C)alkyl and (2-4C)alkynyl;

R₁a is selected from (1-4C)alkyl, hydroxy(2-4C)alkyl, dihydroxy(1-4C)alkyl and trihydroxy(1-4C)alkyl.

In a further aspect of the invention is provided a compound of the formula (Id) or a pharmaceutically-acceptable salt or in-vivo hydrolysable ester thereof, wherein

R₂b and R₆b are independently selected from hydrogen and fluorine;

RT is selected from hydrogen, halogen, (1-4C)alkyl, halo(1-4C)alkyl, dihalo(1-4C)alkyl and (2-4C)alkynyl;

R₁a is selected from (1-4C)alkyl, dihydroxy(1-4C)alkyl and trihydroxy(1-4C)alkyl.

In a further aspect of the invention is provided a compound of the formula (Id) or a pharmaceutically-acceptable salt or in-vivo hydrolysable ester thereof, wherein

R₂b and R₆b are independently selected from hydrogen and fluorine;

RT is selected from hydrogen, chloro, bromo, fluoro, methyl, fluoromethyl, chloromethyl, bromomethyl, difluoromethyl, dichloromethyl, ethynyl and propynyl;

R₁a is selected from (1-4C)alkoxy(1-4C)alkyl, di[(1-4C)alkoxy](1-4C)alkyl, (1-4C)alkoxy-hydroxy(1-4C)alkyl, 3-dioxolan-4-yl, 2-methyl-1,3-dioxolan-4-yl, 2,2-dimethyl-1,3-dioxolan-4-yl, 2,2-dimethyl-1,3-dioxan-4-yl, 2,2-dimethyl-1,3-dioxan-5-yl and 1,3-dioxan-2-yl.

In a further aspect of the invention is provided a compound of the formula (Id) or a pharmaceutically-acceptable salt or in-vivo hydrolysable ester thereof, wherein

R₂b and R₆b are independently selected from hydrogen and fluorine;

RT is selected from hydrogen, chloro, bromo, fluoro, methyl, fluoromethyl, chloromethyl, bromomethyl, difluoromethyl, dichloromethyl, ethynyl and propynyl;

R₁a is selected from halomethoxy(1-4C)alkyl and 2-, 3-, or 4-pyridyl(1-4C)alkyloxymethyl,

In a further aspect of the invention is provided a compound of the formula (Id) or a pharmaceutically-acceptable salt or in-vivo hydrolysable ester thereof, wherein

R₂b and R₆b are independently selected from hydrogen and fluorine;

RT is selected from hydrogen, chloro, bromo, methyl and fluoromethyl;

R₁a is selected from hydroxyethyl and 1,2-dihydroxyethyl.

In a further aspect of the invention is provided a compound of the formula (Ie) or a pharmaceutically-acceptable salt or in-vivo hydrolysable ester thereof, 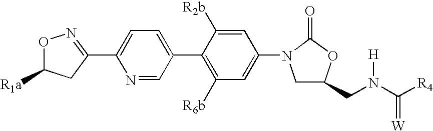
wherein

W is O;

R₂b and R₆b are independently selected from hydrogen and fluorine;

R₁a is selected from (1-4C)alkyl, hydroxy(2-4C)alkyl, dihydroxy(1-4C)alkyl and trihydroxy(1-4C)alkyl;

R₄ is selected from methyl, ethyl, dichloromethyl and cyclopropyl.

In a further aspect of the invention is provided a compound of the formula (Ie) or a pharmaceutically-acceptable salt or in-vivo hydrolysable ester thereof, wherein

W is O;

R₂b and R₆b are independently selected from hydrogen and fluorine;

R₁a is selected from (1-4C)alkyl, dihydroxy(1-4C)alkyl and trihydroxy(1-4C)alkyl;

R₄ is selected from methyl, ethyl, dichloromethyl and cyclopropyl.

In a further aspect of the invention is provided a compound of the formula (Ie) or a pharmaceutically-acceptable salt or in-vivo hydrolysable ester thereof, wherein

W is O;

R₂b and R₆b are independently selected from hydrogen and fluorine;

R₁a is selected from halomethoxy(1-4C)alkyl and 2-, 3-, or 4-pyridyl(1-4C)alkyloxymethyl;

R₄ is selected from methyl, ethyl, dichloromethyl and cyclopropyl.

In a further aspect of the invention is provided a compound of the formula (If) or a pharmaceutically-acceptable salt or in-vivo hydrolysable ester thereof, 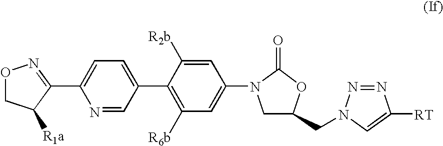
wherein

R₂b and R₆b are independently selected from hydrogen and fluorine;

RT is selected from hydrogen, halogen, (1-4C)alkyl, halo(1-4C)alkyl, dihalo(1-4C)alkyl and (2-4C)alkynyl;

R₁a is selected from (1-4C)alkyl, hydroxy(2-4C)alkyl, dihydroxy(1-4C)alkyl and trihydroxy(1-4C)alkyl.

In a further aspect of the invention is provided a compound of the formula (If) or a pharmaceutically-acceptable salt or in-vivo hydrolysable ester thereof, wherein

R₂b and R₆b are independently selected from hydrogen and fluorine;

RT is selected from hydrogen, halogen, (1-4C)alkyl, halo(1-4C)alkyl, dihalo(1-4C)alkyl and (2-4C)alkynyl;

R₁a is selected from (1-4C)alkyl, dihydroxy(1-4C)alkyl and trihydroxy(1-4C)alkyl.

In a further aspect of the invention is provided a compound of the formula (If) or a pharmaceutically-acceptable salt or in-vivo hydrolysable ester thereof, wherein

R₂b and R₆b are independently selected from hydrogen and fluorine;

RT is selected from hydrogen, chloro, bromo, fluoro, methyl, fluoromethyl, chloromethyl, bromomethyl, difluoromethyl, dichloromethyl, ethynyl and propynyl;

R₁a is selected from (1-4C)alkoxy(1-4C)alkyl, di[(1-4C)alkoxy](1-4C)alkyl, (1-4C)alkoxy-hydroxy(1-4C)alkyl, 3-dioxolan-4-yl, 2-methyl-1,3-dioxolan-4-yl, 2,2-dimethyl-1,3-dioxolan-4-yl, 2,2-dimethyl-1,3-dioxan-4-yl, 2,2-dimethyl-1,3-dioxan-5-yl and 1,3-dioxan-2-yl.

In a further aspect of the invention is provided a compound of the formula (If) or a pharmaceutically-acceptable salt or in-vivo hydrolysable ester thereof, wherein

R₂b and R₆b are independently selected from hydrogen and fluorine;

RT is selected from hydrogen, chloro, bromo, fluoro, methyl, fluoromethyl, chloromethyl, bromomethyl, difluoromethyl, dichloromethyl, ethynyl and propynyl;

R₁a is selected from halomethoxy(1-4C)alkyl and 2-, 3-, or 4-pyridyl(1-C)alkyloxymethyl,

In a further aspect of the invention is provided a compound of the formula (If) or a pharmaceutically-acceptable salt or in-vivo hydrolysable ester thereof, wherein

R₂b and R₆b are independently selected from hydrogen and fluorine;

RT is selected from hydrogen, chloro, bromo, methyl and fluoromethyl;

R₁a is selected from hydroxyethyl and 1,2-dihydroxyethyl.

In a further aspect of the invention is provided a compound of the formula (Ig) or a pharmaceutically-acceptable salt or in-vivo hydrolysable ester thereof, 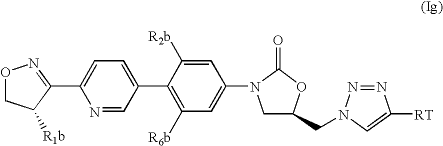
wherein

R₂b and R₆b are independently selected from hydrogen and fluorine;

RT is selected from hydrogen, halogen, (1-4C)alkyl, halo(1-4C)alkyl, dihalo(1-4C)alkyl and (2-4C)alkynyl;

R₁a is selected from (1-4C)alkyl, hydroxy(2-4C)alkyl, dihydroxy(1-4C)alkyl and trihydroxy(1-4C)alkyl.

In a further aspect of the invention is provided a compound of the formula (Ig) or a pharmaceutically-acceptable salt or in-vivo hydrolysable ester thereof, wherein

R₂b and R₆b are independently selected from hydrogen and fluorine;

RT is selected from hydrogen, halogen, (1-4C)alkyl, halo(1-4C)alkyl, dihalo(1-C)alkyl and (2-4C)alkynyl;

R₁a is selected from (1-4C)alkyl, dihydroxy(1-4C)alkyl and trihydroxy(1-4C)alkyl.

In a further aspect of the invention is provided a compound of the formula (Ig) or a pharmaceutically-acceptable salt or in-vivo hydrolysable ester thereof, wherein

R₂b and R₆b are independently selected from hydrogen and fluorine;

RT is selected from hydrogen, chloro, bromo, fluoro, methyl, fluoromethyl, chloromethyl, bromomethyl, difluoromethyl, dichloromethyl, ethynyl and propynyl;

R₁a is selected from (1-4C)alkoxy(1-4C)alkyl, di[(1-4C)alkoxy](1-4C)alkyl, (1-4C)alkoxy-hydroxy(1-4C)alkyl 3-dioxolan-4-yl, 2-methyl-1,3-dioxolan-4-yl, 2,2-dimethyl-1,3-dioxolan-4-yl, 2,2-dimethyl-1,3-dioxan-4-yl, 2,2-dimethyl-1,3-dioxan-5-yl and 1,3-dioxan-2-yl.

In a further aspect of the invention is provided a compound of the formula (Ig) or a pharmaceutically-acceptable salt or in-vivo hydrolysable ester thereof, wherein

R₂b and R₆b are independently selected from hydrogen and fluorine;

RT is selected from hydrogen, chloro, bromo, fluoro, methyl, fluoromethyl, chloromethyl, bromomethyl, difluoromethyl, dichloromethyl, ethynyl and propynyl;

R₁a is selected from halomethoxy(1-4C)alkyl and 2-, 3-, or 4-pyridyl(1-4C)alkyloxymethyl.

In a further aspect of the invention is provided a compound of the formula (Ig) or a pharmaceutically-acceptable salt or in-vivo hydrolysable ester thereof, wherein

R₂b and R₆b are independently selected from hydrogen and fluorine;

RT is selected from hydrogen, chloro, bromo, methyl and fluoromethyl;

R₁a is selected from hydroxyethyl and 1,2-dihydroxyethyl.

In a further aspect of the invention is provided a compound of the formula (Ih) or a pharmaceutically-acceptable salt or in-vivo hydrolysable ester thereof, wherein 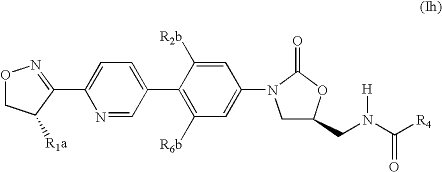
wherein

W is O;

R₂b and R₆b are independently selected from hydrogen and fluorine;

R₁a is selected from (1-4C)alkyl, hydroxy(2-4C)alkyl, dihydroxy(1-4C)alkyl and trihydroxy(1-4C)alkyl;

R₄ is selected from methyl, ethyl, dichloromethyl and cyclopropyl.

In a further aspect of the invention is provided a compound of the formula (Ih) or a pharmaceutically-acceptable salt or in-vivo hydrolysable ester thereof, wherein

W is O;

R₂b and R₆b are independently selected from hydrogen and fluorine;

R₁a is selected from (1-4C)alkyl, dihydroxy(1-4C)alkyl and trihydroxy(1-4C)alkyl;

R₄ is selected from methyl, ethyl, dichloromethyl and cyclopropyl.

In a further aspect of the invention is provided a compound of the formula (Ih) or a pharmaceutically-acceptable salt or in-vivo hydrolysable ester thereof, wherein

W is O;

R₂b and R₆b are independently selected from hydrogen and fluorine;

R₁a is selected from halomethoxy(1-4C)alkyl and 2-, 3-, or 4-pyridyl(1-4C)alkyloxymethyl;

R₄ is selected from methyl, ethyl, dichloromethyl and cyclopropyl.

In a further aspect of the invention is provided a compound of the formula (Ij) or a pharmaceutically-acceptable salt or in-vivo hydrolysable ester thereof, wherein

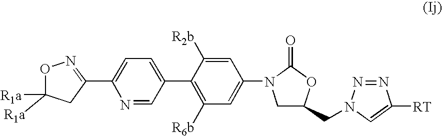

R₂b and R₆b are independently selected from hydrogen and fluorine;

RT is selected from hydrogen, halogen, (1-4C)alkyl, halo(1-4C)alkyl, dihalo(1-4C)alkyl and (2-4C)alkynyl;

each R₁a is independently selected from (1-4C)alkyl, hydroxy(1-4C)alkyl, dihydroxy(1-4C)alkyl and trihydroxy(1-4C)alkyl.

In a further aspect of the invention is provided a compound of the formula (Ij) or a pharmaceutically-acceptable salt or in-vivo hydrolysable ester thereof, wherein

R₂b and R₆b are independently selected from hydrogen and fluorine;

RT is selected from hydrogen, chloro, bromo, fluoro, methyl, fluoromethyl, chloromethyl, bromomethyl, difluoromethyl dichloromethyl, ethynyl and propynyl;

each R₁a is independently selected from (1-4C)alkoxy(1-4C)alkyl, di[(1-4C)alkoxy](1-4C)alkyl, and (1-4C)alkoxy-hydroxy(1-4C)alkyl.

In a further aspect of the invention is provided a compound of the formula (Ij) or a pharmaceutically-acceptable salt or in-vivo hydrolysable ester thereof, wherein

R₂b and R₆b are independently selected from hydrogen and fluorine;

RT is selected from hydrogen, chloro, bromo, fluoro, methyl, fluoromethyl, chloromethyl, bromomethyl, difluoromethyl, dichloromethyl, ethynyl and propynyl;

one R₁a is selected from (1-4C)alkyl, hydroxy(1-4C)alkyl, dihydroxy(1-4C)alkyl and trihydroxy(1-4C)alkyl; and

the second R₁a is selected from (1-4C)alkoxy(1-4C)alkyl, di[(1-4C)alkoxy](1-4C)alkyl, and (1-4C)alkoxy-hydroxy(1-4C)alkyl.

In a further aspect of the invention is provided a compound of the formula (Ij) or a pharmaceutically-acceptable salt or in-vivo hydrolysable ester thereof, wherein

R₂b and R₆b are independently selected from hydrogen and fluorine;

RT is selected from hydrogen, chloro, bromo, fluoro, methyl, fluoromethyl, chloromethyl, bromomethyl, difluoromethyl, dichloromethyl, ethynyl and propynyl;

one R₁a is selected from (1-4C)alkyl, hydroxy(1-4C)alkyl, dihydroxy(1-4C)alkyl and trihydroxy(1-4C)alkyl; and

the second R₁a is selected from halomethoxy(1-4C)alkyl and 2-, 3-, or 4-pyridyl(1-4C)alkyloxymethyl.

In a further aspect of the invention is provided a compound of the formula (Ij) or a pharmaceutically-acceptable salt or in-vivo hydrolysable ester thereof, wherein

R₂b and R₆b are independently selected from hydrogen and fluorine;

RT is selected from hydrogen, chloro, bromo, methyl and fluoromethyl;

both R₁a are hydroxymethyl or both are hydroxyethyl.

In a further aspect of the invention is provided a compound of the formula (Ij) or a pharmaceutically-acceptable salt or in-vivo hydrolysable ester thereof, wherein

R₂b and R₆b are independently selected from hydrogen and fluorine;

R₁b is selected from hydrogen, chloro, bromo, methyl and fluoromethyl;

one R₁a is hydroxymethyl and the other is methoxymethyl.

In a further aspect of the invention is provided a compound of the formula (In) or a pharmaceutically-acceptable salt or in-vivo hydrolysable ester thereof, wherein 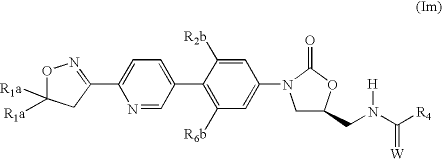
wherein

W is O;

R₂b and R₆b are independently selected from hydrogen and fluorine;

R₄ is selected from methyl, ethyl, dichloromethyl and cyclopropyl;

each R₁a is independently selected from (1-4C)alkyl, hydroxy(1-4C)alkyl, dihydroxy(1-4C)alkyl and trihydroxy(1-4C)alkyl.

In a further aspect of the invention is provided a compound of the formula (Im) or a pharmaceutically-acceptable salt or in-vivo hydrolysable ester thereof, wherein

W is O;

R₂b and R₆b are independently selected from hydrogen and fluorine;

R₄ is selected from methyl, ethyl, dichloromethyl and cyclopropyl;

each R₁a is independently selected from (1-4C)alkoxy(1-4C)alkyl, di[(1-C)alkoxy](1-4C)alkyl, and (1-4C)alkoxy-hydroxy(1-4C)alkyl.

In a further aspect of the invention is provided a compound of the formula (Im) or a pharmaceutically-acceptable salt or in-vivo hydrolysable ester thereof, wherein

W is O;

R₂b and R₆b are independently selected from hydrogen and fluorine;

R₄ is selected from methyl, ethyl, dichloromethyl and cyclopropyl;

one R₁a is selected from (1-4C)alkyl, hydroxy(1-4C)alkyl, dihydroxy(1-4C)alkyl and trihydroxy(1-4C)alkyl; and

the second R₁a is selected from (1-4C)alkoxy(1-4C)alkyl, di[(1-4C)alkoxy](1-4C)alkyl, and (1-4C)alkoxy-hydroxy(1-4C)alkyl.

In a further aspect of the invention is provided a compound of the formula (Im) or a pharmaceutically-acceptable salt or in-vivo hydrolysable ester thereof, wherein

W is O;

R₂b and R₆b are independently selected from hydrogen and fluorine;

R₄ is selected from methyl, ethyl, dichloromethyl and cyclopropyl;

one R₁a is selected from (1-4C)alkyl, hydroxy(1-4C)alkyl, dihydroxy(1-4C)alkyl and trihydroxy(1-4C)alkyl; and

the second R₁a is selected from halomethoxy(1-4C)alkyl and 2-, 3-, or 4-pyridyl(1-4C)alkyloxymethyl.

In a further aspect of the invention is provided a compound of the formula (Im) or a pharmaceutically-acceptable salt or in-vivo hydrolysable ester thereof, wherein

W is O;

R₂b and R₆b are independently selected from hydrogen and fluorine;

R₄ is selected from methyl, ethyl, dichloromethyl and cyclopropyl;

both R₁a are hydroxymethyl or both are hydroxyethyl.

In a further aspect of the invention is provided a compound of the formula (Im) or a pharmaceutically-acceptable salt or in-vivo hydrolysable ester thereof, wherein

W is O;

R₂b and R₆b are independently selected from hydrogen and fluorine;

R₄ is selected from methyl, ethyl, dichloromethyl and cyclopropyl;

one R₁a is hydroxymethyl and the other is methoxymethyl.

Particular compounds of the present invention include each individual compound described in the Examples, especially Examples 2,4 and 5.

Process Section:

In a further aspect the present invention provides a process for preparing a compound of invention or a pharmaceutically-acceptable salt or an in-vivo hydrolysable ester thereof. It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon. Resins may also be used as a protecting group.

The protecting groups may be removed at any convenient stage in the synthesis using conventional techniques well known in the chemical art.

A compound of the invention, or a pharmaceutically-acceptable salt or an in vivo hydrolysable ester thereof, may be prepared by any process known to be applicable to the preparation of chemically-related compounds. Such processes, when used to prepare a compound of the invention, or a pharmaceutically-acceptable salt or an in vivo hydrolysable ester thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March or Houben-Weyl, Methoden der Organischen Chemie). The preparation of such starting materials is described within the accompanying non-limiting Examples. Alternatively, necessary starting materials are obtainable by analogous procedures to those illustrated which are within the ordinary skill of an organic chemist. Information on the preparation of necessary starting materials or related compounds (which may be adapted to form necessary starting materials) may also be found in the certain Patent Application Publications, the contents of the relevant process sections of which are hereby incorporated herein by reference; for example WO 94-13649; WO 98-54161; WO 99-64416; WO 99-64417; WO 00-21960; WO 01-40222.

The skilled organic chemist will be able to use and adapt the information contained and referenced within the above references, and accompanying Examples therein and also the Examples herein, to obtain necessary starting materials, and products. For example, the skilled chemist will be able to apply the teaching herein for compounds of formula (I) in which two central phenyl groups are present (that is when group C is group D) to prepare compounds in which group C is represented by any of groups E to O as hereinbefore defined. Similarly, in the processes illustrated below the skilled chemist will be able to apply the teaching as necessary to prepare compounds in which for instance both rings A and B are isoxazoline and those compounds in which one of rings A and B is isoxazoline and the other oxazolidinone.

Thus, the present invention also provides that the compounds of the invention and pharmaceutically-acceptable salts and in vivo hydrolysable esters thereof, can be prepared by a process (a) to (k); and thereafter if necessary:

i) removing any protecting groups;

ii) forming a pro-drug (for example an in-vivo hydrolysable ester); and/or

iii) forming a pharmaceutically-acceptable salt;

wherein said processes (a) to (k) are as follows (wherein the variables are as defined above unless otherwise stated):

a) by modifying a substituent in, or introducing a substituent into another compound of the invention by using standard chemistry (see for example, Comprehensive Organic Functional Group Transformations (Pergamon), Katritzky, Meth-Cohn & Rees or Advanced Organic Chemistry (Wiley-Interscience), Jerry March or Houben-Weyl, Methoden der Organischen Chemie)); for example:

an acylamino group may be converted into a thioacylamino group;

an acylamino group or thioacylamino group may be converted into another acylamino or thioacylamino; heterocyclyl for instance tetrazolyl or thiazolyl, or heterocyclylamino group (optionally substituted or protected on the amino-nitrogen atom), a heterocyclyl group linked through nitrogen (optionally substituted on a carbon other than a carbon adjacent to the inking nitrogen atom), for instance an optionally 4-substituted 1,2,3-triazol-1-yl group; or an amidino group; such conversions of the acylamino group taking place either directly or through through the intermediacy of one or more derivatives such as an amino group;

an acyloxy group may be converted into a hydroxy group or into the groups that may be obtained from a hydroxy group (either directly or through the intermediacy of a hydroxy group);

an alkyl halide such as alkylbromide or alkyliodide may be converted into an alkyl fluoride or nitrite;

an alkyl sulfonate such as alkyl methanesulfonate may be converted into an alkyl fluoride or nitrile;

an alkylthio group such as methylthio may be converted into a methanesulfinyl or methanesulfonyl group;

an arylthio group such as phentlthio may be converted into a benzenesulfinyl or benzenesulfonyl group;

an amidino or guanidino group may be converted into a range of 2-substituted 1,3-diazoles and 1,3-diazines;

an amino group may be converted for instance into acylamino or thioacylamino for instance an acetamide (optionally substituted), alkyl- or dialkyl-amino and thence into a further range of N-alkyl-amine derivatives, sulfonylamino, sulfinylamino, amidino, guanidino, arylamino, heteroarylamino, N-linked heterocyclic for instance an optionally 4-substituted 1,2,3-triazol-1-yl group;

an aryl- or heteroary-halide group such as an aryl- or hetero-aryl chloride or bromide or iodide may be converted by transition metal mediated coupling, especially Pd(0) mediated coupling into a range of aryl-, heteroaryl, alkenyl, alkynyl, acyl, alkylthio, or alkyl- or dialkyl-amino substituted aryl or heteroaryl groups;

an aryl- or heteroary-sulfonate group such as an aryl- or hetero-aryl trifluoromethanesulfonate may be converted by transition metal mediated coupling, especially Pd(0) mediated coupling into a range of aryl-, heteroaryl, alkenyl, alkynyl, acyl, alkylthio, or alkyl- or dialkyl-amino substituted aryl or heteroaryl groups;

an aryl- or heteroary-halide group such as an aryl- or hetero-aryl chloride or bromide or iodide may be converted by transition metal mediated coupling, especially Pd(0) mediated coupling into a range of trialkyltin, dialkymboronate, trialkoxysilyl, substituted aryl or heteroaryl groups useful as intermediates for the synthesis of compounds of the invention;

an azido group may be converted for instance into a 1,2,3-triazolyl or amine and thence by methods that are well known in the art into any of the range common amine derivatives such

as acylamino for instance acetamido group;

a carboxylic acid group may be converted into trifloromethyl, hydroxymethyl, alkoxycarbonyl, aminocarbonyl optionally substituted on nitrogen, formyl, or acyl groups; a cyano group may be converted into a tetrazole, or an imidate, an amidine, an amidrazone, an N-hydroxyamidrazone, an amide, a thioamide, an ester, or an acid and thence by methods that are well known in the art into any of the range of heterocycles derived from such nitrile derivatives;

a hydroxy group may be converted for instance into an alkoxy, cyano, azido, alkylthio, keto and oximino, fluoro, bromo, chloro, iodo, alkyl- or aryl-sulfonyloxy for instance trifluoromethanesulfonate, metianesulfonate, or tosylsulfonate, silyloxy; acylamino or thioacylamino, for instance an acetamide (optionally substituted or protected on the amido-nitrogen atom); acyloxy, for instance an acetoxy; phosphono-oxy, heterocyclylamino (optionally substituted or protected on the amino-nitrogen atom), for instance an isoxazol-3-ylamino or a 1,2,5-thiadiazol-3-ylamino; heterocyclyl linked through nitrogen (optionally substituted on a carbon other than a carbon atom adjacent to the inking nitrogen ring atom), for instance an optionally 4-substituted 1,2,3-triazol-1-yl; or amidino, for instance an 1-(N-cyanoimino)ethylamino group; such conversions of the hydroxy group taking place directly (for instance by acylation or Mitsunobu reaction) or through the intermediacy of one or more derivatives (for instance a mesylate or an azide);

a silyloxy group may be converted into a hydroxy group or into the groups that may be obtained from a hydroxy group (either directly or through the intermediacy of a hydroxy group);

a keto group may be converted into a hydroxy, thiocarbonyl, oximino, or difluoro group; a nitro-group may be converted into an amino group and thence by methods that are well known in the art into any of the range common amine derivatives. such as acylamino for instance acetamido group;

an optionally substituted aromatic or heteroaromatic ring C′ may be converted into another aromatic or heteroaromatic ring C′ by introduction of a new substituent (R₂a to R₆a or R₂a′ or R₆a′) or by refunctionalisation of an existing substituent (R₂a to R₆a or R₂a′ or R₆a′);

a heterocyclylamino group (optionally substituted or protected on the amino-nitrogen atom) may be converted into another heterocyclyl amino group (optionally substituted or protected on the amino-nitrogen atom) by refunctionalisation, for instance by protection or deprotection, of the amino-nitrogen atom, by introduction of a new ring substituent, or by refunctionalisation of an existing ring substituent;

a heterocyclyl group linked through nitrogen (optionally substituted on a carbon other than a carbon atom adjacent to the linking nitrogen ring atom) may be converted into another heterocyclyl group linked through nitrogen (optionally substituted on a carbon other than a carbon atom adjacent to the linking nitrogen ring atom) by introduction of a new ring substituent or by refunctionalisation of an existing ring substituent, for instance by modifying the 4-substituent of a 4-substituted 1,2,3-triazol-1-yl group;
for instance, examples drawn from the methods for conversion of a hydroxy group into an optionally substituted triazole group are illustrated by the scheme: 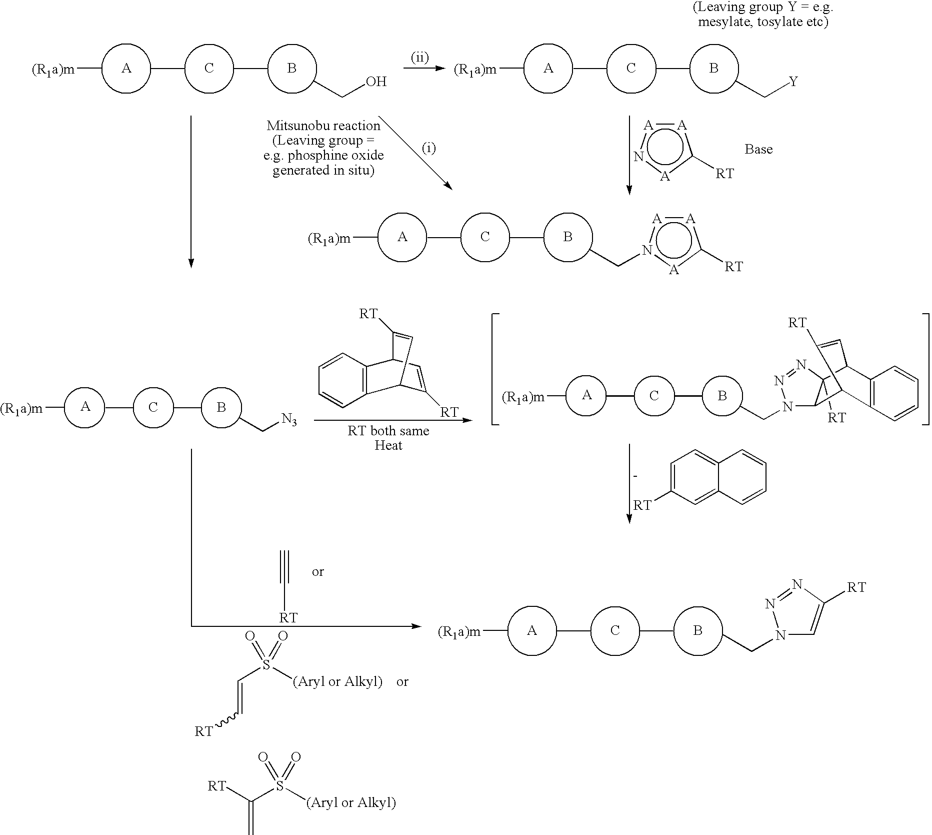
examples drawn from the range of regioselective methods that proceed under very mild conditions are further illustrated by processes (f), (g), and (h);
b) by reaction of a molecule of a compound of formula (IIa) (wherein X is a leaving group useful in palladium coupling, for example boronate, trimethyl tin, iodo and bromo) with a molecules of a compound of formula (IIb) (wherein X′ is a leaving group useful in palladium coupling, for example boronate, trimethyl tin, iodo and bromo) wherein X and X′ are chosen such that an aryl-aryl, heteroaryl-aryl, or heteroaryl-heteroaryl bond replaces the aryl-X (or heteroaryl-X) and aryl-X′ (or heteroaryl-X′) bonds. Such methods are now well known, see for instance see for instance J. K Stille, Angew Chem. Int. Ed. Eng., 1986, 25, 509-524; N. Miyaura and A Suzuki, Chem. Rev., 1995, 95, 2457-2483, D. Baranano, G. Mann, and J. F. Hartwig, Current Org. Chem, 1997, 1, 287-305, S. P. Stanforth, Tetrahedron, 54 1998, 263-303, and P. R. Parry, C. Wang, A. S. Batsanov, M. R Bryce, and B. Tarbit, J. Org. Chem., 2002, 67, 7541-7543; 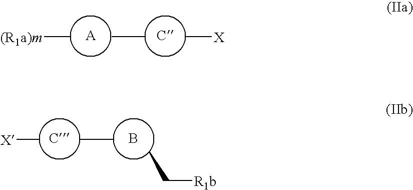
the leaving groups X and X′ may be chosen to be the same and lead to symmetrical molecules of formula (I) or different and chosen to lead to symmetrical or unsymmetrical molecules of formula (I);
for example 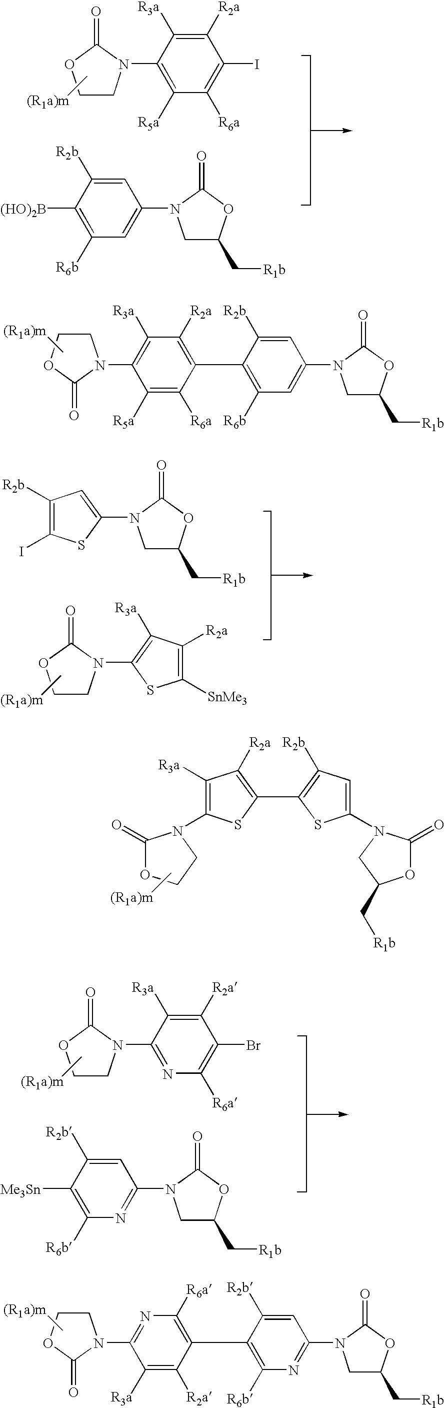 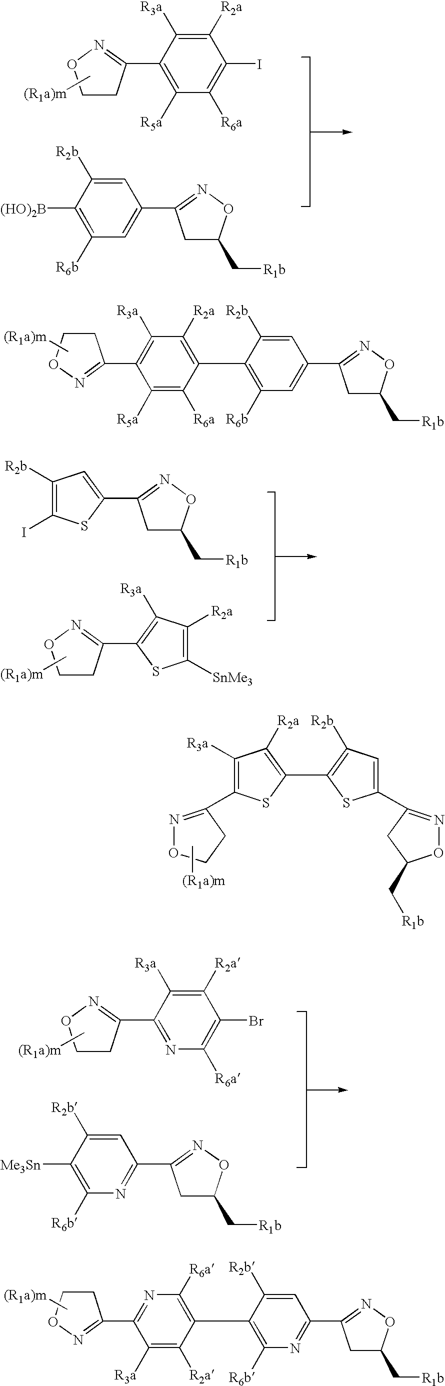
similarly, this chemistry may be applied to two dissimilar molecules of formula (II), for example those in which ring A is not the same as ring B, wherein X is suitably selected to enable unsymmetrical coupling so that an aryl-aryl, heteroaryl-aryl, or heteroaryl-heteroaryl bond replaces the aryl-X (or heteroaryl-X) and the aryl-X′ (or heteroaryl-X′) bonds; for example 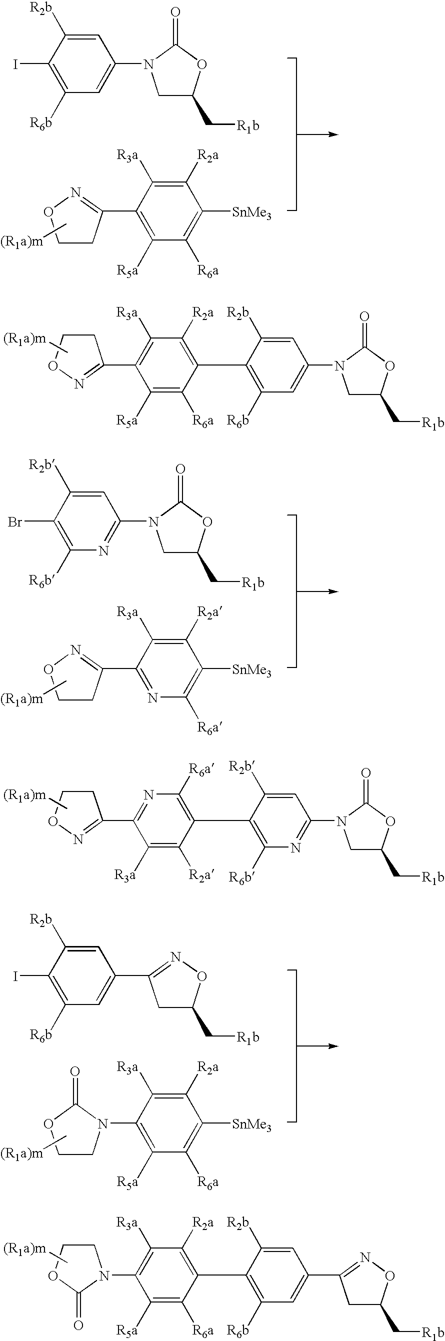 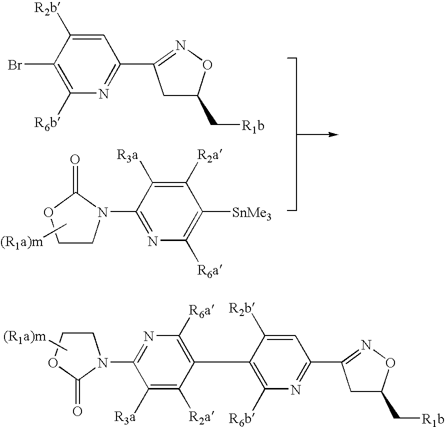
furthermore, this chemistry may also be applied to two dissimilar molecules of formula (II), for example those in which ring C′ is not the same as ring C″, wherein X and X′ are suitably selected to enable unsymmetrical coupling so that an aryl-aryl, heteroaryl-aryl, or heteroaryl-heteroaryl bond replaces the two different aryl-X (or heteroaryl-X) and the aryl-X′ (or heteroaryl-X′) bonds;
for example 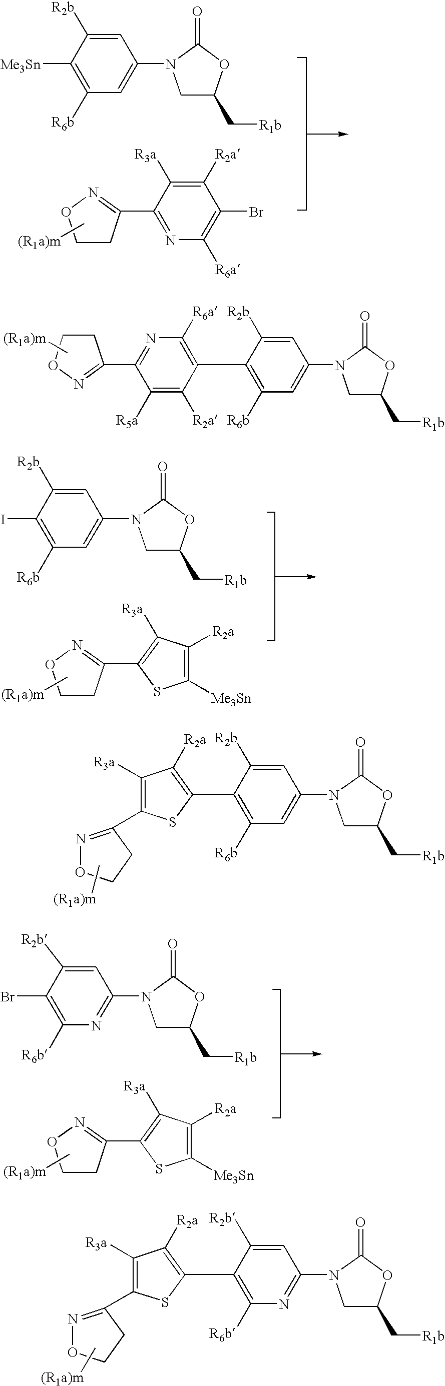 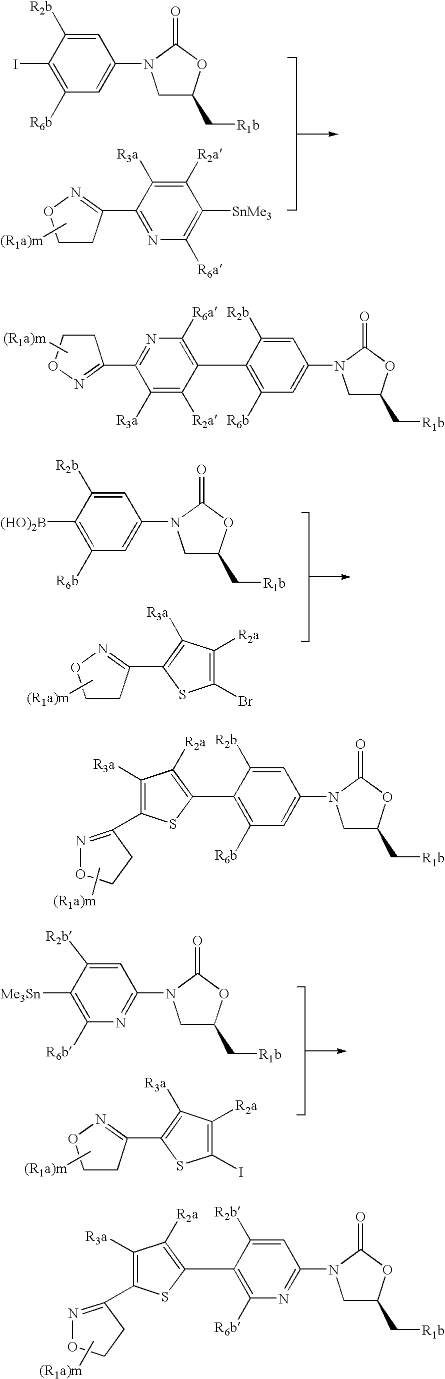 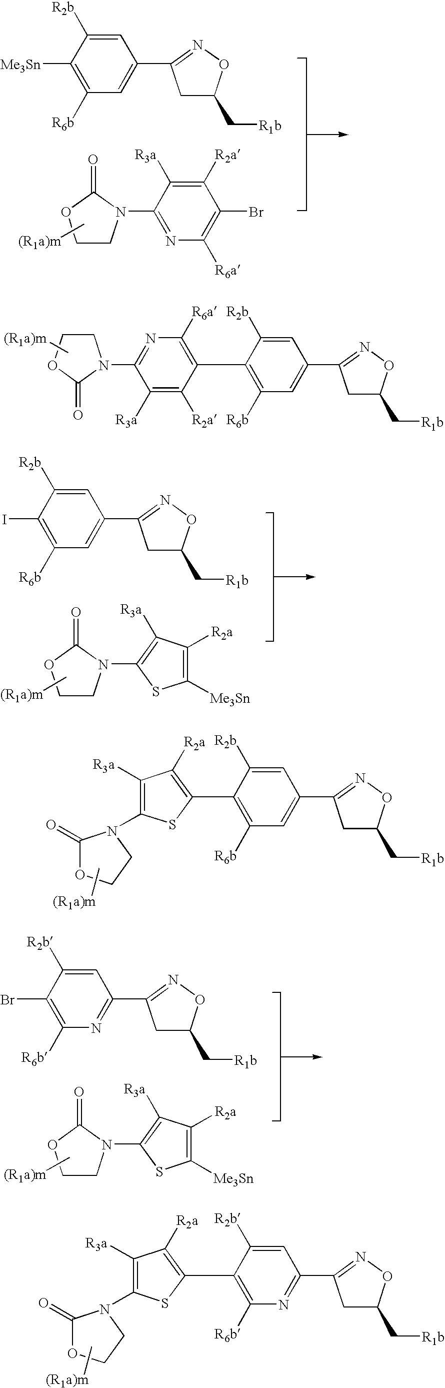
the aryl isoxazolines and aryl oxazolidinones required as reagents for process (b) or as intermediates for the preparation of reagents for process (b) may be prepared by standard organic methods, for instance by methods analogous to those set out in process sections (c) and (h), methods for the introduction and interconversion of Groups X and X′ are well known in the art;
c) by reaction of a compound of formula (IIIa) with a compound of formula (IIIb): 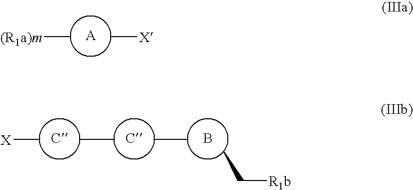
where X and X′ are replaceable substituents—such as chloride, bromide, iodide, trifluoromethylsulfonyloxy, trimethylstannyl, trialkoxysilyl, or a boronic acid residue; and wherein the substituents X and X′ are chosen to be complementary pairs of substituents known in the art to be suitable as complementary substrates for coupling reactions catalysed by transition metals such as palladium(0);

d) by reaction of a (hetero)biaryl derivative (IVa) or (IVb) carbamate with an appropriately substituted oxirane (wherein 0, 1, or 2 of R₁a′-R₁a″″ are substitutents as defined for R₁a and the remainder are hydrogen) to form an oxazolidinone ring at the undeveloped aryl position; 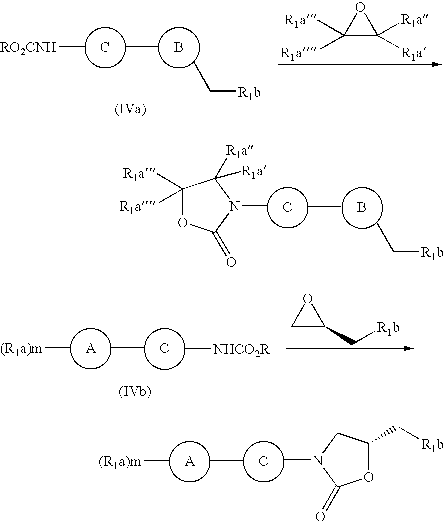
variations on this process in which the carbamate is replaced by an isocyanate or by an amine or/and in which the oxirane is replaced by an equivalent reagent X—C(R₁a′)(R₁a″)C(R₁a′″)(O-optionally protected)(R₁a″″) or X—CH₂CH(O-optionally protected)CH₂R₁b where X is a displaceable group are also well known in the art; for example, 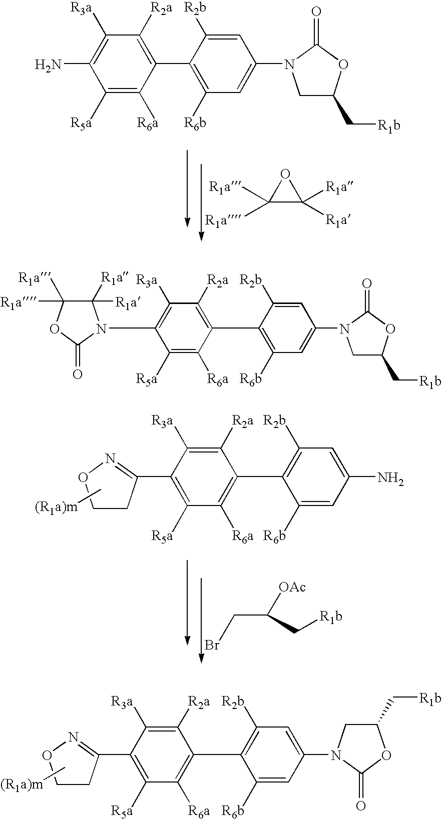
e) by reaction of a (hetero)biaryl derivative (Va) or (Vb) to form an isoxazoline ring at the undeveloped aryl position; 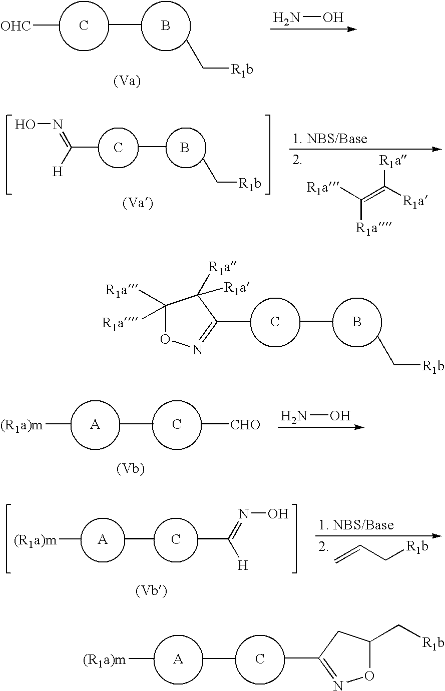
variations on this process in which the reactive intermediate (a nitrile oxide Va″ or Vb″) is obtained other than by oxidation of an oxime (Va′) or (Vb′) are well known in the art; 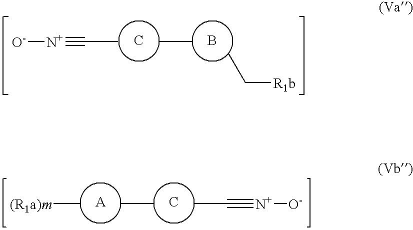
for example, oxidation of an appropriately substituted biphenylcarboxaldehyde oxime in the presence of an appropriately substituted allyl derivative gives an isoxazoline of the required structure; 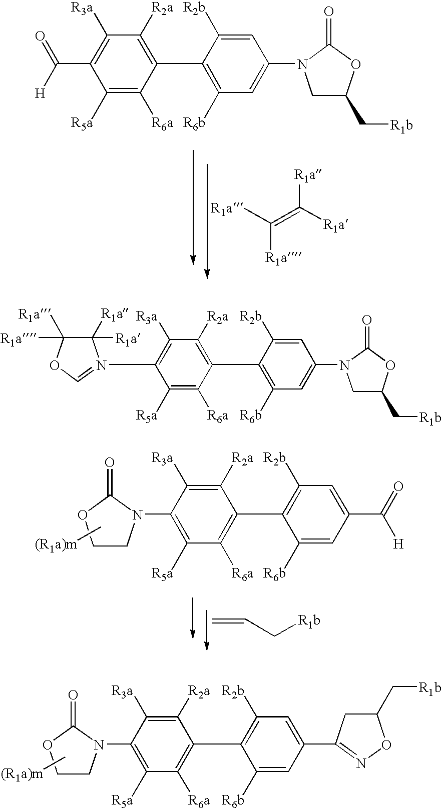
enantioselective synthesis of 2-isoxazolines via asymmetric cycloaddition of nitrile oxides to olefins has been achieved by the use of chiral auxiliaries; for instance, when the alcohol is an allyl alcohol the desired stereochemistry at ring B can be obtained in reactions conducted in the presence of (R,R)-diisopropyl tartrate (or (S, S)-diisopropyl tartrate depending on the desired stereochemistry) as a chiral auxiliary (Yutaka Ukaji et al. Chem. Letters, 1993, 1847-1850). Other chiral auxiliaries may also be employed with other olefins (see for instance Takahiko Akayama et al, Tet. Letters, 1992, 33, 5763-5766; and Jeffrey Stack et al., Tetrahedron, 1993, 49, 995-1008 and references therein); 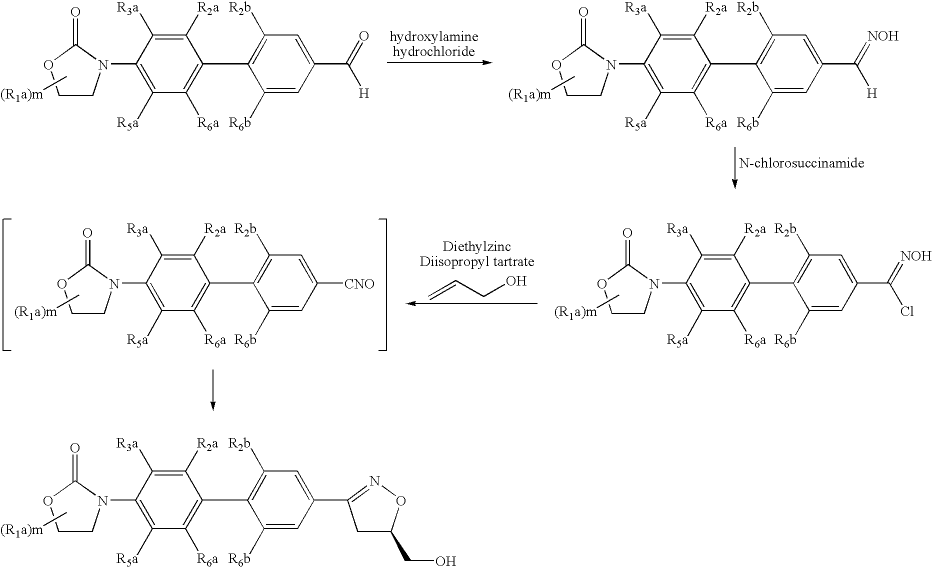
(f) for HET as optionally substituted 1,2,3-triazoles, compounds of the formula (I) may be made by cycloaddition via the azide (wherein e.g. Y in (II) is azide) to acetylenes, or to acetylene equivalents such as optionally substituted cylcohexa-1,4-dienes or optionally substituted ethylenes bearing eliminatable substituents such as arylsulfonyl;
(g) for BET as 4-substituted 1,2,3-triazole compounds of formula (I) may be made by reacting aminomethyloxazolidinones with 1,1-dihaloketone sulfonylhydrazones (Sakai, Kunihazu; Hida, Nobuko; Kondo, Kiyosi; Bull. Chem. Soc. Jpn., 59, 1986, 179-183; Sakai, Kunikazu; Tsunemoto, Daiei; Kobori, Takeo; Kondo, Kiyoshi; Hido, Noboko EP 103840 A2 19840328); for instance 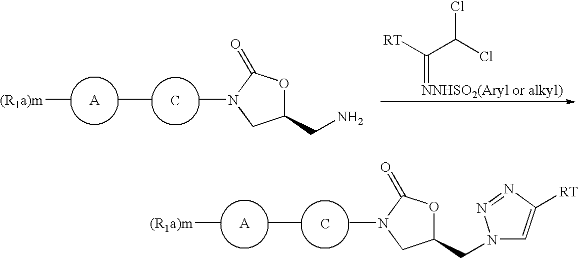
(h) for HET as 4-substituted 1,2,3-triazole compounds of formula (I) may also be made by reacting azidomethyl oxazolidinones with terminal alkynes using Cu(I) catalysis in e.g. aqueous alcoholic solution at ambient temperatures to give 4-substituted 1,2,3-triazoles (V.V. Rostovtsev, L. G. Green, V. V. Fokin, and K. B. Sharpless, Angew. Chem Into Ed., 2002, 41, 2596-2599); for instance 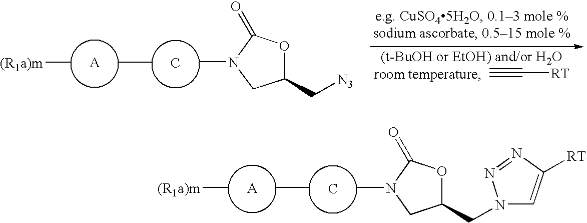
(j) for BET as 4-halogenated 1,2,3-triazole compounds of formula (I) may also be made by reacting azidomethyl oxazolidinones with halovinylsulfonyl chlorides at a temperature between 0° C. and 100° C. either neat or in an inert diluent such as chlorobenzene, chloroform or dioxan; for instance 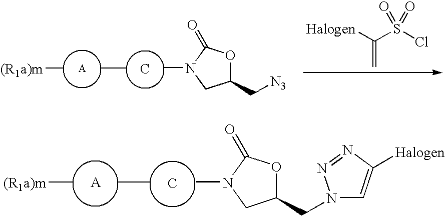
(k) for R₁b as NHCOCH₃ compounds of formula (I) may be prepared by conventional methods described in the prior art, see for example Upjohn Patent Application WO 97/37980; or for example as illustrated below.

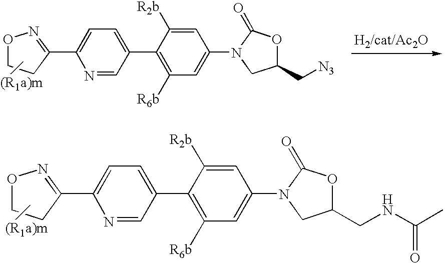

The removal of any protecting groups, the formation of a pharmaceutically-acceptable salt and/or the formation of an in-vivo hydrolysable ester are within the skill of an ordinary organic chemist using standard techniques. Furthermore, details on the these steps, for example the preparation of in-vivo hydrolysable ester prodrugs has been provided, for example, in the section above on such esters.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further feature of the invention there is provided a compound of the invention, or a pharmaceutically-acceptable salt, or in-vivo hydrolysable ester thereof for use in a method of treatment of the human or animal body by therapy.

According to a further feature of the present invention there is provided a method for producing an antibacterial effect in a warmblooded animal, such as man, in need of such treatment, which comprises administering to said animal an effective amount of a compound of the present invention, or a pharmaceutically-acceptable salt, or in-vivo hydrolysable ester thereof.

The invention also provides a compound of the invention, or a pharmaceutically-acceptable salt, or in-vivo hydrolysable ester thereof, for use as a medicament; and the use of a compound of the invention of the present invention, or a pharmaceutically-acceptable salt, or in-vivo hydrolysable ester thereof, in the manufacture of a medicament for use in the production of an antibacterial effect in a warm blooded animal, such as man.

In order to use a compound of the invention, an in-vivo hydrolysable ester or a pharmaceutically-acceptable salt thereof, including a pharmaceutically-acceptable salt of an in-vivo hydrolysable ester, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the invention, an in-vivo hydrolysable ester or a pharmaceutically-acceptable salt thereof, including a pharmaceutically-acceptable salt of an in-vivo hydrolysable ester, and a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration as eye-drops, for administration by inhalation (for example as a finely divided powder or a liquid aerosol), for administration by insufflation (for example as a finely divided powder) or for parenteral administration (for example as a sterile aqueous or oily solution for intravenous, subcutaneous, sub-lingual, intramuscular or intramuscular dosing or as a suppository for rectal dosing).

In addition to the compounds of the present invention, the pharmaceutical composition of this invention may also contain (ie through co-formulation) or be co-administered (simultaneously, sequentially or separately) with one or more known drugs selected from other clinically useful antibacterial agents (for example, β-lactams, macrolides, quinolones or aminoglycosides) and/or other anti-infective agents (for example, an antifungal triazole or amphotericin). These may include carbapenems, for example meropenem or imipenem, to broaden the therapeutic effectiveness. Compounds of this invention may also be co-formulated or co-administered with bactericidal/permeability-increasing protein (BPI) products or efflux pump inhibitors to improve activity against gram negative bacteria and bacteria resistant to antimicrobial agents. Compounds of this invention may also be co-formulated or co-administered with a vitamin, for example Vitamin B, such as Vitamin B2, Vitamin B6, Vitamin B12 and folic acid. Compounds of the invention may also be formulated or co-administered with cyclooxygenase (COX) inhibitors, particularly COX-2 inhibitors.

In one aspect of the invention, a compound of the invention is co-formulated with an antibacterial agent which is active against gram-positive bacteria.

In another aspect of the invention, a compound of the invention is co-formulated with an antibacterial agent which is active against gram-negative bacteria.

In another aspect of the invention, a compound of the invention is co-administered with an antibacterial agent which is active against gram-positive bacteria.

In another aspect of the invention, a compound of the invention is co-administered with an antibacterial agent which is active against gram-negative bacteria.

The compositions of the invention may be obtained by conventional procedures using conventional pharmaceutical excipients, well known in the art. Thus, compositions intended for oral use may contain, for example, one or more colouring, sweetening, flavouring and/or preservative agents. A pharmaceutical composition to be dosed intravenously may contain advantageously (for example to enhance stability) a suitable bactericide, antioxidant or reducing agent, or a suitable sequestering agent.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropybnethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol. Solubility enhancing agents, for example cyclodextrins may be used.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 50 mg to 5 g of active agent compounded with an appropriate and convenient amount of excipients which may vary from about 5 to about 98 percent by weight of the total composition. Dosage unit forms will generally contain about 200 mg to about 2 g of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

A suitable pharmaceutical composition of this invention is one suitable for oral administration in unit dosage form, for example a tablet or capsule which contains between 1 mg and 1 g of a compound of this invention, preferably between 100 mg and 1 g of a compound. Especially preferred is a tablet or capsule which contains between 50 mg and 800 mg of a compound of this invention, particularly in the range 100 mg to 500 mg.

In another aspect a pharmaceutical composition of the invention is one suitable for intravenous, subcutaneous or intramuscular injection, for example an injection which contains between 0.1% w/v and 50% w/v (between 1 mg/ml and 500 mg/ml) of a compound of this invention.

Each patient may receive, for example, a daily intravenous, subcutaneous or intramuscular dose of 0.5 mgkg⁻¹ to 20 mgkg⁻¹ of a compound of this invention, the composition being administered 1 to 4 times per day. In another embodiment a daily dose of 5 mgkg⁻¹ to 20 mgkg⁻¹ of a compound of this invention is administered. The intravenous, subcutaneous and intramuscular dose may be given by means of a bolus injection. Alternatively the intravenous dose may be given by continuous infusion over a period of time. Alternatively each patient may receive a daily oral dose which may be approximately equivalent to the daily parenteral dose, the composition being administered 1 to 4 times per day.

In the above other, pharmaceutical composition, process, method, use and medicament manufacture features, the alternative and preferred embodiments of the compounds of the invention described herein also apply.

Antibacterial Activity:

The pharmaceutically-acceptable compounds of the present invention are useful antibacterial agents having a good spectrum of activity in vitro against standard Gram-positive organisms, which are used to screen for activity against pathogenic bacteria. Notably, the pharmaceutically-acceptable compounds of the present invention show activity against enterococci pneumococci and methicillin resistant strains of S. aureus and coagulase negative staphylococci together with haemophilus and moraxella strains. The antibacterial spectrum and potency of a particular compound may be determined in a standard test system.

The (antibacterial) properties of the compounds of the invention may also be demonstrated and assessed in-vivo in conventional tests, for example by oral and/or intravenous dosing of a compound to a warm-blooded mammal using standard techniques.

The following results were obtained on a standard in-vitro test system The activity is described in terms of the minimum inhibitory concentration (MIC) determined by the agar-dilution technique with an inoculum size of 10⁴ CFU/spot. Typically, compounds are active in the range 0.01 to 256 μg/ml.

Staphylococci were tested on agar, using an inoculum of 10⁴ CFU/spot and an incubation temperature of 37° C. for 24 hours—standard test conditions for the expression of methicillin resistance.

Streptococci and enterococci were tested on agar supplemented with 5% defibrinated horse blood, an inoculum of 10⁴ CFU/spot and an incubation temperature of 37° C. in an atmosphere of 5% carbon dioxide for 48 hours—blood is required for the growth of some of the test organisms. Fastidious Gram negative organisms were tested in Mueller-Hinton broth, supplemented with hemin and NAD, grown aerobically for 24 hours at 37° C., and with an innoculum of 5×10⁴ CFU/well

For example, the following results were obtained for the compound of Example 2:

| Organism | | MIC (μg/ml) |
|---|---|---|
| Staphylococcus aureus: | MSQS | 0.5 |
| | MRQR | 0.5 |
| Streptococcus pneumoniae | | 0.13 |
| Haemophilus influenzae | | 4 |
| Moraxella catarrhalis | | 0.5 |
| Linezolid Resistant Streptococcus pneumoniae | | 1 |
| Enterococcus faecium | | 0.25 |

MSQS = methicillin sensitive and quinolone sensitive
MRQR = methicillin resistant and quinolone resistant

Certain intermediates and/or Reference Examples described hereinafter are within the scope of the invention and may also possess useful activity, and are provided as a further feature of the invention.

The invention is now illustrated but not limited by the following Examples in which unless otherwise stated:—

(i) evaporations were carried out by rotary evaporation in vacuo and work-up procedures were carried out after removal of residual solids by filtration;

(ii) operations were carried out at ambient temperature, that is typically in the range 18-26° C. and without exclusion of air unless otherwise stated, or unless the skilled person would otherwise work under an inert atmosphere;

(iii) column chromatography (by the flash procedure) was used to purify compounds and was performed on Merck Kieselgel silica (Art. 9385) unless otherwise stated;

(iv) yields are given for illustration only and are not necessarily the maximum attainable;

(v) the structure of the end-products of the invention were generally confirmed by NMR and mass spectral techniques [proton magnetic resonance spectra were generally determined in DMSO-d₆ unless otherwise stated using a Varian Gemini 2000 spectrometer operating at a field strength of 300 MHz, or a Bruker AM250 spectrometer operating at a field strength of 250 MHz; chemical shifts are reported in parts per million downfield from tetramethysilane as an internal standard (8 scale) and peak multiplicities are shown thus: s, singlet; d, doublet; AB or dd, doublet of doublets; dt, doublet of triplets; di, doublet of multiplets; t, triplet, m, multiplet; br, broad; fast-atom bombardment (FAB) mass spectral data were generally obtained using a Platform spectrometer (supplied by Micromass) run in electro spray and, where appropriate, either positive ion data or negative ion data were collected]; optical rotations were determined at 589 nm at 20° C. for 0.1M solutions in methanol using a Perkin Elmer Polarimeter 341;

(vi) each intermediate was purified to the standard required for the subsequent stage and was characterised in sufficient detail to confirm that the assigned structure was correct; purity was assessed by HPLC, TLC, or NMR and identity was determined by intra-red spectroscopy (IR), mass spectroscopy or NMR spectroscopy as appropriate;

(vii) in which the following abbreviations may be used:—

DMF is N,N-dimethylformamide; DMA is N,N-dimethylacetamide; TLC is thin layer chromatography; HPLC is high pressure liquid chromatography; MPLC is medium pressure liquid chromatography; DMSO is dimethylsulfoxide; CDCl₃ is deuterated chloroform; MS is mass spectroscopy; ESP is electrospray; EI is electron impact; CI is chemical ionisation; APCI is atmospheric pressure chemical ionisation; EtOAc is ethyl acetate; MeOH is methanol; phosphoryl is (HO)₂—P(O)—O—; phosphiryl is (HO)₂—P—O—; Bleach is “Clorox” 6.15% sodium hypochlorite; EDAC is 1-[3-(dimethylamino)propyl]-3-ethylcarbodiimide; THF is tetrahydrofuran; TFA is trifluoroacetic acid; RT is room temperature; cf.=compare (viii) temperatures are quoted as ° C.

(ix) MP carbonate resin is a solid phase resin for use in acid Scaveging, available from Argonaut Technologies, chemical structure is PS—CH₂N(CH₂CH₃)₃⁺ (CO₃²⁻)_{0.5}

### EXAMPLE 1

### (5R)-3-{4′-[5,5-bis({[tert-Butyl(dimethyl)silyl]oxy}methyl)-4,5-dihydroisoxazol-3-yl]-2-fluoro-1,1′-biphenyl-4-yl}-5-(1H-1,2,3-triazol-1-ylmethyl)-1,3-oxazolidin-2-one

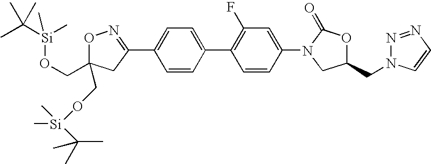

A mixture of (5R)-3-(3-fluoro-4-iodophenyl)-5-(1H-1,2,3-triazol-1-ylmethyl)-1,3-oxazolidin-2-one (388 mg, 1.00 mM), tris(dibenzylideneacetone)dipalladium (0) (37 mg, 0.040 mM, 0.04 equiv), and tri-2-furylphosphine (18 mg, 0.078 mM, 0.08 equiv) was degassed and then maintained under argon. Anhydrous N-methylpyrrolidinone (4 mL) was added to give a solution that was treated with 5,5-bis({[tert-butyl(dimethyl)silyl]oxy)methyl)-3-[4-(trimethylstannyl)phenyl]-4,5-dihydroisoxazole (718 mg, 1.20 mM) and the reaction mixture was degassed again. The reaction mixture was heated at 90° C. for ca. 64 hours, then allowed to cool. The cool reaction mixture was partitioned between ethyl acetate and water. The organic phase was dried (MgSO₄) and concentrated under vacuum to give a crude product that was purified by chromatography on silica gel [elution with 10% hexanes:ethylacetate] to give the title compound (376 mg).

MS (ESP): 696, 697 (M, M+1) for C₃₅H₅₀FN₅O₅Si₂

NMR (DMSO-d₆) δ: 0.03 (s, 6H); 0.05 (s, 6H); 0.83 (s, 18H); 3.22 (s, 2H); 3.67-3.75 (m, 4H); 3.95 (dd, 1H); 4.28 (t, 1H); 4.85 (d, 2H); 5.18 (m 1H); 7.38 (dd, 1H); 7.52-7.77 (m, 7H); 8.18 (s, 1H).

The intermediates for this compound were prepared as follows:

### 5,5-bis({[tert-Butyl(dimethyl)silyl]oxy}methyl)-3-[4-(trimethyl]stannyl)phenyl]-4,5-dihydroisoxazole

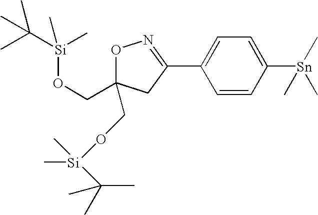

A mixture of 3-(4-bromophenyl)-5,5-bis({[tert-butyl(dimethyl)silyl]oxy}methyl)-4,5-dihydroisoxazole (2.80 g, 5.44 mM), bis(triphenylphosphine)palladium(II) chloride (190 mg, 0.27 mM), and 1,4-dioxane (20 mL) was degassed and then maintained under argon. The mixture was treated with hexamethylditin (2.00 g, 6.10 mM) and the reaction mixture was heated at 90° C. for ca. 20 hours. The reaction mixture was adsorbed onto silica gel and eluted with 10% ethyl acetate:hexanes to give the title compound (1.60 g).

MS (ESP): 598, 600 (M, M+2) for C₂₆H₄₉NO₃Si₂Sn

NMR (DMSO-d₆) δ: 0.05 (s, 6H); 0.07 (s, 6H); 0.28 (s, 18H); 0.86 (s, 18H); 3.19 (s, 2H); 3.68-3.74 (m, 4H); 7.55-7.61(m, 4H).

### 3-(4-Bromophenyl)-5,5-bis({[tert-butyl(dimethyl)silyl]oxy}methyl)-4,5-dihydroisoxazole

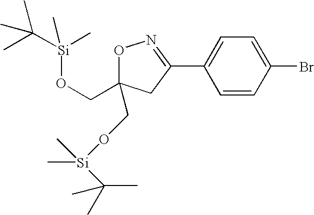

Triethylamine (2.00 mL, 14.26 mM) and then N,N-dimethylaminopyridine (290 mg, 2.38 mM) and then a solution of tert-butyldimethylsilyl chloride in dichloromethane (1.0 M, 1.31 mL, 1.31 mM) was added to a mixture of 3-(4-bromophenyl)-5,5-bis(hydroxymethyl)-4,5-dihydroisoxazole (1.70 g, 5.94 mM) and dichloromethane (20 mL). The reaction mixture was stirred at room temperature for ca. 16 h. The reaction was washed with water, dried over MgSO₄, and concentrated under vacuum. The crude material was purified by chromatography on silica gel [elution with 25% ethyl acetate:hexanes] to give the title compound (3.5 g).

MS (APCI): 514, 516 (M, M+1) for C₂₃H₄₀BrNO₃Si₂

NMR (DMSO-d₆) δ: 0.07 (s, 6H); 0.09 (s, 6H); 0.88 (s, 18H); 3.22 (s, 2H); 3.75 (d, 4H); 7.48-7.73 (m, 4H).

### 3-(4-Bromophenyl)-5,5-bis(hydroxymethyl)-4,5-dihydroisoxazole

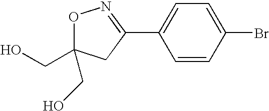

A solution of 2-methylene1,3-propanediol (2.00 g, 22.70 mM) in dichloromethane (20 mL) was treated at 0° C. with a solution of diethylzinc in hexane (1.0 M, 25.00 mL, 25.00 mM) and then slowly with a solution of 4-bromo-N-hydroxybenzenecarboximidoyl chloride in dichloromethane (20 mL). The reaction mixture was allowed to warm to room temperature and kept at room temperature for ca. 5 h. The mixture was poured into an saturated aqueous solution of ammonium chloride and extracted (twice) with dichloromethane. The combined organic phase was dried (MgSO₄) and concentrated under vacuum to give the title compound (2.1 g) that was used without further purification.

MS (APCI): 286, 288 (M, M+2) for C₁₁H₁₂BrNO₃

NMR (DMSO-d₆) δ: 3.28 (s, 2H); 3.49 (d, 4H); 5.02 (t, 2H); 7.59-7.67 (m, 4H).

### Acetic acid (5R)-3-(3-fluorophenyl)-1,3-oxazolidin-2-on-5-ylmethyl ester

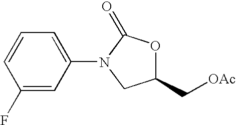

(5R)-3-(3-Fluorophenyl)-5-hydroxymethyl-1,3-oxazolidin-2-one (40 g, 0.189 M, see Upjohn WO 94-13649) was suspended by stirring in dry dichloromethane (400 mL) under nitrogen. Triethylamine (21 g, 0.208 M) and 4-dimethylaminopyridine (0.6 g, 4.9 mM) were added, followed by dropwise addition of acetic anhydride (20.3 g, 0.199 M) over 30 minutes, and stirring continued at ambient temperature for 18 hours. Saturated aqueous sodium bicarbonate (250 mL) was added, the organic phase separated, washed with 2% sodium dihydrogen phosphate, dried (magnesium sulfate), filtered and evaporated to give the desired product (49.6 g) as an oil.

MS (ESP): 254 (MH⁺) for C₁₂H₁₂FNO₄

NMR (CDCl₃) δ: 2.02 (s, 3H); 3.84 (dd, 1H); 4.16 (t, 1H); 4.25 (dd, 1H); 4.32 (dd, 1H); 4.95 (m, 1H); 6.95 (td, 1H); 7.32 (d, 1H); 7.43 (t, 1H); 7.51 (d, 1H).

### Acetic acid (5R)-3-(3-fluoro-4-iodo-phenyl)-1,3-oxazolidin-2-one-5-ylmethyl ester

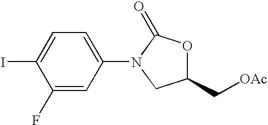

Acetic acid (5R)-3-(3-fluoro-phenyl)-1,3-oxazolidin-2-one-5-ylmethyl ester (15.2 g, 60 mM) was dissolved in a mixture of chloroform (100 mL) and acetonitrile (100 mL) under nitrogen, and silver trifluoroacetate (16.96 g, 77 mM) added. Iodine (18.07 g, 71 mM) was added in portions over 30 minutes to the vigorously stirred solution, and stirring continued at ambient temperature for 18 hours. As reaction was not complete, a further portion of silver trifluoroacetate (2.64 g, 12 mM) was added and stirring continued for 18 hours. After filtration, the mixture was added to sodium thio sulfate solution (3%, 200 mL) and dichloromethane (200 mL), and the organic phase separated, washed with sodium thiosulfate (200 mL), saturated aqueous sodium bicarbonate (200 mL), brine (200 mL), dried (magnesium sulfate), filtered and evaporated. The crude product was suspended in isohexane (100 mL), and sufficient diethyl ether added to dissolve out the brown impurity while stirring for 1 hour. Filtration gave the desired product (24.3 g) as a cream solid.

MS (ESP): 380 (MH⁺) for C₁₂H₁₁FINO₄

NMR (DMSO-d₆) δ: 2.03 (s, 3H); 3.82 (dd, 1H); 4.15 (t, 1H); 4.24 (dd, 1H); 4.30 (dd, 1H); 4.94 (, 1H); 7.19 (dd, 1H); 7.55 (dd, 1H); 7.84 (t, 1H).

### (5R)-3-(3-Fluoro-4-iodophenyl)-5-hydroxymethyl-1,3-oxazolidin-2-one

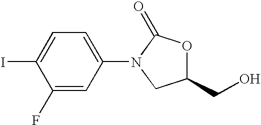

Acetic acid (5R)-3-(3-fluoro-4-iodophenyl)-1,3-oxazolidin-2-one-5-ylmethyl ester (30 g, 79 mM) was treated with potassium carbonate (16.4 g, 0.119 mM) in a mixture of methanol (800 mL) and dichloromethane (240 mL) at ambient temperature for 25 minutes, then immediately neutralised by the addition of acetic acid (10 mL) and water (500 mL). The precipitate was filtered, washed with water, and dissolved in dichloromethane (1.2 L), the solution washed with saturated sodium bicarbonate, and dried (magnesium sulfate). Filtration and evaporation gave the desired product (23 g).

MS (ESP): 338 (MH⁺) for C₁₀H₉FINO₃

NMR (DMSO-d₆) δ: 3.53 (m, 1H); 3.67 (m, 1H); 3.82 (dd, 1H); 4.07 (t, 1H); 4.70 (m, 1H); 5.20 (t, 1H); 7.21 (dd, 1H); 7.57 (dd, 1H); 7.81 (t, 1H).

### (5R)-5-Azidomethyl-3-(3-fluoro-4-iodophenyl)-1,3-oxazolidin-2-one

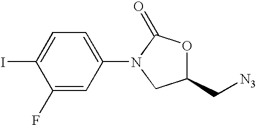

Methanesulfonyl chloride (17.9 mL) was added dropwise to a stirred solution of (5R)-3-(3-fluoro-4-iodophenyl)-5-hydroxymethyl-1,3-oxazolidin-2-one (55.8 g) and triethylamine (46.1 mL) in dry dichloromethane (800 mL) under an atmosphere of dry nitrogen and maintained below room temperature by an ice-bath. The stirred reaction mixture was allowed to warm to room temperature during 3 hours and then washed sequentially with water and brine and then dried (Na₂SO₄). Solvent was removed under reduced pressure to give the intermediate mesylate as a yellow solid (68 g) that was used without further purification.

A stirred solution in DMF (800 mL) of a mixture of the intermediate mesylate (68 g) and sodium azide (32.3 g) was heated at 75° C. overnight. The mixture was allowed to cool to room temperature, diluted with water, and extracted twice with ethyl acetate. The combined extracts were washed sequentially with water and brine, and then dried (Na₂SO₄). Solvent was removed under reduced pressure to give a yellow oil that was purified by column chromatography on silica-gel [elution with ethyl acetate:hexanes (1:1)) to give the product azide as an off-white solid (49 g). The product could be further purified by trituration with ethyl acetate/hexanes.

¹H-NMR (DMSO-d₆) δ: 3.57-3.64 (dd, 1H); 3.70-3.77 (dd, 1H); 3.81-3.87 (dd, 1); 4.06 (t, 1H); 4.78-4.84 (m, 1H); 7.05-7.09 (ddd, 1H); 7.45 (dd, 1H); 7.68-7.74 (dd, 1H).

### (5R)-3-(3-Fluoro-4-iodophenyl)-5-(1H-1,2,3-triazol-1-ylmethyl)-1,3-oxazolidin-2-one

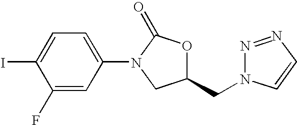

A stirred solution in dioxan (300 mL) of a mixture of the (5R)-5-azidomethyl-3-(3-fluoro-4-iodophenyl)-1,3-oxazolidin-2-one (30 g) and bicyclo[2.2.1]heptadiene (30 mL) was heated under reflux overnight. The mixture was allowed to cool to room temperature and then evaporated to dryness under reduced pressure to give a brown solid. The brown solid was purified by column chromatography on silica-gel [elution with a gradient from 98:2 to 95:5 methanol:chloroform] to give the product triazole as a pale yellow solid (20 g). The product could be further purified by trituration with dichloromethane/hexanes (1:1) to give an off-white solid.

¹H-NMR (DMSO-d₆) δ: 3.86-3.92 (dd, 1H); 4.23 (t, 1H); 4.83 (d, 2H); 5.11-5.19 (m, 1H); 7.12-7.16 (dd, 1H); 7.47-7.51 (dd, 1H); 7.76 (s, 1H); 7.79-7.85 (dd, 1H); 8.16 (s, 1H).

### EXAMPLE 2

### (5R)-3-{4′-(5,5-bis(Hydroxymethyl)-4,5-dihydroisoxazol-3-yl]-2-fluoro-1,1′-biphenyl-4-yl}-5-(1H-1,2,3-triazol-1-ylmethyl)-1,3-oxazolidin-2-one

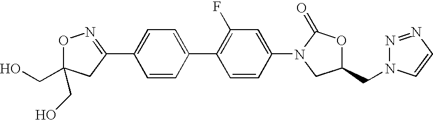

A solution of tetrabutylammonium fluoride (TBAF) in THF (1.0 M, 1.62 mL, 1.62 mM) was added to a solution of (5R)-3-{4′-[5,5-bis(([tert-butyl(dimethyl)silyl]oxy}methyl)-4,5-dihydroisoxazol-3-yl]-2-fluoro-1,1′-biphenyl-4-yl}-5-(1H-1,2,3-triazol-1-ylmethyl)-1,3-oxazolidin-2-one (376 mg, 0.54 mM) in THF (4 mL). The reaction mixture was stirred at room temperature for 3 h and then water was added. The mixture was extracted with ethyl acetate and the organic phase dried (MgSO₄) and concentrated under vacuum. The crude product was purified by chromatography on silica gel [elution with 5% methanol:ethyl acetate] to give the title compound (116 mg).

MS (ESP): 468 (M+1) for C₂₃H₂₂FN₅O₅

NMR (DMSO-d₆) δ: 3.27 (s, 2H); 3.51 (d, 4H); 3.97 (dd, 1H); 4.30 (t, 1H); 4.87 (d, 2H); 5.03 (t, 2H); 5.19 (m, 1H); 7.39 (dd, 1H); 7.53-7.78 (m, 7H); 8.19 (s, 1H).

### EXAMPLE 3

### (5R)-3-{4′-[5,5-bis({[tert-Butyl(dimethyl)silyl]oxy}methyl)-4,5-dihydroisoxazol-3-yl]-2-fluoro-1,1′-biphenyl-4-yl}-5-[(4-methyl-1H-1,2,3-txiazol-1-yl)methyl]-1,3-oxazolidin-2-one

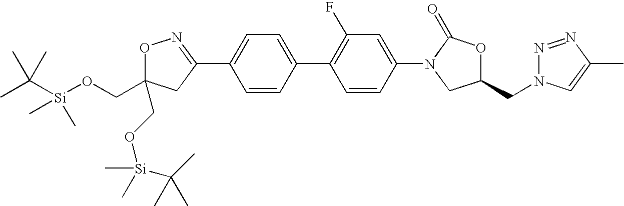

The title compound was prepared from 5,5-bis({[tert-butyl(dimethyl)silyl]oxy}methyl)-3-[4-(trimethylstannyl)phenyl]-4,5-dihydroisoxazole (900 mg, 1.50 mM) and (5R)-3-(3-fluoro-4-iodophenyl)-5-[(4-methyl-1H-1,2,3-triazol-1-yl)methyl]-1,3-oxazolidin-2-one (402 mg, 1.00 mM) using essentially the same procedure as that described for Example 1, (200 mg).

MS (ESP): 710, 711 (M, M+1) for C₃₆H₅₂FN₅O₅Si₂

NMR (DMSO-d₆) δ: 0.03 (s, 6H); 0.05 (s, 6H); 0.83 (18H); 2.22 (s, 3H); 3.22 (s, 2H); 3.67-3.75 (m, 4H); 3.93 (dd, 1H); 4.27 (t, 1H); 4.75 (d, 2H); 5.13 (m, 1H); 7.39 (dd, 1H); 7.53-7.75 (m, 6H); 7.87 (s, 1H).

The intermediates for this compound were prepared as follows:

### (5R)-3-(3-Fluorophenyl)-5-[(4-methyl-1H-1,2,3-triazol-1-yl)methyl]-1,3-oxazolidin-2-one

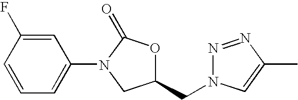

A stirred solution of N,N-diisopropylethylamine (3.20 mL, 18.35 mM) and (5S)-5-(aminomethyl)-3-(3-fluorophenyl)-1,3-oxazolidin-2-one (0.77 g, 3.57 mM, see Dong Pharmaceuticals WO 0194342) in anhydrous methanol (25 mL) was treated at 0° C. with N′-[2,2-dichloro-1-methylethylidene]-4-methylbenzenesulfonohydrazide (1.28 g, 4.58 M). The reaction mixture was allowed to warm and stirred at room temperature overnight. The reaction mixture was then concentrated under vacuum to give a crude product was purified by chromatography on silica gel [elution with 2% methanol:dichloromethane] to give the title compound (0.71 g).

MS (ESP): 277 (M+1) for C₁₃H₁₃FN₄O₂

NMR (DMSO-d₆) δ: 2.24 (s, 3H); 3.90 (dd, 1H); 4.25 (t, 1H); 4.77 (d, 2H); 5.13 (m, 1H); 6.99 (m, 1H); 7.28 (d, 1H); 7.42-7.48 (m, 2H); 7.89 (s, 1H).

### (5R)-3-(3-Fluoro-4-iodophenyl)-5-[(methyl-1H-1,2,3-triazol-1-yl)methyl]-1,3-oxazolidin-2-one

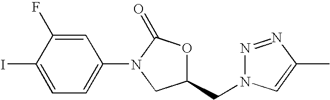

Iodine (0.55 g, 2.17 mM) was added over 1.5 h to a mixture of silver trifluoroacetate (0.52 g, 2.35 mM) and a solution of (5R)-3-(3-fluorophenyl)-5-[(4-methyl-1H-1,2,3-triazol-1-yl)methyl]-1,3-oxazolidin-2-one (0.50 g, 1.81 mM) in dichloromethane (15 mL). The reaction mixture was stirred overnight and then the precipitated solids were isolated from the reaction mixture by filtration. The filtrate was treated with additional portions of silver trifluoroacetate (0.38 g, 1.72 mM) and iodine (0.27 g, 1.06 mM), and refiltered after an additional 24 h. The retained solid from the filtrations was extracted with methanol and the methanol extract was concentrated under vacuum to give the title compound (0.31 g).

MS (ESP): 403 (M+1) for C₁₃H₁₂FIN₄O₂

NMR (DMSO-d₆) δ: 2.24 (s, 3H); 3.89 (dd, 1H); 4.23 (t, 1H); 4.76 (d, 2H); 5.12 (m, 1H); 7.17 (dd, 1H); 7.51 (dd, 1H); 7.84 (t, 1H); 7.88 (s, 1H).

### EXAMPLE 4

### (5R)-3-{4′-5,5-bis(Hydroxymethyl)-4,5-dihydroisoxazol-3-yl]-2-fluoro-1,1′-biphenyl-4-yl}-5-[(4-methyl-1H-1,2,3-triazol-1-yl)methyl]-1,3-oxazolidin-2-one

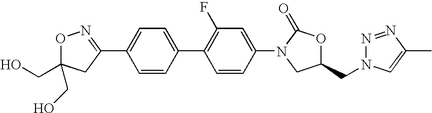

The title compound was obtained from (5R)-3-{4′-[5,5-bis({[tert-butyl(dimethyl)silyl]oxy}methyl)-4,5-dihydroisoxazol-3-yl]-2-fluoro-1,1′-biphenyl-4-yl}-5-[(4-methyl-1H-1,2,3-triazol-1-yl)methyl]-1,3-oxazolidin-2-one (200 mg, 0.28 mM) using essentially the same procedure as that described for Example 1, (49 mg)

MS (ESP): 482 (M+1) for C₂₄H₂₄FN₅O₅

NMR (DMSO-d₆) δ: 2.23 (s, 3H); 3.26-3.33 (2H, overlapping with H₂O peak); 3.51(d, 4H); 3.94(dd, 1H); 4.28 (t, 1H); 4.78 (d, 2H); 5.04 (t, 2H); 5.14 (m, 1H); 7.40 (dd, 1H); 7.54-7.77 (m, 6H); 7.89 (s, 1H).

### EXAMPLE 5

### N-[((5S)-3-{4′-[5,5-bis(Hydroxymethyl)-4,5-dihydroisoxazol-3-yl]-1,1′-biphenyl-4-yl}-2-oxo-1,3-oxazolidin-5-yl)methyl]acetamide

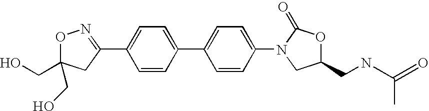

The title compound was obtained from (5S)-3-{4′-[5,5-bis({[tert-butyl(dimethyl)silyl]oxy}methyl)-4,5-dihydroisoxazol-3-yl]-2-fluoro-1,1′-biphenyl-4-yl}-5-acetamidomethyl)-1,3-oxazolidin-2-one using essentially the same procedure as Example 2 (93 mg).

MS (ESP): 440, 441 (M, M+1) for C₂₃H₂₅N₃O₆

NMR (DMSO-d₆) δ: 1.84 (s, 3H); 3.26 (s, 2H); 3.44 (t, 2H); 3.51 (d, 4H); 3.80 (dd, 1H); 4.18 (t, 1H); 4.75 (m, 1H); 5.03 (t, 2H); 7.64-7.80 (m, 8H); 8.28 (t, 1H).

The starting material for this compound were prepared from (5S)-3-(3-fluoro-4-iodophenyl)-5-(acetamidomethyl)-1,3-oxazolidin-2-one and 3-(4-bromophenyl)-5,5-bis({[tert-butyl(dimethyl)silyl]oxy}methyl)-4,5-dihydroisoxazole using essentially the same procedure as that described for Example 1

### EXAMPLE 6

### [3-(2′-Fluoro-4′-{(5R)-5-[(4-methyl-1H-1,2,3-triazol-1-yl)methyl]-2-oxo-1,3-oxazolidin-3-yl}-1,1′-biphenyl-4-yl)-4,5-dihydroisoxazol-5-yl]acetonitrile

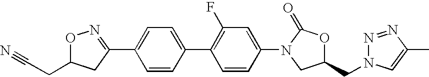

The title compound was obtained from (5R)-3-[3-fluoro-4-(trimethylstannyl)phenyl]-5-[(4-methyl-1H-1,2,3-triazol-1-yl)methyl]-1,3-oxazolidin-2-one (0.98 g, 2.23 mM and [3-(4-bromophenyl)-4,5-dihydroisoxazol-5-yl]acetonitrile (0.40 g, 1.51 mM) using essentially the same procedure as that described for Example 1, (30 mg).

MS (ESP): 461 (M+1) for C₂₄H₂₁FN₆O₃

NMR (DMSO-d₆) δ: 2.22 (s, 3H); 2.97 (dd, 2H); 3.22-3.27 (m overlapping with H₂O, 1H); 3.68 (dd, 1H); 3.93 (dd, 1H); 4.27 (t, 1H); 4.77 (d, 2H); 5.03 (m, 1H); 5.13 (m, 1H); 7.39 (dd, 1H); 7.53-7.79 (m, 1H); 7.87 (s, 1H).

The intermediates for this compound were prepared as follows:

### [3-(4-Bromophenyl)-4,5-dihydroisoxazol-5-yl]methyl methanesulfonate

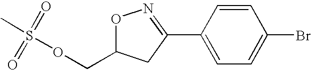

A solution of [3-(4-bromophenyl)-4,5-dihydroisoxazol-5-yl]-methanol (84.30 g, 0.33 M) (AstraZeneca WO 01/40222 A1) in anhydrous dichloromethane (500 mL) was maintained at 0° C. and treated with triethylamine (64.10 mL, 0.46 M) and then dropwise methanesulfonyl chloride (30.65 mL, 0.40 M). The reaction mixture was stirred for 2 hours at 0° C. and then treated with aqueous sodium bicarbonate (200 mL). The phases were separated and the aqueous phase was extracted with dichloromethane (2×200 mL). The organic phases were combined, dried (sodium sulfate) and concentrated in vacuo to give the title compound (110 g) sufficiently pure for further use.

NMR (DMSO-d₆) δ: 3.08 (s, 3H); 3.27 (dd, 1H); 3.47 (dd, 1H); 4.37 (m, 2H); 5.02 (m, 1H); 7.53 (m, 4H).

### [3-(4-Bromophenyl)-4,5-dihydroisoxazol-5-yl]acetonitrile

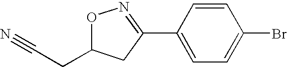

A mixture of [3-(4-bromophenyl)-4,5-dihydroisoxazol-5-yl]methyl methanesulfonate (0.50 g, 1.50 mM, sodium cyanide (0.15 g, 3.00 mM), and N,N-dimethylformamide was heated at 75° C. for ca. 16 h. The reaction mixture was diluted with ethyl acetate and the washed with water. The organic phase was dried (MgSO₄) and concentrated under vacuum to give the title compound (0.40 g) sufficiently pure for further use.

MS (ESP): 265, 267 (M, M+2) for C₁₁H₉BrN₂O

NMR (DMSO-d₆) δ: 2.94-2.97 (m, 2H); 3.21 (dd, 1H); 3.63 (dd, 1H); 5.00 (m, 1H); 7.60-7.68 (m, 4H).

### (5R)-3-[3-Fluoro-4-(trimethylstannyl)phenyl]-5-[(4-methyl-1H-1,2,3-triazol-1-yl)methyl]-1,3-oxazolidin-2-one

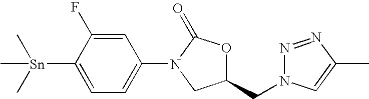

A mixture of (5R)-3-(3-fluoro-4-iodophenyl)-5-[(4-methyl-1H-1,2,3-triazol-1-yl)methyl]-1,3-oxazolidin-2-one (5.12 g, 12.70 mM) and bis(triphenylphospine)palladium(II)chloride (0.45 g, 0.05 mM) was degassed and maintained under argon. The reaction mixture was treated with dioxane (50 mL) and then with hexamethylditin (5.00 g, 15.30 mM) and the reaction was degassed again and maintained under argon. The reaction mixture was heated at 90° for 20 hours. The cool reaction mixture was adsorbed onto silica-gel, and purified by flash chromatography [elution with a gradient from 50% hexanes:ethyl acetate to 100% ethyl acetate] to give the title compound (3.91 g).

MS (ESP): 440 (MH⁺) for C₁₆H₂₁FN₄O₂Sn

NMR (DMSO-d₆) δ: 0.09 (t, 9H); 2.00 (s, 3H); 3.65 (dd, 1H); 4.00 (t, 1H); 4.53 (d, 2H); 4.88 (m, 1H); 7.03 (dd, 1H); 7.11 (dd, 1H); 7.18 (dd, 1H); 7.64 (s, 1H).

### EXAMPLE 7

### (5R)-3-[4′-(4,5-dihydroisoxazol-3-yl)-2-fluoro-1,1′-biphenyl-4-yl]-5-[(4-methyl-1H-1,2,3-triazol-1-yl)methyl]-1,3-oxazolidin-2-one

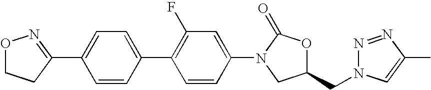

The title compound was prepared from (5R)-3-(3-fluoro-4-iodophenyl)-5-[(4-methyl-1H-1,2,3-triazol-1-yl)methyl]-1,3-oxazolidin-2-one (603 mg, 1.50 mM) and 3-[4-(trimethylstannyl)phenyl]-4,5-dihydroisoxazole (558 mg, 1.80 mM) using essentially the same procedure as that used for Example 1, (394 mg).

MS (ESP): 422 (M+1) for C₂₂H₂₀FN₅O₃

NMR (DMSO-d₆) δ: 2.22 (s, 3H); 3.41 (t, 2H); 3.92 (dd, 1H); 4.27 (t, 1H); 4.40 (t, 2H); 4.77 (d, 2H); 5.13 (m, 1H); 7.39 (dd, 1H); 7.53-7.81 (m, 6H); 7.88 (s, 1H).

The intermediate for this compound was prepared as follows:

### 3-[4-(Trimethylstannyl)phenyl]-4,5-dihydroisoxazole

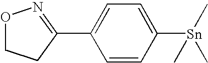

A solution of 3-(4-bromophenyl)-4,5-dihydroisoxazole (1.40 g, 6.19 mM) in 1,4-dioxane (30 mL) (P. L. Scott; A. F. Hagarty, R. J. MacConaill, Tetrahedron Lett., 1972, 13, 1213) was treated with bis(triphenylphosphine)palladium(II) chloride (217 mg, 0.31 mM) and the solution was degassed and maintained under argon. The mixture was treated with hexamethylditin (3.00 g, 9.16 mM) and the reaction mixture was heated at 90° C. for ca. 20 hours. The reaction mixture was adsorbed onto silica-gel and purified by chromatography [elution with a gradient from 5% to 10% ethyl acetate:hexanes] to give the title compound (1.70 g).

MS APCI): 310, 312 (M, M+2) for C₁₂H₁₇NOSn

NMR (DMSO-d₆) δ: 0.27 (s, 9H); 3.29-3.38 (m, 2H overlapping with H₂O); 4.36 (t, 2H); 7.54-7.63) m, 4H).

### EXAMPLE 8

### (5R)-3-[4′-(4,5-dihydroisoxazol-3-yl)-2-fluoro-1,1′-biphenyl-4-yl]-5-(1H-1,2,3-triazol-1-ylmethyl)-1,3-oxazolidin-2-one

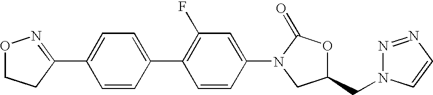

The title compound was prepared from (5R)-3-(3-fluoro-4-iodophenyl)-5-(1H-1,2,3-triazol-1-ylmethyl)-1,3-oxazolidin-2-one (582 mg, 1.50 mM) and 3-[4-(trimethylstannyl)phenyl]-4,5-dihydroisoxazole (558 mg, 1.80 mM) using essentially the same procedure as that used for Example 1, (176 mg).

MS APCI): 408 (M+1) for C₂₁H₁₈FN₅O₃

NMR (DMSO-d₆) δ: 3.41 (t, 2H); 3.95 (dd, 1H); 4.29 (t, 1H); 4.40 (t, 2H); 4.86 (d, 2H); 5.18 (m, 1H); 7.38 (dd, 1H); 7.52-7.78 (m, 7H); 8.18 (s, 1H).

### EXAMPLE 9

### (5R)-3-{4′-[5,5-Bis(hydroxymethyl)-4,5-dihydroisoxazol-3-yl]-2,2′-difluoro-1,1′-biphenyl-4-yl}-5-(1H-1,2,3-triazol-1-ylmethyl)-1,3-oxazolidin-2-one

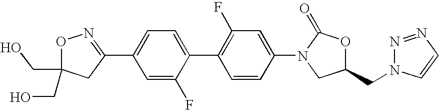

(5R)-3-{4′-[5,5-Bis({[tert-butyl(dimethyl)silyl]oxy}methyl)-4,5-dihydroisoxazol-3-yl]-2,2′-difluoro-1,1′-biphenyl-4-yl}-5-(1H-1,2,3-triazol-1-ylmethyl)-1,3-oxazolidin-2-one (0.691 g, 0.968 mM) was dissolved in tetrahydrofuran (5 ml) and a 1 N solution of tetrabutylamnxnonium fluoride in tetrahydrofuran (0.2 mL) was added. The reaction was stirred at room temperature for fifteen minutes. Water was added resulting in a white precipitate that was filtered. The solid was dissolved in acetone and hexanes was added resulting in a precipitate. The desired product was collected as an off-white solid (0.185 g).

MS (ESP): 486 (MH⁺) for C₂₃H₂₁F₂N₅O₅

300 MHz NMR (DMSO-d₆) δ: 3.51 (s, 2H); 3.52 (s, 2H); 3.97 (dd, 1H); 4.31 (t, 1H); 4.87 (d, 2H); 5.05 (t, 2H); 5.15-5.23 (m, 1H); 7.41 (dd, 1H); 7.49-7.62 (m, 5H); 7.78 (s, 1H); 8.20 (s, 1H).

The intermediates for the above were prepared as follows:

### (5R)-3-{4′-[5,5-Bis({[tert-butyl(dimethyl)silyl]oxy}methyl)-4,5-dihydroisoxazol-3-yl]-2,2′-difluoro-1,1′-biphenyl-4-yl}-5-(1H-1,2,3-triazol-1-ylmethyl)-1,3-oxazolidin-2-one

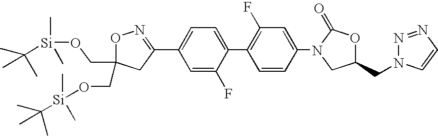

3-(4-Bromo-3-fluorophenyl)-5,5-bis({[tert-butyl(dimethyl)silyl]oxy}methyl)-4,5-dihydroisoxazole (0.694 g, 2.17 mM), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (see Example 13, 0.561 g, 1.45 mM, potassium carbonate (0.651 g, 4.64 mM), and tetrakis(triphenylphosphine)palladium(0) (0.168 g, 0.145 mM) were combined and the flask was degassed. N,N-Dimethylformamide (5 mL) and water (0.5 mL) were added and the reaction was heated to 80° C. for four hours. The mixture was concentrated then chromatographed using 50-75% ethyl acetate/hexanes. Relevant fractions were collected and concentrated to give the desired product as a light yellow solid (0.691 g).

MS (ESP): 714 (MH⁺) for C₃₅H₄₉F₂N₅O₅Si₂

300 MHz NMR (DMSO-d₆) δ: 0.00 (s, 6H); 0.02 (s, 6H); 0.78 (s, 18H); 3.13-3.37 (hidden by water peak, 3H); 3.68 (bs, 3H); 3.92 (m, 1H); 4.26 (t, 1H); 4.81 (d, 2H); 5.13 (m, 1H); 7.41-7.64 (m, 6H); 7.90 (s, 1H); 8.15 (s, 1H).

### 3-(4-Bromo-3-fluorophenyl)-5,5-bis({[tert-butyl(dimethyl)silyl]oxy}methyl)-4,5-dihydroisoxazole

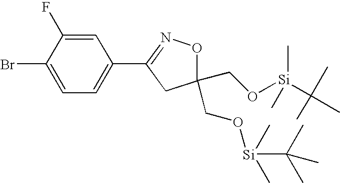

3-(4-Bromo-3-fluorophenyl)-5,5-bis(hydroxymethyl)-4,5-dihydroisoxazole (0.50 g, 1.56 mM) was stirred in dichloromethane (5 mL). 4-(Dimethylamino) pyridine (0.039 g, 0.312 mM) and triethylamine (0.380 g, 3.74 mM) was added. A 1 N solution of tert-butyldimethylsilyl chloride in dichloromethane (0.512 g, 3.44 mM) was added dropwise and the reaction was stirred overnight. The yellow solution was diluted with water and extracted using dichloromethane. The organic layer was dried (magnesium sulfate), filtered and concentrated. The light yellow oil was chromatographed using 50% ethyl acetate/hexanes. Desired fractions were collected and concentrated to give the title compound as a white solid (0.694 g).

MS (ESP): (MH⁺) for C₂₃H₃₉BrFNO₃Si₂

300 MHz NMR (DMSO-d₆) δ: 0.02 (s, 6H); 0.04 (s, 6H); 0.81 (s, 18H); 3.18 (s, 2H); 3.69 (d, 4H); 7.44 (dd, 1H); 7.62 (dd, 1H); 7.77 (t, 1H).

### 3-(4-Bromo-3-fluorophenyl)-5,5-bis(hydroxymethyl)-4,5-dihydroisoxazole

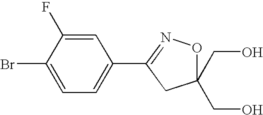

2-Methylene-1,3-propanediol (2.20 g, 25.0 mM) was stirred in dichloromethane (20 mL) and cooled to 0° C. A 1 N solution of diethylzinc in hexanes (3.40 g, 27.5 mM) was added followed by a solution of 4-bromo-3-fluoro-N-hydroxybenzenecarboximidoyl chloride (6.30 g, 25.0 mM) in dichloromethane (40 mL). The reaction was allowed to warm to room temperature and was complete after four hours. The solution was diluted with ammonium chloride and extracted using dichloromethane. The organic layer was dried (magnesium sulfate), filtered and concentrated to give the desired product as a yellow solid (4.72 g).

MS (ESP): 305 (MH+) for C₁₁H₁₁BrPNO₃

300 MHz NMR (DMSO-d₆) δ: 3.29 (s, 2H); 3.55 (s, 2H); 3.57 (s, 2H); 5.10 (t, 2H); 7.52 (d, 1H); 7.68 (d, 1H); 7.86 (t, 1H).

### 4-Bromo-3-fluorobenzaldehyde oxime

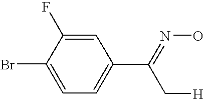

4-Bromo-3-fluorobenzaldehyde oxime 4-Bromo-3-fluorobenzaldehyde (4.06 g, 20 mM) was dissolved in methanol (30 mL) and water (30 mL). Hydroxylamine hydrochloride (2.65 g, 25 mM) was added followed by sodium carbonate (0.834 g, 12 mM) in water (30 mL). The reaction was stirred at room temperature overnight. The white slurry was extracted using ethyl acetate to give a yellow solution. The organic layer was dried (magnesium sulfate), filtered and concentrated to give the desired product as a yellow solid (4.36 g).

MS (ESP): 220 (MH⁺) for C₇H₅BrFNO

300 MHz NMR (DMSO-d₆) δ: 7.38 (s, 1H); 7.55 (d, 1H); 7.74 (t, 1H); 8.15 (s, 1H); 11.52 (s, 1H).

### 4-Bromo-3-fluoro-N-hydroxybenzenecarboximidoyl chloride

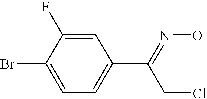

4-Bromo-3-fluorobenzaldehyde oxime (4.36 g, 20 mM) was dissolved in DMF (16 mL). Hydrogen chloride gas was bubbled into the reaction for several minutes, then N-chlorosuccinimide (2.93 g, 22 mM) was added in portions to the reaction mixture. The mixture was stirred overnight. The yellow solution was diluted with water and extracted using ethyl acetate. The organic layer was washed with water several times, dried (magnesium sulfate), filtered and concentrated to give the desired product as a light yellow solid (4.96 g).

300 MHz NMR (DMSO-d₆) δ: 7.54 (d, 1H); 7.68 (d, 1H); 7.81 (t, 1H); 7.93 (s, 1H).

### EXAMPLE 10

### (5R)-3-{4′-[5,5-Bis(hydroxymethyl)-4,5-dihydroisoxazol-3-yl]-2,2′-difluoro-1,1′-biphenyl-4-yl}-5-{[4-(fluoromethyl)-1H-1,2,3-triazol-1-yl]methyl-1,3-oxazolidin-2-one

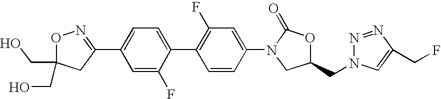

Using essentially the same procedure as Example 9 above but starting with (5R)-3-{4′-[5,5-bis({[tert-butyl(dimethyl) silyl]oxy}methyl)-4,5-dihydroisoxazol-3-yl]-2,2′-difluoro-1,1′-biphenyl-4-yl}-5-{[4-(fluoromethyl)-1H-1,2,3-triazol-1-yl]methyl}-1,3-oxazolidin-2-one (0.523 g, 0.7 mM) gave the title compound as a light brown solid (0.170 g).

MS (ESP): 518 (MH⁺) for C₂₄H₂₂F₃N₅O₅

300 MHz NMR (DMSO-d₆) δ: 3.56 (s, 4H); 3.99-4.04 (m, 1H); 4.36 (t, 1H); 4.94 (d, 2H); 5.10-5.24 (m, 3H); 5.44 (s, 1H); 5.60 (s, 1H); 7.45-7.66 (m, 6H); 8.44 (d, 1H).

The intermediate for the above was prepared as follows:

### (5R)-3-{4′-[5,5-Bis({[tert-butyl(dimethyl)silyl]oxy}methyl)-4,5-dihydroisoxazol-3-yl]-2,2′-difluoro-1,1′-biphenyl-4-yl}-5-{[4-(fluoromethyl)-1H-1,2,3-triazol-1-yl]methyl}-1,3-oxazolidin-2-one

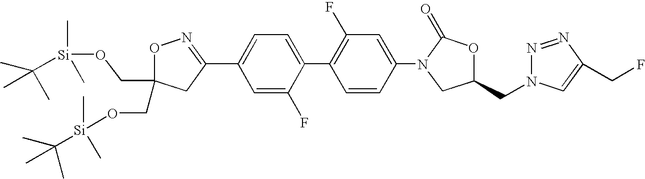

Using essentially the same procedure as the intermediate for Example 1, but starting with (5R)-5-{[4-(fluoromethyl)-1H-1,2,3-triazol-1-yl]methyl}-3-[3-fluoro-4-(4,4,5,5-tetramethyl-1,3,2-dioxaborolan-2-yl)phenyl]-1,3-oxazolidin-2-one (0.661 g, 1.55 mM) gave the title compound as a light yellow solid (0.523 g).

MS (ESP): (MH⁺) for C₃₆H₅₀F₃N₅O₅Si₂

300 MHz NMR (DMSO-d₆) δ: 0.04 (s, 6H); 0.05 s, 6H); 0.83 (s, 18H); 3.22 (s, 1H); 3.71 (bs, 3H); 3.93 (s, 2H); 3.96 (m, 1H); 4.31 (m, 1H); 4.87 (d, 2H); 5.18 (m, 1H); 5.38 (s, 1H); 5.54 (s, 1H); 7.46 (d, 1H); 7.51-7.64 (m, 5H); 8.38 (d, 1H).

### EXAMPLE 11

### N-[{(5S)-3-(4-{6-[5-(Chloromethyl)-4,5-dihydroisoxazol-3-yl]pyridin-3-yl}-3-fluorophenyl)-2-oxo-1,3-oxazolidin-5-yl]methyl}acetamide

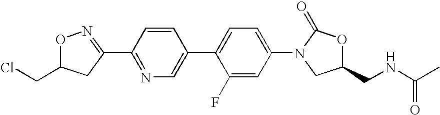

N-{[(5S)-3-(4-{6-[5-(Hydroxymethyl)-4,5-dihydroisoxazol-3-yl]pyridin-3-yl}-3-fluorophenyl)-2-oxo-1,3-oxazolidin-5-yl]methyl)acetamide (Example 60, WO2003/022824) (320 mg, 0.75 mM), and triphenylphosphine (293 mg, 1.12 mM) were suspended in 10 ml of acetonitrile. Carbon tetrachloride (0.7 ml, 7.27 mM) was added and the mixture was heated at 65° C. for 30 min. to give a clear solution. The mixture was cooled to room temperature and submitted directly to chromatography (silica gel; elution with 4% methanol in dichloromethane) to give the title compound as an off-white solid (186 mg).

MS (electrospray): 447 (M+1) for C₂₁H₂₀ClN₄O₄F

¹H-NMR (300 MHz, DMSO-d₆) δ: 1.83 (s, 3H); 3.41 (m, 3H); 3.63 (dd, 1H); 3.86 (m, 3H); 4.18 (t, 1H); 4.76 (m, 1H); 5.07 (m, 1H); 7.47 (bd, 1H); 7.65 (dd, 1H); 7.72 (d, 1H); 8.00 (d, 1H); 8.07 (d, 1H); 8.26 (t, 1H); 8.83 (brs, 1H).

### EXAMPLE 12

### N-{[(5S)-3-(3-Fluoro-4-{6-[5-(morpholin-4-ylmethyl)-4,5-dihydroisoxazol-3-yl]pyridin-3-yl}phenyl)-2-oxo-1,3-oxazolidin-5-yl]methyl}acetamide

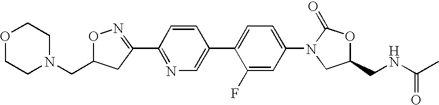

N-{[(5S)-3-(4-{6-[5-(Chloromethyl)-4,5-dihydroisoxazol-3-yl]pyridin-3-yl}-3-fluorophenyl)-2-oxo-1,3-oxazolidin-5-yl]methyl}acetamide (Example 11) (150 mg, 0.34 mM), morpholine (0.3 ml, 3.43 mM) and tetrabutylammonium iodide (5 mg, 0.014 mM) were dissolved in anhydrous DMSO (1 ml) and heated at 95° C. for 1 day. The mixture was cooled to room temperature then diluted with acetonitrile (5 ml) and diethyl ether (5 ml). The precipitate was collected, rinsed with diethyl ether and dried in vacuo to give the title compound (125 mg).

MS (electrospray): 498(M+1) for C₂₅H₂₈N₅O₅F

¹H-NMR (300 MHz DMSO-d₆) δ: 1.83 (s, 3H); 2.55 (m, 2H); 3.23 (m, 2H); 3.38-3.60 (m, 8H); 3.78 (dd, 1H); 4.18 (t, 1H); 4.77 (m, 1H); 4.94 (m, 1H); 7.47 (dd, 1H); 7.65 (dd, 1H); 7.72 (d, 1H); 7.99 (d, 1H); 8.06 (dd, 1); 8.26 (t, 1H); 8.82 (brs, 1H).

### REFERENCE EXAMPLE 13

### (5R)-3-(3-fluoro-4-{6-[5-(hydroxymethyl)-4,5-dihydroisoxazol-3-yl]-1-oxidopyridin-3-yl}phenyl)-5-(1H-1,2,3-triazol-1-ylmethyl)-1,3-oxazolidin-2-one

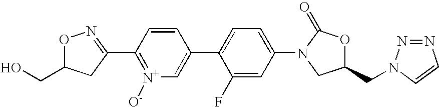

[3-(5-Bromo-1-oxidopyridin-2-yl)-4,5-dihydroisoxazol-5-yl]methanol (150 mg, 0.55 mMol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (320 mg, 0.82 mMol), potassium carbonate (300 mg, 2.17 mMol), and tetrakis(triphenylphosphino)palladium(0) (63 mg, 0.05 mMol) were combined and suspended in THF (5 ml) and water (0.5 ml). The mixture was heated at 75° C. for 3 hours, then diluted with ethyl acetate and water. The solids were collected on a filter, rinsed with ethyl acetate, then water and dried in vacuo to give the pure product as a tan solid, 115 mg.

MS (electrospray): 455 (M+1) for C₂₁H₁₉FN₆O₅

¹H-NMR (300 MHz, DMSO-d₆) δ: 3.39-3.66 (m, 4H); 3.95 (dd, 1H); 4.29 (t, 1H); 4.77 (m, 1H); 4.86 (d, 2H); 5.02 (t, 1H); 5.18 (m, 1H); 7.41 (dd, 1H); 7.55-7.62 (m, 2H); 7.68-7.83 (m, 3H); 8.18 (s, 1H); 8.53 (s, 1H).

The intermediates for the above compound were prepared as follows:

### [3-(5-bromo-1-oxidopyridin-2-yl)-4,5-dihydroisoxazol-5-yl]methanol

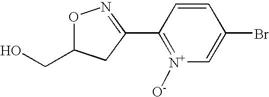

[3-(5-Bromopyridin-2-yl)-4,5-dihydroisoxazol-5-yl]methanol (0.5 g, 1.94 mMol) was dissolved in dichloromethane (10 ml) and 3-chloroperbenzoic acid (wet, 70%: 0.77 g, 4.05 mMol) was added. The mixture was warned to 40° C. for 1 hour, then an additional portion of 3-chloroperbenzoic acid (wet, 70%: 0.77 g, 4.05 mMol) was added followed by continued heating at 40° C. for 3 hours. The solution was concentrated and purified by chromatography (silica gel; elution with 25 to 75% acetonitrile in dichloromethane) to give the title compound as a white solid, 373 mg.

MS (electrospray): 274 (M+1) for C₉H₉BrN₂O₃

¹H-NMR (300 MHz, DMSO-d₆) δ: 3.45-3.65 (m, 4H); 4.75 (m, 1H); 5.05 (t, 1H); 7.65 (m, 2H); 8.70 (s, 1H).

### [3-(5-Bromo-pyridin-2-yl)-4,5-dihydro-isoxazol-5-yl]-methanol

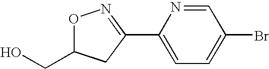

5-Bromo-pyridine-2-carbaldehyde oxime (60 g, 298.5 mmol) and allyl alcohol (49.7 ml) were added to tetrahydrofuran (200 ml) and then bleach (2016 ml) was added. The reaction was allowed to stir for four hours followed by extraction with tetrahydrofuran (2×200 ml). The organic layers were combined, dried over sodium sulfate, and concentrated in vacuo to give the desired product (38.8 g).

¹H-NMR (300 MHz, DMSO-d₆) δ: 3.2 (dd, 1H); 3.41 (dd, 1H); 3.55 (m, 2H); 4.8 (m, 1H); 5.02 (d, 1H); 7.84 (d, 1H); 8.16 (d, 1H); 8.8 (s, 1H).

### 5-Bromo-pyridine-2-carbaldehyde oxime

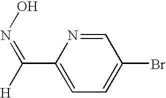

5-Bromo-pyridine-2-carbaldehyde (CAS# 31181-90-5, 60 g, 322 mmol) was added to methanol (700 ml) and then water was added (700 ml) followed by addition of hydroxylamine hydrochloride (28 g, 403 mmol). Sodium carbonate (20.5 g, 193.2 mmol) in water (200 ml) was then added and the reaction was allowed to stir for 30 minutes. Water (500 ml) was then added and the precipitate was filtered and washed with water (2×300 ml) to give the desired product (60 g).

¹H-NMR (300 MHz, DMSO-d₆) δ: 7.75 (d, 1H); 8.09 (t, 2H), 8.72 (s, 1H); 11.84 (s, 1H).

### (5R)-3-[3-Fluoro-4-(4,4,5,5-tetramethyl-1,3,2-dioxaborolan-2-yl)phenyl]-5-(1H-1,2,3-triazol-1-ylmethyl)-1,3-oxazolidin-2-one

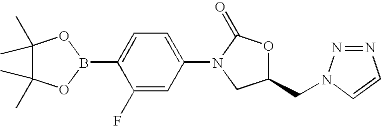

(5R)-3-(3-Fluoro-4-iodophenyl)-5-(1H-1,2,3-triazol-1-ylmethyl)-1,3-oxazolidin-2-one (2 g, 5.15 mmol) (cf. Example 1), bis(pinacolato)diboron, 2.62 g (10.3 mmol), potassium acetate, 2.5 g (25.5 mmol), and 1,1′-[bis(diphenylphosphino)ferrocene]dichloropalladium(II) dichoromethane complex, 0.38 g (0.52 mmol) were suspended in DMSO, 15 ml. The mixture was heated at 80° C. for 40 minutes to give a clear black solution. Ethyl acetate (150 ml) was then added and the mixture was filtered through celite, washed with saturated NaCl (2×100 ml), dried over sodium sulfate and evaporated. The residue was purified by chromatography (silica gel, 40 to 100% ethyl acetate in hexane, followed by 1-5% acetonitrile in ethyl acetate) to give the product as a crystalline tan solid, 1.97 g (98%).

¹H-NMR (300 MHz, DMSO-d₆) δ: 1.28 (s, 12H), 3.91 (dd, 1H); 4.23 (t, 1H); 4.83 (d, 2H); 5.14 (m, 1H); 7.27 (dd, 1H); 7.37 (dd, 1H); 7.62 (t, 1H); 7.75 (s, 1H); 8.16 (s, 1H).

### EXAMPLE 14

### (5R)-3-(3-Fluoro-4-{6-[5-(3-hydroxy-1,1-dioxidotetrahydro-3-thienyl)-4,5-dihydroisoxazol-3-yl]pyridin-3-yl}phenyl)-5-(1H-1,2,3-txiazol-1-ylmethyl)-1,3-oxazolidin-2-one

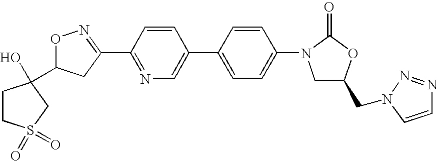

Tetrahydrothiophen-3-one (3.125 g, 30.5 mmol) was dissolved in THF (15 ml) and cooled to 0° C. Vinylmagnesium bromide (1M THF solution, 32.1 ml, 32.1 mmol) was added and the solution was stirred at 0° C. for 1.5 hours. The mixture was diluted with ethyl acetate, washed with water, then saturated brine, dried over sodium sulfate and evaporated to yield 3-vinyltetrahydrothiophene-3-ol as a dark orange oil (3.18 g).

5-Bromo-N-hydroxypyridine-2-carboximidoyl chloride (1.51 g, 6.42 mmol) and 3-vinyltetrahydrothiophene-3-ol (2.50 g, 19.3 mmol) were combined in ethyl acetate (25 ml) and cooled to 0° C. A solution of triethylamine (0.982 ml, 7.06 mmol) in ethyl acetate (7 ml) was added dropwise over 10 minutes. The mixture was stirred at 0° C. for 3 hours, then diluted with 50 ml ethyl acetate. The suspension was filtered, the solids were rinsed with ethyl acetate and the filtrate was concentrated to yield a thick oil which was purified by flash chromatography (silica gel, 15 to 50% ethyl acetate in hexanes). Evaporation of the appropriate fractions gave 3-[3-(5-bromopyridin-2-yl)-4,5-dihydroisoxazol-5-yl]tetrahydrothiophene-3-ol as a thick clear oil (438 mg). This material was oxidized in the next step without further characterization.

3-[3-(5-Bromopyridin-2-yl)-4,5-dihydroisoxazol-5-yl]tetrahydrothiophene-3-ol (438 mg, 1.33 mmol) was dissolved in acetonitrile (9 ml); water (6 ml), and potassium peroxomonosulfate (Oxone, 3.06 g, 4.98 mmol) were added and the mixture was stirred at room temperature for 4 hours. The solution was diluted with ethyl acetate, washed with water and dried over sodium sulfate. Evaporation yielded crude 3-[3-(5-bromopyridin-2-yl)-4,5-dihydroisoxazol-5-yl]tetrahydrothiophene-3-ol 1,1-dioxide as a tan solid (310 mg).

¹H-NMR (300 MHz, DMSO-D6) δ ppm 2.24-2.62 (m, 2H); 2.69-2.94 (m, 2H); 2.99-3.19 (m, 2H); 3.35-3.56 (m, 2H); 4.66-4.92 (m, 1H); 5.51 & 5.50 (2×d, 1H); 7.84 (dd, 1H); 8.12 (dd, 1H); 8.79 (d, 1H); 11.95 (bs, 1H)

3-[3-(5-Bromopyridin-2-yl)-4,5-dihydroisoxazol-5-yl]tetrahydrothiophene-3-ol 1,1-dioxide (310 mg, 0.858 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (366 mg, 0.944 mMol), potassium carbonate (711 mg, 5.15 mMol), and tetrakis(triphenylphosphino)palladium(0) (99 mg, 0.085 mMol) were suspended in DMF (7 ml) and water (0.5 ml). The mixture was heated at 85° C. for 2.5 hours, diluted with water, and extracted with ethyl acetate three times. The organic phase was dried over sodium sulfate, evaporated and purified by flash column chromatography (silica gel, 0.5 to 5% methanol in dichloromethane) yielding crude material, which was further purified by reverse phase preparative HPLC (C18/acetonitrile/water/0.1% trifluoroacetic acid). Evaporation of the appropriate fractions gave (5R)-3-(3-fluoro-4-{6-[5-(3-hydroxy-1,1-dioxidotetrahydro-3-thienyl)-4,5-dihydroisoxazol-3-yl]pyridin-3-yl}phenyl)-5-(1H-1,2,3-triazol-1-ylmethyl)-1,3-oxazolidin-2-one as an off-white solid (25 mg).

MS (electrospray): 543(M+1) for C₂₄H₂₃FN₆O₆S

¹H-NMR (400 MHz, DMSO-d₆) δ: 2.15 (m, 2H); 3.13-3.29 (m, 4H); 3.43-3.57 (m, 2H); 3.96 (dd, 1H); 4.30 (t, 1H); 4.82 (m, 1H); 4.86 (d, 2H); 5.18 (m, 1H); 7.42 (dd, 1H); 7.59 (dd, 1H); 7.69 (t, 1H); 7.76 (s, 1H); 7.99 (d, 1H); 8.07 (d, 1H); 8.18 (s, 1H); 8.83 (s, 1H).

### EXAMPLE 15

### (5R)-3-(3-Fluoro-4-{6-[5-(1-hydroxy-1-methylethyl)-4,5-dihydroisoxazol-3-yl]pyridin-3-yl}phenyl)-5-(1H-1,2,3-triazol-1-ylmethyl)-1,3-oxazolidin-2-one

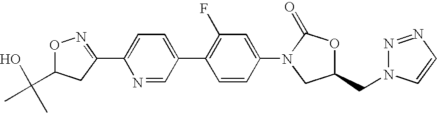

5-Bromo-N-hydroxypyridine-2-carboximidoyl chloride (1.0 g, 4.26 mmol) and 2-methyl-3-butene-2-ol (4.5 ml, 43 mmol) were combined in ethyl acetate (10 ml) and cooled to 0° C. A solution of triethylamine (0.71 ml, 5.1 mmol) in ethyl acetate (4 ml) was added dropwise over 10 minutes. The mixture was allowed to come slowly to room temperature over 4 hours, then diluted to 50 ml with ethyl acetate. The suspension was filtered, the solids were rinsed with ethyl acetate and the filtrate was concentrated to yield a thick oil which was sonnicated with hexane, filtered and dried under vacuum to give crude 2-[3-(5-bromopyridin-2-yl)-4,5-dihydroisoxazol-5-yl]propan-2-ol as a grey solid, 1.1 g. This material was used in the following step without further purification.

2-[3-(5-Bromopyridin-2-yl)-4,5-dihydroisoxazol-5-yl]propan-2-ol (200 mg, 0.70 mMol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (300 mg, 0.77 mMol), potassium carbonate (600 mg, 4.34 mMol), and tetrakis(triphenylphosphino)palladium(0) (85 mg, 0.074 mMol) were suspended in DMF (4 ml) and water (0.4 ml). The mixture was heated at 80° C. for 1.5 hours, then diluted with water. The solids were collected on a filter, dissolved in methanol, adsorbed on silica gel and purified by flash chromatography (silica gel, 1-10% methanol/dichloromethane) to yield a solid which was triturated with ether to give (5R)-3-(3-fluoro-4-{6-[5-(1-hydroxy-1-methylethyl)-4,5-dihydroisoxazol-3-yl]pyridin-3-yl}phenyl)-5-(1H-1,2,3-triazol-1-ylmethyl)-1,3-oxazolidin-2-one as a white solid (116 mg). Mp 197° C.

MS (electrospray): 467 (M+1) for C₂₃H₂₃FN₆O₄

¹H-NMR (400 MHz, DMSO-d₆) δ: 1.11 (s, 3H); 1.12 (s, 3H); 3.40 (d, 2H); 3.97 (dd, 1H); 4.30 (t, 1H); 4.53 (t, 1H); 4.64 (s, 1H); 4.86 (d, 2); 5.18 (m, 1H); 7.42 (dd, 1H); 7.58 (dd, 1H); 7.69 (t, 1H); 7.76 (s, 1H); 7.98 (d, 1H); 8.05 (d, 1H); 8.18 (s, 1H); 8.81 (s, 1H).

### EXAMPLE 16

### (5R)-3-(4-{6-[4,5-Bis(hydroxymethyl)-4,5-dihydroisoxazol-3-yl]pyridin-3-yl}-3-fluorophenyl-5-(1H-1,2,3-triazol-1-ylmethyl)-1,3-oxazolidin-2-one

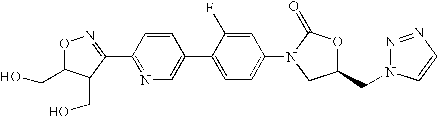

3-(5-Bromo-2-pyridyl)-4,5-bis(hydroxymethyl)-4,5-dihydroisoxazole (0.346 g, 1.21 mM), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (0.291 g, 0.75 nm and potassium carbonate (0.337 g, 2.4 mM) were stirred in N,N-dimethylformamide (5 mL). Tetrakis(triphenylphosphino)palladium(0) (0.087 g, 0.075 mM) was added followed by water (0.5 mL). The reaction was heated to 80° C. for two hours. Water was added to the mixture resulting in a precipitate that was filtered. The filtrate was extracted using ethyl acetate. The organic layer was dried (magnesium sulfate), filtered and concentrated. The yellow oil was diluted with dimethyl sulfoxide (1.5 mL) and purified using Gilson HPLC. Relevant fractions were collected and lyophilized to give the desired product as a yellow solid (0.101 g).

MS (ESP): 469 (MH⁺) for C₂₂H₂₁FN₆O₅

300 MHz NMR (DMSO-d₆) δ: 3.74 (s, 3H); 3.3.45-3.58 (bidden by water peak, 2H); 3.97 (m, 1H); 4.28 (t, 1H); 4.66 (m, 1H); 4.85 (d, 2H); 5.16 (m, 1H); 7.39 (d, 1H); 7.56 (d, 1H); 7.67 (t, 1H); 7.76 (s, 1H); 7.95-8.05 (m, 2H); 8.16 (s, 1H); 8.78 (s, 1H).

The intermediate for the above was prepared as follows:

### 3-(5-Bromo-2-pyridyl)-4,5-bis(hydroxymethyl)-4,5-dihydroisoxazole

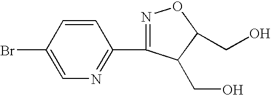

5-Bromo-N-hydroxypyridine-2-carboximidoyl chloride (0.500 g, 2.12 mM) was dissolved in tetrahydrofuran (5 mL) and stirred at 0° C. 2-Butene-1,4-diol (0.748 g, 8.49 mM was added followed by the dropwise addition of triethylamine (0.236 g, 2.33 mM) in tetrahydrofuran (5 mL). The reaction was let warm to room temperature and stirred overnight. The yellow mixture was diluted with water and extracted using ethyl acetate. The organic layer was washed with water, dried (magnesium sulfate), filtered, and concentrated to give the desired product as a yellow solid (0.346 g).

MS (SP): 288 (MH⁺) for C₁₀H₁₁BrN₂O₃

300 MHz NMR (DMSO-d₆) δ: 3.57 (t, 2H); 3.79-3.98 (m, 3H); 4.68 (m, 1H); 4.83 (t, 1H); 5.03 (t, 1H); 7.85 (d, 1H); 8.10 (dd, 1H); 8.76 (ds, 1H).

### EXAMPLE 17

### (5R)-3-(4-{6-[5-(2,2-Diethoxyethyl)-4,5-dihydroisoxazol-3-yl]pyridin-3-yl}3-fluorophenyl)-5-(1H-1,2,3-triazol-1-ylmethyl)-1,3-oxazolidin-2-one

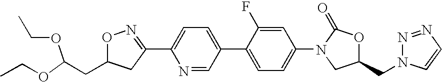

5-Bromo-2-[5-(2,2-diethoxyethyl)-4,5-dihydroisoxazol-3-yl]pyridine (0.70 g, 2.13 mM), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (0.550 g, 1.42 mM) and potassium carbonate (0.636 g, 4.54 mM were combined and stirred in N,N-dimethylformamide (10 mL). Tetrakis (triphenylphosphino)palladium(0) (0.162, 0.142 mM was added followed by water (1 mL). The reaction was heated to 80° C. for six hours then diluted with water and extracted using ethyl acetate. The organic layer was dried (magnesium sulfate), filtered and concentrated. The yellow oil was chromatographed using ethyl acetate, concentrated and washed with water several times. The organic layer was dried (magnesium sulfate), filtered and concentrated. The yellow solid was dissolved in dichloromethane and purified on prep TLC plates using 80% ethyl acetate/hexanes. Relevant bands were cut, washed with ethyl acetate, filtered, and concentrated to give the desired product as a white solid (0.085 g).

MS (ESP): 525 (MH⁺) for C₂₆H₂₉FN₆O₅

300 MHz NMR (DMSO-d₆) δ: 0.96-1.22 (m, 6H); 1.78-2.00 (m, 2H); 3.39-3.72 (m, 6H); 3.96 (m, 1H); 4.30 (t, 1H); 4.65 (t, 1H); 4.70-4.94 (m, 3H); 5.10-5.25 (m, 1H); 7.42 (d, 1H); 7.59 (d, 1H); 7.69 (t, 1H); 7.77 (s, 1H); 7.90-8.10 (m, 2H); 8.18 (s, 1H); 8.82 (s, 1H).

The intermediate for the above compound was prepared as follows:

### 5-Bromo-2-[5-(2,2-diethoxyethyl)-4,5-dihydroisoxazol-3-yl]pyridine

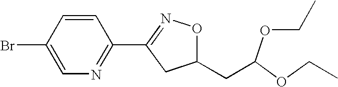

5-Bromo-N-hydroxypyridine-2-carboximidoyl chloride (0.540 g, 2.30 m was stirred in tetrahydrofuran (10 mL). 3-Butenal-diethylacetal (1.00 g, 6.93 mM) was added followed by sodium hypochlorite (15 mL) and stirred overnight. The aqueous layer was extracted using ethyl acetate. The organic layer was washed with brine, dried (magnesium sulfate), filtered, and concentrated. The yellow oil was chromatographed using 15% ethyl acetate/hexanes. Relevant fractions were collected to give the desired product as a yellow oil (0.704 g).

MS (ESP): 344 (MH⁺) for C₁₄H₁₉BrN₂O₃

300 MHz NMR (DMSO-d₆) δ: 1.04-1.20 (m, 6H); 1.86-1.95 (m, 2H); 3.17 (dd, 1H); 3.35-3.65 (m, 5H); 4.63 (t, 1H); 4.74-4.82 (m, 1H); 7.86 (d, 1H); 8.12 (dd, 1H); 8.78 (ds, 1H).

### EXAMPLE 18

### (5R)-3-(3-Fluoro-4-{6-[5,5-bis(hydroxymethyl)-4,5-dihydroisoxazol-3-yl]pyridin-3-yl}phenyl)-5-{[4-(fluoromethyl)-1H-1,2,3-txiazol-1-yl]methyl}-1,3-oxazolidin-2-one

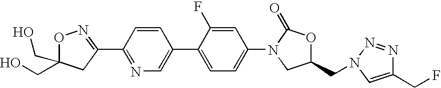

3-(5-Bromo-2-pyridinyl)-5,5(4H)-isoxazoledimethanol (400 mg, 1.39 mmol), (5R)-5-{[4-(fluoromethyl)-1H-1,2,3-triazol-1-yl]methyl}-3-[3-fluoro-4-(4,4,5,5-tetramethyl-1,3,2-dioxaborolan-2-yl)phenyl]-1,3-oxazolidin-2-one (cf. Example 13) (703 mg, 1.67 mmol), potassium carbonate (768 mg, 5.56 mmol), and tetrakis(triphenylphosphino)palladium(0) (80 mg, 0.07 mmol) were combined and suspended in DMF (8 ml) and water (1 ml). The mixture was heated at 80° C. for 2 hours, then was poured into cold water (20 ml). The solids formed were collected, rinsed with water and washed with dichloromethane (5 ml), the solids were further purified by column chromatography, eluted with 8% methanol in dichloromethane to give the title compound as a white solid (275 mg)

MS (ESP): 501.15 (M+1) for C₂₃H₂₂F₂N₆O₅

¹H-NMR(300 Mz) (DMSO-d₆) δ: 3.34 (m, overlap with solvent peak, 2H); 3.51 (d, 4H); 3.95 (dd, 1H); 4.29 (t, 1H); 4.88 (d, 2H); 5.02 (t, 2H); 5.18 (m, 1H); 5.50 (d, br, 2H); 7.41 (dd, 1H); 7.58 (dd, 1H); 7.69 (t, 1H); 8.0 (overlapping m, 2H); 8.41 (s, br, 1H); 8.85 (s, br, 1H)ppm

The intermediates for the above example were prepared as follows;

### 3-(5-Bromo-2-pyridinyl-5,5(4H)-isoxazoledimethanol

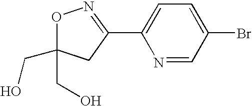

2-[5,5-Bis({[tert-butyl(dimethyl)silyl]oxy}methyl)-4,5-dihydroisoxazol-3-yl)-5-bromopyridine (10.2 g, 19.8 mmol) was dissolved in anhydrous tetrahydrofuran (30 ml), cooled down to 0° C., Tetrabutylammonium fluoride (49.4 mL, 49.4 mmol) was added drop wise to the solution. The reaction mixture was allowed to warm up to room temperature while stirring for ninety minutes. Ethyl acetate (100 ml) and water (50 ml) were added into the mixture, and the two layer were separated, the organic phase was again washed with brine, dried over anhydrous magnesium sulfate, concentrated under vacume and purified by column chromatography, eluted with 50% hexanes in ethyl acetate to give the title compound as a white solid (4.49 g).

MS (ESP): 288 (M+1) for C₁₀H₁₁BrN₂O₃

¹H-NMR (300 Mz) (DMSO-d₆) δ: 3.26 (s, 2H); 3.50 (q, 4H); 5.03 (m, 2H); 7.83 (d, 1H); 8.10 (d, 1H); 8.77 (s, 1H).

### (5R)-5-{[4-(Fluoromethyl)-1H-1,2,3-triazol-1-yl]methyl}-3-[3-fluoro-4-(4,4,5,5-tetramethyl-1,3,2-dioxaborolan-2-yl)phenyl]-1,3-oxazolidin-2-one

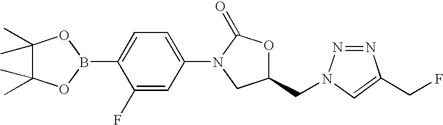

(5R)-3-(3-Fluoro-4-iodophenyl)-5-{[4-(fluoromethyl)-1H-1,2,3-triazol-1-yl]methyl}-1,3-oxazolidin-2-one (cf. Example 1) (4.0 g, 9.5 mmol), bis(pinacolato)diboron (6.0 g, 23.75 mmol), potassium acetate (3.24 g, 33.25 mmol), and 1,1′-[bis(diphenylphosphino)ferrocene]dichloropalladium(I) dichoromethane complex (0.695 g, 0.95 mmol) were suspended in DMSO (25 ml). The mixture was heated at 80° C. for 90 minutes to give a clear black solution. After cooling down to room temperature, ethyl acetate (250 ml) was then added and the mixture was filtered through celite, washed with saturated NaCl (2×100 ml), dried over sodium sulfate and concentrated to dryness. The dark residue was dissolved in dichloromethane (30 ml), followed by slow addition of hexanes (100 ml), the resulting precipitate was filtered and washed with 5% dichloromethane in hexanes and collected as the desired product (2.73 g) which was used directly as an intermediate without further purification.

### EXAMPLE 19

### N-{[(5S-3-(3-Fluoro-4-{6-[5,5-bis(hydroxymethyl)-4,5-dihydroisoxazol-3-yl]pyridin-3-yl}phenyl)-2-oxo-1,3-oxazolidin-5-yl]methyl}acetamide

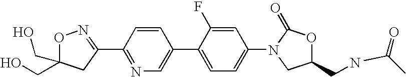

3-(5-Bromo-2-pyridinyl)-5,5(4H)-isoxazoledimethanol (300 mg, 1.045 mmol), N-({(5S)-3-[3-fluoro-4-(4,4,5,5-tetramethyl-1,3,2-dioxaborolan-2-yl)phenyl]-2-oxo-1,3-oxazolidin-5-yl}methyl)acetamide (cf. Example 13) (434 mg, 1.15 mmol), potassium carbonate (577 mg, 4.18 mmol), and tetrakis(triphenylphosphino)palladium(0) (60 mg, 0.05 mmol) were combined and suspended in DMF (8 ml) and water (1 ml). The mixture was heated at 80° C. for 2 hours, then was poured into cold water (80 ml). The solids formed were collected, rinsed with water and washed with dichloromethane (5 ml), the solids were further purified by column chromatography, eluted with 8% methanol in dichloromethane to give the title compound as a white solid (140 mg)

MS (ESP): 459.13 (M+1) for C₂₂H₂₃FN₄O₆

¹H NMR (300 Mz) (DMSO-d₆) δ: 1.82 (s, 3H); 3.30 (m, 2H); 3.40 (m, 2H); 3.53 (m, 4H); 3.80 (dd, 1H); 4.19 (t, 1H); 4.78 (m, 1H); 5.02 (m, 2H); 7.45 (dd, 1H); 7.70 (m, 2H); 8.0 (overlapping m, 2H); 8.21 (m, 1H); 8.85 (s, 1H) ppm

The intermediate for the above was prepeared as follows:

### 3-(5-Bromo-2-pyridinyl-5,5(4H)-isoxazoledimethanol

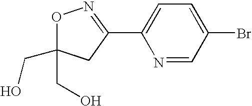

2-[5,5-Bis({[tert-butyl(dimethyl)silyl]oxy}methyl)-4,5-dihydroisoxazol-3-yl]-5-bromopyridine (10.2 g, 19.8 mmol) was dissolved in anhydrous tetrahydrofuran (30 ml), cooled down to 0° C., Tetrabutylammonium fluoride (49.4 mL, 49.4 mmol) was added dropwise to the solution. The reaction mixture was allowed to warm up to room temperature while stirring for ninety minutes. Ethyl acetate (100 ml) and water (50 ml) were added into the mixture, and the two layer were separated, the organic phase was again washed with brine, dried over anhydrous magnesium sulfate, concentrated under vacume and purified by column chromatography, eluted with 50% hexanes in ethyl acetate to give the title compound as a white solid (4.49 g).

MS (ESP): 288 (M+1) for C₁₀H₁₁BrN₂O₃

¹H-NMR (300 Mz) (DMSO-d₆) δ: 3.26 (s, 2H); 3.50 (q, 4H); 5.03 (m, 2H); 7.83 (d, 1H); 8.10 (d, 1H); 8.77 (s, 1H).

### N-({(5S)-3-[3-fluoro-4-(4,4,5,5-tetramethyl-1,3,2-dioxaborolan-2-yl)phenyl]-2-oxo-1,3-oxazolidin-5-yl}methyl)acetamide

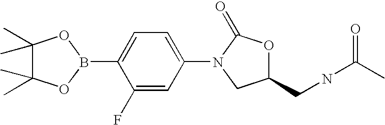

N-{[(5S)-3-(3-Fluoro-4-iodophenyl)-2-oxo-1,3-oxazolidin-5-yl]methyl}acetamide (1.0 g, 2.65 mmol), bis(pinacolato)diboron (1.68 g, 6.6 mmol), potassium acetate (0.9 g, 9.27 mmol), and 1,1′-[bis(diphenylphosphino)ferrocene]dichloropalladium(II) dichoromethane complex (0.194 g, 0.265 mmol) were suspended in DMSO (10 ml). The mixture was heated at 80° C. for 90 minutes to give a clear black solution. After cooling down to room temperature, ethyl acetate (150 ml) was added and the mixture was filtered through celite, washed with saturated brine (2×100 ml), dried over sodium sulfate and concentrated to dryness. The dark residue was dissolved in dichloromethane (5 ml), followed by slow addition of hexanes (20 ml), the resulting precipitate was filtered and washed with 5% dichloromethane in hexanes and collected as the desired product (0.99 g) which was used directly as an intermediate without further purification.

### EXAMPLE 20

### (5R)-3-(3-Fluoro-4-{5-[5-(2-hydroxyethyl)-4,5-dihydroisoxazol-3-yl]pyridin-3-yl}phenyl)-5-(1H-1,2,3-triazol-1-ylmethyl)-1,3-oxazolidin-2-one

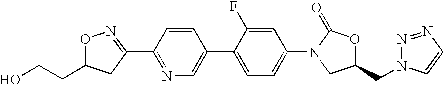

Using essentially the same procedure as for Example 16 but starting with 2-[3-(5-bromopyridin-2-yl)-4,5-dihydroisoxazol-5-yl]ethanol (0.305 g, 1.10 mM) gave the title compound as an off-white solid (0.075 g).

MS (ESP): 452 (MH⁺) for C₂₂H₂₁FN₆O₄

300 MHz NMR (DMSO-d₆) δ: 2.98 (t, 2H); 3.27-3.40 (2H, hidden by water peak); 3.73-3.77 (m, 2H); 3.96-3.99 (m, 1H); 4.30 (t, 1H); 4.86 (d, 2H); 4.93 (t, 1H); 5.18-5.21 (m, 1H); 6.86 (s, 1H); 7.43 (d, 1H); 7.68 (d, 1H); 7.72 (t, 1H); 7.77 (s, 1H); 8.07-8.16 (m, 2H); 8.18 (s, 1H); 8.88 (s, 1H).

The intermediate for the above compound was prepared as follows:

### 2-[3-(5-Bromopyridin-2-yl)-4,5-dihydroisoxazol-5-yl]ethanol

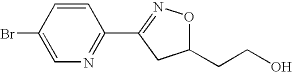

5-Bromo-N-hydroxypyridine-2-carboximidoyl chloride (1.00 g, 4.25 mM was stirred in tetrahydrofuran (20 mL). 3-Buten-1-ol (0.764 g, 10.6 mM was added followed by sodium hypochlorite (30 mL) and stirred overnight. The aqueous layer was extracted using ethyl acetate. The organic layer was washed with brine, dried (magnesium sulfate), filtered, and concentrated. The yellow oil was chromatographed using 10-50% ethyl acetate/hexanes. Relevant fractions were concentrated to a brown oil that was purified using prep TLC plates using 50% ethyl acetate/hexanes to give the desired product as a yellow solid (0.352 g).

MS (ESP): 272 (MH⁺) for CO₁₀H₁₁BrN₂O₂

300 MHz NMR (DMSO-d₆) δ: 2.96 (t, 2H); 3.26-3.40 (2H, bidden by water peak); 3.74 (q, 2H); 4.92 (t, 1H); 6.81 (s, 1H); 7.95 (d, 1H); 8.20 (d, 1H); 8.83 (s, 1H).

### EXAMPLE 21

### tert-Butyl 3-(5-{2-fluoro-4-[(5R)-2-oxo-5-(1H-1,2,3-triazol-1-ylmethyl-1,3-oxazolidin-3-yl]phenyl}pyridin-2-yl)-4,5-dihydroisoxazole-5-carboxylate

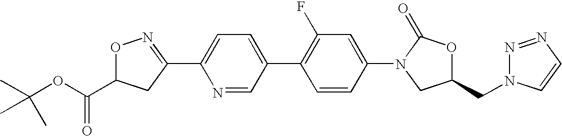

tert-Butyl 3-(5-bromopyridin-2-yl)-4,5-dihydroisoxazole-5-carboxylate (1.37 g, 4.20 mMol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (1.96 g, 5.04 mMol) (cf. Example 13), potassium carbonate (3.5 g, 25.4 mMol), and tetrakis(triphenylphosphino)palladium(0) (440 mg, 0.38 mMol) were suspended in DMF (20 ml) and water (2 ml). The mixture was heated at 80° C. for 45 minutes, diluted with water, and extracted with ethyl acetate. The organic phase was washed with saturated sodium chloride, dried over sodium sulfate, evaporated and purified by flash chromatography (silica gel, 0.5-5% MeOH/CH₂Cl₂) to yield a solid which was triturated with ether to give tert-butyl 3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazole-5-carboxylate as an off-white solid (1.2 g). Mp 165-168° C.

MS (electrospray): 509 (M+1) for C₂₅H₂₅FN₆O₅

¹H-NMR (400 MHz, DMSO-d₆) δ: 1.44 (s, 9H); 3.59 (dd, 1H); 3.80 (dd, 1H); 3.96 (dd, 1H); 4.30 (t, 1H); 4.86 (d, 2H); 5.19 (m, 2H); 7.42 (dd, 1H); 7.59 (dd, 1H); 7.69 (t, 1H); 7.77 (s, 1H); 8.01 (d, 1H); 8.08 (d, 1H); 8.18 (s, 1H); 8.83 (s, 1H).

The intermediate for Example 21 was prepared as follows:

5-Bromo-N-hydroxypyridine-2-carboximidoyl chloride (1.0 g, 4.26 mmol) and tert-butyl acrylate (3 ml, 20.5 mmol) were combined in ethyl acetate (10 ml) and cooled to 0° C. A solution of triethylamine (0.71 ml, 5.1 mmol) in ethyl acetate (2 ml) was added dropwise over 10 minutes. The mixture was stirred 45 minutes at 0° C., the suspension was filtered, the solids were rinsed with ethyl acetate and the filtrate was concentrated to yield crude tert-butyl 3-(5-bromopyridin-2-yl)-4,5-dihydroisoxazole-5-carboxylate as a thick yellow oil, 1.37 g. This material was used without further purification.

### EXAMPLE 22

### 3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazole-5-carboxylic acid

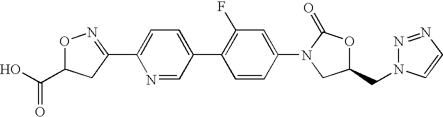

tert-Butyl 3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazole-5-carboxylate (Example 21)(0.2 g, 0.39 mmol) was dissolved in trifluoroacetic acid (3 ml) and stirred at room temperature for 1 hour. The solution was evaporated to give a residue, which was triturated with a 1:5 mixture of methanol:diethyl ether. The resulting solid material was dried in vacuo to yield the title compound as an off-white solid (160 mg). Mp 190-194° C.

MS (electrospray: 453 (M+1) for C₂₁H₁₇FN₆O₅

¹H-NMR (400 MHz DMSO-d₆) δ: 3.63 (dd, 1H); 3.79 (dd, 1H); 3.96 (dd, 1H); 4.30 (t, 1H); 4.86 (d, 2H); 5.18 (m, 1H); 5.24 (dd, 1H); 7.42 (dd, 1H); 7.59 (dd, 1H); 7.70 (t, 1H); 7.76 (s, 1H); 8.01 (d, 1H); 8.08 (d, 1H); 8.18 (s, 1M); 8.84 (s, 1H).

### EXAMPLE 23

### 3-(5-{2-Fluoro-4-[(5R)-2-oxo-5-(1H-1,2,3-triazol-1-ylmethyl)-1,3-oxazolidin-3-yl]phenyl}pyridin-2-yl)-N,N-dimethyl-4,5-dihydroisoxazole-5-carboxamide

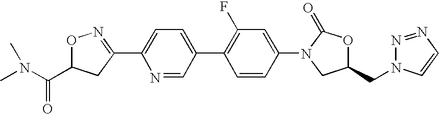

3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazole-5-carboxylic acid (Example 22) (110 mg, 0.24 mmol), pentafluorophenol (90 mg, 0.49 mmol), 4-(dimethylamino)pyridine (3 mg, 0.025 mmol) and DMF (1 ml) were combined to give a clear solution. 1-[3-(dimethylamino)propyl]-3-ethylcarbodiimide hydrochloride (90 mg, 0.47 mmol) was added, the solution was stirred at room temperature for 1.5 hours and diluted with ethyl acetate. The mixture was washed with water and saturated sodium chloride, dried over sodium sulphate and evaporated to give the pentafluorophenyl ester as a thick oil (150 mg). The pentafluorophenyl ester was combined with dimethylamine (2M THF solution, 1.25 ml, 2.5 mmol), dioxane (1 ml) and DMF (0.5 ml). The mixture was warmed to 60° C. for 5 hours, stirred at room temperature for 3 days, evaporated, redissolved in methanol and adsorbed on silica gel. Purification by flash chromatography (silica gel, 0.5-5% MeOH/CH₂Cl₂) gave a solid which was triturated with ether and dried in vacuo to give the title compound as an off-white solid (55 mg). Mp 180-190° C.

MS (electrospray): 480 (M+1) for C₂₃H₂₂FN₇O₄

¹H-NMR (400 MHz, DMSO-d₆) δ: 2.89 (s, 3H); 3.12 (s, 3H); 3.60 (dd, 1H); 3.87 (dd, 1H); 3.96 (dd, 1H); 4.30 (t, 1H); 4.86 (d, 2H); 5.18 (m, 1H); 5.66 (dd, 1H); 7.42 (dd, 1H); 7.59 (dd, 1H); 7.70 (t, 1H); 7.77 (s, 1H); 7.99 (d, 1H); 8.07 (d, 1H); 8.18 (s, 1H); 8.84 (s, 1H).

### EXAMPLE 24

### 3-(5-{2-Fluoro-4-[(5R)-2-oxo-5-(1H-1,2,3-triazol-1-ylmethyl)-1,3-oxazolidin-3-yl]phenyl}pyridin-2-yl)-N-methyl-4,5-dihydroisoxazole-5-carboxamide

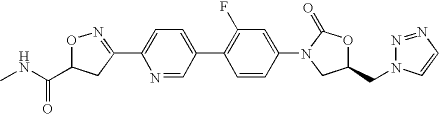

3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazole-5-carboxylic acid (Example 22) (250 mg, 0.55 mmol), pentafluorophenol (200 mg, 1.09 mmol), 4-(dimethylamino)pyridine (12 mg, 0.10 mmol) and DMF (2 ml) were combined to give a clear solution. 1-[3-(dimethylamino)propyl]-3-ethylcarbodiimide hydrochloride (200 mg, 1.04 mmol) was added, the solution was stirred at room temperature for 3 hours and diluted with ethyl acetate. The mixture was washed with water and dried over sodium sulphate and evaporated to give the pentafluorophenyl ester as a thick oil. The pentafluorophenyl ester was combined with methylamine (2M THF solution, 3 ml, 6 mmol) and dioxane (3 ml). The mixture was warmed to 60° C. in a sealed vessel for 1.5 hours, evaporated, redissolved in methanol and adsorbed on silica gel. Purification by flash chromatography (silica gel, 0.5-5% methanol dichloromethane) gave a solid, which was triturated with ether and dried in vacuo to give the title compound as a light yellow solid (141 mg). Mp 185-195° C.

MS (electrospray): 466 (M+1) for C₂₂H₂₀FN₇O₄

¹H-NMR (400 MHz, DMSO-d₆) δ: 2.63 (d, 3H); 3.61 (dd, 1H); 3.73 (dd, 1H); 3.96 (dd, 1H); 4.30 (t, 1H); 4.86 (d, 2H); 5.15 (dd, 1H); 5.18 (m, 1H); 7.42 (dd, 1H); 7.59 (dd, 1H); 7.69 (t, 1H); 7.76 (s, 1H); 8.00 (d, 1H); 8.08 (d, 1H); 8.18 (s, 1H); 8.22 (m, 1H); 8.84 (s, 1H).

### EXAMPLE 25

### (5R)-3-{3-Fluoro-4-[6-(5-{[(2-hydroxyethyl)sulfonyl]methyl}-4,5-dihydroisoxazol-3-yl)pyridin-3-yl]phenyl}-5-(1H-1,2,3-triazol-1-ylmethyl)-1,3-oxazolidin-2-one

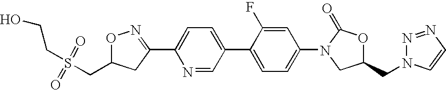

2-({[3-(5-Bromopyridin-2-yl)-4,5-dihydroisoxazol-5-yl]methyl}sulfonyl)ethanol (309 mg, 0.88 mMol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (377 mg, 0.97 mMol), potassium carbonate (731 mg, 5.297 mMol), and tetrakis(triphenylphosphino)palladium(0) (102 mg, 0.088 mMol) were suspended in DMF (5 ml) and water (0.5 ml). The mixture was heated at 85° C. for 1 hour, diluted with water, and extracted with ethyl acetate three times. The organic phase was dried over sodium sulfate, evaporated and purified by flash column chromatography (silica gel, 0.5 to 5% methanol in dichloromethane) the title compound as an off-white solid (84 mg): melting point: 210° C.

MS (electrospray): 531(M+1) for C₂₃H₂₃FN₆O₆S

¹H-NMR (400 MHz DMSO-d₆) δ: 3.34-3.49 (m, 3H); 3.56 (dd, 1H); 3.67-3.79 (m, 2H); 3.81 (q, 2H); 3.96 (dd, 1H); 4.30 (t, 1H); 4.86 (d, 2H); 5.16 (t, 2H); 5.19 (m, 1H); 7.42 (dd, 1H); 7.59 (dd, 1H); 7.70 (t, 1H); 7.77 (s, 1H); 8.01 (d, 1H); 8.08 (d, 1H); 8.18 (s, 1H); 8.84 (s, 1H).

The intermediates for Example 25 were prepared as follows:

[3-(5-Bromopyridin-2-yl)-4,5-dihydroisoxazol-5-yl]methanol (5 g, 19.46 mmol) was dissolved in dichloromethane (100 ml). Triphenylphosphine (7.66 g, 29.2 mmol) and carbon tetrachloride (9.36 ml, 97.28 mmol) were added and the mixture was stirred at room temperature for 2 hours. Additional portions of triphenylphosphine (1.5 g, 5.73 mmol) and carbon tetrachloride (2.5 ml, 30 mmol) were added and stirring was continued for 2 more hours. The solution was concentrated and purified by flash chromatography (silica gel, 7:3 hexane: methylene chloride) followed by precipitation from methylene chloride solution with hexane to yield 5-bromo-2-[5-(chloromethyl)-4,5-dihydroisoxazol-3-yl]pyridine as a white solid (2.05 g). This material was contaminated with triphenylphosphine oxide, and was used in the next step without further purification.

5-Bromo-2-[5-(chloromethyl)-4,5-dihydroisoxazol-3-yl]pyridine (500 mg, 1.82 mmol), 2-mercaptoethanol (157 mg, 1.99 mmol), potassium carbonate (502 mg, 3.64 mmol) and DMF (20 ml) were combined and warmed to 50° C. for 2.5 hours. An additional portion of 2-mercaptoethanol (78 mg, 0.99 mmol) was added and the mixture was warmed at 50° C. for 18 hours more, and then stirred at room temperature for 72 hours. The mixture was diluted with ethyl acetate, washed with water, dried over sodium sulphate and evaporated. Purification by column chromatography (silica gel, 10 to 100% ethyl acetate in hexanes) yielded 2-({[3-(5-bromopyridin-2-yl)-4,5-dihydroisoxazol-5-yl]methyl}thio)ethanol as a thick yellow oil. This material (300 mg, 0.943 mmol) was dissolved in acetonitrile (5 ml); water (4 ml), and potassium peroxomonosulfate (Oxone, 759 mg, 1.226 mmol) were added and the mixture was stirred at room temperature for 4 hours. The solution was diluted with ethyl acetate, washed with water and dried over sodium sulfate. Evaporation yielded crude 2-({[3-(5-bromopyridin-2-yl)-4,5-dihydroisoxazol-5-yl]methyl]sulfonyl)ethanol as a thick oil (309 mg). ¹H-NMR (300 MHz, DMSO-d₆) δ: 3.21-3.42 (m, 4H); 3.54 (dd, 1H); 3.61-3.74 (m, 2H); 3.80 (q, 2H); 5.19 (m, 2H); 7.87 (d, 1H); 8.14 (dd, 1H); 8.80 (d, 1H).

### EXAMPLE 26

### (5R)-3-[3-Fluoro-4-(6-{5-[hydroxy(phenyl)methyl]-4,5-dihydroisoxazol-3-yl}pyridin-3-yl)phenyl]-5-(1H-1,2,3-triazol-ylmethyl)-1,3-oxazolidin-2-one (Isomer A) and Example 27: Isomer B

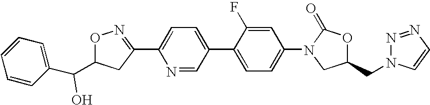

[3-(5-Bromopyridin-2-yl)-4,5-dihydroisoxazol-5-yl](phenyl)methanol, isomer A (107 mg, 0.32 mMol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (137 mg, 0.353 mMol), potassium carbonate (266 mg, 1.92 mmol), and tetrakis(triphenylphosphino)palladium(0) (37 mg, 0.032 mmol) were suspended in DMF (5 ml) and water (0.5 ml). The mixture was heated at 85° C. for 2 hours, diluted with water, and extracted with ethyl acetate. The organic phase was washed with saturated sodium chloride, dried over sodium sulfate, evaporated and purified by flash chromatography (silica gel, 0.5-5% MeOH/CH₂Cl₂) to yield Isomer A of the title compound as a pale yellow solid (87 mg). Mp 190° C.

MS (electrospray): 515 (M+1) for C₂₇H₂₃FN₆O₄

¹H-NMR (300 MHz, DMSO-d₆) δ: 3.27 (dd, 1H); 3.48 (dd, 1H); 3.96 (dd, 1H;) 4.29 (t, 1H); 4.74-4.98 (m, 4H); 5.11-5.25(m, 1H); 5.80 (d, 1H); 7.20-7.49 (m, 6H); 7.58 (d, 1H); 7.68 (t, 1H); 7.74-7.81 (m, 1H); 7.97 (d, 1H); 8.04 (d, 1H); 8.11-8.26 (m, 1H); 8.73-8.85 (m, 1H)

[3-(5-Bromopyridin-2-yl)-4,5-dihydroisoxazol-5-yl](phenyl)methanol, isomer B (130 mg, 0.39 mMol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (167 mg, 0.429 mMol), potassium carbonate (322 mg, 2.34 mmol), and tetrakis(triphenylphosphino)palladium(0) (45 mg, 0.039 mMol) were suspended in DMF (5 ml) and water (0.5 ml). The mixture was heated at 85° C. for 1.5 hours, diluted with water, and extracted with ethyl acetate. The organic phase was washed with saturated sodium chloride, dried over sodium sulfate, evaporated and purified by flash chromatography (silica gel, 0.5-5% MeOH/dichloromethane) to yield Isomer B of the title compound as an off-white solid (131 mg). Mp 182° C.

MS (electrospray: 515 (M+1) for C₂₇H₂₃FN₆O₄

¹H-NMR (300 MHz, DMSO-d₆) δ: 3.22 (dd, 1H); 3.33 (dd, 1H); 3.95 (dd, 1H); 4.29 (t, 1 H); 4.69 (t, 1H); 4.86 (d, 2H); 4.92 (m, 1H); 5.18 (m, 1H); 5.71 (d, 1H); 7.21-7.45 (m, 6H); 7.58 (d, 1H); 7.67 (t, 1H); 7.76 (s, 1H); 7.93 (d, 1H); 8.03 (d, 1H); 8.18 (s, 1H); 8.77 (s, 1H)

The intermediates were prepared as follows:

Benzaldehyde (1 g, 9.42 mmol) was dissolved in THF (8 ml) and cooled to 0° C. Vinylmagnesium bromide (1M THF solution, 9.89 ml, 9.89 mmol) was added and the solution was stirred at 0° C. for 1 hour. The mixture was diluted with ether, washed with water, then saturated NaCl, dried over sodium sulfate and evaporated to yield 1-phenylprop-2-en-1-ol as a pale yellow oil (1.16 g).

¹H-NMR (300 MHz, DMSO-d₆) δ: 5.05 (m, 2H); 5.24 (dt, 1H); 5.49 (d, 1H); 5.88-5.99 (m, 1H); 7.19-7.36 (m, 5H).

5-Bromo-N-hydroxypyridine-2-carboximidoyl chloride (189 mg, 2.08 mmol) and 1-phenylprop-2-en-1-ol (558 mg, 4.16 mmol) were combined in ethyl acetate (100 ml) and cooled to 0° C. A solution of triethylamine (0.40 ml, 2.29 mmol) in ethyl acetate (4 ml) was added dropwise over 10 minutes. The mixture was stirred at 0° C. for 1 hour, then diluted to 40 ml with ethyl acetate. The suspension was filtered, the solids were rinsed with ethyl acetate and the filtrate was concentrated to yield a thick oil which was purified by flash chromatography (silica gel, 5-50% ethyl acetate/hexanes) to resolve the product diastereomers into 2 racemic mixtures. The relative stereochemistry of the resolved compounds was not determined, the racemates were designated as isomer A (tlc Rf=0.4, silica gel, 80:20 hexanes:ethyl acetate) and isomer B (tlc Rf=0.25, silica gel, 80:20 hexanes:ethyl acetate). Yield of [3-(5-bromopyridin-2-yl)-4,5-dihydroisoxazol-5-yl](phenyl)methanol: isomer A (169 mg), isomer B (174 mg).

Isomer A: ¹H-NMR (300 MHz, DMSO-D6) δ ppm 3.24 (dd, 1H); 3.41 (dd, 1H); 4.78 (t, 1H); 4.87 (m, 1H); 5.78 (d, 1H); 7.23-7.43 (m, 5H); 7.83 (d, 1H); 8.10 (dd, 1H); 8.76 (d, 1H)

Isomer B: ¹H-NMR (300 MHz, DMSO-D6) δ ppm 3.18 (dd, 1H); 3.29 (dd, 1H); 4.67 (t, 1H); 4.92 (m, 1H); 5.70 (d, 1H); 7.22-7.43 (m, 5H); 7.79 (d, 1H); 8.08 (dd, 1H); 8.73 (d, 1H)

### EXAMPLE 28

### (5R)-3-(3-Fluoro-4-{6-[5-(1-hydroxycyclopentyl)-4,5-dihydroisoxazol-3-yl]pyridin-3-yl}phenyl)-5-(1H-1,2,3-triazol-1-ylmethyl)-1,3-oxazolidin-2-one

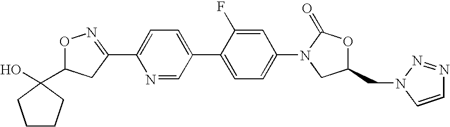

1-[3-(5-Bromopyridin-2-yl)-4,5-dihydroisoxazol-5-yl]cyclopentanol (86 mg, 0.276 mMol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (118 mg, 0.304 mMol), potassium carbonate (229 mg, 1.66 mmol), and tetrakis(triphenylphosphino)palladium(0) (32 mg, 0.028 mMol) were suspended in DMF (5 ml) and water (0.5 ml). The mixture was heated at 85° C. for 1.5 hours, diluted with water, and extracted with ethyl acetate. The organic phase was washed with saturated sodium chloride, dried over sodium sulfate, evaporated and purified by flash chromatography (silica gel, 0.5-5% MeOH/CH₂Cl₂) to yield (5R)-3-(3-fluoro-4-{6-[5-(1-hydroxycyclopentyl)-4,5-dihydroisoxazol-3-yl]pyridin-3-yl}phenyl)-5-(1H-1,2,3-triazol-1-ylmethyl)-1,3-oxazolidin-2-one as a beige solid (82 mg). Mp 225° C.

MS (electrospray): 493 (M+1) for C₂₅H₂₅FN₆O₄

¹H-NMR (300 MHz, DMSO-d₆) δ: 1.49-1.78 (m, 8H); 3.37-3.46 (m, 2H); 3.96 (dd, 1H); 4.29 (t, 1H); 4.53 (s, 1H); 4.67 (t, 2H); 4.86 (d, 2H); 5.18 (m, 1H); 7.42 (dd, 1H); 7.58 (dd, 1H); 7.68 (t, 1H); 7.76 (s, 1H); 7.98 (d, 1H); 8.05 (d, 1H); 8.18 (s, 1H); 8.81 (s, 1H).

The intermediates were prepared as follows:

Cyclopentanone (3.16 ml, 35.7 mmol) was dissolved in THF (15 ml) and cooled to 0° C. Vinylmagnesium bromide (1M THF solution, 37.4 ml, 37.4 mmol) was added and the solution was stirred at 0° C. for 1 hour. The mixture was diluted with ethyl acetate, washed with water, then saturated NaCl, dried over sodium sulfate and evaporated to yield 1-vinylcyclopentanol as a pale yellow oil (3.12 g).

5-Bromo-N-hydroxypyridine-2-carboximidoyl chloride (1.6 g, 6.81 mmol) and 1-vinylcyclopentanol (1.53 g, 13.62 mmol) were combined in ethyl acetate (15 ml) and cooled to 0° C. A solution of triethylamine (1.04 ml, 7.49 mmol) in ethyl acetate (5 ml) was added dropwise over 10 minutes. The mixture was stirred at 0° C. for 1 hour, then diluted to 40 ml with ethyl acetate. The suspension was filtered, the solids were rinsed with ethyl acetate and the filtrate was concentrated to yield a thick oil which was purified by flash chromatography (silica gel, 15-50% ethyl acetate/hexanes). Evaporation of the appropriate fractions yielded 1-[3-(5-bromopyridin-2-yl)-4,5-dihydroisoxazol-5-yl]cyclopentanol as a red oil (858 mg).

¹H-NMR (300 MHz, DMSO-D6) δ ppm 1.40-1.75 (m, 6H); 1.85-2.17 (m, 2H); 3.29-3.42 (m, 2H); 4.51 (s, 1H); 4.65 (t, 1H); 7.82 (d, 1H); 8.10 (dd, 1H); 8.76 (d, 1H)

### EXAMPLE 29

### 1-[3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]-2-methyl]propyl 2-naphthylacetate (Isomer A)

### EXAMPLE 30

### Isomer B

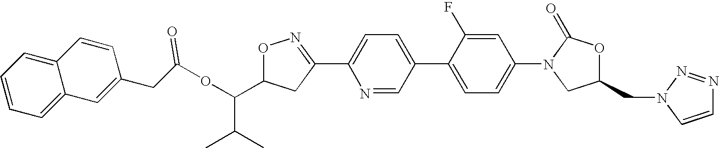

1-[3-(5-Bromopyridin-2-yl)-4,5-dihydroisoxazol-5-yl]-2-methylpropyl 2-naphthylacetate (451 mg, 0.97 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (412 mg, 1.062 mMol), potassium carbonate (800 mg, 5.79 mMol), and tetrakis(triphenylphosphino)palladium(0) (112 mg, 0.097 mMol) were suspended in DMF (5 ml) and water (0.5 ml). The mixture was heated at 85° C. for 1.5 hours, diluted with water, and extracted with ethyl acetate. The organic phase was washed with saturated sodium chloride, dried over sodium sulfate, evaporated and purified by flash chromatography (silica gel, 0.5-5% MeOH/CH₂Cl₂) to yield 1-[3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]-2-methylpropyl 2-naphthylacetate as a light yellow solid (575 mg). A portion (100 mg) of the diastereomeric product mixture was partially resolved by reverse phase preparative HPLC (Phenomenex 4 micron Synergi MAX-RP C12, 4.6×100 mm, isocratic elution 45:55 acetonitrile: water, 0.1% tifluoroacetic acid, 20 ml/min.) into 2 co-eluting isomeric mixtures, A (eluted from column first) and B (eluted second).

Isomer mixture A: off-white solid (20 mg) Mp 102° C.:

MS (electrospray): 649 (M+1) for C₃₆H₃₃FN₆O₅

¹H-NMR (300 MHz DMSO-d₆) δ: 0.87 (2×d, 61); 1.87 (m, 1H); 3.19 (dd, 1H); 3.46 (dd, 1H); 3.80 (s, 2H); 3.97 (dd, 1H); 4.31 (t, 1H); 4.87 (d, 1H); 4.92-5.01 (m, 2H); 5.19 (m, 1H); 7.38 (m, 2H); 7.45 (dd, 1H); 7.58-7.74 (m, 7 M); 7.77 (s, 1H); 7.85 (d, 1H); 8.00 (d, 1H); 8.19 (s, 1H); 8.75 (s, 1H)

Isomer mixture B: off-white solid (22 mg) Mp 85° C.:

MS (electrospray): 649 (M+1) for C₃₆H₃₃FN₆O₅

¹H-NMR (300 MHz DMSO-d₆) δ: 0.87 & 0.92 (2×d, 6H); 2.06 (m 1H); 3.02 (dd, 1H); 3.51 (dd, 1H); 3.81 (dd, 2H); 3.98 (dd, 1H); 4.31 (t, 1H); 4.87 (m, 3H); 5.00 (m, 1H); 5.19 (m, 1H); 7.29 (dd, 1H); 7.39 (m, 2H); 7.44 (dd, 1H); 7.58-7.75 (m, 6H); 7.77 (s, 1H); 7.90 (d, 1H); 8.00 (d, 1H); 8.19 (s, 1H); 8.70 (s, 1H)

The intermediates were prepared as follows:

Isobutyraldehyde (2.0 g, 27.7 mmol) was dissolved in THF (14 ml) and cooled to 0° C. Vinylmagnesium bromide (1M THF solution, 29.1 ml, 29.1 mmol) was added and the solution was stirred at 0° C. for 30 minutes. The mixture was diluted with diethyl ether, washed with water, then saturated brine, dried over sodium sulfate and evaporated to yield 4-methylpent-1-en-3-ol as a pale yellow oil (2.9 g), contaminated with diethyl ether. The material was used in the next step without further purification. 5-Bromo-N-hydroxypyridine-2-carboximidoyl chloride (1.64 g, 6.99 mmol) and 4-methylpent-1-en-3-ol (1.40 g, 14.0 mmol) were combined in ethyl acetate (15 ml) and cooled to 0° C. A solution of triethylamine (1.07 ml, 7.69 mmol) in ethyl acetate (5 ml) was added dropwise over 10 minutes. The mixture was stirred at 0° C. for 1 hour, then diluted to 40 ml with ethyl acetate. The suspension was filtered, the solids were rinsed with ethyl acetate and the filtrate was concentrated to yield an orange oil which was purified by flash chromatography (silica gel, 15-80% ethyl acetate/hexanes). Evaporation of the appropriate fractions yielded 1-{3-[5-(bromomethyl)pyridin-2-yl]-4,5-dihydroisoxazol-5-yl}-2-methylpropan-1-ol as a white solid (1.03 g).

¹H-NMR (300 MHz DMSO-D6) δ ppm 0.91 (m, 6H); 1.64-1.85 (m, 1H); 3.11-3.17 (ddd, 1 H); 3.21-3.45 (m, 3H); 4.67-4.81 (m, 1H); 4.82 & 4.98 (2×d, 1H); 7.83 (dm 1H); 8.11 (ddd, 1H); 8.76 (t, 1H) 1-{3-[5-(Bromomethyl)pyridin-2-yl]-4,5-dihydroisoxazol-5-yl}-2-methylpropan-1-ol (614 mg, 2.05 mmol) and 2-naphthylacetic acid (1.53 g, 8.21 mmol) were dissolved in DMF (10 ml), diisopropylcarbodiimide (1.28 ml, 8.21 mMol), and 4-dimethylaminopyridine (5 mg, 0.04 mMol) were added and the solution was stirred at room temperature for 30 minutes. The mixture was diluted with water, and extracted with ethyl acetate. The organic phase was washed with saturated sodium chloride, dried over sodium sulfate, evaporated and purified by flash chromatography (silica gel, 15% ethyl acetate/hexanes) to yield 1-[3-(5-bromopyridin-2-yl)-4,5-dihydroisoxazol-5-yl]-2-methylpropyl 2-naphthylacetate as an off-white solid (483 mg).

¹H-NMR (300 MHz, DMSO-d₆) δ: 0.86 (m, 6H); 1.85 & 2.05 (2×m, 1H); 2.89 & 3.09 (2×dd, 1H); 3.39 (m, 1H); 3.79 (dd, 2H); 4.80-5.01 (m, 2H); 7.28 (d, 1H); 7.40-7.46 (m, 2 H); 7.60-7.77 (m, 5H); 8.00 (dd, 1H); 8.60 (dd, 1H)

### EXAMPLE 31

### R)-3-(3-Fluoro-4-{6-[5-(1-hydroxy-2-methylpropyl)-4,5-dihydroisoxazol-3-yl]pyridin-3-yl}phenyl)-5-(1H-1,2,3-triazol-1-ylmethyl)-1,3-oxazolidin-2-one (Isomer A)

### EXAMPLE 32

### Isomer B

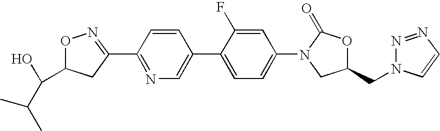

1-[3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]-2-methylpropyl 2-naphthylacetate (diastereomeric product mixture A plus B), 419 mg, 0.646 mmol) was dissolved in methanol (50 ml) and ethanol (25 ml). Potassium carbonate (534 mg, 3.88 mmol) and water (4 ml) were added and the mixture was stirred at room temperature for 18 hours. The solution was diluted with water, and extracted twice with ethyl acetate. The organic phase was dried over sodium sulfate, evaporated and purified by flash chromatography (silica gel, 0.5-5% MeOH/CH₂Cl₂) to yield (5R)-3-(3-fluoro-4-(6-[5-(1-hydroxy-2-methylpropyl)-4,5-dihydroisoxazol-3-yl]pyridin-3-yl}phenyl)-5-(1H-1,2,3-triazol-1-ylmethyl)-1,3-oxazolidin-2-one as an orange solid (200 mg). The diastereomeric product mixture was partially resolved by reverse phase preparative HPLC (Phenomenex 4 micron Synergi MAX-RP C12, 4.6×100 mm, gradient elution 30 to 50% acetonitrile/water, 0.1% trifluoroacetic acid, 20 ml/min.) into 2 co-eluting isomeric mixtures, A (eluted from column first) and B (eluted second).

Isomer mixture A: off-white solid (30 mg) Mp 212° C.:

MS (electrospray): 481 (M+1) for C₂₄H₂₅FN₆O₄

¹H-NMR (300 MHz, DMSO-d₆) δ: 0.93 (d, 6H); 1.82 (m, 1H); 3.15 (m, 1H); 3.46 (m, 2H); 3.96 (dd, 1H); 4.29 (t, 1H); 4.79 (m, 1H); 4.86 (d, 2H); 5.18 (m, 1H); 7.42 (dd, 1H); 7.59 (dd, 1H); 7.69 (t, 1H); 7.77 (s, 1H); 7.98 (d, 1H); 8.04 (d, 1H); 8.18 (s, 1H); 8.81 (s, 1H)

Isomer mixture B: off-white solid (58 mg) Mp 155° C.:

MS (electrospray): 481 (M+1) for C₂₄H₂₅FN₆O₄

¹H-NMR (300 MHz, DMSO-d₆) δ: 0.91 (2×d, 6H); 1.73 (m, 1H); 3.42 (d, 2H); 3.96 (dd, 1H); 4.29 (t, 1H); 4.72 (ddd, 1H); 4.86 (d, 2H); 5.18 (m, 1H); 7.42 (dd, 1H); 7.59 (dd, 1H); 7.69 (t, 1H); 7.76 (s, 1H); 7.98 (d, 1H); 8.05 (d, 1H); 8.18 (s, 1H); 8.82 (s, 1H).

### EXAMPLE 33

### (5R)-3-{3-Fluoro-4-[6-(5-{[(2-pyridin-4-ylethyl)amino]methyl}-4,5-dihydroisoxazol-3-yl)pyridin-3-yl]phenyl}-5-(1H-1,2,3-triazol-1-ylmethyl)-1,3-oxazolidin-2-one

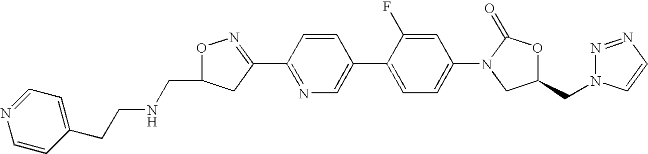

{[3-(5-Bromopyridin-2-yl)-4,5-dihydroisoxazol-5-yl]methyl}(2-pyridin-4-ylethyl)amine (200 mg, 0.557 mMol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (238 mg, 0.613 mMol), potassium carbonate (461 mg, 3.34 mMol), and tetrakis(triphenylphosphino)palladium(0) (64 mg, 0.056 mMol) were suspended in DMF (5 ml) and water (0.5 ml). The mixture was heated at 85° C. for 1.5 hours, diluted with water, and extracted twice with ethyl acetate. The organic phase was dried over sodium sulfate, evaporated and purified by flash chromatography (silica gel, 0.5-5% MeOH/CH₂Cl₂) to (5R)-3-{3-fluoro-4-[6-(5-{[(2-pyridin-4-ylethyl)amino]methyl)-4,5-dihydroisoxazol-3-yl)pyridin-3-yl]phenyl}-5-(1H-1,2,3-triazol-1-ylmethyl)-1,3-oxazolidin-2-one as an off-white solid (170 mg). Mp 181° C.

MS (electrospray): 543 (M+1) for C₂₈H₂₇FN₈O₃

¹H-NMR (300 MHz, DMSO-d₆) δ: 2.68-2.85 (m, 6H); 3.27 (dd, 1H); 3.47 (dd, 1H); 3.96 (t, 1H); 4.30 (t, 1H); 4.82 (m, 1H); 4.86 (d, 2H); 5.18 (m, 1H); 7.23 (dd, 2H); 7.42 (dd, 1H); 7.59 (dd, 1H); 7.69 (t, 1H); 7.77 (s, 1H); 7.98 (d, 1H); 8.06 (d, 1H); 8.18 (s, 1H); 8.40 (dd, 2H); 8.81 (s, 1H)

The intermediates were prepared as follows:

[3-(5-Bromopyridin-2-yl)-4,5-dihydroisoxazol-5-yl]methanol (5 g, 19.46 mmol) was dissolved in dichloromethane (100 ml). Triphenylphosphine (7.66 g, 29.2 mmol) and carbon tetrachloride (9.36 ml, 97.28 mmol) were added and the mixture was stirred at room temperature for 2 hours. Additional portions of triphenylphosphine (1.5 g, 5.73 mmol) and carbon tetrachloride (2.5 ml, 30 mmol) were added and stirring was continued for 2 more hours. The solution was concentrated and purified by flash chromatography (silica gel, 7:3 hexane:dichloromethane) followed by precipitation from dichloromethane solution with hexane to yield 5-bromo-2-[5-(chloromethyl)-4,5-dihydroisoxazol-3-yl]pyridine as a white solid (2.05 g). This material was contaminated with triphenylphosphine oxide, and was used in the next step without further purification.

5-Bromo-2-[5-(chloromethyl)-4,5-dihydroisoxazol-3-yl]pyridine (300 mg, 1.09 mmol), 2-pyridin-4-ylethanamine (1.33 g, 10.9 mmol) and tetrabutylammonium iodide (−5 mg, catalytic) were combined in DMSO (1 ml). The mixture was warmed to 90° C. for 18 hours, diluted with water, and extracted with ethyl acetate. The organic phase was dried over sodium sulphate, concentrated and purified by flash chromatography (silica gel, 0.5-5% MeOH/CH₂Cl₂) to yield {[3-(5-bromopyridin-2-yl)-4,5-dihydroisoxazol-5-yl]methyl}(2-pyridin-4-ylethyl)amine as an oily solid (207 mg).

¹H-NMR (300 MHz, DMSO-D6) δ ppm 2.80-2.90 (m, 6H); 3.25 (dd, 1H); 3.53 (dd, 1H); 4.97 (m, 1H); 7.27 (dd, 2H); 7.85 (d, 1H); 8.12 (dd, 1H); 8.45 (dd, 2H); 8.78 (d, 1H)

### EXAMPLE 34

### (5R)-3-(3-Fluoro-4-{6-[5-(4-hydroxy-1-methylpiperidin-4-yl)-4,5-dihydroisoxazol-3-yl]pyridin-3-yl}phenyl)-5-(1H-1,2,3-triazol-1-ylmethyl)-1,3-oxazolidin-2-one

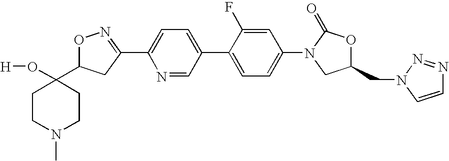

14-[3-(5-Bromopyridin-2-yl)-4,5-dihydroisoxazol-5-yl]-1-methylpiperidin-4-ol (340 mg, 1.00 mMol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (427 mg, 1.10 mMol), potassium carbonate (827 mg, 5.99 mMol), and tetrakis(triphenylphosphino)palladium(0) (115 mg, 0.090 mMol) were suspended in DMF (5 ml) and water (0.5 ml). The mixture was heated at 85° C. for 2.5 hours, diluted with water, and extracted with ethyl acetate three times. The organic phase was dried over sodium sulfate, evaporated and purified by reverse phase preparative HPLC (C18/acetonitrile/water/0.1% trifluoroacetic acid). Evaporation of the appropriate fractions yielded (5R)-3-(3-fluoro-4-{6-[5-(4-hydroxy-1-methylpiperidin-4-yl)-4,5-dihydroisoxazol-3-yl]pyridin-3-yl}phenyl)-5-(1H-1,2,3-triazol-1-ylmethyl)-1,3-oxazolidin-2-one as an orange solid (280 mg). Mp 73° C.

MS (electrospray): 522 (M+1) for C₂₆H₂₈FN₇O₄

¹H-NMR (300 MHz DMSO-d₆) δ: 1.60-1.90 (m, 4H); 2.79 (d, 2H); 3.10 (m, 2H); 3.33 (d, 2H); 3.48 (d, 2H); 3.96 (dd, 1H); 4.30 (t, 1H); 4.59 (t, 1H); 4.86 (d, 2H); 5.19 (m, 1H); 7.41 (dd, 1H); 7.58 (dd, 1H); 7.68 (t, 1H); 7.77 (s, 1H); 7.99 (d, 1H); 8.07 (d, 1H); 8.19 (s, 1H); 8.82 (s, 1H); 9.49 (bs, 1H)

The intermediates were prepared as follows:

1-Methyl-4-piperidone (3.26 ml, 26.5 mmol) was dissolved in THF (15 ml) and cooled to 0° C. Vinylmagnesium bromide (1M THF solution, 27.8 ml, 27.8 mmol) was added and the solution was stirred at 0° C. for 1.5 hours. The mixture was diluted with ethyl acetate, washed with water, then saturated NaCl, dried over sodium sulfate and evaporated to yield 1-methyl-4-vinylpiperidin-4-ol as a pale yellow oil (1.50 g). 5-Bromo-N-hydroxypyridine-2-carboximidoyl chloride (830 mg, 3.53 mmol) and 1-methyl-4-vinylpiperidin-4-ol (1.50 g, 10.6 mmol) were combined in ethyl acetate (20 ml) and cooled to 0° C. A solution of triethylamine (0.54 ml, 3.88 mmol) in ethyl acetate (7 ml) was added dropwise over 10 minutes. The mixture was stirred at 0° C. for 1 hour, then 18 hours at room temperature, then diluted with 50 ml ethyl acetate. The suspension was filtered, the solids were rinsed with ethyl acetate and the filtrate was concentrated to yield a thick oil which was purified by reverse phase preparative HPLC (C18/acetonitrile/water/0.1% trifluoroacetic acid). Evaporation of the appropriate fractions yielded 4-[3-(5-bromopyridin-2-yl)-4,5-dihydroisoxazol-5-yl]-1-methylpiperidin-4-ol as a pale yellow solid (609 mg).

¹H-NMR (300 MHz, DMSO-D6) δ ppm 1.57-1.88 (1, 4H); 2.78 (d, 2H); 3.08 (m, 2H); 3.33 (s, 3H); 3.42 (d, 2H); 4.58 (t, 1H); 5.16 (s, 1H); 7.85 (d, 1H); 8.13 (dd, 1H); 8.79 (d, 1H); 9.16 (bs, 1H)

### EXAMPLE 35

### (5R)-3-{3-Fluoro-4-[6-(5-{[(2-pyridin-4-ylethyl)sulfonyl]methyl}-4,5-dihydroisoxazol-3-yl)pyridin-3-yl]phenyl}-5-(1H-1,2,3-triazol-1-ylmethyl)-1,3-oxazolidin-2-one

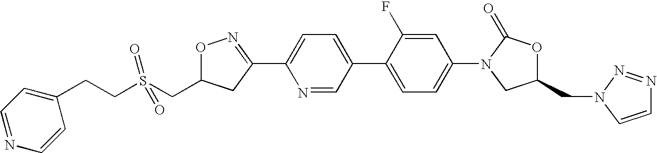

5-Bromo-2-(5-{[(2-pyridin-4-ylethyl)sulfonyl]methyl}-4,5-dihydroisoxazol-3-yl)pyridine (173 mg, 0.423 mMol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (180 mg, 0.464 mMol), potassium carbonate (349 mg, 2.53 mMol), and tetrakis(triphenylphosphino)palladium(0) (49 mg, 0.042 mmol) were suspended in DMF (5 ml) and water (0.5 ml). The mixture was heated at 85° C. for 3 hours, diluted with water, and extracted with ethyl acetate three times. The organic phase was dried over sodium sulfate, evaporated and purified by flash column chromatography (silica gel, 0.5 to 5% methanol in dichloromethane) yielding (5R)-3-{3-fluoro-4-[6-(5-{[(2-pyridin-4-ylethyl)sulfonyl]methyl}-4,5-dihydroisoxazol-3-yl)pyridin-3-yl]phenyl}-5-(1H-1,2,3-triazol-1-ylmethyl)-1,3-oxazolidin-2-one as an off-white solid (55 mg): melting point: 195° C.

MS (electrospray): 592(M+1) for C₂₈H₂₆FN₇O₅S

¹H-NMR (300 MHz, DMSO-d₆) δ: 3.08 (m, 2H); 3.44 (dd, 1H); 3.52-3.64 (m, 3H); 3.69-3.85 (m, 2H); 3.96 (dd, 1H); 4.30 (t, 1H); 4.86 (d, 2H); 5.21 (m, 2H); 7.35 (d, 2H); 7.42 (dd, 1H); 7.59 (dd, 1H); 7.70 (t, 1H); 7.77 (s, 1H); 8.02 (d, 1H); 8.09 (d, 1H); 8.18 (s, 1H); 8.50 (d, 2H); 8.84 (s, 1H).

The intermediates were prepared as follows:

[3-(5-Bromopyridin-2-yl)-4,5-dihydroisoxazol-5-yl]methanol (5 g, 19.46 mmol) was dissolved in dichloromethane (100 ml). Triphenylphosphine (7.66 g, 29.2 mmol) and carbon tetrachloride (9.36 ml, 97.28 mmol) were added and the mixture was stirred at room temperature for 2 hours. Additional portions of triphenylphosphine (1.5 g, 5.73 mmol) and carbon tetrachloride (2.5 ml, 30 mmol) were added and stirring was continued for 2 more hours. The solution was concentrated and purified by flash chromatography (silica gel, 7:3 hexane:dichloromethane) followed by precipitation from dichloromethane solution with hexane to yield 5-bromo-2-[5-(chloromethyl)-4,5-dihydroisoxazol-3-yl]pyridine as a white solid (2.05 g). This material was contaminated with triphenylphosphine oxide, and was used in the next step without further purification.

5-Bromo-2-[5-(chloromethyl)-4,5-dihydroisoxazol-3-yl]pyridine (500 mg, 1.82 mmol), 2-pyridin-4-ylethanethiol (759 mg, 5.45 mmol), potassium carbonate (753 mg, 5.45 mmol) and DMF (20 ml) were combined and warmed to 50° C. for 1 day. The mixture was diluted with ethyl acetate, washed with water, dried over sodium sulphate and evaporated. Purification by column chromatography (silica gel, 10 to 50% ethyl acetate in hexanes) yielded 5-bromo-2-(5-{[(2-pyridin-4-ylethyl)thio]methyl}-4,5-dihydroisoxazol-3-yl)pyridine as a thick yellow oil. This material (200 mg, 0.536 mmol) was dissolved in acetonitrile (5 ml); water (4 ml), and potassium peroxomonosulfate (Oxone, 529 mg, 0.697 mmol) were added and the mixture was stirred at room temperature for 2 hours. The solution was diluted with ethyl acetate, washed with water and dried over sodium sulfate. Evaporation yielded crude 5-bromo-2-(5-{[(2-pyridin-4-ylethyl)sulfonyl]methyl)-4,5-dihydroisoxazol-3-yl)pyridine as a thick oil (175 mg). ¹H-NMR (300 MHz DMSO-4) δ: 3.08 (m, 2H); 3.38 (dd, 1H); 3.50-3.63 (m, 3H); 3.69 (dd, 1H); 3.80 (dd, 1H); 5.21 (m, 1H); 7.38 (dd, 2H); 7.88 (d, 1H); 8.14 (dd, 1H); 8.52 (dd, 2H); 8.80 (d, 1H).

### REFERENCE EXAMPLE 36

### (5R)-3-[4-[6-[4,5-Dihydro-5-(hydroxymethyl)-3-isoxazolyl]-3-pyridinyl]-3-fluorophenyl]-5-(1H-1,2,3-triazol-1-ylmethyl)-1,3-oxazolidin-2-one

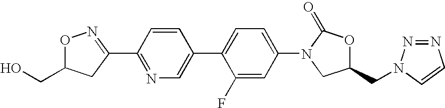

[3-(5-Bromo-pyridin-2-yl)-4,5-dihydro-isoxazol-5-yl]-methanol (2 g, 7.75 mmol) (cf. Example 13), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (cf. Example 13) (2 g, 5.15 mmol), potassium carbonate (2.3 g, 16.7 mmol), and tetrakis(triphenylphosphino)palladium(0) (0.6 g, 0.52 mmol) were combined and suspended in DMF (25 ml) and water (2.5 ml). The mixture was heated at 80° C. for 2 hours, then diluted with water to 100 ml. The solids were collected, rinsed with water and resuspended in warm DMSO (20 ml). The suspension was diluted with dichloromethane (100 ml) and ether (50 ml). The solid was collected, rinsed with ether and methanol, and dried in vacuo to give the pure product as a light yellow solid, 975 mg.

MS (electrospray): 439 (M+1) for C₂₁H₁₉FN₆O₄

¹H-NMR (300 MHz, DMSO-d₆) δ: 3.36-3.58 (m, 3H); 3.95 (dd, 1H); 4.29 (t, 1H); 4.78 (m, 1H); 4.86 (d, 2H); 5.02 (t, 1H); 5.18 (m, 1H); 7.41 (dd, 1H); 7.58 (dd, 1H); 7.69 (t, 1H); 7.77 (s, 1H); 7.98 (d, 1H); 8.05 (dd, 1H); 8.18 (s, 1H).

### EXAMPLE 37

### [3-(5-{2-Fluoro-4-[(5R)-2-oxo-5-(1H-1,2,3-triazol-1-ylmethyl)-1,3-oxazolidin-3-yl]pheny}pyridin-2-yl)-4,5-dihydroisoxazol-5-yl]methyl N,N-dimethylglycinate

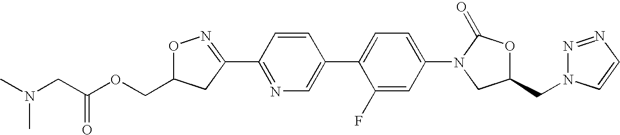

(5R)-3-(3-Fluoro-4-{6-[5-(hydroxymethyl)-4,5-dihydroisoxazol-3-yl]pyridin-3-yl}phenyl)-5-(1H-1,2,3-triazol-1-ylmethyl)-1,3-oxazolidin-2-one (250 mg, 0.57 mMol) (Example 36), N,N-dimethylglycine (150 mg, 1.46 mMol), 1-[3-(dimethylamino)propyl]-3-ethylcarbodiimide hydrochloride (220 mg, 1.15 mMol), and 4-dimethylaminopyridine (5 mg, 0.04 mMol) were suspended in 4 ml of DMF at room temperature. The mixture was stirred overnight and then concentrated. The residue was purified by chromatography (silica gel; elution with 1 to 10% methanol in dichloromethane) to give slightly impure material. The sample was dissolved in dichloromethane, treated with alcoholic HCl solution and precipitated with ether. The solid was collected, rinsed with ether and dried in vacuo to yield the hydrochloride salt of the title compound as a hygroscopic light orange solid (250 mg).

MS (electrospray): 524 (M+1) for C₂₅H₂₆FN₇O₅

¹H-NMR (300 MHz, DMSO-d₆) δ: 2.83 (s, 6H); 3.34-3.42 (m, 2H); 3.58-3.68 (dd, 1H); 4.22-4.46 (m, 51); 4.86 (d, 2H); 5.06 (m, 1H); 5.19 (m, 1H); 7.43 (d, 1H); 7.58 (d, 1H); 7.69 (t, 1); 7.77 (s, 1H); 7.99-8.09 (dd, 2H); 8.19 (s, 1H); 8.83 (s, 1H).

### EXAMPLE 38

### [3-(5-{2-Fluoro-4-[(5R)-2-oxo-5-(1H-1,2,3-triazol-ylmethyl-1,3-oxazolidin-3-yl]phenyl}pyridin-2-yl)-4,5-dihydroisoxazol-5-yl]methyl pentadecanoate

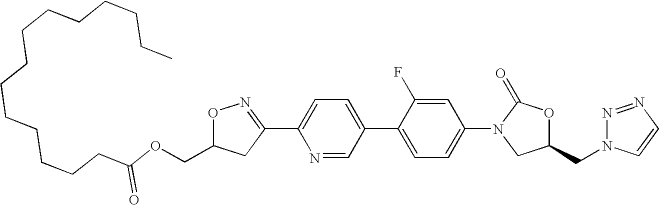

(5R)-3-(3-Fluoro-4-{6-[S-(hydroxymethyl)-4,5-dihydroisoxazol-3-yl]pyridin-3-yl}phenyl)-5-(1H-1,2,3-triazol-1-ylmethyl)-1,3-oxazolidin-2-one (Example 36) (150 mg, 0.33 mmol), pentadecanoic acid (157 mg, 0.51 mmol), 1-ethyl-3-(3-dimethylaminopropyl) carbodiimide hydrochloride (131 mg, 0.69 mmol), and 4-dimethylaminopyridine (14 mg, 0.08 mmol) were added to DMF (5 ml) and allowed to stir at room temperature overnight. EtOAc (50 ml) was then added and the organic layers were washed with water (2×20 ml), dried over Na₂SO₄, and concentrated in vacuo to yield a crude residue. The residue was purified by column chromatography using 0-5% MeOH/dichloromethane to yield the product as a white solid (100 mg).

MS (electrospray): 663.24 (MH⁺) for C₃₆H₄₇FN₆O₅

¹H-NMR (Dichloromethane-d₂) δ: 0.67 (t, 3H); 1.09 (s, 21H); 1.43 (m, 3H); 2.12 (t, 2H); 3.16 (dd, 1H); 3.41 (dd, 1H); 3.81 (dd, 1H); 4.05 (m, 3H); 4.62 (t, 2H); 4.80 (m, 1H); 4.90 (m, 1H); 7.08 (dd, 1H); 7.34 (m, 2H); 7.54 (s, 1H); 7.64 (s, 1H); 7.73 (d, 1H); 7.88 (d, 1H); 8.59 (s, 1H).

### EXAMPLE 39

### [3-(5-{2-Fluoro-4-[(5R)-2-oxo-5-(1H-1,2,3-triazol-1-ylmethyl)-1,3-oxazolidin-3-yl]pheny}pyridin-2-yl)-4,5-dihydroisoxazol-5-yl]methyl 3,6,9,12-tetraoxatridec-1-yl carbonate

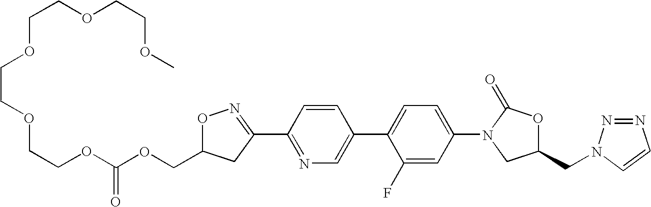

Tetraethyleneglycol monomethylether (300 mg, 2.27 mmol) was dissolved in dichloromethane (3 ml) and cooled to 0° C. Phosgene (20% in toluene: 1.2 ml, 2.27 mMol) was added and the solution was allowed to slowly come to room temperature overnight. The solution was concentrated in vacuo to give the chloroformate intermediate as a clear oil. The flask containing the chloroformate was cooled on an ice bath and (5R)-3-(3-fluoro-4-{6-[5-(hydroxymethyl)-4,5-dihydroisoxazol-3-yl]pyridin-3-yl}phenyl)-5-(1H-1,2,3-triazol-1-ylmethyl)-1,3-oxazolidin-2-one (Example 36) (200 mg, 0.46 mMol), DMF (5 ml) and pyridine (0.3 ml, 3.7 mMol) were added sequentially. The mixture was allowed to come to room temperature over 10 minutes, then stirred for 20 minutes more. Ethyl acetate was added, followed by washing with saturated NaCl. The organic layer was dried over sodium sulfate, evaporated and purified by chromatography (silica gel; elution with 1 to 10% methanol in dichloromethane). The product containing fractions were pooled, evaporated, dissolved in a minimum amount of dichloromethane and precipitated with ether. The solid was collected on a filter and rinsed with 1:1 ether:hexane. The title compound was thus obtained as a hygroscopic white solid, 160 mg.

MS (electrospray): 673 (M+1) for C₃₁H₃₇FN₆O₁₀

¹H-NMR (300 MHz DMSO-d₆) δ: 3.21 (s, 3H); 3.30-3.63 (m, 16H); 3.96 (dd, 1H); 4.17-4.34 (m, 5H); 4.86 (d, 2H); 5.04 (m, 1H); 5.19 (m, 1H); 7.42 (dd, 1H); 7.58 (dd, 1H); 7.69 (t, 1H); 7.76 (s, 1H); 7.99-8.08 (dd, 2H); 8.18 (s, 1H); 8.82 (s, 1H).

### EXAMPLE 40

### [3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl piperidine-4-carboxylate

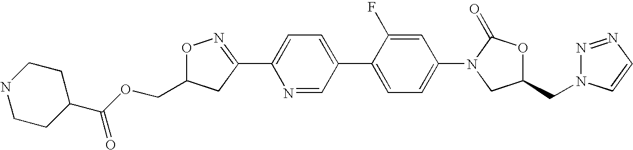

(5R)-3-(3-Fluoro-4 {6-[5-(hydroxymethyl)-4,5-dihydroisoxazol-3-yl]pyridin-3-yl}phenyl)-5-(1H-1,2,3-triazol-1-ylmethyl)-1,3-oxazolidin-2-one (Example 36) (200 mg, 0.46 mmol), Boc-piperidine-4-carboxylic acid (157 mg, 0.69 mmol), 1-ethyl-3-(3-dimethylaminopropyl)carbodiimide hydrochloride (175 mg, 0.91 mmol), and 4 dimethylaminopyridine (14 mg, 0.11 mmol) were added to DMF (5 ml). The reaction was allowed to stir at room temperature for 2 hours followed by addition of EtOAc (50 ml). The organic layers were washed with distilled water (3×20 ml), dried over Na₂SO₄, and concentrated in vacuo to yield a crude residue. The residue was purified by column chromatography using 0-2% MeOH/dichloromethane to yield a white powder (150 mg). The white powder (150 mg) was added to 50% TFA/dichloromethane (10 ml) and allowed to stir for 30 minutes. The reaction was concentrated in vacuo to yield the product as a white powder (150 mg).

MS (electrospray): 550.24 (MH⁺) for C₂₇H₂₈FN₇O₅

¹H-NMR (DMSO-d₆) δ: 3.38 (m, 2H); 3.77 (s, 2H); 3.95 (m, 1H); 4.29 (t, 1H); 4.85 (d, 2H); 5.20 (m, 2); 7.38 (d, 1H); 7.56 (d, 1H); 7.66 (t, 1H); 7.75 (s, 1H); 8.00 (m, 2H); 8.18 (s, 1H); 8.80 (s, 1H).

### EXAMPLE 41

### Diammonium salt of [3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl phosphate

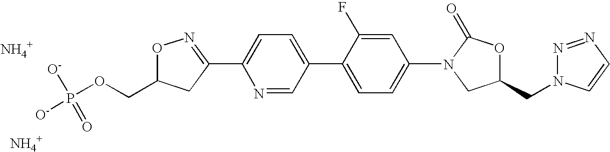

Di-tert-butyl [3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl phosphate (235 mg, 0.37 mmol) was added to dioxane (10 ml) followed by addition of 4N HCl in dioxane (3 ml) and the mixture was allowed to stir for 45 minutes. Ether (50 ml) was then added and the precipitate was collected by filtration. The precipitate was added to distilled water (5 ml) followed by NH₄OH (0.2 ml). The solution was then filtered through a 45-micron filter and lyophilized to yield the product (180 mg).

MS (electrospray): 519.08 (MH⁺) for C₂₁H₂₀FN₆O₇P

¹H-NMR (300 MHz, DMSO-d₆) δ: 3.38 (m, 2H); 3.77 (s, 2H); 3.95 (m, 1H); 4.29 (t, 1H); 4.85 (d, 2H); 5.20 (m, 2H); 7.38 (d, 1H); 7.56 (d, 1H); 7.66 (t, 1H); 7.75 (s, 1H); 8.00 (m, 2H); 8.18 (s, 1H); 8.80 (s, 1H).

The intermediate for the above was prepared as follows:

### Di-tert-butyl [3-(5-{2-fluoro-4-[(5R)-2-oxo-5-(1H-1,2,3-triazol-1-ylmethyl-1,3-oxazolidin-3-yl]phenyl}pyridin-2-yl)-4,5-dihydroisoxazol-5-yl]methyl phosphate

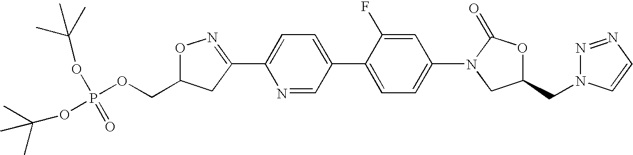

[3-(5-Bromopyridin-2-yl)-4,5-dihydroisoxazol-5-yl]methanol (3.5 g, 13.6 mmol) was dissolved in THF (100 ml) and cooled to 0° C. Di-tert-butyl N,N-diethylphosphoramidite (4.43 g, 17.7 mmol) was then added followed by addition of tetrazole (1.24 g, 17.7 mmol). The reaction was allowed to stir for 30 minutes and then cooled to −40° C. 3-chloroperoxybenzoic acid (5 g, 20.4 mmol) in dichloromethane (100 ml) was then added drop wise using an addition funnel. The reaction was then placed in a 25° C. water bath and allowed to stir for 30 minutes. The reaction was then cooled to 0° C., quenched with a 10% sodium bisulfite solution (50 ml) and extracted with ether (3×50 ml). The organic layers were collected, washed with a saturated sodium bicarbonate solution (2×30 ml), dried over Na₂SO₄, and concentrated in vacuo to yield a crude residue. The residue was purified by column chromatography 15% EtOAc/Hexane to yield [3-(5-bromopyridin-2-yl)-4,5-dihydroisoxazol-5-yl]methyl di-tert-butyl phosphate as a clear oil (2 g). [3-(5-bromopyridin-2-yl)-4,5-dihydroisoxazol-5-yl]methyl di-tert-butyl phosphate (0.8 g, 1.785 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (0.6 g, 1.54 mmol), potassium carbonate (1.5 g, 10.7 mmol), and tetrakis(triphenylphosphine)palladium(0) (0.2 g, 0.18 mmol) were added to DMF (10 ml) and distilled water (1 ml) and heated to 85° C. for 45 minutes. The reaction was filtered through celite and washed with EtOAc (3×20 ml). The organic layers were then collected, washed with distilled water (3×20 ml), dried over Na₂SO₄, and concentrated in vacuo. The residue was purified by column chromatography 0-5% MeOH/CH₂Cl₂ to yield the product as a white solid (600 mg).

¹H-NMR (300 MHz, DMSO-d₆) δ: 1.35 (s, 18H); 3.35 (d, 1H); 3.57 (m, 1H); 3.76 (m, 3H); 4.29 (t, 1H); 4.84 (d, 2H); 5.00 (m, 1H); 5.21 (m, 1H); 7.39 (d, 1H); 7.60 (d, 1H); 7.70 (t, 1H); 7.57 (s, 1H); 8.02 (m, 2H); 8.18 (s, 1H); 8.82 (s, 1H).

### EXAMPLE 42

### [3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl pivalate

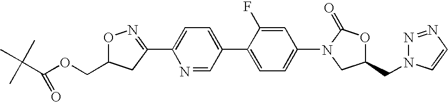

(5R)-3-(3-Fluoro-4-{6-[5-(hydroxymethyl)-4,5-dihydroisoxazol-3-yl]pyridin-3-yl}phenyl)-5-(1H-1,2,3-triazol-1-ylmethyl)-1,3-oxazolidin-2-one (Example 36) (240 mg, 0.55 mMol), trimethylacetic acid (140 mg, 1.37 mMol), EDAC-HCl (210 mg, 1.09 mMol), and 4-dimethylaminopyridine (5 mg, 0.04 mMol) were dissolved in 4 ml of DMF and stirred at room temperature for 5 hours. Further portions of trimethylacetic acid (140 mg, 1.37 mMol) and EDAC-HCl (210 mg, 1.09 mMol) were added, and the mixture was stirred for 1 day more. Third portions of trimethylacetic acid (140 mg, 1.37 mMol), and EDAC-HCl (210 mg, 1.09 mMol) were added, followed by pyridine (0.6 ml). The mixture was then warmed to 50° C. for 7 hours, after which tlc indicated partial completion. Ethyl acetate was added, and the solution was washed with water, then saturated brineand dried over sodium sulfate. Evaporation and purification by chromatography (silica gel; elution with 1 to 3% methanol in dichloromethane) gave material which was triturated with 1:1 ether: hexane to give the title compound as a white crystalline solid (80 mg).

MS (electrospray): 523 (M+1) for C₂₆H₂₇FN₆O₅

¹H-NMR (300 MHz, DMSO-d₆) δ: 3.59 (dd, 1H); 3.96 (dd, 1H); 4.14 (dd, 1H); 4.23-4.43 (m, 2H); 4.86 (d, 2H); 5.02 (m, 1H); 5.18 (m, 1H); 7.42 (dd, 1H); 7.59 (dd, 1H); 7.69 (t, 1H); 7.76 (s, 1H); 7.98-8.07 (dd, 2H); 8.18 (s, 1H); 8.82 (s, 1H).

### EXAMPLE 43

### [(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl N,N-diethyl-β-alaninate

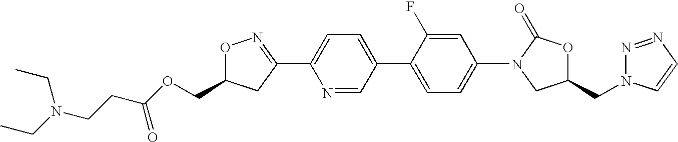

(5R)-3-(3-Fluoro-4 {&[(5S)-5-(hydroxymethyl)-4,5-dihydroisoxazol-3-yl]pyridin-3-yl}phenyl)-5-(1H-1,2,3-triazol-1-ylmethyl)-1,3-oxazolidin-2-one (Example 36) (0.25 g, 0.57 mmol), N,N-diethyl-p-alanine hydrochloride (0.24 g, 1.43 mmol), 4-dimethylaminopyridine (0.02 g, 0.16 mmol), and 1-[3-(dimethylamino)propyl]-3-ethylcarbodiimide hydrochloride (0.25 g, 1.30 mmol) were combined in DMF (4 ml). The suspension was allowed to stir for one hour at room temperature. The mixture was then diluted with acetonitrile:ether (1:1) and filtered. The solids were dissolved in a minimum amount of methanol and submitted directly to purification via chromatography (silica gel, 5 to 20% methanol in dichloromethane). Evaporation of the product containing fractions and trituration of the resulting solid with diethyl ether yielded the title compound as a white solid (70 mg), melting point: 167° C.

MS (electrospray): 566 (MH⁺) for C₂₈H₃₂FN₇O₅

¹H-NMR (300 MHz DMSO-d₆) δ: 1.13 (bt, 6H); 2.82 (bm, 2H); 3.08 (bm, 2H); 3.60 (dd, 1H); 3.96 (dd, 1H); 4.15-4.35 (m, 4H); 4.86 (d, 2H); 5.02 (m, 1H); 5.19 (m, 1H); 7.42 (dd, 1H); 7.58 (dd, 1H); 7.68 (t, 1H); 7.76 (s, 1H); 8.00 (d, 1H); 8.07 (d, 1H); 8.18 (s, 1H); 8.83 (s, 1H).

### EXAMPLE 44

### [3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl methyl succinate

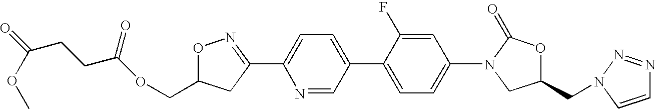

(5R)-3-(3-Fluoro-4-{6-[5-(hydroxymethyl)-4,5-dihydroisoxazol-3-yl]pyridin-3-yl}phenyl)-5-(1H-1,2,3-triazol-1-ylmethyl)-1,3-oxazolidin-2-one (Example 36) (120 mg, 0.27 mmol) was dissolved in 10 mL anhydrous dimethylformamide and triethylamine (140 μL, 1 mmol) was added. Methyl 4-chloro-4-oxobutanoate (100 μL, 0.54 mmol) was slowly added and the mixture was stirred for 2 hours at 40° C. The reaction was quenched with saturated aqueous sodium hydrogencarbonate solution and extracted with dichloromethane (3×100 mL). The combined organic layers were dried over sodium sulfate, concentrated to dryness and purified by preparative HPLC using a gradient from 35 to 70% acetonitrile in water containing 0.1% trifluoroacetic acid to give 27 mg (18%) of the diastereomeric title compound as a trifluoroacetate salt.

MS (APCI): 553 (M+1) for C₂₆H₂₅N₆O₇F

NMR (300 MHZ) (CDCl₃) δ: 2.67 (m, 4H); 3.42 (dd, 1H); 3.62 (m, 4H); 4.04 (t, 1H); 4.31 (m, 3H); 4.85 (d, 2H); 5.14 (m, 2H); 7.49 (m, 2H); 7.82 (d, 2H); 7.99 (d, 1H); 8.16 (d, 1H); 8.83 (s, 1H); 1H in the aromatic range not detected, probably underneath solvent peak

NMR (300 MHZ) (DMSO-d₆) δ: 2.57 (m, 4H); 3.32 (dd, 1H); 3.97 (m, 1H); 4.23 (m, 3H); 4.86 (d, 2H); 5.10 (m, 1H); 5.14 (m, 1H) 7.42 (d, 1H); 7.58 (d, 1H); 7.70 (t, 1H); 7.78 (s, 1H); 8.03 (m, 2H); 8.20 (s, 1H), 8.83 (s, 1H), 5H (methyl- and methylene protons) in the 3.3 ppm range not detected, probably underneath water peak

¹⁹F-NMR (300 MHZ) (DMSO-d₆) δ: −115.98 ppm, −74.00 ppm (trifluoroacetate)

### EXAMPLE 45

### Ethyl[3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl succinate

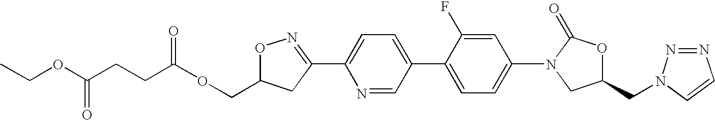

(5R)-3-(3-Fluoro-4-{6-[5-(hydroxymethyl)-4,5-dihydroisoxazol-3-yl]pyridin-3-yl}phenyl)-5-(1H-1,2,3-triazol-1-ylmethyl)-1,3-oxazolidin-2-one (Example 36) (120 mg, 0.27 mmol) was dissolved in 10 mL anhydrous dimethylformamide and triethylamine (140 μL, 1 mmol) was added. Ethyl 4-chloro-4-oxobutanoate (115 μL, 0.54 mmol) was slowly added and the mixture was stirred for 2 hours at 40° C. The reaction was quenched with aqueous saturated sodium hydrogencarbonate solution and extracted with dichloromethane (3×100 mL). The combined organic layers were dried over sodium sulfate, concentrate to dryness and purified by preparative HPLC using a gradient from 35 to 70% acetonitrile in water containing 0.1% trifluoroacetic acid to give 22 mg (15%) of the diastereomeric title compound containing 5 mol % trifluoroacetate salt.

MS (APCI): 567 (M+1) for C₂₇H₂₇N₆O₇F

NMR (300 MHZ) (DMSO-d₆) δ: 1.14 (t, 3H); 2.53 (m, 4H); 3.31 (dd, 1H); 3.61 (dd, 1H); 4.00 (m, 3H); 4.13 (dd, 1H); 4.25 (dd, 2H), 4.86 (d, 2H); 4.99 (m, 1H); 5.18 (m, 1H); 7.45 (dd, 1H); 7.57 (m, 1H); 7.69 (t, 1H); 7.77 (s, 1H); 8.02 (d, 1H); 8.05 (d, 1H); 8.18 (s, 1H); 8.82 (s, 1H)

¹⁹F-NMR (300 MHZ) (DMSO-d₆) δ: −116.00; −73.37 (trifluoroacetate)

### EXAMPLE 46

### [(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl nicotinate

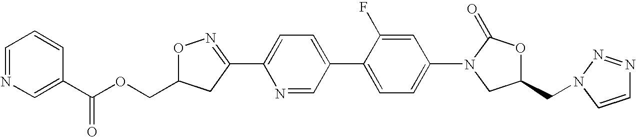

(5R)-3-(3-Fluoro-4-{6-[5-(hydroxymethyl)-4,5-dihydroisoxazol-3-yl]pyridin-3-yl}phenyl)-5-(1H-1,2,3-triazol-1-ylmethyl)-1,3-oxazolidin-2-one (Example 36) (120 mg, 0.27 mmol) was suspended in 2 mL of anhydrous dimethylformamide and triethylamine (160 μL, 1.2 mmol) was added. Nicotinoyl chloride hydrochloride (59 mg, 0.32 mmol) was added and the mixture was slowly warmed to 40° C. Within 10 minutes the solution turned dark and consumption of starting material was observed by thin layer chromatography. The solvent was removed in vacuo and the product isolated by preparative HPLC using a gradient from 5 to 95% of acetonitrile in water containing 0.1% trifluoroacetate. The combined HPLC fractions were concentrated, treated with aqueous saturated sodium hydrogencarbonate solution, extracted with ethylacetate and concentrated to dryness to give 15 mg (11%) of a white solid.

MS (APCI): 544 (M+1) for C₂₇H₂₂N₇O₅F

NMR (300 MHZ) (DMSO-d₆) δ: 3.45 (dd, 1H); 3.66 (dd, 1H); 3.95 (dd, 1H); 4.29 (t, 1H); 4.46 (dd, 1H); 4.55 (dd, 1H); 4.85 (d, 2H), 5.17 (m, 2H); 7.42 (dd, 1H); 7.57 (m, 2H); 7.70 (t, 1H); 7.77 (s, 1H); 8.02 (t, 1H); 8.08 (d, 1H); 8.18 (s, 1H); 8.23 (dd, 1H); 8.78 (d, 1H); 8.83 (s, 1H); 9.01 (s, 1H)

¹⁹F-NMR (300 MHZ) (DMSO-d₆) δ: −115.98 ppm; no trifluoroacetate peak observed

### EXAMPLE 47

### {[3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methoxy}methyl pivalate

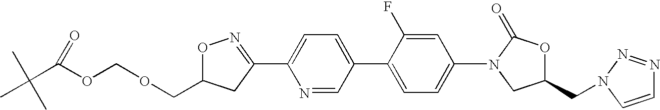

(5R)-3-(3-Fluoro-4-([5-(hydroxymethyl)-4,5-dihydroisoxazol-3-yl(pyridin-3-yl}phenyl)-5-(1H-1,2,3-triazol-1-ylmethyl)-1,3-oxazolidin-2-one (Example 36) (120 mg, 0.27 mmol) was dissolved in 8 mL anhydrous dimethylformamide and sodium hydride (13.2 mg, 0.34 mmol, based on 60% purity) in 2 mL of anhydrous dimethylformamide was added at −20° C. Chloromethyl pivalate (44 μL, 0.30 mmol) was slowly added and the mixture allowed to room temperature and then warmed to 40° C. for 1 hour. Then, the mixture was quenched with 1 mL of saturated aqueous sodium hydrogencarbonate solution, the solvent removed in vacuo and purified by preparative HPLC using a gradient from 55 to 75% acetonitrile in water containing 0.1% trifluoroacetic acid to yield 27 mg (20%) of the title compound as a yellow salt in a 1:1 ratio with triflouroacetate.

MS (APCI): 553 (M+1) for C₂₇H₂₉N₆O₆F

NMR (300 MHZ) (DMSO-d₆) δ: 1.17 (s, 9H); 3.29 (dd, 1H); 3.54 (dd, 1H); 3.77 (m, 2H); 3.98 (m, 2H); 4.31 (t, 2H); 4.87 (m, 2H), 5.29 (m, 2H); 7.43 (dd, 1H); 7.60 (dd, 1H); 7.68 (t, 1H); 7.71 (s, 1H); 8.04 (dd, 2H); 8.20 (s, 1H); 8.83 (s, 1H)

¹⁹F-NMR (300 MHZ) (DMSO-d₆) δ: −116.00 ppm; −72.55 ppm (trifluoroacetate)

### EXAMPLE 48

### [3-(5-{2-Fluoro-4-[(5R)-2-oxo-5-(1H-1,2,3-triazol-1-ylmethyl)-1,3-oxazolidin-3-yl]pheny}pyridin-2-yl)-4,5-dihydroisoxazol-5-yl]methyl 4-nitrobenzoate

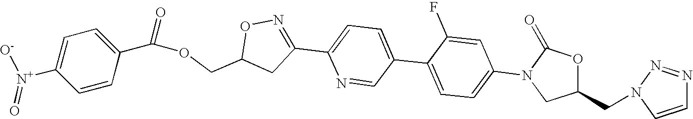

(5R)-3-(3-Fluoro-4-{6-[5-(hydroxymethyl)-4,5-dihydroisoxazol-3-yl]pyridin-3-yl}phenyl)-5-(1H-1,2,3-triazol-1-ylmethyl)-1,3-oxazolidin-2-one Example 36) (100 mg, 0.23 mmol) was suspended with 2 mL of anhydrous dimethylformamide and triethylamine (80 μL, 0.58 mmol) was added. 4-nitrobenzoyl chloride (80 mg, 0.54 mmol) was added and the mixture was stirred for 2 hours at 50° C. The reaction was quenched with methanol (1 mL), the solvent removed in vacuo and the product isolated by preparative thin layer chromatography using 10% (v/v) of methanol in dichloromethane as eluent to give 40 mg (30%) of the title compound as an off white solid.

MS (APCI): 588 (M+1) for C₂₈H₂₂N₇O₇P

NMR (300 MHZ) (DMSO-d₆) δ: 3.48 (dd, 1H); 3.70 (dd, 1H); 3.97 (dd, 1H); 4.31 (t, 1H); 4.49 (dd, 1H), 4.55 (dd, 1H); 4.88 (d, 2H); 5.20 (m, 2H); 7.45 (dd, 1H); 7.60 (dd, 1H); 7.71 (t, 1H); 7.79 (s, 1H); 8.03 (d, 1H); 8.07 (d, 1H); 8.16 (s, 1H); 8.19 (d, 2H); 8.31 (d, 2H); 8.85 (s, 1H)

### EXAMPLE 49

### 4-{[3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methoxy}-4-oxobutanoic acid

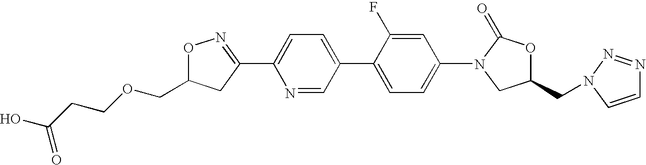

(5R)-3-(3-Fluoro-4-{6-[5-(hydroxymethyl)-4,5-dihydroisoxazol-3-yl]pyridin-3-yl}phenyl)-5-(1H-1,2,3-triazol-1-ylmethyl)-1,3-oxazolidin-2-one (Example 36) (212 mg, 0.48 mmol) was suspended in 3 mL of anhydrous dimethylformamide. Anhydrous pyridine (700 μL, 8.7 mmol), 4-dimethylaminopyridine (DMAP) (30 mg, 0.25 mmol) and succinic anhydride (125 mg, 1.25 mmol) were added and the solution was stirred for 16 hours at room temperature. The reaction was quenched with methanol (1 mL), solvents were removed in vacuo and the product purified by chromatography on silicagel using a gradient from 0 to 20% methanol in dichloromethane followed by an aqueous wash and lyophilisation to remove residual dimethylformamide to yield 120 mg (50%) of the title compound as an off white salt.

MS (APCI): 539 (M+1) for C₂₅H₂₃N₆O₇F

NMR (300 MHZ) (DMSO-d₆) δ: 2.42 (d, 2H); 3.30 (dd, 2H); 3.57 (t, 1H); 3.96 (dd, 1H) 4.23 (m, 2); 4.86 (d, 2H); 5.00 (m, 1H); 5.18 (m, 1H); 7.43 (dd, 1H); 7.58 (dd, 1H); 7.69 (t, 1H); 7.77 (s, 1H); 8.02 (d, 1H); 8.07 (d, 1H); 8.18 (s, 1H); 8.82 (s, 1H); 2 methylene protons overlap with solvent peak, 2 methylene protons enhanced by residual HOD peak.

¹⁹F-NMR (300 MHZ) (DMSO-d₆) δ: −115.94

### EXAMPLE 50

### (5S)-3-{4′-[5,5-Bis(hydroxymethyl)-4,5-dihydroisoxazol-3-yl]-2,2′-difluorobiphenyl-4-yl}-5-[(1,2,5-thiadiazol-3-ylamino)methyl]-1,3-oxazolidin-2-one

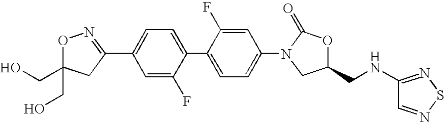

tert-Butyl [((5R)-3-{4′-[5,5-bis(hydroxymethyl)-4,5-dihydroisoxazol-3-yl]-2,2′-difluorobiphenyl-4-yl}-2-oxo-1,3-oxazolidin-5-yl)methyl]1,2,5-thiadiazol-3-ylcarbamate (506.0 mg, 0.82 mmol) was dissolved in dichloromethane (10 ml) and cooled to 0° C. Trifluoroacetic acid (4 ml) was added and the reaction mixture was stirred at 0° C. for 3 hours. The reaction mixture was concentrated in vacuo. The residue was partitioned between ethyl acetate (100 ml) and aqueous saturated sodium hydrogen carbonate solution (100 ml). The organic layer was dried over magnesium sulphate, filtered and then concentrated in vacuo. The resulting oil was dissolved in dichloromethane (2 ml) and subjected to chromatography (SiO₂ 20 g bond elut columns, 0 to 10% methanol/dichloromethane) to yield 251 mg (59%) of (5S)-3-{4′-[5,5-bis(hydroxymethyl)-4,5-dihydroisoxazol-3-yl]-2,2′-difluorobiphenyl-4-yl}-5-[(1,2,5-thiadiazol-3-ylamino)methyl]-1,3-oxazolidin-2-one as a white solid.

MS (ESP+): (M+H)^{+ 518.12} for C₂₃H₂₁F₂N₅O₅S

NMR (DMSO-d₆) δ: 3.28 (s, 2H), 3.53 (d, 4H), 3.72 (m, 2H), 3.91 (q, 1H), 4.26 (t, 1H); 4.98 (m, 1H), 5.06 (t, 2H); 7.47 to 7.68 (m, 6H); 7.80 (t, 1H), 8.08 (s, 1H).

The intermediates for this compound were prepared as follows:

### tert-Butyl [((5R)-3-{4′-[5,5-bis(hydroxymethyl)-4,5-dihydroisoxazol-3-yl]-2,2′-difluorobiphenyl-4-yl}-2-oxo-1,3-oxazolidin-5-yl)methyl]1,2,5-thiadiazol-3-ylcarbamate

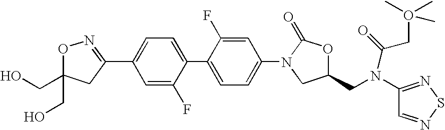

tert-Butyl {[(5R)-3-(3-fluoro-4-iodophenyl)-2-oxo-1,3-oxazolidin-5-yl]methyl} 1,2,5-thiadiazol-3-ylcarbamate (542 mg, 1.04 mmol) (cf. Example 3 above), {3-[3-fluoro-4-(trimethylstannyl)phenyl]-4,5-dihydroisoxazole-5,5-diyl}dimethanol (485 mg, 1.25 mmol) and copper (I) iodide (82 mg, 0.42 mmol) were dissolved in dry 1-methyl-2-pyrrolidinone (10 ml) and the reaction mixture placed under an atmosphere of argon.

Tetrakis(triphenylphosphine)palladium(0) (120 mg, 0.1 mmol) was added and the reaction mixture stirred for 48 hours at 90° C. The reaction mixture was cooled to room temperature then poured into water (100 ml). The product was extracted into ethyl acetate (100 ml). The ethyl acetate layer was separated, dried over magnesium sulphate, filtered then concentrated in vacuo. The crude product was then dissolved in diceloromethane (2 ml) and subjected to chromatography (SiO₂ 50 g bond elute column, 50 to 100% ethyl acetate/hexane) to yield 512 mg (80%) of the desired compound as a yellow oil.

MS (ESP+): (M+H)⁺ 618.21 for C₂₈H₂₉F₂N₅O₇S

### 3-[3-fluoro-4-(trimethylstannyl)phenyl]-4,5-dihydroisoxazole-5,5-diyl}dimethanol

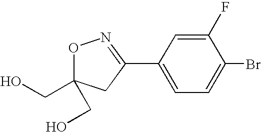

2-Methylene-1,3-propanediol (2.20 g, 25.0 mM) was stirred in dichloromethane (20 mL) and cooled to 0° C. A 1 N solution of diethylzinc in hexanes (3.40 g, 27.5 mM) was added followed by a solution of 4-bromo-3-fluoro-N-hydroxybenzenecarboximidoyl chloride (6.30 g, 25.0 mM) in dichloromethane (40 mL). The reaction was allowed to warm to room temperature and was complete after four hours. The solution was diluted with ammonium chloride and extracted using dichloromethane. The organic layer was dried (magnesium sulfate), filtered and concentrated to give the desired product as a yellow solid (4.72 g).

MS (ESP): 305 (MH⁺) for C₁₁H₁₁BrFNO₃

300 MHz NMR (DMSO-d₆) δ: 3.29 (s, 2H); 3.55 (s, 2H); 3.57 (s, 2H); 5.10 (t, 2H); 7.52 (d, 1H); 7.68 (d, 1H); 7.86 (t, 1H).

### EXAMPLE 51

### (5R)-3-[4-(6-{(5S)-5-[(1R)-1,2-Dihydroxyethyl]-4,5-dihydroisoxazol-3-yl}pyridin-3-yl)-3-fluorophenyl]-5-(1H-1,2,3-triazol-1-ylmethyl)-1,3-oxazolidin-2-one

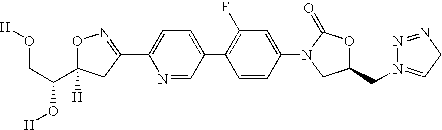

(5R)-3-[4-(6-{(5S)-5-[(4R)-2,2-Dimethyl-1,3-dioxolan-4-yl]-4,5-dihydroisoxazol-3-yl}pyridin-3-yl)-3-fluorophenyl]-5-(1H-1,2,3-triazol-1-ylmethyl)-1,3-oxazolidin-2-one (0.225 g, 0.44 mmol) was dissolved in tetrahydrofuran (10 ml) and 1N HCl (10 ml, 10 mmol) and heated to 50° C. in an oil bath for 90 minutes. The reaction was cooled to room temperature, concentrated in vacuo, with acetonitrile added repeatedly as a co-solvent to minimize the amount of water present, leaving a yellow solid. The crude product was dissolved in a mixture of methanol (30 ml) and dichloromethane (10 ml), and then MP-carbonate resin (1.5 g, 4.6 mmol) was added. The mixture was placed in an ice bath and stirred at 0° C. for one hour. The MP-carbonate resin was filtered off and the filtrate was concentrated in vacuo. The resultant crude product was adsorbed onto silica gel (1.5 g) and purified by column chromatography using a 5-gram Isolute silica gel column on the FlashMaster II system, using a gradient from 0% to 5% methanol in dichloromethane with a solvent flow rate of 10 ml/minute, to give the title product (0.072 g, 34.8% yield) as a white solid.

MS (APCI): 469.2 (MH⁺) for C₂₂H₂₁FN₆O₅

MS (ESP): 469.09 (MH⁺) for C₂₂H₂₁FN₆O₅

¹H-NMR (500 Mz)(DMSO-d₆) δ: 3.40 (m, 4H); 3.65 (m, 1H); 3.96 (dd, 1H); 4.29 (t, 1H); 4.68 (t, 1H); 4.76 (m, 1H); 4.86 (d, 2H); 5.09 (d, 1H); 5.18 (m, 1H); 7.42 (dd, 1H); 7.58 (dd, 1H); 7.69 (t, 1H); 7.77 (s, 1H); 7.98 (d, 1H); 8.04 (m, 1H); 8.18 (s, 1H); 8.81 (s, 1H).

¹⁹F-NMR (500 Mz)(DMSO-d₆) δ: −115.96 (s, 1F)

### EXAMPLE 52

### (5R)-3-[4-(6-{(5R)-5-[(1R)-1,2-Dihydroxyethyl]-4,5-dihydroisoxazol-3-yl}pyridin-3-yl)-3-fluorophenyl]-5-(1H-1,2,3-triazol-ylmethyl)-1,3-oxazolidin-2-one

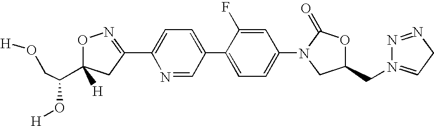

5-Bromo-2-{(5R)-5-[(4R)-2,2-dimethyl-1,3-dioxolan-4-yl]-4,5-dihydroisoxazol-3-yl}pyridine (340 mg, 1.04 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (366 mg, 0.94 mmol), K₂CO₃ (780 mg, 5.65 mmol), and tetrakis(triphenylphosphine)palladium (0) (109 mg, 0.094 mmol) were added to DMF (8 ml) and distilled water (0.8 ml). The reaction was heated to 85° C. for 30 minutes and then cooled to room temperature. Ethyl acetate (25 ml) was then added and the mixture was filtered through a 45-micron filter. The filtrate was concentrated in vacuo to yield a crude residue. The residue was purified by column chromatography using 0-4% MeOH/CH₂Cl₂ to yield a white powder (180 mg). The white powder (180 mg) was added to THF (20 ml) followed by addition of 1N HCl (5 ml) and the reaction was allowed to stir for 4 hours. Trifluoroacetic acid (2 ml) was then added and the reaction was allowed to stir for an additional 30 minutes. The reaction mixture was then concentrated in vacuo to yield a crude residue. The residue was then purified by column chromatography using 0-2% MeOH/CH₂Cl₂ to yield the product as a white solid (50 mg).

MS (ESP): 469.11 (for C₂₂H₂₁FN₆O₅

¹H-NMR (50 MHz)(DMSO-d₆) δ: 3.38 (dd, 1H); 3.48 (m, 4H); 3.95 (m, 1H); 4.29 (t, 1H); 4.69 (t, 1H); 4.79 (t, 1H); 4.86 (d, 2H); 4.98 (d, 1H); 5.18 (m, 1H); 7.42 (d, 1H); 7.58 (d, 1H); 7.69 (t, 1H); 7.78 (s, 1H); 7.98 (d, 1H); 8.06 (d, 1H); 8.18 (s, 1H); 8.81 (s, 1H).

The intermediates for Examples 51 and 52 were prepared as follows:

### 5-Bromo-2-{5-[(4R)-2,2-dimethyl-1,3-dioxolan-4-yl]-4,5-dihydroisoxazol-3-yl}-3-fluoropyridine

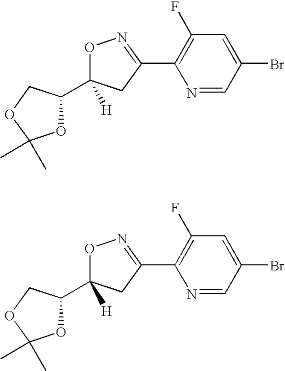

(4S)-2,2-Dimethyl-4-vinyl-1,3-dioxolane (R. J. Crawford, S. B. Lutener, R. D. Cockcroft, Can. J Chem; 54, 3364 (1976)) (2.08 g, 16.2 mmol) was combined with 5-bromo-N-hydroxypyridine-2-carboximidoyl chloride (2.55 g, 10.8 mmol) under a nitrogen atmosphere. Anhydrous tetrahydrofuran (15 ml) was added and mixed for fifteen minutes, followed by the slow addition of a solution of diisopropylethylamine (3.8 m]L, 21.6 mmol) in anhydrous tetrahydrofuran (15 ml) via dropping funnel at room temperature. The reaction was stirred at room temperature for three hours, then diluted with ethyl acetate (300 ml), washed with water (1×100 ml), brine (1×50 ml), and dried over anhydrous magnesium sulfate. The solvents were removed in vacuo, producing a crude product mixture which was dissolved in dichloromethane (10 ml), applied to a pre-wettened 70-gram Isolute silica gel column and eluted with a gradient from 20:80 to 50:50 ethyl acetate:hexanes. The pure product was recovered as a mixture of diastereomers (in a ratio of approximately 75:25 by ¹H NMR and chiral column analyses, with the major product being the (+)-diastereomer). The two diastereomers were separated on silica gel using a very slow gradient from 10:90 to 20:80 ethyl acetate:hexanes (Rf in 20:80 ethyl acetate hexanes: major=0.44, minor=0.32). The diastereomers were analysed by ¹H NMR and optical rotation. The stereochemistry assignments were made using information from the following sources: Gravestock, M. B., Paton, R. M., Todd, C. J., Tetrahedron: Asymmetry, 1995, 6, 11, pages 2723-2730; and the PhD Thesis of Christine J. Todd, University of Edinburgh, 1995, “Application of Nitrile Oxide-Isoxazoline Chemistry for the Synthesis of 2-Ulosonic Acid Analogues”

### Analyses of 5-bromo-2-1 (5S)-5-[(4R)-2,2-dimethyl-1,3-dioxolan-4-yl]-4,5-dihydroisoxazol-3-yl)pyridine

MS (APCI): 327.0, 329.0 (MH⁺) for C₁₃H₁₅BrN₂O₃

MS (ESP): 327.20, 329.20 (MH⁺) for C₁₃H₁₅BrN₂O₃

Optical Rotation: (589 nm, 20° C.) [α]=+113.6 (c=0.25 in methanol)

¹H-NMR (500 Mz)(CDCl₃) δ: 1.34 (s, 3H); 1.42 (s, 3H); 3.50 (s, 1H); 3.52 (d, 1H); 3.91 (m, 1H); 4.14 (m, 2H); 4.73 (m, 1H); 7.83 (dd, 1H); 7.88 (d, 1H); 8.65 (d, 1H).

### Analyses of 5-bromo-2-{(5R)-5-[(4R)-2,2-dimethyl-1,3-dioxolan-4-yl]-4,5-dihydroisoxazol-3-yl}pyridine

MS (APCI): 327.0, 329.0 (MH⁺) for C₁₃H₁₅BrN₂O₃

MS (ESP): 327.20, 329.20 (MH⁺) for C₁₃H₁₅BrN₂O₃

Optical Rotation: (589 nm, 20° C.) [α]=−146.4 (c=0.25 in methanol)

¹H-NMR (500 Mz)(CDCl₃) δ: 1.35 (s, 3H); 1.44 (s, 3H); 3.33 (dd, 1H); 3.51 (dd, 1H); 3.86 (dd, 1H); 4.09 (dd, 1H); 4.30 (m, 1H); 4.83 (m, 1H); 7.84 (dd, 1H); 7.90 (d, 1H); 8.64 (d, 1H).

### (5R)-3-[3-Fluoro-4-(4,4,5,5-tetramethyl-1,3,2-dioxaborolan-2-yl)phenyl]-5-(1H-1,2,3-triazol-1-ylmethyl)-1,3-oxazolidin-2-one

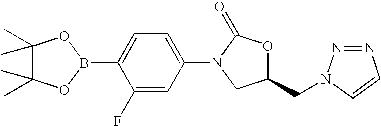 see Example 13.

### (5R)-3-[4-(6-{(5S)-5-[(4R)-2,2-Dimethyl-1,3-dioxolan-4-yl]-4,5-dihydroisoxazol-3-yl}pyridin-3-yl)-3-fluorophenyl]-5-(1H-1,2,3-triazol-1-ylmethy)-1,3-oxazolidin-2-one

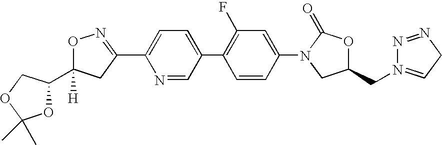

5-Bromo-2-{(5S)-[(4R)-2,2-dimethyl-1,3-dioxolan-4-yl]-4,5-dihydroisoxazol-3-yl}-3-fluoropyridine (0.468 g, 1.43 mmol), and (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (0.505 g, 1.30 mmol) were dissolved in anhydrous N,N-dimethylformamide (10 ml). Potassium carbonate (0.90 g, 6.50 mmol) was added, followed by water (1 ml), and then tetrakis(triphenylphosphine)palladium (0) (0.15 g, 0.13 mmol). The reaction was heated to 85° C. for 60 minutes. The reaction was then cooled to room temperature, diluted with ethyl acetate (15 ml), stirred at room temperature for ten minutes, and the resulting precipitate was filtered off. The filtrate was concentrated in vacuo to remove the ethyl acetate and N,N-dimethylformamide. The resultant thick black oil was dissolved in dichloromethane (15 ml) and purified by column chromatography, using a 50-gram Isolute silica gel column (pre-wettened with dichloromethane), eluting with 0-4% methanol in dichloromethane. The title product (0.265 g, 40.0% yield) was recovered as a white solid.

MS (APCI): 509.2 (MH⁺) for C₂₅H₂₅FN₆O₅

MS (ESP): 509.09 (MH⁺) for C₂₅H₂₅FN₆O₅

¹H-NMR (500 Mz)CDCl₃) δ: 1.35 (s, 3H); 1.43 (s, 3H); 3.56 (s, 1H); 3.58 (d, 1H); 3.92 (dd, 1H); 4.00 (dd, 1H); 4.17 (m, 3H); 4.75 (m, 1H); 4.82 (d, 2H); 5.11 (m, 1H); 7.22 (dd, 1H); 7.43 (t, 1H); 7.46 (dd, 1H); 7.77 (dd, 2H); 7.86 (m, 1H); 8.04 (d, 1H); 8.74 (s, 1H).

¹⁹F-NMR (300 Mz)(CDCl₃) δ: −114.23 (s, 1F)

### 5-Bromo-N-hydroxypyridine-2-carboximidoyl chloride

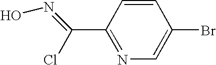

5-Bromopyridine-2-carbaldehyde oxime (49.5 g, 246.3 mmol) was dissolved in DMF (150 ml) followed by addition of N-chlorosuccinimide (39.5 g, 295.5 mmol). HCl gas was then bubbled in the solution for 20 seconds to initiate the reaction, which was then allowed to stir for 1 hr. The reaction was poured into distilled water (1 L) and the precipitate was collected by vacuum filtration. The filter cake was washed with distilled water (2×500 ml) and then dried overnight in a vacuum oven at 60° C. (−30 inches Hg) to yield the product as a white powder (55 g).

¹H-NMR (300 Mz)(CDCl₃) δ: 7.73 (d, 1H); 8.09 (d, 1H); 8.73 (s, 1H); 12.74 (s, 1H).

NOTE: Lachrymator.

### EXAMPLE 53

### (5R)-3-[4-(6-{(5S)-[(1S)-1,2-Dihydroxyethyl]-4,5-dihydroisoxazol-3-yl}pyridin-3-yl)-3-fluorophenyl]-5-(1H-1,2,3-triazol-1-ylmethyl)-1,3-oxazolidin-2-one

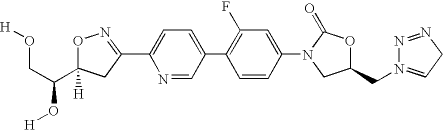

(5R)-3-[4-(6-{(5S)-5-[(4S)-2,2-Dimethyl-1,3-dioxolan-4-yl]-4,5-dihydroisoxazol-3-yl}pyridin-3-yl)-3-fluorophenyl]-5-(1H-1,2,3-triazol-1-ylmethyl)-1,3-oxazolidin-2-one (0.31 g, 0.61 mmol) was dissolved in tetrahydrofuran (6 ml) and 1N HCl (6 ml, 6 mmol) and heated to 50° C. for three hours. The reaction was cooled to room temperature concentrated in vacuo, with acetonitrile added repeatedly as a co-solvent to minimize the amount of water present, leaving a yellow solid. The crude product was dissolved in a mixture of methanol (10 ml) and dichloromethane (10 ml), and MP-carbonate resin (2.1 g, 6.1 mmol) was added. The mixture was stirred at room temperature for one hour. The MP-carbonate was filtered off, and the solvents were removed in vacuo. The pure product (0.24 g, 84.0% yield) was recovered as a light yellow solid.

MS (APCI): 469.2 (MH⁺) for C₂₂H₂₁FN₆O₅

MS (ESP): 469.13 (MH⁺) for C₂₂H₂₁FN₆O₅

¹H-NMR (500 Mz)(DMSO-d₆) δ: 3.38 (m, 1H); 3.49 (m, 4H); 3.96 (dd, 1H); 4.29 (t, 1H); 4.69 (t, 1H); 4.80 (m, 1H); 4.86 (d, 2H); 4.98 (d, 1H); 5.18 (m, 1H); 7.42 (dd, 1H); 7.59 (dd, 1H); 7.68 (t, 1H); 7.77 (s, 1H); 7.97 (d, 1H); 8.04 (m, 1H); 8.18 (s, 1H); 8.81 (s, 1H).

¹⁹F-NMR (300 Mz)(DMSO-d₆) δ: −115.96 (s, 1F)

### EXAMPLE 54

### (5R)-3-[4-(6-{(5R)S-5-[(1S)-1,2-Dihydroxyethyl]-4,5-dihydroisoxazol-3-yl}pyridin-3-yl)-3-fluorophenyl]-5-(1H-1,2,3-triazol-1-ylmethyl)-1,3-oxazolidin-2-one

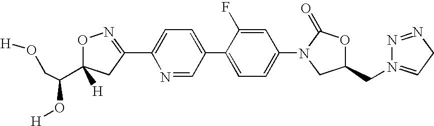

5-Bromo-2-{(5R)-5-[(4S)-2,2-dimethyl-1,3-dioxolan-4-yl]-4,5-dihydroisoxazol-3-yl}pyridine (464 mg, 1.41 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (500 mg, 1.29 mmol), K₂CO₃ (1067 mg, 7.73 mmol), and tetrakis(triphenylphosphine)palladium (0) (149 mg, 0.128 mmol) were added to DMF (8 ml) and distilled water (0.88 ml). The reaction was heated to 85° C. for 30 minutes and then cooled to room temperature. Ethyl acetate (25 ml) was then added and the mixture was filtered through a 45-micron filter. The filtrate was concentrated in vacuo to yield a crude residue. The residue was purified by column chromatography using 0-4% MeOH/CH₂Cl₂ to yield a white powder (331 mg). The white powder (331 mg) was added to THF (20 ml) followed by addition of 1N HCl (20 ml) and then heated at 500C for 1 hour. The reaction mixture was then concentrated in vacuo to yield a crude residue. The residue was then purified by column chromatography using 0-2% MeOH/CH₂Cl₂ to yield the product as a white solid (91.5 mg).

MS (ESP): 469.15 (MH⁺) for C₂₂H₂₁FN₆O₅

¹H-NMR (500 Mz)(DMSO-d₆) δ: 3.41 (m, 5H); 3.96 (m, 1H); 4.29 (dd, 1H); 4.68 (t, 1H); 4.77 (m, 1H); 4.86 (d, 2H); 5.10 (d, 1H); 5.19 (m, 1H); 7.42 (d, 1H); 7.58 (d, 1H); 7.69 (t, 1H); 7.77 (s, 1H); 7.98 (d, 1H); 8.04 (d, 1H); 8.17 (s, 1H); 8.82 (s, 1H).

Intermediates for Examples 53 and 54 were prepared as follows:

### (5R)-3-[4-(6-{(5S)-5-[(4S)-2,2-Dimethyl-1,3-dioxolan-4-yl]-4,5-dihydroisoxazol-3-yl}pyridin-3-yl)-3-fluorophenyl]-5-(1H-1,2,3-triazol-1-ylmethyl)-1,3-oxazolidin-2-one

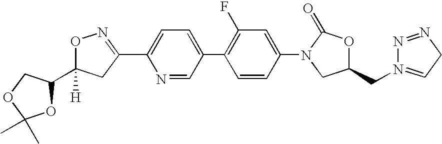

5-Bromo-2-{(5S)-5-[(4S)-2,2-dimethyl-1,3-dioxolan-4-yl]-4,5-dihydroisoxazol-3-yl}pyridine (0.453 g, 1.38 mmol) and (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (0.489 g, 1.26 mmol) were dissolved in anhydrous N,N-dimethylformamide (10 ml). Potassium carbonate (0.87 g, 6.29 mmol) was added, followed by tetrakis(triphenylphosphine)paliadium (O) (0.145 g, 0.13 mmol), and then water (1 ml). The reaction was heated to 85° C. for 50 minutes. The reaction was then cooled to room temperature, diluted with ethyl acetate (35 ml), stirred at room temperature for fifteen minutes, and the resulting precipitate was filtered off. The filtrate was diluted with ethyl acetate (350 ml) and washed with water (100 ml), then brine (75 ml), and then concentrated in vacuo. The resultant crude product was adsorbed onto silica gel (5 g) and purified by column chromatography, using a 50-gram Isolute silica gel column (pre-wettened with dichloromethane), eluting with 0-1% methanol in dichloromethane. The title product (0.34 g, 53.1% yield) was recovered as a light yellow solid; the product was found to contain 3-4 mol % of the oxidized (5R)-3-[4-(6-{5-[(4S)-2,2-dimethyl-1,3-dioxolan-4-yl]isoxazol-3-yl}pyridin-3-yl)-3-fluorophenyl]-5-(1H-1,2,3-triazol-1-ylmethyl)-1,3-oxazolidin-2-one by-product as an impurity.

MS (APCI): 509.2 (MH⁺) for C₂₅H₂₅FN₆O₅

MS (ESP): 509.12 (MH⁺) for C₂₅H₂₅FN₆O₅

### (5R)-3-[4-(6-{(5R)-5-[(4S)-2,2-Dimethyl-1,3-dioxolan-4-yl]-4,5-dihydroisoxazol-3-yl}pyridin-3-yl)-3-fluorophenyl]-5-(1H-1,2,3-triazol-1-ylmethyl)-1,3-oxazolidin-2-one

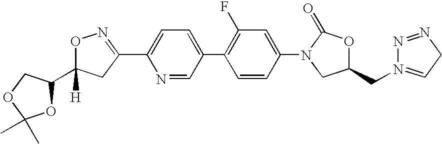

Prepared from 5-Bromo-2-{(5R)-5-[(4S)-2,2-dimethyl-1,3-dioxolan-4-yl]-4,5-dihydroisoxazol-3-yl}pyridine by an analogous process to that described for the (5S) isomer

### 5-Bromo-2-[(5R)-5-{(4S)-2,2-dimethyl-1,3-dioxolan-4-yl]-4,5-dihydroisoxazol-3-yl}pyridine and 5-bromo-2-{(5S)-5-[(4S)-2,2-diethyl-1,3-dioxolan-4-yl]-4,5-dihydroisoxazol-3-yl}pyridine

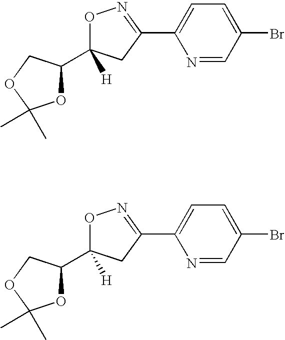

5-Bromo-N-hydroxypyridine-2-carboximidoyl chloride (5 g, 21.3 mmol) and (4R)-2,2-dimethyl-4-vinyl-1,3-dioxolane (5.5 g, 42.55 mmol) were added to THF (30 ml) and then cooled to 0° C. Triethylamine (3.3 ml) in THF (30 ml) was then added drop wise with an addition funnel over 30 minutes. The reaction was allowed to stir for one hour at 0° C. EtOAc (40 ml) was then added and the precipitate was filtered. The filtrate was concentrated in vacuo to yield a crude solid (6.6 g). The crude solid was purified by column chromatography using 0-10% EtOAc/Hexane to yield the S,R isomer (2.5 g) and the S,S isomer (0.6 g) as white solids. The stereochemistry assignments were made using information from the following sources: Gravestock, M. B., Paton, R. M., Todd, C. J., Tetrahedron: Asymmetry, 1995, 6, 11, pages 2723-2730; and the PhD Thesis of Christine J. Todd, University of Edinburgh, 1995, “Application of Nitrile Oxide-Isoxazoline Chemistry for the Synthesis of 2-Ulosonic Acid Analogues”.

(5R): ¹H-NMR (500 Mz)(CDCl₁) δ: 1.37 (s, 3H); 1.45 (s, 3H); 3.53 (d, 2H); 3.93 (m, 1H); 4.17 (m, 2H); 4.76 (m, 1H); 7.83 (m, 2H); 8.67 (s, 1H).

Optical Rotation: (589 nm, 20° C.) [α]=−118.4 (c=2.5 mg/ml in methanol)

(5S): ¹H-NMR (500 Mz)(CDCl₃) δ: 1.35 (s, 3H); 1.44 (s, 3H); 3.32 (dd, 1H); 3.50 (dd, 1H); 3.86 (dd, 1H); 4.09 (dd, 1H); 4.31 (m, 1H); 4.83 (m, 1H); 7.83 (dd, 1H); 7.90 (d, 1H); 8.64 (d, 1H).

Optical Rotation: (589 inn 20° C.) [α]=+145.6 (c=2.5 mg/ml in methanol)

### EXAMPLE 55

### (5R)-3-(4-{6-[5,5-Bis(hydroxymethyl)-4,5-dihydroisoxazol-3-yl]pyridin-3-yl}-3-fluorophenyl)-5-(1H-1,2,3-triazol-1-ylmethyl)-1,3-oxazolidin-2-one

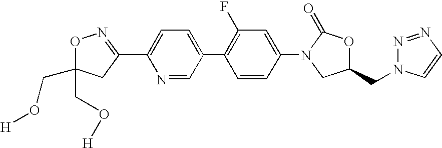

(5R)-3-(4-{6-[5,5-Bis({[tert-butyl(dimethyl)silyl]oxy}methyl)-4,5-dihydroisoxazol-3-yl]pyridin-3-yl}-3-fluorophenyl)-5-(1H-1,2,3-triazol-1-ylmethyl)-1,3-oxazolidin-2-one (0.21 g, 0.30 mmol) was dissolved in anhydrous tetrahydrofuran (10 ml) under a nitrogen atmosphere. Tetrabutylammonium fluoride (0.31 ml, 0.31 mmol) was added drop wise and the reaction was stirred at room temperature for ninety minutes. Ethyl acetate (40 ml) and water (10 ml) were added, followed by brine (20 ml), and the two phases were separated. The ethyl acetate layer was washed with brine, dried over anhydrous magnesium sulfate and concentrated in vacuo. The crude product was found to contain tetrabutylammonium salts, and was dissolved in a mixture or methanol and methylene chloride, adsorbed onto silica gel (1 g) and purified by column chromatography using a 20-gram Isolute silica gel column on the FlashMaster II system using a gradient from 0% to 5% methanol in dichloromethane with a solvent flow rate of 15 my/minute. The recovered product (0.102 g) was recrystallised from tetrahydrofuran, to give the title product (>98% pure) (0.033 g, 23.6% yield).

MS (APCI): 469.2 (MH⁺) for C₂H₂₁FN₆O₅

MS (ESP): 469.16 (MH⁺) for C₂₂H₂₁FN₆O₅

¹H-NMR (300 Mz)(DMSO-d₆) δ: 3.31 (2H, hidden under water peak); 3.51 (broad s, 4H); 3.96 (dd, 1H); 4.29 (t, 1H); 4.86 (d, 2H); 5.02 (broad s, 2H); 5.19 (m, 1H); 7.41 (dd, 1H); 7.58 (dd, 1H); 7.69 (t, 1H); 7.77 (s, 1H); 7.96 (d, 1H); 8.04 (d, 1H); 8.18 (s, 1H); 8.81 (s, 1H).

¹⁹F-NMR (300 Mz)(DMSO-d₆) δ: −115.96 (s, 1F)

The intermediates for Example 55 were prepared as follows;

### (5R)-3-(4-{6-[5,5-Bis({[tert-butyl(dimethyl)silyl]oxy methyl)-4,5-dihydroisoxazol-3-yl]pyridin-3-yl}-3-fluorophenyl-5-(1H-1,2,3-triazol-1-ylmethyl)-1,3-oxazolidin-2-one

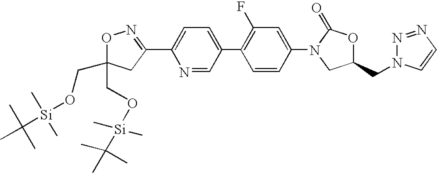

2-[5,5-Bis({[tert-butyl(dimethyl)silyl]oxy)methyl)-4,5-dihydroisoxazol-3-yl]-5-bromopyridine (0.28 g, 0.54 mmol) and (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (0.32 g, 0.81 mmol) were dissolved in anhydrous N,N-dimethylformamide (10 ml). Potassium carbonate (1 N solution) (1.6 ml, 1.63 mmol) was added, followed by water (1 ml), and then tetrakis(triphenylphosphine)palladium (0) (0.094 g, 0.08 mmol). The reaction was heated to 85° C. for 90 minutes. The reaction was then cooled to room temperature, diluted with ethyl acetate (120 ml) and washed with water (2×50 ml), brine (1×40 ml), dried over anhydrous magnesium sulfate and concentrated in vacuo, leaving N,N-dimethylformamide solution (3 ml). The crude product solution was then diluted with dichloromethane (5 ml) and purified by column chromatography using a 20-gram Isolute silica gel column (pre-wettened with dichloromethane) eluting with 0-2% methanol in dichloromethane. The title product (0.205 g, 60.5% yield) was recovered as a white solid.

MS (APCI): 697.2 (MH⁺) for C₃₄H₄₉FN₆O₅Si₂

MS (ESP): 697.08 (MH⁺) for C₃₄H₄₉FN₆O₅Si₂

¹H-NMR (300 Mz)(DMSO-d₆) δ: 0.03 (s, 6H); 0.05 (s, 6H); 0.83 (s, 18H); 3.28 (s, 2H); 3.73 (m, 4H); 3.96 (dd, 1H); 4.29 (t, 1H); 4.86 (d, 2H); 5.18 (m, 1H); 7.41 (dd, 1H); 7.58 (m, 2H); 7.69 (t, 1H); 7.77 (d, 1H); 8.04 (dt, 1H); 8.18 (d, 1H); 8.81 (broad s, 1H).

¹⁹F-NMR (300 Mz)(DMSO-d₆) δ: −115.97 (s, 1F)

### 2-[5,5-Bis({[tert-butyl(dimethyl)silyl]oxy}methyl-4,5-dihydroisoxazol-3-yl]-5-bromopyridine

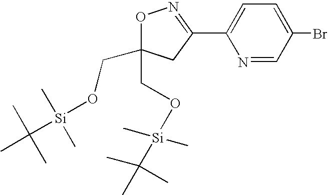

2,2,3,3,9,9,10,10-Octamethyl-6-methylene-4,8-dioxa-3,9-disilaundecane (0.685 g, 1.94 mmol) was combined with 5-bromo-N-hydroxypyridine-2-carboximidoyl chloride (0.30 g, 1.3 mmol) under a nitrogen atmosphere. Anhydrous tetrahydrofuran (8 ml) was added, followed by the slow addition of diisopropylethylamine (0.45 ml, 2.6 mmol) via syringe at room temperature. The reaction was stirred overnight at room temperature, then diluted with ethyl acetate (200 ml), washed with water (1×100 ml), brine (1×75 ml), and dried over anhydrous magnesium sulfate. The solvents were removed in vacuo, producing a crude product mixture. The product was dissolved in dichloromethane (10 in), applied to a pre-wettened 50-gram Isolute silica gel column and eluted with 20:80 ethyl acetate:hexanes. The product eluted in two fractions, the first of which included excess 2,2,3,3,9,9,10,10-octamethyl-6-methylene-4,8-dioxa-3,9-disilaundecane, and the second fraction, which was found to be pure (0.28 g, 42.6% yield).

MS (APCI): 515.2, 517.1 (MH⁺) for C₂₂H₃₉BrN₂O₃Si₂

¹H-NMR (30 Mz (CDCl₃): 0.04 (s, 6H); 0.06 (s, 6H); 0.85 (s, 18H); 3.32 (s, 2H); 3.73 (q, 4H); 7.81 (m, 1H); 7.87 (m, 1H); 8.64 (m, 1H).

### 2,2,3,3,9,9,10,10-Octamethyl-6-methylene-4,8-dioxa-3,9-disilaundecane

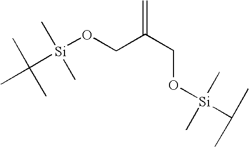

2-Methylene-1,3-propanediol (10 g, 11.3 mmol) was dissolved in anhydrous N,N-dimethylformamide (15 ml) under a nitrogen atmosphere. Imidazole (1.93 g, 28.4 mmol) was added, the reaction stirred at room temperature for ten minutes, followed by addition of tert-butyldimethylsilylchloride (3.76 g, 25.0 mmol). The reaction mixture was stirred overnight at room temperature, then diluted with ethyl acetate (350 ml), washed with water (2×100 ml), then a brine solution (1×100 ml), and then dried over anhydrous magnesium sulfate. The product was carried on without further purification into the next reaction.

¹H-NMR (300 Mz)(CDCl₃) δ: 0.05 (s, 12H); 0.89 (s, 18H); 4.14 (t, 4H); 5.06 (m, 2H).

### EXAMPLE 56

### (2R)-2-[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]-2-hydroxyethyl 3-methoxypropanoate

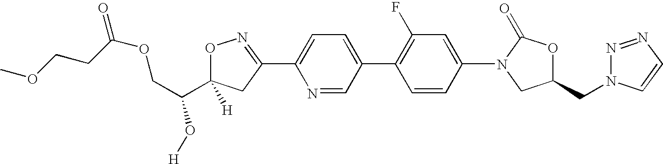

(5R)-3-[4-(6-{(5S)-5-[(1R)-1,2-Dihydroxyethyl]-4,5-dihydroisoxazol-3-yl)pyridin-3-yl)-3-fluorophenyl]-5-(1H-1,2,3-triazol-1-ylmethyl)-1,3-oxazolidin-2-one (Example 51, 0.2 g, 0.43 mmol) was dissolved in DMF (3 ml) and pyridine (0.6 ml, 7.4 mmol) was added. The solution was cooled to 0° C. and 3-methoxypropanoic anhydride (0.12 g, 0.63 mmol) dissolved in dichloromethane (0.5 ml) was added. The solution was allowed to stir and slowly come to room temperature for 18 hours, after which the mixture was cooled again to 0° C. A second portion of 3-methoxypropanoic anhydride (0.25 g, 1.32 mmol) was added and the solution was allowed to stir and slowly come to room temperature for 3 hours. The mixture was then diluted with ethyl acetate, washed with water, and dried over magnesium sulfate. The residue obtained upon filtration and evaporation was purified via chromatography (silica gel, 10 to 30% acetonitrile in ethyl acetate), the monoacylated product was separated from the less polar bis-acylated material, which was also produced in a small amount. Evaporation of the product containing fractions and trituration of the resulting solid with diethyl ether yielded the title compound as a white solid (0.078 g), melting point: 130° C.

MS (ESP): 555 (MH⁺) for C₂₆H₂₇FN₆O₇

¹H-NMR (500 MHz CDCl₃) δ: 2.64 (t, 2H); 3.36 (s, 3H); 3.56 (dd, 1H); 3.65-3.70 (m, 3H); 3.99-4.07 (m, 2H); 4.19-4.27 (m, 2H); 4.39 (dd, 1H); 4.78-4.82 (m, 3H); 5.11 (m, 1H); 7.23 (dd, 1H); 7.42 (d, 1H); 7.47 (dd, 1H); 7.76 (s, 1H); 7.79 (s, 1H); 7.90 (bd, 1H); 8.06 (bd, 1H); 8.76 (s, 1H).

### EXAMPLE 57

### (2R)-2-[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]-2-hydroxyethyl nicotinate

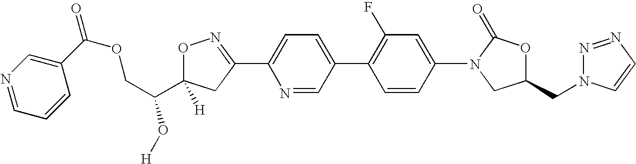

(5R)-3-[4-(6-{(5S)-5-[(1R)-1,2-Dihydroxyethyl]-4,5-dihydroisoxazol-3-yl}pyridin-3-yl)-3-fluorophenyl]-5-(1H-1,2,3-triazol-1-ylmethyl)-1,3-oxazolidin-2-one (Example 51, 0.2 g, 0.43 mmol) and nicotinic acid (0.063 g, 0.51 mmol) were dissolved in a mixture of DMF (2 ml) and pyridine (0.2 ml, 2.5 mmol). The solution was cooled to 0° C. and diisopropylcarbodiimide (0.27 ml, 1.73 mmol) was added. The solution was allowed to stir for 8 hours at 0° C., then diluted with ethyl acetate and washed with water. The aqueous layer was extracted with THF: ethyl acetate (1:1) and the pooled organic layers were dried over magnesium sulfate. The residue obtained upon filtration and evaporation was purified via chromatography (silica gel, 1 to 5% methanol in dichloromethane), the monoacylated product was separated from the less polar bis-acylated material, which was also produced in a small amount. Evaporation of the product containing fractions and trituration of the resulting solid with diethyl ether yielded the title compound as an off-white solid (0.095 g), melting point: 210° C.

MS (ESP): 574 (MH⁺) for C₂₈H₂₄FN₇O₆

¹H-NMR (500 MHz, DMSO-d₆) δ: 3.53-3.55 (m, 2H); 3.94-3.99 (m, 2H); 4.28-4.32 (m, 2H); 4.42 (dd, 1H); 4.85-4.90 (m, 3H); 5.18 (m, 1H); 5.68 (d, 1H); 7.43 (dd, 1H); 7.56-7.60 (m, 2H); 7.69 (t, 1H); 7.77 (s, 1H); 7.99 (d, 1H); 8.05 (bd, 1H); 8.18 (s, 1H); 8.33 (bd, 1H); 8.82 (m, 2H); 9.14 (bs, 1H).

### EXAMPLE 58

### (2R)-2-[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]-2-hydroxyethyl 2-methoxyethyl carbonate

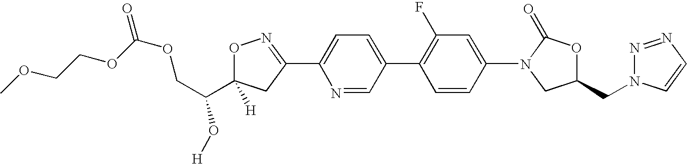

(5R)-3-[4-(6-{((S)-5-[(1R)-1,2-Dihydroxyethyl]-4,5-dihydroisoxazol-3-yl}pyridin-3-yl)-3-fluorophenyl]-5-(1H-1,2,3-triazol-1-ylmethyl)-1,3-oxazolidin-2-one (Example 51, 0.2 g, 0.43 mmol) was dissolved in DMF (3 ml) and pyridine (0.5 ml, 6.2 mmol) was added. The solution was cooled to 0° C. and 2-methoxyethylchloroformate (0.07 ml, 0.6 mmol) was added. The solution was allowed to stir for 1 hour at 0° C., then a second portion of 2-methoxyethylchloroformate (0.07 ml, 0.6 mmol) was added. The reaction was allowed to proceed for an additional 45 minutes at 0° C., then quenched by the addition of 1 ml methanol. After stirring for 5 minutes, the mixture was poured into water and extracted with ethyl acetate. The organic layer was washed with brine and dried over magnesium sulfate. The residue obtained upon filtration and evaporation was purified via chromatography (silica gel, 1 to 10% methanol in dichloromethane), the monoacylated product was separated from the less polar bis-acylated material, which was also produced in a small amount. Evaporation of the product containing fractions and trituration of the resulting solid with diethyl ether yielded the title compound as an off-white solid (0.052 g), melting point: 125° C.

MS (ESP): 571 (MH⁺) for C₂₆H₂₇FN₆O₈

¹H-NMR (500 MHz, DMSO-d₆) δ: 3.25 (s, 3H); 3.46-3.53 (m, 4H); 3.82 (m, 1H); 3.96 (dd, 1H); 4.07 (dd, 1H); 4.17-4.21 (m, 3H); 4.30 (t, 1H); 4.70-4.75 (m, 1H); 4.86 (d, 2H); 5.18 (m, 1H); 5.61 (d, 1H); 7.42 (dd, 1H); 7.59 (dd, 1H); 7.69 (t, 1H); 7.77 (s, 1H); 7.99 (d, 1H); 8.06 (bd, 1H); 8.18 (s, 1H); 8.83 (s, 1H).

### EXAMPLE 59

### Phosphoric acid mono-(1R)-[(5R)-2-(3-{(5S)-[2-fluoro-4-(2-oxo-5-[1,2,3]triazol-1-ylmethyl-oxazolidin-3-yl)-phenyl]-pyridin-2-yl}-4,5-dihydro-isoxazol-5-yl)-2-phosphonooxy-ethyl]ester, tetrakis ammonium salt

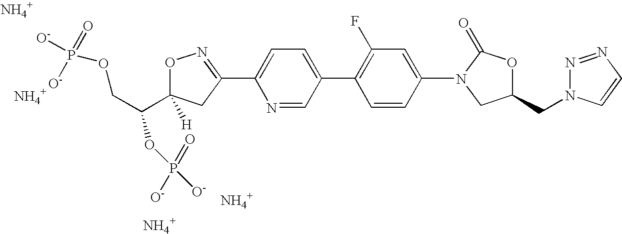

Phosphoric acid di-tert-butyl ester-(1R)-2-(di-tert-butoxy-phosphoryloxy)-(5R)-2-(3-{(5S)-[2-fluoro-4-(2-oxo-5-[1,2,3]triazole-1-ylmethyl-oxazolidin-3-yl)-phenyl]-pyridin-2-yl}-4,5-dihydro-isoxazol-5-yl)-ethyl ester (0.732 g) was taken up in methanol (12 mL). To this was added a solution of 4 N HCl in dioxane (7 mL) and the resulting yellow-colored solution was allowed to stir at room temperature for 3 hours. The solvent was removed in vacuo to afford a yellow foam which was then take up in toluene and dichloromethane and evaporated. The resulting yellow foam was triturated in methanol and diethyl ether and filtered to afford a yellow solid, the intermediate diphosphonic acid (0.333 g). The intermediate was then dissolved in water (8 mL) and concentrated aqueous ammonium hydroxide solution (4 mL) and lyophilized to afford a yellow solid (0.361 g). The solid was then triturated in methanol and filtered to afford a light yellow powder (0.269 g).

Mp 175-180° C. (decomp.)

MS (APCI): 629.12 (MH⁺) for C₂₂H₂₃FN₆O₁₁P₂

¹H-NMR (D₂O) δ: 3.59 (m, 1H); 3.69 (m, 1H); 4.06 (m, 3H); 4.31 (m, 2H); 4.90 (m, 1H); 4.93 (m, 1H); 5.11 (m 1H); 5.22 (m, 1H); 7.15 (d, 1H); 7.28 (d, 1H); 7.53 (s, 1H); 7.74 (s, 1H); 7.90 (s, 1H); 8.06 (m, 2H); 8.68 (s, 1H).

### Phosphoric acid di-tert-butyl ester-(1R)-2-(di-tert-butoxy-phosphoryloxy-(5R)-2-(3-{(5S)-[2-fluoro-4-(2-oxo-5-[1,2,3]triazole-1-ylmethyl-oxazolidin-3-yl)-phenyl-pyridin-2-yl}-4,5-dihydro-isoxazol-5-yl)-ethyl ester

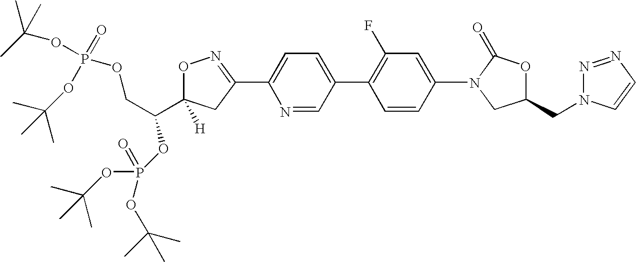

(5R)-3-[4-(6-((5S)-5-[(1R)-1,2-Dihydroxyethyl]-4,5-dihydroisoxazol-3-yl}pyridin-3-yl)-3-fluorophenyl]-5-(1H-1,2,3-triazol-1-ylmethyl)-1,3-oxazolidin-2-one (Example 51, 0.282 g, 0.60 mmol) was taken up in 3.5 mL N)N-dimethylformamide. After cooling to 0° C. (external ice-water bath), di-tert-butyl diethylamidophosphite (1.1 mL, 3.7 mmol) was added via syringe followed by 1.1 mL of a 3 wt % solution of 1H-tetrazole in acetonitrile (3.7 mmol). After stirring at 0° C. for 8 minutes the ice water bath was removed and the reaction was allowed to stir for 2 hours. The reaction mixture was then cooled to −78° C. (external dry ice-acetone bath) before adding m-chloroperbenzoic acid (0.906 g, 3.7 mmol). The reaction was stirred at −78° C. for 40 minutes before quenching with aqueous sodium thiosulfate solution. The dry ice-acetone bath was removed and the reaction mixture was allowed to warm to room temperature. The reaction mixture was diluted with ethyl acetate and water and the layers were separated. The aqueous phase was extracted twice with ethyl acetate and the combined organic layers were washed twice with saturated aqueous sodium bicarbonate and once with brine. The organic layers were dried over magnesium sulfate, filtered and concentrated in vacuo to afford a light yellow oil (0.912 g). The crude product was purified by flash chromatography on silica gel using a gradient of 5% methanol in dichloromethane to 7.5% methanol in dichloromethane to afford the title product (0.732 g).

MS (APCI): 853.3 (MH⁺) for C₃₈H₅₅FN₆O₁₁P₂

¹H-NMR (DMSO-d₆) δ: 1.23 (s, 9H); 1.25 (s, 9H); 1.29 (s, 18H); 3.44 (d, 1H); 3.48 (s, 1H); 3.62 (m, 1H); 3.82 (m, 1H); 3.98 (m, 1H); 4.16 (m, 1H); 4.29 (m, 1H); 4.72 (d, 2H); 4.82 (m, 1H); 5.04 (m, 1H); 7.28 (dd, 1H); 7.45 (dd, 1H); 7.56 (t, 1H); 7.63 (d, 1H); 7.86 (d, 1H); 7.94 (m, 1H); 8.05 (d, 1H); 8.69 (s, 1H).

### EXAMPLE 60

### (5R)-3-{3-Fluoro-4-[6-((5S)-5-{[(2-hydroxyethyl)thio]methyl}-4,5-dihydroisoxazol-3-yl)pyridin-3-yl]phenyl}-5-(1H-1,2,3-triazol-1-ylmethyl)-1,3-oxazolidin-2-one

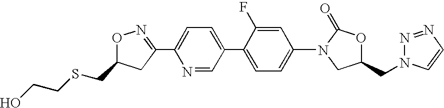

(5R)-3-(3-Fluoro-4-{6-[(5S)-5-(hydroxymethyl)-4,5-dihydroisoxazol-3-yl]pyridin-3-yl}phenyl)-5-(1H-1,2,3-triazol-1-ylmethyl)-1,3-oxazolidin-2-one (0.3 g, 0.68 mmol) was dissolved in DMF (5 ml) with warming, then allowed to cool to room temperature. Triphenylphosphine (0.27 g, 1.03 mmol) and carbon tetrachloride (0.6 ml, 6.21 mmol) were added and the mixture was stirred at room temperature for 45 minutes. The solution was diluted with ethyl acetate, washed twice with water, then with saturated sodium chloride, dried over sodium sulfate and evaporated. Purification by column chromatography (silica gel, 1 to 10% methanol in dichloromethane) yielded (5R)-3-(4-{6-[(5S)-5-(chloromethyl)-4,5-dihydroisoxazol-3-yl]pyridin-3-yl}-3-fluorophenyl)-5-(1H-1,2,3-triazol-1-ylmethyl)-1,3-oxazolidin-2-one (0.31 g). This material was contaminated with triphenylphosphine oxide, and was used in the next step without further purification.

(5R)-3-(4-{6-[(5S)-5-(chloromethyl)-4,5-dihydroisoxazol-3-yl]pyridin-3-yl}-3-fluorophenyl)-5-(1H-1,2,3-triazol-1-ylmethyl)-1,3-oxazolidin-2-one (300 mg, <0.66 mmol), 2-mercaptoethanol (0.1 ml, 1.43 mmol), potassium carbonate (280 mg, 2.03 mmol), tetrabutyl ammonium iodide (1-2 mg, catalytic amount) and DMF (2 ml) were combined and warmed to 50° C. for 16 hours. An additional portion of 2-mercaptoethanol (0.1 ml, 1.43 mmol) was added and the mixture was warmed at 50° C. for 24 hours more. The mixture was diluted with acetonitrile, filtered and evaporated. Purification by column chromatography (silica gel, 2 to 10% acetonitrile in ethyl acetate) gave a solid which was sonnicated and triturated with 3 ml 1:1 ethyl acetate:ether. (5R)-3-{3-fluoro-4-[6-((5S)-5-{[(2-hydroxyethyl)thio]methyl}-4,5-dihydroisoxazol-3-yl)pyridin-3-yl]phenyl}-5-(1H-1,2,3-triazol-1-ylmethyl)-1,3-oxazolidin-2-one was thus obtained as an off-white solid (154 mg): melting point: 162° C.

MS (electrospray): 499 (M+1) for C₂₃H₂₃FN₆O₄S

¹H-NMR (400 MHz DMSO-d₆) δ: 2.66 (t, 2H); 2.79-2.89 (m, 2H); 3.31 (m, 3H); 3.55 (bt, 2H); 3.58 (dd, 1H); 3.96 (dd, 1H); 4.30 (t, 1H); 4.79 (bt, 1H); 4.86 (d, 2H); 4.96 (m, 1H); 5.19 (m, 1H); 7.42 (dd, 1H); 7.59 (dd, 1H); 7.69 (t, 1H); 7.77 (s, 1H); 7.99 (d, 1H); 8.06 (d, 1H); 8.18 (s, 1H); 8.82 (s, 1H).

The intermediates for this example were prepared as follows:

### (5R)-3-(3-Fluoro-4-{6-[(5S)-5-(hydroxymethyl)-4,5-dihydroisoxazol-3-yl]pyridin-3-yl}phenyl)-5-(1H-1,2,3-triazol-1-ylmethyl)-1,3-oxazolidin-2-one

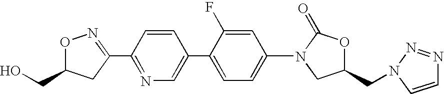

[(5S)-3-(5-Bromopyridin-2-yl)-4,5-dihydroisoxazol-5-yl]methanol (0.277 g, 1.08 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (see Example 13, 0.35 g, 0.9 mmol), potassium carbonate (0.622 g, 4.5 mmol), and tetrakis(triphenylphosphino)palladium(0) (0.1 g, 0.09 mmol) were combined and suspended in DMF (7 ml) and water (1 ml). The mixture was heated at 75° C. for 2 hours, then was poured into cold water (30 ml). The solids formed were collected, rinsed with water and washed with dichloromethane (2×10 ml), the solids were then dissolved in warm trifluoroethanol (2 ml), and further purified by column chromatography, eluting with 8% methanol in dichloromethane to give the title compound as a white solid (0.193 g).

MS (ESP): 439.22 (M+1) for C₂₁H₁₉FN₆O₄

NMR (300 Mz)(DMSO-d₆) δ: 3.36-3.58 (m, 4H); 3.95 (dd, 1H); 4.29 (t, 1H); 4.78 (m, 1H); 4.86 (d, 2H); 5.02 (t, 1H); 5.18 (m, 1H); 7.41 (dd, 1H); 7.58 (dd, 1H); 7.69 (t, 1H); 7.77 (s, 1H); 7.98 (d, 1H); 8.05 (dd, 1H); 8.18 (s, 1H); 8.78 (s, 1H).

### [(5S)-3-(5-Bromopyridin-2-yl)-4,5-dihydroisoxazol-5-yl]methanol

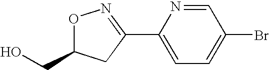

[(5S)-3-(5-Bromopyridin-2-yl)-4,5-dihydroisoxazol-5-yl]methyl butyrate (16.88 g, 0.051 mol) was dissolved in methanol (110 ml). 50% Aqueous sodium hydroxide (3.6 ml, 0.068 mol) was added. The solution was stirred at RT for 15 minutes, 1M HCl (75 ml) was added, followed by concentration in vacuo to ˜100 ml total volume. Water (−50 ml) was added, and the white precipitate was collected and rinsed with water. The filtrate was extracted twice with ethyl acetate, the organic layers were pooled, dried over sodium sulfate and evaporated. The solid residue was collected and rinsed with 10:1 hexane:ethyl acetate, then combined with the initial precipitate before drying in vacuo to give the title compound as a white crystalline solid, 12.3 g (93%). Chiral HPLC analysis indicated <0.5% of the (−) isomer was present. [α]_{D}=+139 (c=0.01 g/ml in methanol).

### (5S)-3-(5-Bromopyridin-2-yl-4,5-dihydroisoxazol-5-yl]methyl butyrate

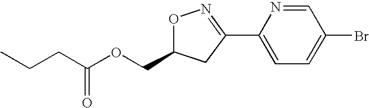 (+) Isomer assigned as (5S) based on comparison with Chem. Lett. 1993 p. 1847.

Racemic [3-(5-bromopyridin-2-yl)-4,5-dihydroisoxazol-5-yl]methyl butyrate (80 g, 0.244 mol) was dissolved in acetone (4 L), and 0.1 M potassium phosphate buffer (pH˜7) (4 L) was added with vigorous stirring to give a clear yellow solution. PS-lipase (1.45 g, Sigma cat no L-9156) was added and the mixture was gently stirred at ambient temp. for 42 hrs. The solution was divided into 3 equal volumes of ˜2.6 L and each was extracted with dichloromethane (2×1 L), the pooled organic phases were dried over sodium sulfate and evaporated. The unreacted [(5S)-3-(5-bromopyridin-2-yl)-4,5-dihydroisoxazol-5-yl]methyl butyrate was isolated via flash column chromatography (9:1 hexane:ethyl acetate) as a clear yellow oil, 36.4 g (45.5%).

### [3-(5-Bromopyridin-2-yl)-4,5-dihydroisoxazol-5-yl]methyl butyrate

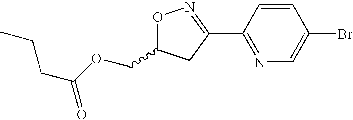

5-Bromo-N-hydroxypyridine-2-carboximidoyl chloride (46 g, 195.7 mmol) was added to EtOAc (200 ml) followed by addition of allyl butyrate (145 ml, 1020.4 mmol) and the solution was cooled to 0° C. Triethylamine (30 ml, 215.8 mmol) in EtOAc (100 ml) was then added dropwise over 1 hour. The reaction was then allowed to stir for 1 hour at 0° C. and then EtOAc (1 L) was added. The precipitate was removed by vacuum filtration and the filtrate was concentrated in vacuo to yield the product (65 g).

¹H-NMR (DMSO-d₆) δ: 0.81 (t, 3H); 1.43 (m, 2H); 2.24 (t, 2H); 3.21 (dd, 1H); 3.54 (dd, 1H); 4.13 (dd, 1H); 4.23 (dd, 1H); 5.01 (m, 1H); 7.85 (dd, 1H); 8.12 (dd, 1H); 8.81 (d, 1H).

### 5-Bromo-N-hydroxypyridine-2-carboximidoyl chloride

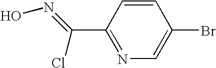

5-Bromopyridine-2-carbaldehyde oxime (49.5 g, 246.3 mmol) was dissolved in DMF (150 ml) followed by addition of N-chlorosuccinimide (39.5 g, 295.5 mmol). HCl gas was then bubbled in the solution for 20 seconds to initiate the reaction, which was then allowed to stir for 1 hr. The reaction was poured into distilled water (1 L) and the precipitate was collected by vacuum filtration. The filter cake was washed with distilled water (2×500 ml) and then dried overnight in a vacuum oven at 60° C. (−30 inches Hg) to yield the product as a white powder (55 g).

¹H-NMR (300 Mz)(CDCl₃) δ: 7.73 (d, 1H); 8.09 (d, 1H); 8.73 (s, 1H); 12.74 (s, 1H).

NOTE: Lachrymator.

### EXAMPLE 61

### (5R)-3-[3-fluoro-4-[(5S)-5-[(trifluoromethoxy)methyl]-4,5-dihydroisoxazol-3-yl)-3-pyridinyl]phenyl]-5-(1H-1,2,3-triazol-1-ylmethyl)oxazolidin-2-one

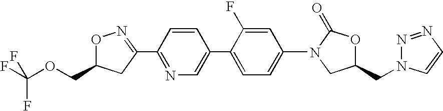

5-Bromo-2-{(5S)-5-[trifluoromethoxy)methyl]-4,5-dihydroisoxazol-3-yl}pyridine (520 mg, 1.6 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (620 mg, 1.6 mmol) and sodium carbonate (678 mg, 6.4 mmol) were dissolved/suspended in N,N-dimethyl formamide/water (10 mL, 10:1). It was degassed, flushed with nitrogen and tetrakis (triphenylphospine) palladium (0) (180 mg, 0.16 mmol) was added. It was heated at 70° C. for 5 hours, cooled to room temperature, and the solvent was evaporated. Chromatography on silica gel with dichloromethane/DMF (30:1 to 20:1) gave 478 mg product (59%) as a colorless solid, mp 185° C.

MS (ESP): 507.44 (MH⁺) for C₂₂H₁₈F₄N₆O₄

¹H-NMR (DMSO-d₆) δ: 3.26-3.37 (m, 1H); 3.63 (dd, 1H); 3.96 (dd, 1H); 4.21-4.36 (m, 3H); 4.86 (d, 2H); 5.07 (, 1H); 5.19 (m, 1H); 7.42 (dd, 1H); 7.59 (dd, 1H); 7.69 (dd, 1H); 7.76 (brs, 1H); 8.02 (dd, 1H); 8.07 (m, 1H); 8.18 (brs, 1H); 8.83 (s, 1H).

The intermediates for Example 61 were prepared as follows:

### 5-Bromo-2-{(5S)-5-[trifluoromethoxy)methyl]-4,5-dihydroisoxazol-3-yl}pyridine

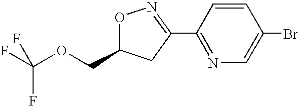

1,3-Dibromo-5,5-dimethylhydantoin (1.65 g, 5.77 mmol) was suspended in dry dichloromethane (6 mL) and cooled to −78° C. HF/py (65-70%, 4 mL) was added, followed by drop wise addition of a solution of O-[[(5S)-3(5-bromopyridin-2-yl)-4,5-dihydroisoxazol-5-yl]methyl}S-methyl dithiocarbonate (748 mg, crude, −1.94 mmol, as obtained below) in dichloromethane (6 mL). It was warmed to 0° C. and stirred for 45 minutes. The reaction mixture was diluted with dichloromethane, washed with a buffer consisting of: 0.5 M NaHCO₃ and 0.25 M Na₂SO₃ (buffer pH adjusted to pH 10 with KOH) and dried over sodium sulfate. Chromatography on silica gel with hexanes/ethyl acetate 6:1 gave the product as a colourless solid 520 mg (82%).

¹H-NMR (DMSO-d₆) δ: 3.25 (dd, 1H); 3.58 (dd, 1H); 4.21 (dd, 1H); 4.30 (dd, 1H); 5.05 (m, 1H); 7.86 (d, 1H); 8.13 (dd, 1H); 8.78 (d, 1H).

### O-[[(5S)-3-(5-Bromopyridin-2-yl)-4,5-dihydroisoxazol-5-yl]methyl}S-methyl dithiocarbonate

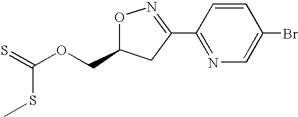

5-Bromo-2-[(5S)-5-hydroxymethyl-4,5-dihydroisoxazol-3-yl]pyridine (500 mg, 1.94 mmol) and nBu₄NHSO₄ (66 mg, 10 mol %) were dissolved in a 2-phase system consisting of carbon disulfide (4 mL) and 50% aqueous sodium hydroxide (4 mL). Methyl iodide (134 μL, 2.14 mmol) was added and it was stirred vigorously at room temp. for 30 minutes. It was diluted with dichloromethane and water, the organic phase was washed with water and dried over sodium sulfate. The crude methyl dithiocarbonate was obtained together with the phase transfer catalyst (748 mg, quant.) and was used without further purification for the next step.

MS (ESP): 347.25/349.25 (MH⁺) for C₁₁H₁₁BrN₂O₂S₂

¹H-NMR (DMSO-d₆) δ: 2.46 (s, 3H); 3.35 (dd, 1H); 3.61 (dd, 1H); 4.67 (dd, 1H); 4.78 (dd, H); 5.19 (m, 1H); 7.86 (d, 1H); 8.13 (dd, 1H); 8.78 (d, 1H).

### 5-Bromo-2-[(5S)-5-hydroxymethyl-4,5-dihydroisoxazol-3-yl]pyridine

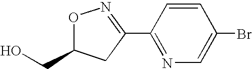 see preparation of intermediate 11 from intermediates 8, 9 and 10 in Example 63 below

### EXAMPLE 62

### (5R)-3-[3-fluoro-4-[(5S)-5-[(2,2,2-trifluoroethoxy)methyl]-4,5-dihydroisoxazol-3-yl)-3-pyridinyl]phenyl}-S-(1H-1,2,3-triazol-ylmethyl)oxazolidin-2-one

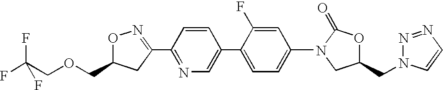

5-Bromo-2-{(5S)-5-[(2,2,2-trifluoroethoxy)methyl]-4,5-dihydroisoxazol-3-yl}pyridine (230 mg, 0.68 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (263 mg, 0.68 mmol), sodium carbonate (287 mg, 2.7 mmol) and tetrakis (triphenylphospine) palladium (0) (78 mg, 0.068 mmol) were reacted as described for Example 1. Chromatography on silica gel with dichloromethane/DMF (30:1) gave 180 mg product (51%) as a colourless solid, mp 199° C.

MS (ESP): 521.47 (MH⁺) for C₂₃H₂₀F₄N₆O₄

¹H-NMR (DMSO-d₆) δ: 3.26-3.37 (m, 1H); 3.55 (dd, 1H); 3.70-3.84 (m, 2H); 3.96 (dd, 1H); 4.15 (dd, 2H); 4.29 (dd, 1H); 4.86 (d, 2H); 4.95 (m, 1H); 5.19 (m, 1H); 7.42 (dd, 1H); 7.59 (dd, 1H); 7.69 (dd, 1H); 7.76 (brs, 1H); 8.01 (dd, 1H); 8.05 (n, 1H); 8.18 (brs, 1H); 8.82 (brs, 1H).

The intermediates for Example 62 were prepared as follows:

### 5-Bromo-2-{(5S)-5-[(2,2,2-trifluoroethoxy)methyl]-4,5-dihydroisoxazol-3-yl}pyridine

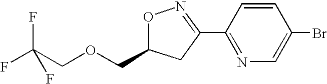

5-Bromo-2-[(5S)-5-hydroxymethyl-4,5-dihydroisoxazol-3-yl]pyridine (500 mg, 1.94 mmol) and 1,1-(azodicarbonyl)dipiperidine (981 mg, 3.89 mmol) were dissolved in benzene (10 mL), tris n-butyl phosphine (960 μL, 3.89 mmol) was added and it was stirred for 10 minutes at room temperature. 2,2,2-Trifluoroethanol (1.42 mL, 19.4 mmol) was added. It was stirred for 10 minutes, then diluted with benzene (10 mL) and stirred over night. The solvent was evaporated under vacuum and the residue was taken up in toluene (40 mL). It was filtered and the filtrate was applied onto a silica gel column, eluting with hexanes/acetone 5:1. Crude product was rechromatographed on silica gel with hexanes/ethyl acetate 5:1 to give the product as a colourless solid upon drying under high vacuum, 230 mg, 35%.

¹H-NMR (DMSO-d₆) δ: 3.20 (dd, 1H); 3.49 (dd, 1H); 3.70-3.81 (m, 2H); 4.12 (dd, 2H); 4.94 (m, 1H); 7.85 (d, 1H); 8.12 (dd, 1H); 8.78 (d, 1H).

### 5-Bromo-2-[(5S)-5-hydroxymethyl-4,5-dihydroisoxazol-3-yl]pyridine

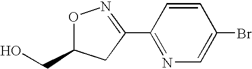 see preparation of intermediate 11 from intermediates 8, 9 and 10 in Example 63 below

### EXAMPLE 63

### (5R)-3-[3-Fluoro-4-[(5S)-5-{((2-methoxyethoxy)methoxy]methyl}-4,5-dihydroisoxazol-3-yl)-3-pyridinyl]phenyl]-5-(1H-1,2,3-triazol-1-ylmethyl)oxazolidin-2-one

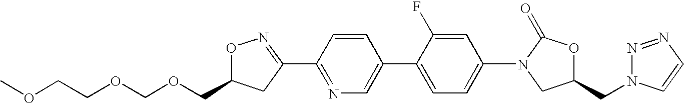

(5R)-3-[3-Fluoro-4-[((5S)-5-hydroxymethyl-4,5-dihydroisoxazol-3-yl)-3-pyridinyl]phenyl]-5-(1H-1,2,3-triazol-1-ylmethyl)oxazolidin-2-one (250 mg, 0.57 mmol was dissolved/suspended in dry DMF (5 mL). Diisopropyl ethyl amine (238 μL, 1.37 mmol) was added, followed by 2-methoxyethoxymethyl chloride (“MEMCl”, 78 μL, 0.68 mmol) and it was stirred at room temperature over night. More diisopropyl ethyl amine (250 μL, 1.44 mmol) and MBMCl (100 μL, 0.88 mmol) were added and it was stirred for another 6 hours. Then once again MBMCl (90 μL, 0.79 mmol) was added and it was stirred over night. The solvent was evaporated under vacuum. Chromatography on silica gel with acetone/hexanes 3:1 and precipitation from dichloromethane/hexanes gave the product as off-white solid, 261 mg, 87%.

### (5R)-3-[3-Fluoro-4-[((5S)-5-hydroxymethyl-4,5-dihydroisoxazol-3-yl)-3-pyridinyl]phenyl]-5-(1H-1,2,3-triazol-1-ylmethyl)oxazolidin-2-one

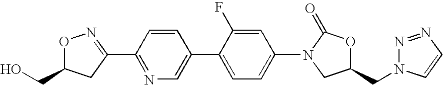

[(5S)-3-(5-Bromopyridin-2-yl)-4,5-dihydroisoxazol-5-yl]methanol (Intermediate 11, 0.277 g, 1.08 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 0.35 g, 0.9 mmol), potassium carbonate (0.622 g, 4.5 mmol), and tetrakis(triphenylphosphino)palladium(0) (0.1 g, 0.09 mmol) were combined and suspended in DMF (7 ml) and water (1 ml). The mixture was heated at 75° C. for 2 hours, then was poured into cold water (30 ml). The solids formed were collected, rinsed with water and washed with dichloromethane (2×10 ml), the solids were then dissolved in warm trifluoroethanol (2 ml), and further purified by column chromatography, eluting with 8% methanol in dichloromethane to give the title compound as a white solid (0.193 g).

MS (ESP): 439.22 (M+1) for C₂₁H₁₉FN₆O₄

NMR (300 Mz)(DMSO-d₆) δ: 3.36-3.58 (m, 4H); 3.95 (dd, 1H); 4.29 (t, 1H); 4.78 (m, 1H); 4.86 (d, 2H); 5.02 (t, 1H); 5.18 (m, 1H); 7.41 (dd, 1H); 7.58 (dd, 1H); 7.69 (t, 1H); 7.77 (s, 1H); 7.98 (d, 1H); 8.05 (dd, 1H); 8.18 (s, 1H); 8.78 (s, 1H).

### Intermediate 1: Acetic acid (5R)-3-(3-fluoro-phenyl)-2-oxo-oxazolidin-5-ylmethyl ester

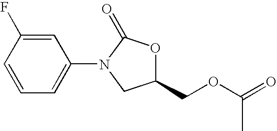

(5R)-3-(3-Fluorophenyl)-5-hydroxymethyloxazolidin-2-one (40 g, 0.189 mol, see Upjohn WO 94-13649) was suspended by stirring in dry dichloromethane (400 ml) under nitrogen. Triethylamine (21 g, 0.208 mol) and 4-dimethylaminopyridine (0.6 g, 4.9 mmol) were added, followed by dropwise addition of acetic anhydride (20.3 g, 0.199 mol) over 30 minutes, and stirring continued at ambient temperature for 18 hours. Saturated aqueous sodium bicarbonate (250 ml) was added, the organic phase separated, washed with 2% sodium dihydrogen phosphate, dried (magnesium sulfate), filtered and evaporated to give the desired product (49.6 g) as an oil.

MS ASP): 254 (MH⁺) for C₁₂H₁₂FNO₄

NMR (300 MHz) (CDCl₃) δ: 2.02 (s, 3H); 3.84 (dd, 1H); 4.16 (t, 1H); 4.25 (dd, 1H); 4.32 (dd, 1H); 4.95 (m, 1H); 6.95 (td, 1H); 7.32 (d, 1H); 7.43 (t, 1H); 7.51 (d, 1H).

### Intermediate 2: Acetic acid (5R)-3-(3-fluoro-4-iodo-phenyl)-2-oxo-oxazolidin-5-ylmethyl ester

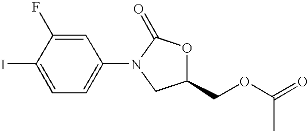

Acetic acid (5R)-3-(3-fluoro-phenyl)-2-oxo-oxazolidin-5-ylmethyl ester (Intermediate 1, 15.2 g, 60 mmol) was dissolved in a mixture of chloroform (100 ml) and acetonitrile (100 ml) under nitrogen, and silver trifluoroacetate (16.96 g, 77 mmol) were added. Iodine (18.07 g, 71 mmol) was added in portions over 30 minutes to the vigorously stirred solution, and stirring continued at ambient temperature for 18 hours. As reaction was not complete, a further portion of silver trifluoroacetate (2.64 g, 12 mmol) was added and stirring continued for 18 hours. After filtration, the mixture was added to sodium thiosulfate solution (3%, 200 ml) and dichloromethane (200 ml), and the organic phase separated, washed with sodium thiosulfate (200 ml), saturated aqueous sodium bicarbonate (200 ml), brine (200 ml), dried (magnesium sulfate), filtered and evaporated. The crude product was suspended in isohexane (100 ml), and sufficient diethyl ether added to dissolve out the brown impurity while stirring for 1 hour. Filtration gave the desired product (24.3 g) as a cream solid.

MS (ESP): 380 (MH⁺) for C₁₂H₁₁FINO₄

NMR (300 MHz) (DMSO-d₆) δ: 2.03 (s, 3H); 3.82 (dd, 1H); 4.15 (t, 1H); 4.24 (dd, 1H); 4.30 (dd, 1H); 4.94 (m, 1H); 7.19 (dd, 1H); 7.55 (dd, 1H); 7.84 (t, 1H).

### Intermediate 3: (5R)-3-(3-Fluoro-4-iodophenyl)-5-hydroxymethyloxazolidin-2-one

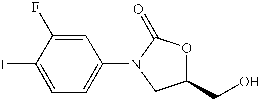

Acetic acid (5R)-3-(3-fluoro-4-iodophenyl)-2-oxo-oxazolidin-5-ylmethyl ester (Intermediate 2, 30 g, 79 mmol) was treated with potassium carbonate (16.4 g, 0.119 mmol) in a mixture of methanol (800 ml) and dichloromethane (240 ml) at ambient temperature for 25 minutes, then immediately neutralised by the addition of acetic acid (10 ml) and water (500 ml). The precipitate was filtered, washed with water, and dissolved in dichloromethane (1.2 L), the solution washed with saturated sodium bicarbonate, and dried (magnesium sulfate). Filtration and evaporation gave the desired product (23 g).

MS (ESP): 338 (MH⁺) for C₁₀H₉FINO₃

NMR (300 MHz)(DMSO-d₆) δ: 3.53 (m, 1H); 3.67 (m, 1H); 3.82 (dd, 1H); 4.07 (t, 1H); 4.70 (m, 1H); 5.20 (t, 1H); 7.21 (dd, 1H); 7.57 (dd, 1H); 7.81 (t, 1H).

### Intermediate 4: [(5R)-3-(3-Fluoro-4-iodophenyl)-2-oxo-1,3-oxazolidin-5-yl]methyl methanesulfonate

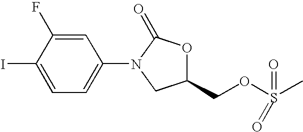

(5R)-3-(3-Fluoro-4-iodophenyl)-5-(hydroxymethyl)-1,3-oxazolidin-2-one (Intermediate 3, 25.0 g, 74.2 mmol) was stirred in dichloromethane (250 ml) at 0° C. Triethylamine (10.5 g, 104 mmol) was added followed by methanesulfonyl chloride (11.2 g, 89.0 mmol) and the reaction was stirred overnight, slowly warming to room temperature. The yellow solution was diluted with sodium bicarbonate and the compound was extracted using dichloromethane (3×250 ml). The organic layer was dried (magnesium sulfate), filtered and concentrated to give the desired product as a light yellow solid (30.3 g).

MS (ESP): 416 (MH⁺) for C₁₁H₁₁FINO₅S

¹H-NMR (300 MHz) (DMSO-d₆): 3.24 (s, 3H); 3.82 (dd, 1H); 4.17 (t, 1H); 4.43-4.52 (m, 2H); 4.99-5.03 (m, 1H); 7.21 (dd, 1H); 7.55 (dd, 1H); 7.83 (t, 1H).

### Intermediate 5: (5R)-5-(Azidomethyl)-3-(3-fluoro-4-iodophenyl-1,3-oxazolidin-2-one

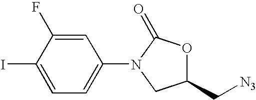

[(5R)-3-(3-Fluoro-4-iodophenyl)-2-oxo-1,3-oxazolidin-5-yl]methyl methanesulfonate (Intermediate 4, 6.14 g, 14.7 mmol) was dissolved in NM-dimethylformamide (50 ml). Sodium azide (1.92 g, 29.6 mmol) was added and the reaction was stirred at 75° C. overnight. The yellow mixture was poured into half-saturated sodium bicarbonate and extracted using ethyl acetate. The organic layer was washed three times with water, dried (magnesium sulfate), filtered, and concentrated to give the title compound as a yellow solid (4.72 g).

MS (ESP): 363 (MH⁺) for C₁₀H₈FIN₄O₂

¹H-NMR (300 MHz) (DMSO-d₆): 3.72-3.82 (m, 3H); 4.14 (t, 1H); 4.89-4.94 (m, 1H); 7.22 (dd, 1H); 7.57 (dd, 1H); 7.83 (t, 1H).

### Intermediate 6: (5R)-3-(3-Fluoro-4-iodophenyl-5-(1H-1,2,3-triazol-1-ylmethyl)-1,3-oxazolidin-2-one

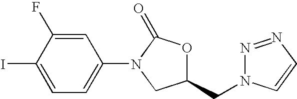

(5R)-5-(Azidomethyl)-3-(3-fluoro-4-iodophenyl)-1,3-oxazolidin-2-one (Intermediate 5, 30.3 g, 72.9 mmol) was stirred in 1,4-dioxane. Bicyclo[2.2.1]hepta-2,5-diene (40.3 g, 437 mmol) was added and the reaction was heated at 100° C. overnight. The resulting brown mixture was filtered and the desired product was obtained as a light brown solid (14.8 g).

MS (ESP): 389 (MH⁺) for C₁₂H₁₀FIN₄O₂

¹H-NMR (300 Mz) (DMSO-d₆): 3.90 (dd, 1H); 4.23 (t, 1H); 4.84 (d, 2H); 5.11-5.18 (m, 1H), 7.14 (dd, 1H); 7.49 (dd, 1H); 7.76 (s, 1H); 7.82 (t, 1H); 8.17 (s, 1H).

### Intermediate 7: (5R)-3-[3-Fluoro-4-(4,4,5,5-tetramethyl-1,3,2-dioxaborolan-2-yl)phenyl-5-(1H-1,2,3-triazol-1-ylmethyl)-1,3-oxazolidin-2-one

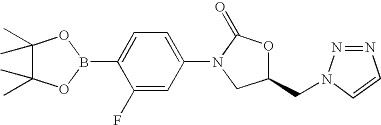

(5R)-3-(3-Fluoro-4-iodophenyl)-5-(1H-1,2,3-triazol-1-ylmethyl)-1,3-oxazolidin-2-one (Intermediate 6, 2 g, 5.15 mmol), bis(pinacolato)diboron, 2.62 g (10.3 mmol), potassium acetate, 2.5 g (25.5 mmol), and 1,1′-[bis(diphenylphosphino)ferrocene]dichloropalladium(II) dichoromethane complex, 0.38 g (0.52 mmol) were suspended in DMSO, 15 ml The mixture was heated at 80° C. for 40 minutes to give a clear black solution. Ethyl acetate (150 ml) was then added and the mixture was filtered through celite, washed with saturated NaCl (2×100 ml), dried over sodium sulfate and evaporated. The dark residue was purified by chromatography (silica gel, 40 to 100% ethyl acetate in hexane, followed by 1-5% acetonitrile in ethyl acetate) to give the product as a crystalline tan solid, 1.97 g (98%). (note—highly colored impurities elute ahead of product band, extended elution required to obtain product).

NMR (300 Mz) (DMSO-d₆) δ: 1.28 (s, 12H), 3.91 (dd, 1H); 4.23 (t, 1H); 4.83 (d, 2H); 5.14 (m, 1H); 7.27 (dd, 1H); 7.37 (dd, 1H); 7.62 (t, 1H); 7.75 (s, 1H); 8.16 (s, 1H).

Alternatively:

(5R)-3-(3-Fluoro-4-iodophenyl)-5-(1H-1,2,3-triazol-1-ylmethyl)-1,3-oxazolidin-2-one (Intermediate 6, 5 g, 12.9 mmol), pinacolborane, 2.9 ml (20 mmol), triethylamine, 5.4 ml (39 mmol), and trans-dichlorobis(triphenylphosphine)palladium (II), 0.92 g (1.3 mmol) were dissolved in dioxane, 70 ml. The mixture was heated at 100° C. for 90 minutes to give a black solution, which was concentrated, dissolved in ethyl acetate, washed with brine, dried over sodium sulfate and evaporated. The residue was purified by chromatography (silica gel, 0 to 5% methanol in dichloromethane with 1% triethylamine) to give the product as a light brown solid, 3.1 g.

### Intermediate 8: 5-Bromo-N-hydroxypyridine-2-carboximidoyl chloride

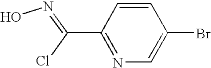

5-Bromopyridine-2-carbaldehyde oxime (49.5 g, 246.3 mmol) was dissolved in DMF (150 ml) followed by addition of N-chlorosuccinimide (39.5 g, 295.5 mmol). HCl gas was then bubbled in the solution for 20 seconds to initiate the reaction, which was then allowed to stir for 1 hr. The reaction was poured into distilled water (1 L) and the precipitate was collected by vacuum filtration. The filter cake was washed with distilled water (2×500 ml) and then dried overnight in a vacuum oven at 60° C. (−30 inches Hg) to yield the product as a white powder (55 g).

¹H-NMR (300 Mz)(CDCl₃) δ: 7.73 (d, 1H); 8.09 (d, 1H); 8.73 (s, 1H); 12.74 (s, 1H).

NOTE: Lachrymator.

### Intermediate 9: [3-(5-Bromopyridin-2-yl)-4,5-dihydroisoxazol-5-yl]methyl butyrate

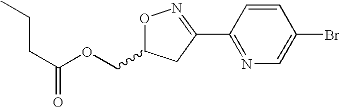

5-Bromo-N-hydroxypyridine-2-carboximidoyl chloride (Intermediate 8, 46 g, 195.7 mmol) was added to EtOAc (200 ml)) followed by addition of allyl butyrate (145 ml, 1020.4 mmol) and the solution was cooled to 0° C. Triethylamine (30 ml, 215.8 mmol) in EtOAc (100 ml) was then added dropwise over 1 hour. The reaction was then allowed to stir for 1 hour at 0° C. and then EtOAc (1 L) was added. The precipitate was removed by vacuum filtration and the filtrate was concentrated in vacuo to yield the product (65 g).

¹H-NMR (DMSO-d₆) δ: 0.81 (t, 3H); 1.43 (m, 2H); 2.24 (t, 2H); 3.21 (dd, 1H); 3.54 (dd, 1H); 4.13 (dd, 1H); 4.23 (dd, 1H); 5.01 (m, 1H); 7.85 (dd, 1H); 8.12 (dd, 1H); 8.81 (d, 1H).

### Intermediate 10: (5S)-3-(5-Bromopyridin-2-yl)-4,5-dihydroisoxazol-5-yl]methylbutyrate

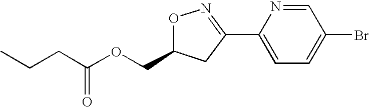 (+) Isomer assigned as (5S) based on comparison with Chem. Lett. 1993 p. 1847.

Racemic [3-(5-bromopyridin-2-yl)-4,5-dihydroisoxazol-5-yl]methyl butyrate (Intermediate 9, 80 g, 0.244 mol) was dissolved in acetone (4 L), and 0.1 M potassium phosphate buffer (pH˜7) (4 L) was added with vigorous stirring to give a clear yellow solution. PS-lipase (1.45 g, Sigma cat no L-9156) was added and the mixture was gently stirred at ambient temp. for 42 hrs. The solution was divided into 3 equal volumes of 2.6 L and each was extracted with dichloromethane (2×1 L), the pooled organic phases were dried over sodium sulfate and evaporated. The unreacted [(5S)-3-(5-bromopyridin-2-yl)-4,5-dihydroisoxazol-5-yl]methyl butyrate was isolated via flash column chromatography (9:1 hexane:ethyl acetate) as a clear yellow oil, 36.4 g (45.5%).

### Intermediate 11: [(5S)-3-(5-Bromopyridin-2-yl)-4,5-dihydroisoxazol-5-yl]methanol

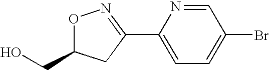

[(5S)-3-(5-Bromopyridin-2-yl)-4,5-dihydroisoxazol-5-yl]methyl butyrate (Intermediate 10, 16.88 g, 0.051 mol) was dissolved in methanol (110 ml). 50% Aqueous sodium hydroxide (3.6 ml, 0.068 mol) was added. The solution was stirred at RT for 15 minutes, 1M HCl (75 ml) was added, followed by concentration in vacuo to ˜100 ml total volume. Water (−50 ml) was added, and the white precipitate was collected and rinsed with water. The filtrate was extracted twice with ethyl acetate, the organic layers were pooled, dried over sodium sulfate and evaporated. The solid residue was collected and rinsed with 10:1 hexane:ethyl acetate, then combined with the initial precipitate before drying in vacuo to give the title compound as a white crystalline solid, 12.3 g (93%). Chiral HPLC analysis indicated <0.5% of the (−) isomer was present. [α]_{D}=+139 (c=0.01 g/ml in methanol).

### EXAMPLE 64

### (5R)-3-(3-Fluoro-4-{6-[(5S)-5-(methoxymethyl)-4,5-dihydroisoxazol-3-yl]pyridin-3-yl}phenyl)-5-(1H-1,2,3-triazol-1-ylmethyl)-1,3-oxazolidin-2-one

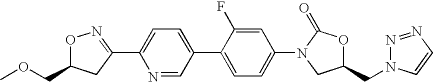

(5R)-3-(3-Fluoro-4-{6-[(5S)-5-(hydroxymethyl)-4,5-dihydroisoxazol-3-yl]pyridin-3-yl}phenyl)-5-(1H-1,2,3-triazol-1-ylmethyl)-1,3-oxazolidin-2-one (see Example 63, 0.20 g, 0.46 mmol), was dissolved in DMF (3 ml) with warming, then cooled to 0° C. Methyl iodide (0.3 ml, 4.8 mmol), then sodium hydride (60% dispersion in mineral oil, 40 mg, 1.0 mmol) were added and the suspension was stirred and allowed to slowly warm to room temperature over 2 hours, then stirred an additional 5 hours. The mixture was carefully diluted with water and extracted with ethyl acetate. The organic layer was washed with saturated sodium chloride, dried over sodium sulfate, evaporated and purified via chromatography (silica gel, 0.5 to 5% methanol in dichloromethane). Evaporation of the product containing fractions and trituration of the resulting solid with diethyl ether:dichloromethane:methanol (−5:5:1) yielded the title compound as a beige solid (100 mg, 48% yield), melting point: 161° C.

MS (electrospray): 453 (MH⁺) for C₂₂H₂₁FN₆O₄

¹H-NMR (400 MHz, DMSO-d₆) δ: 3.25 (dd, 1H); 3.30 (s, 3H); 3.50 (m, 3H); 3.96 (dd, 1H); 4.29 (t, 1H); 4.86 (d, 2H); 4.91 (m, 1H); 5.18 (m, 1H); 7.42 (dd, 1H); 7.59 (dd, 1H); 7.69 (t, 1H); 7.76 (s, 1H); 7.99 (d, 1H); 8.05 (d, 1H); 8.18 (s, 1H); 8.81 (s, 1H).

Alternative Preparation for Example 64:

[(5S)-3-(5-Bromopyridin-2-yl)-4,5-dihydroisoxazol-5-yl]methanol (Intermediate 11 for Example 63, 2.1 g, 8.17 mmol), was dissolved in THF (20 ml), then cooled to 0° C. Methyl iodide (1.5 ml, 24 mmol), then sodium hydride (60% dispersion in mineral oil, 0.56 g, 14 mmol) were added and the suspension was stirred and allowed to slowly warm to room temperature over 18 hours. The mixture was carefully diluted with water and 1M HCl and extracted with dichloromethane. The organic layer was washed with saturated sodium chloride, dried over sodium sulfate, evaporated and purified via chromatography (silica gel, 1 to 20% ethyl acetate in hexanes). Evaporation of the product containing fractions and drying in vacuo yielded [(5S)-3-(5-bromopyridin-2-yl)-4,5-dihydroisoxazol-5-yl]methanol as a waxy white solid (1.83 g).

¹H-NMR (400 MHz, DMSO-d₆) δ: 3.18 (dd, 1H); 3.28 (s, 3H); 3.42-3.52 (m, 3H); 4.89 (m, 1H); 7.84 (d, 1H); 8.11 (dd, 1H); 8.77 (d, 1H).

[(5S)-3-(5-Bromopyridin-2-yl)-4,5-dihydroisoxazol-5-yl]methanol (1.8 g, 6.64 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7 for Example 63, 2.71 g, 6.97 mmol), potassium carbonate (2.9 g, 21 mmol), and tetrakis(triphenylphosphine)palladium(0) (0.5 g, 0.43 mmol) were combined in DMF (25 ml) and distilled water (4 ml) then heated to 80° C. for 1 hour. The reaction was poured into water and the precipitated material was collected and rinsed with water, ether, and ether:methanol (1:1). The resulting paste was dissolved in a minimum amount of 2,2,2-trifluoroethanol and filtered through a silica gel pad (50 g), rinsing with 5% methanol in dichloromethane. The solution was evaporated and further purified by column chromatography 0.5-5% methanol/dichloromethane to yield a crude residue, which was dissolved in dichloromethane (15 ml), then precipitated with ethyl acetate (100 ml). The suspension was warmed and sonnicated, then the resulting solid was collected and rinsed with ethyl acetate and diethyl ether, and dried in vacuo at 70° C. to yield (5R)-3-(3-fluoro-4-{6-[(5S)-5-(methoxymethyl)-4,5-dihydroisoxazol-3-yl]pyridin-3-yl}phenyl)-5-(1H-1,2,3-triazol-1-ylmethyl)-1,3-oxazolidin-2-one as an off-white solid (2.3 g), melting point: 172° C.

MS (electrospray): 453 (MH⁺) for C₂₂H₂₁FN₆O₄

¹H-NMR (400 MHz DMSO-d₆) δ: 3.25 (dd, 1H); 3.30 (s, 3H); 3.50 (m, 3H); 3.96 (dd, 1H); 4.29 (t, 1H); 4.86 (d, 2H); 4.91 (m, 1H); 5.18 (m, 1H); 7.42 (dd, 1H); 7.59 (dd, 1H); 7.69 (t, 1H); 7.76 (s, 1H); 7.99 (d, 1H); 8.05 (d, 1H); 8.18 (s, 1H); 8.81 (s, 1H).

1. A compound of the formula (I), or a pharmaceutically-acceptable salt, or an in-vivo-hydrolysable ester thereof, 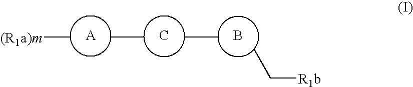
wherein in (I) C is a biaryl group C′-C″ 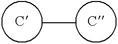
where C′ and C″ are independently aryl or heteroaryl rings such that the group C is represented by any one of the groups D to O below: 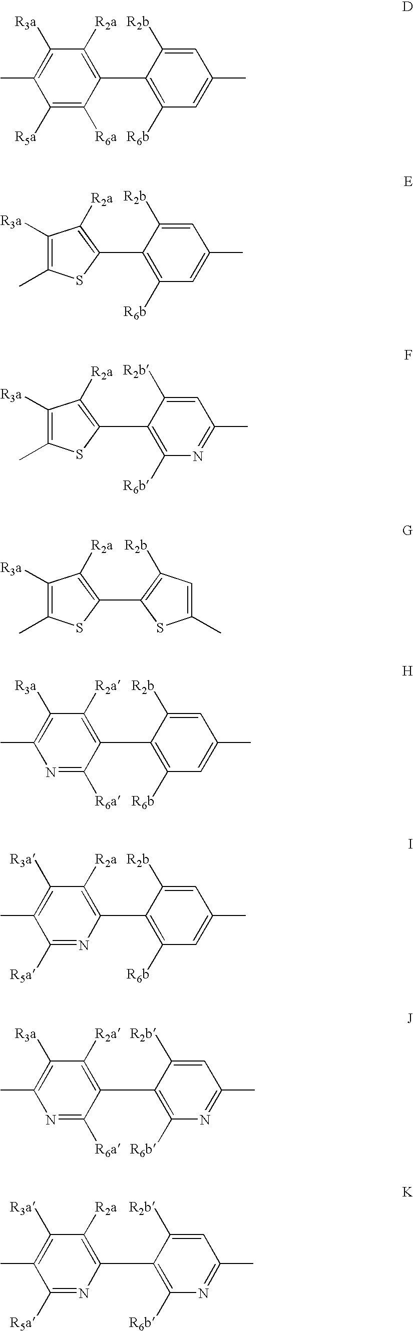 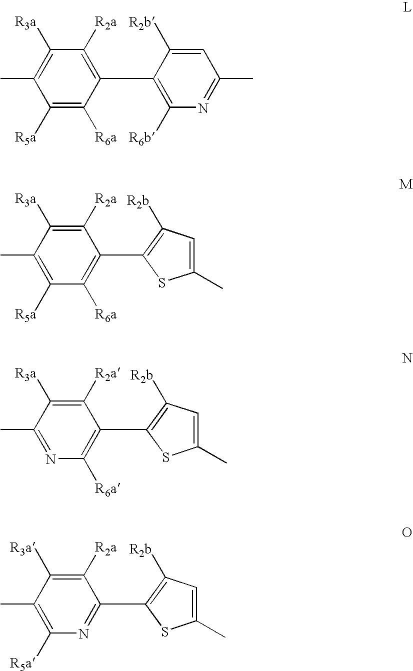
wherein the groups D to O are attached to rings A and B in the orientation [(A-C′) and (C″-B)] shown;
wherein A and B are independently selected from 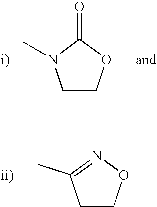
wherein A is linked as shown in (I) via the 3-position to ring C′ of group C and independently substituted in the 4 and 5 positions as shown in (I) by one or more substituents —R₁a)ₘ;
and wherein B is linked as shown in (I) via the 3-position to ring C″ of group C and independently substituted in the 5 position as shown in (i) by substituent —CH₂—R₁b;
R₂b and R₆b are independently selected from H, F, Cl, OMe, SMe, Me, Et and CF₃;
R₂b′ and R₆b′ are independently selected from H, OMe, Me, Et and CF₃;
R₂a and R₆a are independently selected from H, Br; F, Cl, OMe, SMe; Me, Et and CF₃;
R₂a′ and R₆a′ are independently selected from H, OMe, SMe; Me, Et and CF₃;
R₃a and R₅a are independently selected from H, (1-4C)alkyl, Br, F, Cl, OH, (1-4C)alkoxy, —S(O)ₙ(1-4C)alkyl (wherein n=0, 1, or 2), amino, (1-4C)alkylcarbonylamino-, nitro, cyano, —CHO, —CO(1-4C) alkyl, —CONH₂ and —CONH(1-4C)alkyl;
R₃a′, R₅a′ are independently selected from H, (1-4C)alkyl, OH, (1-4C)alkoxy, (1-4C)alkylthio, amino, (1-4C)alkylcarbonylamino-, nitro, cyano, —CHO, —CO(1-4C)alkyl, —CONH₂ and —CONH(1-4C)alkyl;
wherein one of R₃a, R₅a, R₃a′, R₅a′ taken together with a substituent R₁a at position 4 of ring A and rings A and C′ may form a 5-7 membered ring;
wherein any (1-4C)alkyl group may be optionally substituted with F, OH, (1-4C)alkoxy, —S(O)ₙ(1-4C)alkyl (wherein n=0, 1, or 2) or cyano;
wherein when ring C′ is a pyridine ring (ie when group C is group H, I, J, K, N or O) the ring nitrogen may optionally be oxidised to an N-oxide;
R₁a is independently selected from R₁a1 to R₁a5 below:
R₁a1: AR1, AR2, AR2a, AR2b, AR3, AR3a, AR3b, AR4, AR4a, CY1, CY2;
R₁a2: cyano, carboxy, (1-4C)alkoxycarbonyl, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl) and —C(═S)O(1-4C)alkyl; wherein any (1-4C)alkyl, (1-4C)alkanoyl and (3-6C)cycloalkyl substituent may itself be substituted by cyano, hydroxy or halo, provided that, such a substituent is not on a carbon adjacent to a nitrogen atom of the piperazine ring], ethenyl, 2-(1-4C)alkylethenyl, 2-cyanoethenyl, 2-cyano-2-((1-4C)alkyl)ethenyl, 2-nitroethenyl, 2-nitro-2-((1-4C)alkyl)ethenyl, 2-((1-4C)alkylaminocarbonyl)ethenyl, 2-((1-4C)alkoxycarbonyl)ethenyl, 2-(AR1)ethenyl, 2-(AR2)ethenyl, 2-(AR2a)ethenyl;
R₁a3: (1-10C)alkyl
{optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, phosphonate [phosphono, —P(O)(OH)₂, and mono- and di-(1-4C)alkoxy derivatives thereof], phosphinate [—P(OH)₂ and mono- and di-(1-4C)alkoxy derivatives thereof], cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-6C)alkanoyloxy(1-4C)alkoxy, carboxy(1-4C)alkoxy, halo(1-4C)alkoxy, dihalo(1-4C)alkoxy, trihalo(1-4C)alkoxy, morpholino-ethoxy, (N′-methyl)piperazino-ethoxy, 2-, 3-, or 4-pyridyl(1-6C)alkoxy, N-methyl(imidazo-2 or 3-yl)(1-4C)alkoxy, imidazo-1-yl(1-6C)alkoxy, (1-4C)alkylamino, di((1-4C)alkyl)amino, (1-6C)alkanoylamino-, (1-4C)alkoxycarbonylamino-, N-(1-4C)alkyl-N-(1-6C)alkanoylamino-, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyll, —COO(1-4C)alkyl, —S(O)n(1-4C)alkyl (wherein n=1 or 2), —COOAR1, —CS(1-4C)alkyl and —C(═S)O(1-4C)alkyl], (═NORv) wherein Rv is as hereinbefore defined, (1-4C)alkylS(O)ₚNH—, (1-4C)alkylS(O)ₚ-((1-4C)alkyl)N—, fluoro(1-4C)alkylS(O)ₚNH—, fluoro(1-4C)alkylS(O)ₚ((1-4C)alkyl)N—, (1-4C)alkylS(O)_{q}—, CY1, CY2, AR1, AR2, AR3, AR1-O—, AR2-O—, AR3-O—, AR1-S(O)_{q}—, AR2-S(O)_{q}—, AR3-S(O)_{q}—, AR1-NH—, AR2-NH—, AR3-NH— (p is 1 or 2 and q is 0, 1 or 2), and also AR2a, AR2b, AR3a and AR3b versions of AR2 and AR3 containing groups}; wherein any (1-4C)alkyl, (1-4C)alkanoyl and (3-6C)cycloalkyl present in any substituent on R₁a3 may itself be substituted by one or two groups selected from cyano, hydroxy, halo, amino, (1-4C)alkylamino and di(1-4C)alkylamino, provided that such a substituent is not on a carbon adjacent to a heteroatom atom if present;
R₁a4: R¹⁴C(O)O(1-6C)alkyl [wherein R¹⁴ is AR1, AR2, AR2a, AR2b, (1-4C)alkylamino, benzyloxy-(1-4C)alkyl, naphthylmethyl, (1-4C)alkoxy-(1-4C)alkoxy-(1-4C)alkoxy-(1-4C)alkoxy-(1-4C)alkoxy-(1-4C)alkoxy, (1-4C)alkoxy-(1-4C)alkoxy-(1-4C)alkoxy-(1-4C)alkoxy-(1-4C)alkoxy, (1-4C)alkoxy-(1-4C)alkoxy-(1-4C)alkoxy-(1-4C)alkoxy, (1-4C)alkoxy-(1-4C)alkoxy-(1-4C)alkoxy, (1-4C)alkoxy-(1-4C)alkoxy or (1-10C)alkyl {optionally substituted as defined for (R₁a3)], imidazo-1-yl(1-6C)alkyoxy(1-4C)alkyl, morpholino-ethoxy(1-4C)alkyl, (N′-methyl)piperazino-ethoxy(1-4C)alkyl, 2-, 3-, or 4-pyridyl(1-6C)alkyloxy(1-4C)alkyl, 2-, 3-, or 4-pyridyl(1-6C)alkylamino(1-4C)alkyl, 2-, 3-, or 4-pyridyl(1-6C)alkylsulfonyl(1-4C)alkyl, N-methyl(imidazo-2 or 3-yl)(1-4C)alkyloxy(1-4C)alkyl;
R₁a5: F, Cl, hydroxy, mercapto, (1-4C)alkylS(O)p- (p=0, 1 or 2), —NR₁₂R₁₃, —OSO₂(1-4C)alkyl, —O(1-4C)alkanoyl, or —OR₁a3;
m is 0, 1 or 2;
wherein two substituents R₁a both at the 4 or 5 position of ring A taken together may form a 5 to 7 membered spiro ring;
wherein two substituents R₁a at the 4 and 5 positions of ring A taken together may form a 5 to 7 membered fused ring;
provided that if (R₁a)ₘ is a single substituent R₁a at the 5 position of ring A then R₁a is not —CH₂X wherein X is selected from R1 b;
R₁b is independently selected from hydroxy, —OSi(tri-(1-6C)alkyl) (wherein the 3 (1-6C)alkyl groups are independently selected from all possible (1-6C)alkyl groups), —NR₅C(═W)R₄, —OC(═O)R₄, 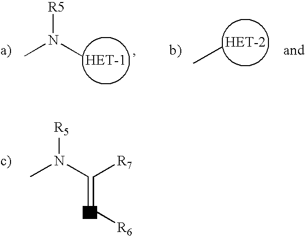
wherein W is O or S;
provided that if group C is group H or group 1, and if one of substituents R₂b and R₆b is H and the other is F, and if all of substituents R₂a, R₆a, R₂a′, R₆a′, R₃a, R₅a, R₃a′, R₅a′ are H at each occurrence, then R₁b is not —NHC(═O)Me;
R₄ is selected from hydrogen, amino, (1-8C)alkyl, (2-6C)alkyl (substituted by 1, 2 or 3 substituents independently selected from methyl, chloro, bromo, fluoro, methoxy, methylthio, azido and cyano), methyl (substituted by 1, 2 or 3 substituents independently selected from methyl, chloro, bromo, fluoro, methoxy, methylthio, hydroxy, benzyloxy, ethynyl, (1-4C)alkoxycarbonyl, azido and cyano), —NHR₁₂, —N(R₁₂)(R₁₃), —OR₁₂ or —SR₁₂, (2-4C)alkenyl, -(1-8C)alkylaryl, mono-, di-, tri- and per-halo(1-8C)alkyl, —(CH₂)ₚ(3-6C)cycloalkyl and —(CH₂)p(3-6C)cycloalkenyl wherein p is 0, 1 or 2;
R₅ is selected from hydrogen, (3-6C)cycloalkyl, phenyloxycarbonyl, tert-butoxycarbonyl, fluorenyloxycarbonyl, benzyloxycarbonyl, (1-6C)alkyl (optionally substituted by cyano or (1-4C)alkoxycarbonyl), —CO₂R₈, —C(═O)R₈, —C(═O)SR₈, —C(═S)R₈, P(O)(OR₉)(OR₁₀) and —SO₂R₁₁, wherein R₈, R₉, R₁₀ and R₁₁ are as defined hereinbelow;
HET-1 is selected from HET-1A and HET-1B wherein:
HET-1A is a C-linked 5-membered heteroaryl ring containing 2 to 4 heteroatoms independently selected from N, O and S; which ring is optionally substituted on a C atom by an oxo or thioxo group; and/or which ring is optionally substituted on any available C atom by one or two substituents selected from RT as hereinafter defined and/or on an available nitrogen atom, (provided that the ring is not thereby quaternised) by (1-4C)alkyl;
HET-1B is a C-linked 6-membered heteroaryl ring containing 2 or 3 nitrogen heteroatoms, which ring is optionally substituted on a C atom by an oxo or thioxo group; and/or which ring is optionally substituted on any available C atom by one, two or three substituents selected from RT as hereinafter defined and/or on an available nitrogen atom, (provided that the ring is not thereby quaternised) by (1-4C)alkyl;
HET-2 is selected from HET-2A and HET-2B wherein
HET-2A is an N-linked 5-membered, fully or partially unsaturated heterocyclic ring, containing either (i) 1 to 3 further nitrogen heteroatoms or (ii) a further heteroatom selected from O and S together with an optional further nitrogen heteroatom; which ring is optionally substituted on a C atom, other than a C atom adjacent to the linking N atom, by an oxo or thioxo group; and/or which ring is optionally substituted on any available C atom, other than a C atom adjacent to the linking N atom, by a substituent selected from RT as hereinafter defined and/or on an available nitrogen atom, other than a N atom adjacent to the linking N atom, (provided that the ring is not thereby quaternised) by (1-4C)alkyl;
HET-2B is an N-linked 6-membered di-hydro-heteroaryl ring containing up to three nitrogen heteroatoms in total (including the linking heteroatom), which ring is substituted on a suitable C atom, other than a C atom adjacent to the linking N atom, by oxo or thioxo and/or which ring is optionally substituted on any available C atom, other than a C atom adjacent to the linking N atom, by one or two substituents independently selected from RT as hereinafter defined and/or on an available nitrogen atom, other than a N atom adjacent to the linking N atom, (provided that the ring is not thereby quatemised) by (1-4C)alkyl;
RT is selected from a substituent from the group:
(RTa1) hydrogen, halogen, (1-4C)alkoxy, (2-4C)alkenyloxy, (2-4C)alkenyl, (2-4C)alkynyl, (1-4C)alkoxycarbonyl, (3-6C)cycloalkyl, (3-6C)cycloalkenyl, (1-4C)alkylthio, amino, azido, cyano and nitro; or
(RTa2) (1-4C)alkylamino, di-(1-4C)alkylamino, and (2-4C)alkenylamino;
or RT is selected from the group
(RTb1) (1-4C)alkyl group which is optionally substituted by one substituent selected from hydroxy, (1-4C)alkoxy, (1-4C)alkylthio, cyano and azido; or
(RTb2) (1-4C)alkyl group which is optionally substituted by one substituent selected from (2-4C)alkenyloxy, (3-6C)cycloalkyl, and (3-6C)cycloalkenyl;
or RT is selected from the group
(RTc) a fully saturated 4-membered monocyclic ring containing 1 or 2 heteroatoms independently selected from O, N and S (optionally oxidised), and linked via a ring nitrogen or carbon atom;
and wherein at each occurrence of an RT substituent containing an alkyl, alkenyl, alkynyl, cycloalkyl or cycloalkenyl moiety in (RTa1) or (RTa2), (RTb1) or (RTb2), or (RTc) each such moiety is optionally substituted on an available carbon atom with one, two, three or more substituents independently selected from F, Cl, Br, OH and CN;
R₆ is cyano, —COR₁₂, —COOR₁₂, —CONHR₁₂, —CON(R₁₂)(R₁₃), —SO₂R₁₂, —SO₂NHR₁₂, —SO₂N(R₁₂)(R₁₃) or NO₂, wherein R₁₂ and R₁₃ are as defined hereinbelow;
R₇ is hydrogen, amino, (1-8C)alkyl, —NHR₁₂, —N(R₁₂)(R₁₃), —OR₁₂ or —SR₁₂, (2-4C)alkenyl, -(1-8C)alkylaryl, mono-, di-, tri- and per-halo(1-8C)alkyl, —(CH₂)p(3-6C)cycloalkyl or —(CH₂)p(3-6C)cycloalkenyl wherein p is 0, 1 or 2;
R₈ is hydrogen, (3-6C)cycloalkyl, phenyl, benzyl, (1-5C)alkanoyl, (1-6C)alkyl (optionally substituted by substituents independently selected from (1-5C)alkoxycarbonyl, hydroxy, cyano, up to 3 halogen atoms and —NR₁₅R₁₆ (wherein R₁₅ and R₁₆ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₅)(R₁₆) group, R₁₅ and R₁₆ may additionally be taken together with the nitrogen atom to which they are attached to form a pyrrolidinyl, piperidinyl or morpholinyl ring);
R₉ and R₁₀ are independently selected from hydrogen and (1-4C)alkyl;
R₁₁ is (1-4C)alkyl or phenyl;
R₁₂ and R₁₃ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₂)(R₁₃) group, R₁₂ and R₁₃ may additionally be taken together with the nitrogen atom to which they are attached to form a pyrrolidinyl, piperidinyl or morpholinyl ring, which ring may be optionally substituted by a group selected from (1-4C)alkyl, (3-6C)cycloalkyl, (1-4C)alkanoyll, —COO(1-4C)alkyl, S(O)n(1-4C)alkyl (wherein n=1 or 2), —COOAR1, —CS(1-4C)alkyl and —C(═S)O(1-4C)alkyl;
AR1 is an optionally substituted phenyl or optionally substituted naphthyl;
AR2 is an optionally substituted 5- or 6-membered, fully unsaturated (i.e with the maximum degree of unsaturation) monocyclic heteroaryl ring containing up to four heteroatoms independently selected from O, N and S (but not containing any O—O, O—S or S—S bonds), and linked via a ring carbon atom, or a ring nitrogen atom if the ring is not thereby quaternised;
AR2a is a partially hydrogenated version of AR2 (i.e. AR2 systems retaining some, but not the full, degree of unsaturation), linked via a ring carbon atom or linked via a ring nitrogen atom if the ring is not thereby quaternised;
AR2b is a fully hydrogenated version of AR2 (i.e. AR2 systems having no unsaturation), linked via a ring carbon atom or linked via a ring nitrogen atom;
AR3 is an optionally substituted 8-, 9- or 10-membered, fully unsaturated (i.e with the maximum degree of unsaturation) bicyclic heteroaryl ring containing up to four heteroatoms independently selected from O, N and S (but not containing any O—O, O—S or S—S bonds), and linked via a ring carbon atom in either of the rings comprising the bicyclic system;
AR3a is a partially hydrogenated version of AR3 (i.e. AR3 systems retaining some, but not the full, degree of unsaturation), linked via a ring carbon atom, or linked via a ring nitrogen atom if the ring is not thereby quaternised, in either of the rings comprising the bicyclic system;
AR3b is a fully hydrogenated version of AR3 (i.e. AR3 systems having no unsaturation), linked via a ring carbon atom, or linked via a ring nitrogen atom, in either of the rings comprising the bicyclic system;
AR4 is an optionally substituted 13- or 14-membered, fully unsaturated (i.e with the maximum degree of unsaturation) tricyclic heteroaryl ring containing up to four heteroatoms independently selected from O, N and S (but not containing any O—O, O—S or S—S bonds), and linked via a ring carbon atom in any of the rings comprising the tricyclic system;
AR4a is a partially hydrogenated version of AR4 (i.e. AR4 systems retaining some, but not the full, degree of unsaturation), linked via a ring carbon atom, or linked via a ring nitrogen atom if the ring is not thereby quaternised, in any of the rings comprising the tricyclic system;
CY1 is an optionally substituted cyclobutyl, cyclopentyl or cyclohexyl ring;
CY2 is an optionally substituted cyclopentenyl or cyclohexenyl ring;
wherein; optional substituents on AR1, AR2, AR2a, AR2b, AR3, AR3a, AR3b, AR4, AR4a, CY1 and CY2 are (on an available carbon atom) up to three substituents independently selected from (1-4C)alkyl {optionally substituted by substituents selected independently from hydroxy, trifluoromethyl, (1-4C)alkyl S(O)_{q}— (q is 0, 1 or 2), (1-4C)alkoxy, (1-4C)alkoxycarbonyl, cyano, nitro, (1-4C)alkanoylamino, —CONRvRw or —NRvRw}, trifluoromethyl, hydroxy, halo, nitro, cyano, thiol, (1-4C)alkoxy, (1-4C)alkanoyloxy, dimethylaminomethyleneaminocarbonyl, di(N-(1-4C)alkyl)aminomethylimino, carboxy, (1-4C)alkoxycarbonyl, (1-4C)alkanoyl, (1-4C)alkylSO₂amino, (2-4C)alkenyl {optionally substituted by carboxy or (1-4C)alkoxycarbonyl}, (2-4C)alkynyl, (1-4C)alkanoylamino, oxo (═O), thioxo (═S), (1-4C)alkanoylamino {the (1-4C)alkanoyl group being optionally substituted by hydroxy}, (1-4C)alkyl S(O)_{q}— (q is 0, 1 or 2) {the (1-4C)alkyl group being optionally substituted by one or more groups independently selected from cyano, hydroxy and (1-4C)alkoxy}, —CONRvRw or —NRvRw [wherein Rv is hydrogen or (1-4C)alkyl; Rw is hydrogen or (1-4C)alkyl];
and further optional substituents on AR1, AR2, AR2a, AR2b, AR3, AR3a, AR3b, AR4, AR4a, CY1 and CY2 (on an available carbon atom), and also on alkyl groups (unless indicated otherwise) are up to three substituents independently selected from trifluoromethoxy, benzoylamino, benzoyl, phenyl {optionally substituted by up to three substituents independently selected from halo, (1-4C)alkoxy or cyano}, furan, pyrrole, pyrazole, imidazole, triazole, pyrimidine, pyridazine, pyridine, isoxazole, oxazole, isothiazole, thiazole, thiophene, hydroxyimino(1-4C)alkyl, (1-4C)alkoxyimino(1-4C)alkyl, halo-(1-4C)alkyl, (1-4C)alkanesulfonamido, —SO₂NRvRw [wherein Rv is hydrogen or (1-4C)alkyl;
Rw is hydrogen or (1-4C)alkyl]; and
optional substituents on AR2, AR2a, AR2b, AR3, AR3a, AR3b, AR4 and AR4a are (on an available nitrogen atom, where such substitution does not result in quaternization) (1-4C)alkyl, (1-4C)alkanoyl {wherein the (1-4C)alkyl and (1-4C)alkanoyl groups are optionally substituted by (preferably one) substituents independently selected from cyano, hydroxy, nitro, trifluoromethyl, (1-4C)alkyl S(O)_{q}— (q is 0, 1 or 2), (1-4C)alkoxy, (1-4C)alkoxycarbonyl, (1-4C)alkanoylamino, —CONRvRw or —NRvRw [wherein Rv is hydrogen or (1-4C)alkyl; Rw is hydrogen or (1-4C)alkyl]}, (2-4C)alkenyl, (2-4C)alkynyl, (1-4C)alkoxycarbonyl or oxo (to form an N-oxide).

2. A compound of claim 1, wherein group C is represented by any one of groups D, E, H and I.

3. A compound of claim 2, wherein R₁a and R₁b are independently selected from —NHCO(1-4C)alkyl, —NHCO(1-4C)cycloalkyl, —NHCS(1-4C)alkyl, —N(R₅)-HET-1 and HET-2.

4. A compound of claim 3, wherein HET-2A is selected from the structures (Za) to (Zf) below: 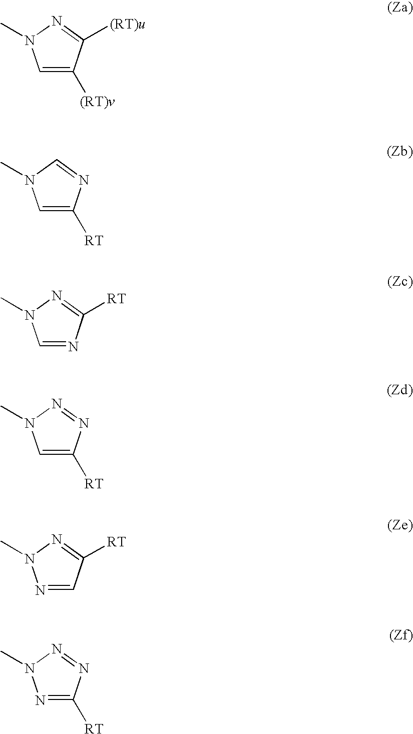
wherein u and v are independently 0 or 1.

5. A compound of claim 4 wherein RT is selected from
(a) hydrogen;
(b) halogen;
(c) cyano;
(d) (1-4C)alkyl;
(e) monosubstituted (1-4C)alkyl;
(f) disubstituted (1-4C)alkyl, and
trisubstituted (1-4C)alkyl.

6. A compound of claim 1 wherein at least one of A and B is an oxazolidinone.

7. A compound of claim 1 wherein A is an isoxazoline and B is an oxazolidinone.

8. A compound of claim 1 wherein group C is represented by Group H.

9. A compound of the formula (Ia) which is a compound of claim 1

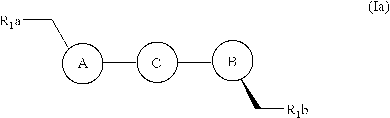

10. A pro-drug of a compound as claimed in any one of the previous claims.

11. A method for producing an antibacterial effect in a warm blooded animal which comprises administering to said animal an effective amount of a compound of claim 1.

12. (canceled)

13. (canceled)

14. A pharmaceutical composition which comprises a compound of claim 1 and a pharmaceutically-acceptable diluent or carrier.

15. A pharmaceutical composition as claimed in claim 14, wherein said composition includes a vitamin.

16. A pharmaceutical composition as claimed in claim 15 wherein said vitamin is Vitamin B.

17. A pharmaceutical composition as claimed in claim 14, wherein said composition comprises a combination of a compound of the formula (I) and an antibacterial agent active against gram-positive bacteria.

18. A pharmaceutical composition as claimed in claim 14, wherein said composition comprises a combination of a compound of the formula (I) and an antibacterial agent active against gram-negative bacteria.

19. A process for the preparation of a compound of formula (I) as claimed in claim 1 or pharmaceutically acceptable salts or in-vivo hydrolysable esters thereof, which process comprises one of processes (a) to U): and thereafter if necessary:
i) removing any protecting groups;
ii) forming a pro-drug (for example an in-vivo hydrolysable ester); and/or
iii) forming a pharmaceutically-acceptable salt;
wherein said processes (a) to (j) are:
(a) modifying a substituent in, or introducing a substituent into another compound of the invention by using standard chemistry;
(b) reaction of a molecule of a compound of formula (IIa) with a molecules of a compound of formula (IIb) wherein X and X′ are leaving groups useful in palladium coupling and are chosen such that an aryl-aryl, heteroaryl-aryl, or heteroaryl-heteroaryl bond replaces the aryl-X (or heteroaryl-X) and aryl-X′ (or heteroaryl-X′) bonds; 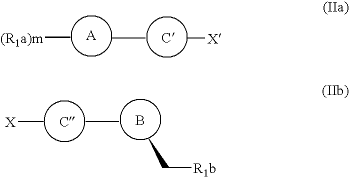
(c) reaction of a compound of formula (IIIa) with a compound of formula (IIIb): 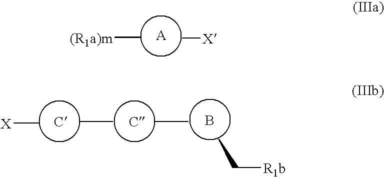
where X and X′ are replaceable substituents and wherein the substituents X and X′ are chosen to be complementary pairs of substituents known in the art to be suitable as complementary substrates for coupling reactions catalysed by transition metals;
(d) reaction of a (hetero)biaryl derivative (IIIa) or (IIIb) carbamate with an appropriately substituted oxirane (wherein 0, 1, or 2 of R₁a′-R₁a″″ are substitutents as defined for R₁a and the remainder are hydrogen) to form an oxazolidinone ring at the undeveloped aryl position; 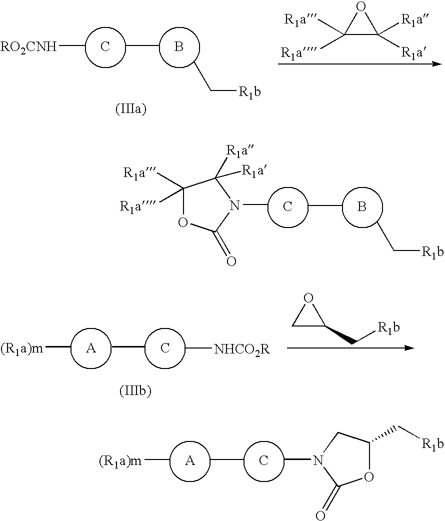
or by variations on this process in which the carbamate is replaced by an isocyanate or by an amine or/and in which the oxirane is replaced by an equivalent reagent X—C(R₁a′)(R₁a″)C(R₁a′″)(O-optionally protected)(R₁a″″) or X—CH₂CH(O-optionally protected)CH₂R₁b where X is a displaceable group;
(e) reaction of a (hetero)biaryl derivative (IVa) or (IVb) to form an isoxazoline ring at the undeveloped aryl position; 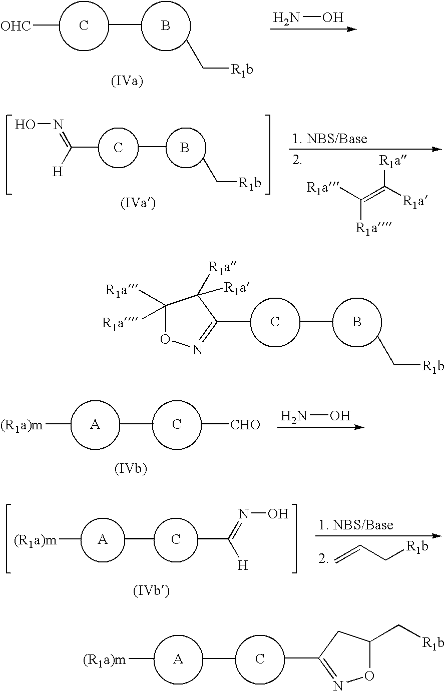
or by variations on this process in which the reactive intermediate (a nitrile oxide IVa″ or IVb″) is obtained other than by oxidation of an oxime (IVa′) or (IVb′); 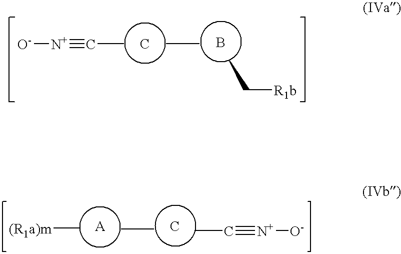
(f) for HET as optionally substituted 1,2,3-triazoles, by cycloaddition via the azide (wherein e.g. Y in (II) is azide) to acetylenes, or to acetylene equivalents or optionally substituted ethylenes bearing eliminatable substituents;
(g) for HET as 4-substituted 1,2,3-triazole compounds of formula (I) by reacting aminomethyloxazolidinones with 1,1-dihaloketone sulfonylhydrazones
(h) for HET as 4-substituted 1,2,3-triazole compounds of formula (I) by reacting azidomethyl oxazolidinones with terminal alkynes using Cu(I) catalysis to give 4-substituted 1,2,3-triazoles
(j) for HET as 4-halogenated 1,2,3-triazole compounds of formula (I) by reacting azidomethyl oxazolidinones with halovinylsulfonyl chlorides at a temperature between 0° C. and 100° C. either neat or in an inert diluent.
